(19) United States
(12) Patent Application Publication
(10) Pub. No.: US 2024/0139166 A1
(43) Pub. Date: May 2, 2024

(54) OPHTHALMIC COMPOSITIONS FOR PRESBYOPIA

(71) Applicant: Sydnexis, Inc., Del Mar, CA (US)

(72) Inventors: Gregory I. OSTROW, San Diego, CA (US); Kenneth J. WIDDER, Rancho Santa Fe, CA (US); David S. BAKER, Carlsbad, CA (US)

(21) Appl. No.: 18/263,952

(22) PCT Filed: Feb. 3, 2022
(86) PCT No.: PCT/US2022/015092

§ 371 Date: Aug. 2, 2023

Related U.S. Application Data
(60) Provisional application No. 63/145,676, filed on Feb. 4, 2021.

(51) Int. Cl. A61K 31/439; A61K 9/00; A61K 31/4178; A61K 31/4409; A61K 45/06

(52) U.S. Cl. CPC A61K 31/439; A61K 9/0048; A61K 31/4178; A61K 31/4409; A61K 45/06

(57) ABSTRACT

Provided herein is an ophthalmic composition. In some embodiments, the ophthalmic composition comprises a low concentration of an ophthalmic agent for treatment of presbyopia. Further disclosed herein is an ophthalmic composition including a low concentration of an ophthalmic agent and deuterated water.

### CROSS-REFERENCE

This application claims the benefit U.S. Provisional Patent Application No. 63/145,676 filed Feb. 4, 2021, which is hereby incorporated by reference in its entirety.

### BACKGROUND OF THE DISCLOSURE

Pharmaceutical formulations have an expiration date which is based on the degradation of the active ingredient.

### SUMMARY OF THE DISCLOSURE

Provided herein are ophthalmic compositions for treating presbyopia comprising aceclidine or a pharmaceutically acceptable salt of aceclidine and deuterated water. In some embodiments of an ophthalmic composition described herein, the aceclidine or the pharmaceutically acceptable salt of aceclidine is present in the ophthalmic composition at a concentration of about 0.25 wt % to about 2.0% wt %. In some embodiments of an ophthalmic composition described herein, the aceclidine or the pharmaceutically acceptable salt of aceclidine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the ophthalmic composition further comprises pilocarpine or a pharmaceutically acceptable salt of pilocarpine. In some embodiments of an ophthalmic composition described herein, the pilocarpine or the pharmaceutically acceptable salt of pilocarpine is present in the ophthalmic composition at a concentration of about 0.5 wt % to about 2.5 wt %, about 0.01 wt % to 0.45 wt %, or about 0.001 wt % to about 2 wt %. In some embodiments of an ophthalmic composition described herein, the pilocarpine or the pharmaceutically acceptable salt of pilocarpine is present in the ophthalmic composition at a concentration of at least about 0.25 wt %, 0.5 wt %, or 1.0 wt %. In some embodiments of an ophthalmic composition described herein, the pilocarpine or the pharmaceutically acceptable salt of pilocarpine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the ophthalmic composition further comprises tropicamide or a pharmaceutically acceptable salt of tropicamide. In some embodiments of an ophthalmic composition described herein, the tropicamide or the pharmaceutically acceptable salt of tropicamide is present in the ophthalmic composition at a concentration about 0.010 wt % to about 0.1 wt % or about 0.025 wt % to about 0.1 wt %. In some embodiments of an ophthalmic composition described herein, the tropicamide or the pharmaceutically acceptable salt of tropicamide is present in the ophthalmic composition at a concentration of at least about 0.02 wt %, 0.5 wt %, or 1.0 wt %. In some embodiments of an ophthalmic composition described herein, the tropicamide or the pharmaceutically acceptable salt of tropicamide is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the ophthalmic composition further comprises pilocarpine or a pharmaceutically acceptable salt of pilocarpine and tropicamide or a pharmaceutically acceptable salt of tropicamide. In some embodiments of an ophthalmic composition described herein, the pilocarpine or the pharmaceutically acceptable salt of pilocarpine is present in the ophthalmic composition at a concentration of about 0.5 wt % to about 2.5 wt %, about 0.01 wt % to 0.45 wt %, or about 0.001 wt % to about 2 wt %. In some embodiments of an ophthalmic composition described herein, the pilocarpine or the pharmaceutically acceptable salt of pilocarpine is present in the ophthalmic composition at a concentration of at least about 0.25 wt %, 0.5 wt %, or 1.0 wt %. In some embodiments of an ophthalmic composition described herein, the pilocarpine or the pharmaceutically acceptable salt of pilocarpine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the tropicamide or the pharmaceutically acceptable salt of tropicamide is present in the ophthalmic composition at a concentration about 0.010 wt % to about 0.1 wt % or about 0.025 wt % to about 0.1 wt %. In some embodiments of an ophthalmic composition described herein, the tropicamide or the pharmaceutically acceptable salt of tropicamide is present in the ophthalmic composition at a concentration of at least about 0.02 wt %, 0.5 wt %, or 1.0 wt %. In some embodiments of an ophthalmic composition described herein, the tropicamide or the pharmaceutically acceptable salt of tropicamide is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the ophthalmic composition has a pH of about 4.2 to about 7.9. In some embodiments, the ophthalmic composition has a pH of about 4.5 to about 7.5. In some embodiments, the ophthalmic composition has a pH of about 5.5 to about 6.5. In some embodiments of an ophthalmic composition described herein, the ophthalmic composition has a pH of one of: less than about 7.3, less than about 7.2, less than about 7.1, less than about 7, less than about 6.8, less than about 6.5, less than about 6.4, less than about 6.3, less than about 6.2, less than about 6.1, less than about 6, less than about 5.9, less than about 5.8, less than about 5.2, less than about 4.8, or less than about 4.5 after an extended period of time under a storage condition. In some embodiments of an ophthalmic composition described herein, the ophthalmic composition comprises one of: at least about 80%, at least about 85%, at least about 90%, at least about 93%, at least about 95%, at least about 97%, at least about 98%, or at least about 99% of the aceclidine or the pharmaceutically acceptable salt of aceclidine based on initial concentration after an extended period of time under a storage condition. In some embodiments of an ophthalmic composition described herein, the ophthalmic composition comprises one of: at least about 80%, at least about 85%, at least about 90%, at least about 93%, at least about 95%, at least about 97%, at least about 98%, or at least about 99% of the pilocarpine or the pharmaceutically acceptable salt of pilocarpine based on initial concentration after an extended period of time under a storage condition. In some embodiments of an ophthalmic composition described herein, the ophthalmic composition comprises one of: at least about 80%, at least about 85%, at least about 90%, at least about 93%, at least about 95%, at least about 97%, at least about 98%, or at least about 99% of the tropicamide or the pharmaceutically acceptable salt of tropicamide based on initial concentration after an extended period of time under a storage condition. In some embodiments of an ophthalmic composition described herein, the ophthalmic composition further has a potency of one of: at least 80%, at least 85%, at least 90%, at least 93%, at least 95%, at least 97%, at least 98%, or at least 99% after an extended period of time under a storage condition. In some embodiments of an ophthalmic composition described herein, the extended period of time is one of: about 1 week, about 2 weeks, about 3 weeks, about 1 month, about 2 months, about 3 months, about 4 months, about 5 months, about 6 months, about 8 months, about 10 months, about 12 months, about 18 months, about 24 months, about 36 months, about 4 years, or about 5 years. In some embodiments of an ophthalmic composition described herein, the storage condition has a storage temperature of about 0° C. to about 30° C., 2° C. to about 10° C., or about 16° C. to about 26° C. In some embodiments of an ophthalmic composition described herein, the ophthalmic composition further comprises an osmolarity adjusting agent. In some embodiments of an ophthalmic composition described herein, the osmolarity adjusting agent is sodium chloride. In some embodiments of an ophthalmic composition described herein, the sodium chloride is present in the ophthalmic composition at a concentration of one of: about 0.01 wt % to about 1.0 wt %, about 0.05 wt % to about 1.5 wt %, about 0.075 wt % to about 2.0 wt %, or about 0.1 wt % to about 3.0 wt %. In some embodiments of an ophthalmic composition described herein, the ophthalmic composition further comprises a buffering agent. In some embodiments of an ophthalmic composition described herein, the buffering agent is selected from borates, borate-polyol complexes, phosphate buffering agents, citrate buffering agents, acetate buffering agents, carbonate buffering agents, organic buffering agents, amino acid buffering agents, or combinations thereof. In some embodiments of an ophthalmic composition described herein, the ophthalmic composition has a dose-to-dose aceclidine concentration variation of one of: less than 50%, less than 40%, less than 30%, less than 20%, less than 10%, or less than 5%. In some embodiments of an ophthalmic composition described herein, the dose-to-dose aceclidine concentration variation is based on one of: 10 consecutive doses, 8 consecutive doses, 5 consecutive doses, 3 consecutive doses, or 2 consecutive doses. In some embodiments of an ophthalmic composition described herein, the ophthalmic composition has a dose-to-dose pilocarpine concentration variation of one of: less than 50%, less than 40%, less than 30%, less than 20%, less than 10%, or less than 5%. In some embodiments of an ophthalmic composition described herein, the dose-to-dose pilocarpine concentration variation is based on one of: 10 consecutive doses, 8 consecutive doses, 5 consecutive doses, 3 consecutive doses, or 2 consecutive doses. In some embodiments of an ophthalmic composition described herein, the ophthalmic composition has a dose-to-dose tropicamide concentration variation of one of: less than 50%, less than 40%, less than 30%, less than 20%, less than 10%, or less than 5%. In some embodiments of an ophthalmic composition described herein, the dose-to-dose tropicamide concentration variation is based on one of: 10 consecutive doses, 8 consecutive doses, 5 consecutive doses, 3 consecutive doses, or 2 consecutive doses. In some embodiments of an ophthalmic composition described herein, the ophthalmic composition further comprises a pH adjusting agent. In some embodiments of an ophthalmic composition described herein, the pH adjusting agent comprises DCl, HCl, NaOH, NaOD, CD₃COOD, C₆D₈O₇, CH₃COOH, C₆H₈O₇, or combinations thereof. In some embodiments of an ophthalmic composition described herein, the ophthalmic composition comprises one of: less than 5% of water (H₂O), less than 4% of H₂O, less than 3% of H₂O, less than 2% of H₂O, less than 1% of H₂O, less than 0.5% of H₂O, less than 0.1% of H₂O, or 0% of H₂O. In some embodiments of an ophthalmic composition described herein, the ophthalmic composition is not formulated as an injectable formulation. In some embodiments of an ophthalmic composition described herein, the ophthalmic composition further comprises one or more sodium phosphate buffers. In some embodiments of an ophthalmic composition described herein, a first sodium phosphate of the one or more sodium phosphate buffers is monosodium phosphate anhydrous. In some embodiments of an ophthalmic composition described herein, the monosodium phosphate anhydrous is present in the ophthalmic composition at a concentration of about 0.004 wt % to about 0.20 wt %. In some embodiments of an ophthalmic composition described herein, a second sodium phosphate buffer of the one or more sodium phosphate buffers is disodium phosphate anhydrous. In some embodiments of an ophthalmic composition described herein, the disodium phosphate anhydrous is present in the ophthalmic composition at a concentration of about 0.050 wt % to about 2.0 wt %. In some embodiments of an ophthalmic composition described herein, the ophthalmic composition comprises a preservative. In some embodiments of an ophthalmic composition described herein, the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof. In some embodiments of an ophthalmic composition described herein, the ophthalmic composition is free of a preservative selected from benzalkonium chloride, cetrimonium, sodium perborate, stabilized oxychloro complex, SofZia, polyquaternium-1, chlorobutanol, edetate disodium, polyhexamethylene biguanide, or combinations thereof. In some embodiments of an ophthalmic composition described herein, the ophthalmic composition is substantially free of a benzalkonium chloride preservative. In some embodiments of an ophthalmic composition described herein, the ophthalmic composition is substantially free of any preservative. In some embodiments of an ophthalmic composition described herein, the ophthalmic composition is essentially free of citrate and acetate buffering agents. In some embodiments of an ophthalmic composition described herein, the ophthalmic composition further comprises EDTA. In some embodiments of an ophthalmic composition described herein, the EDTA is present in the ophthalmic composition at a concentration of 0.01 wt % to about 0.50 wt %. In some embodiments of an ophthalmic composition described herein, the ophthalmic composition is essentially free of procaine and benactyzine, or pharmaceutically acceptable salts thereof. In some embodiments of an ophthalmic composition described herein, the ophthalmic composition further comprises a tonicity adjusting agent. In some embodiments of an ophthalmic composition described herein, the tonicity adjusting agent comprises a halide salt of a monovalent cation. In some embodiments of an ophthalmic composition described herein, the ophthalmic composition further comprises an ophthalmically acceptable viscosity agent. In some embodiments of an ophthalmic composition described herein, the ophthalmically acceptable viscosity agent comprises hydroxyethyl cellulose, hydroxypropyl cellulose, or hydroxypropylmethyl-cellulose (HPMC). In some embodiments of an ophthalmic composition described herein, the ophthalmic composition further comprises 0.004 wt % to about 0.20 wt % citrate. In some embodiments of an ophthalmic composition described herein, the ophthalmic composition is a storage-stabilized composition.

Provided herein are ophthalmic compositions comprising about 0.001 wt % to about 3 wt % pilocarpine or a pharmaceutically acceptable salt of pilocarpine and water, wherein the ophthalmic composition further comprises a buffering agent to provide a pH of about 4.2 to about 7.9. In some embodiments of an ophthalmic composition described herein, the pilocarpine or the pharmaceutically acceptable salt of pilocarpine is present in the ophthalmic composition at a concentration of about 0.5 wt % to about 2.5 wt %, about 0.01 wt % to 0.45 wt %, or about 0.001 wt % to about 2 wt %. In some embodiments of an ophthalmic composition described herein, the pilocarpine or the pharmaceutically acceptable salt of pilocarpine is present in the ophthalmic composition at a concentration of at least about 0.25 wt %, 0.5 wt %, or 1.0 wt %. In some embodiments of an ophthalmic composition described herein, the pilocarpine or the pharmaceutically acceptable salt of pilocarpine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the ophthalmic composition further comprises aceclidine or a pharmaceutically acceptable salt of aceclidine. In some embodiments of an ophthalmic composition described herein, the aceclidine or the pharmaceutically acceptable salt of aceclidine is present in the ophthalmic composition at a concentration of about 0.25 wt % to about 2.0% wt %. In some embodiments of an ophthalmic composition described herein, the aceclidine or the pharmaceutically acceptable salt of aceclidine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the ophthalmic composition further comprises tropicamide or a pharmaceutically acceptable salt of tropicamide. In some embodiments of an ophthalmic composition described herein, the tropicamide or the pharmaceutically acceptable salt of tropicamide is present in the ophthalmic composition at a concentration about 0.010 wt % to about 0.1 wt % or about 0.025 wt % to about 0.1 wt %. In some embodiments of an ophthalmic composition described herein, the tropicamide or the pharmaceutically acceptable salt of tropicamide is present in the ophthalmic composition at a concentration of at least about 0.02 wt %, 0.5 wt %, or 1.0 wt %. In some embodiments of an ophthalmic composition described herein, the tropicamide or the pharmaceutically acceptable salt of tropicamide is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the ophthalmic composition further comprises aceclidine or a pharmaceutically acceptable salt of aceclidine and tropicamide or a pharmaceutically acceptable salt of tropicamide. In some embodiments of an ophthalmic composition described herein, the aceclidine or the pharmaceutically acceptable salt of aceclidine is present in the ophthalmic composition at a concentration of about 0.25 wt % to about 2.0% wt %. In some embodiments of an ophthalmic composition described herein, the aceclidine or the pharmaceutically acceptable salt of aceclidine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the tropicamide or the pharmaceutically acceptable salt of tropicamide is present in the ophthalmic composition at a concentration about 0.010 wt % to about 0.1 wt % or about 0.025 wt % to about 0.1 wt %. In some embodiments of an ophthalmic composition described herein, the tropicamide or the pharmaceutically acceptable salt of tropicamide is present in the ophthalmic composition at a concentration of at least about 0.02 wt %, 0.5 wt %, or 1.0 wt %. In some embodiments of an ophthalmic composition described herein, the tropicamide or the pharmaceutically acceptable salt of tropicamide is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the ophthalmic composition has a pH of about 4.8 to about 6.4. In some embodiments of an ophthalmic composition described herein, the ophthalmic composition comprises one of: at least about 80%, at least about 85%, at least about 90%, at least about 93%, at least about 95%, at least about 97%, at least about 98%, or at least about 99% of the pilocarpine or the pharmaceutically acceptable salt of pilocarpine based on initial concentration after an extended period of time under a storage condition. In some embodiments of an ophthalmic composition described herein, the ophthalmic composition comprises one of: at least about 80%, at least about 85%, at least about 90%, at least about 93%, at least about 95%, at least about 97%, at least about 98%, or at least about 99% of the aceclidine or the pharmaceutically acceptable salt of aceclidine based on initial concentration after an extended period of time under a storage condition. In some embodiments of an ophthalmic composition described herein, the ophthalmic composition comprises one of: at least about 80%, at least about 85%, at least about 90%, at least about 93%, at least about 95%, at least about 97%, at least about 98%, or at least about 99% of the tropicamide or the pharmaceutically acceptable salt of tropicamide based on initial concentration after an extended period of time under a storage condition. In some embodiments of an ophthalmic composition described herein, the ophthalmic composition further has a potency of one of: at least 80%, at least 85%, at least 90%, at least 93%, at least 95%, at least 97%, at least 98%, or at least 99% after an extended period of time under a storage condition. In some embodiments of an ophthalmic composition described herein, the extended period of time is one of: about 1 week, about 2 weeks, about 3 weeks, about 1 month, about 2 months, about 3 months, about 4 months, about 5 months, about 6 months, about 8 months, about 10 months, about 12 months, about 18 months, about 24 months, about 36 months, about 4 years, or about 5 years. In some embodiments of an ophthalmic composition described herein, the storage condition has a storage temperature of about 0° C. to about 30° C., 2° C. to about 10° C., or about 16° C. to about 26° C. In some embodiments of an ophthalmic composition described herein, the ophthalmic composition further comprises an osmolarity adjusting agent. In some embodiments of an ophthalmic composition described herein, the osmolarity adjusting agent is sodium chloride. In some embodiments of an ophthalmic composition described herein, the sodium chloride is present in the ophthalmic composition at a concentration of one of: about 0.01 wt % to about 1.0 wt %, about 0.05 wt % to about 1.5 wt %, about 0.075 wt % to about 2.0 wt %, or about 0.1 wt % to about 3.0 wt %. In some embodiments of an ophthalmic composition described herein, the ophthalmic composition further comprises a buffering agent. In some embodiments of an ophthalmic composition described herein, the buffering agent is selected from borates, borate-polyol complexes, phosphate buffering agents, citrate buffering agents, acetate buffering agents, carbonate buffering agents, organic buffering agents, amino acid buffering agents, or combinations thereof. In some embodiments of an ophthalmic composition described herein, the ophthalmic composition has a dose-to-dose pilocarpine concentration variation of one of: less than 50%, less than 40%, less than 30%, less than 20%, less than 10%, or less than 5%. In some embodiments of an ophthalmic composition described herein, the dose-to-dose pilocarpine concentration variation is based on one of: 10 consecutive doses, 8 consecutive doses, 5 consecutive doses, 3 consecutive doses, or 2 consecutive doses. In some embodiments of an ophthalmic composition described herein, the ophthalmic composition has a dose-to-dose aceclidine concentration variation of one of: less than 50%, less than 40%, less than 30%, less than 20%, less than 10%, or less than 5%. In some embodiments of an ophthalmic composition described herein, the dose-to-dose aceclidine concentration variation is based on one of: 10 consecutive doses, 8 consecutive doses, 5 consecutive doses, 3 consecutive doses, or 2 consecutive doses. In some embodiments of an ophthalmic composition described herein, the ophthalmic composition has a dose-to-dose tropicamide concentration variation of one of: less than 50%, less than 40%, less than 30%, less than 20%, less than 10%, or less than 5%. In some embodiments of an ophthalmic composition described herein, the dose-to-dose tropicamide concentration variation is based on one of: 10 consecutive doses, 8 consecutive doses, 5 consecutive doses, 3 consecutive doses, or 2 consecutive doses. In some embodiments of an ophthalmic composition described herein, the ophthalmic composition further comprises a pH adjusting agent. In some embodiments of an ophthalmic composition described herein, the pH adjusting agent comprises DCl, HCl, NaOH, NaOD, CD₃COOD, C₆D₈O₇, CH₃COOH, C₆H₈O₇, or combinations thereof. In some embodiments of an ophthalmic composition described herein, the ophthalmic composition is not formulated as an injectable formulation. In some embodiments of an ophthalmic composition described herein, the ophthalmic composition further comprises one or more sodium phosphate buffers. In some embodiments of an ophthalmic composition described herein, a first sodium phosphate of the one or more sodium phosphate buffers is monosodium phosphate anhydrous. In some embodiments of an ophthalmic composition described herein, the monosodium phosphate anhydrous is present in the ophthalmic composition at a concentration of about 0.004 wt % to about 0.20 wt %. In some embodiments of an ophthalmic composition described herein, a second sodium phosphate buffer of the one or more sodium phosphate buffers is disodium phosphate anhydrous. In some embodiments of an ophthalmic composition described herein, the disodium phosphate anhydrous is present in the ophthalmic composition at a concentration of about 0.050 wt % to about 2.0 wt %. In some embodiments of an ophthalmic composition described herein, the ophthalmic composition comprises a preservative. In some embodiments of an ophthalmic composition described herein, the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof. In some embodiments of an ophthalmic composition described herein, the ophthalmic composition is free of a preservative selected from benzalkonium chloride, cetrimonium, sodium perborate, stabilized oxychloro complex, SofZia, polyquaternium-1, chlorobutanol, edetate disodium, polyhexamethylene biguanide, or combinations thereof. In some embodiments of an ophthalmic composition described herein, the ophthalmic composition is substantially free of a benzalkonium chloride preservative. In some embodiments of an ophthalmic composition described herein, the ophthalmic composition is substantially free of any preservative. In some embodiments of an ophthalmic composition described herein, the ophthalmic composition is essentially free of citrate and acetate buffering agents. In some embodiments of an ophthalmic composition described herein, the ophthalmic composition further comprises EDTA. In some embodiments of an ophthalmic composition described herein, the EDTA is present in the ophthalmic composition at a concentration of 0.01 wt % to about 0.50 wt %. In some embodiments of an ophthalmic composition described herein, the ophthalmic composition is essentially free of procaine and benactyzine, or pharmaceutically acceptable salts thereof. In some embodiments of an ophthalmic composition described herein, the ophthalmic composition further comprises a tonicity adjusting agent. In some embodiments of an ophthalmic composition described herein, the tonicity adjusting agent comprises a halide salt of a monovalent cation. In some embodiments of an ophthalmic composition described herein, the ophthalmic composition further comprises an ophthalmically acceptable viscosity agent. In some embodiments of an ophthalmic composition described herein, the ophthalmically acceptable viscosity agent comprises hydroxyethyl cellulose, hydroxypropyl cellulose, or hydroxypropylmethyl-cellulose (HPMC). In some embodiments of an ophthalmic composition described herein, the ophthalmic composition further comprises 0.004 wt % to about 0.20 wt % citrate. In some embodiments of an ophthalmic composition described herein, the ophthalmic composition is a storage-stabilized composition.

Provided herein are ophthalmic compositions comprising about 0.010 wt % to about 0.1 wt % tropicamide or a pharmaceutically acceptable salt of tropicamide and water, wherein the ophthalmic composition further comprises a buffering agent to provide a pH of about 4.2 to about 7.9. In some embodiments of an ophthalmic composition described herein, the tropicamide or the pharmaceutically acceptable salt of tropicamide is present in the ophthalmic composition at a concentration about 0.025 wt % to about 0.1 wt %. In some embodiments of an ophthalmic composition described herein, the tropicamide or the pharmaceutically acceptable salt of tropicamide is present in the ophthalmic composition at a concentration of at least about 0.02 wt %. In some embodiments of an ophthalmic composition described herein, the ophthalmic composition has a pH of about 4.8 to about 6.4. In some embodiments of an ophthalmic composition described herein, the ophthalmic composition comprises one of: at least about 80%, at least about 85%, at least about 90%, at least about 93%, at least about 95%, at least about 97%, at least about 98%, or at least about 99% of tropicamide or the pharmaceutically acceptable salt of tropicamide based on initial concentration after an extended period of time under a storage condition. In some embodiments of an ophthalmic composition described herein, the ophthalmic composition further has a potency of one of: at least 80%, at least 85%, at least 90%, at least 93%, at least 95%, at least 97%, at least 98%, or at least 99% after an extended period of time under a storage condition. In some embodiments of an ophthalmic composition described herein, the extended period of time is one of: about 1 week, about 2 weeks, about 3 weeks, about 1 month, about 2 months, about 3 months, about 4 months, about 5 months, about 6 months, about 8 months, about 10 months, about 12 months, about 18 months, about 24 months, about 36 months, about 4 years, or about 5 years. In some embodiments of an ophthalmic composition described herein, the storage condition has a storage temperature of about 0° C. to about 30° C., 2° C. to about 10° C., or about 16° C. to about 26° C. In some embodiments of an ophthalmic composition described herein, the ophthalmic composition further comprises an osmolarity adjusting agent. In some embodiments of an ophthalmic composition described herein, the osmolarity adjusting agent is sodium chloride. In some embodiments of an ophthalmic composition described herein, the sodium chloride is present in the ophthalmic composition at a concentration of one of: about 0.01 wt % to about 1.0 wt %, about 0.05 wt % to about 1.5 wt %, about 0.075 wt % to about 2.0 wt %, or about 0.1 wt % to about 3.0 wt %. In some embodiments of an ophthalmic composition described herein, the ophthalmic composition further comprises a buffering agent. In some embodiments of an ophthalmic composition described herein, the buffering agent is selected from borates, borate-polyol complexes, phosphate buffering agents, citrate buffering agents, acetate buffering agents, carbonate buffering agents, organic buffering agents, amino acid buffering agents, or combinations thereof. In some embodiments of an ophthalmic composition described herein, the ophthalmic composition has a dose-to-dose tropicamide concentration variation of one of: less than 50%, less than 40%, less than 30%, less than 20%, less than 10%, or less than 5%. In some embodiments of an ophthalmic composition described herein, the dose-to-dose tropicamide concentration variation is based on one of: 10 consecutive doses, 8 consecutive doses, 5 consecutive doses, 3 consecutive doses, or 2 consecutive doses. In some embodiments of an ophthalmic composition described herein, the ophthalmic composition further comprises a pH adjusting agent. In some embodiments of an ophthalmic composition described herein, the pH adjusting agent comprises DCl, HCl, NaOH, NaOD, CD₃COOD, C₆D₈O₇, CH₃COOH, C₆H₈O₇, or combinations thereof. In some embodiments of an ophthalmic composition described herein, the ophthalmic composition is not formulated as an injectable formulation. In some embodiments of an ophthalmic composition described herein, the ophthalmic composition further comprises one or more sodium phosphate buffers. In some embodiments of an ophthalmic composition described herein, a first sodium phosphate of the one or more sodium phosphate buffers is monosodium phosphate anhydrous. In some embodiments of an ophthalmic composition described herein, the monosodium phosphate anhydrous is present in the ophthalmic composition at a concentration of about 0.004 wt % to about 0.20 wt %. In some embodiments of an ophthalmic composition described herein, a second sodium phosphate buffer of the one or more sodium phosphate buffers is disodium phosphate anhydrous. In some embodiments of an ophthalmic composition described herein, the disodium phosphate anhydrous is present in the ophthalmic composition at a concentration of about 0.050 wt % to about 2.0 wt %. In some embodiments of an ophthalmic composition described herein, the ophthalmic composition comprises a preservative. In some embodiments of an ophthalmic composition described herein, the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof. In some embodiments of an ophthalmic composition described herein, the ophthalmic composition is free of a preservative selected from benzalkonium chloride, cetrimonium, sodium perborate, stabilized oxychloro complex, SofZia, polyquaternium-1, chlorobutanol, edetate disodium, polyhexamethylene biguanide, or combinations thereof. In some embodiments of an ophthalmic composition described herein, the ophthalmic composition is substantially free of a benzalkonium chloride preservative. In some embodiments of an ophthalmic composition described herein, the ophthalmic composition is substantially free of any preservative. In some embodiments of an ophthalmic composition described herein, the ophthalmic composition is essentially free of citrate and acetate buffering agents. In some embodiments of an ophthalmic composition described herein, the ophthalmic composition further comprises EDTA. In some embodiments of an ophthalmic composition described herein, the EDTA is present in the ophthalmic composition at a concentration of 0.01 wt % to about 0.50 wt %. In some embodiments of an ophthalmic composition described herein, the ophthalmic composition is essentially free of procaine and benactyzine, or pharmaceutically acceptable salts thereof. In some embodiments of an ophthalmic composition described herein, the ophthalmic composition further comprises a tonicity adjusting agent. In some embodiments of an ophthalmic composition described herein, the tonicity adjusting agent comprises a halide salt of a monovalent cation. In some embodiments of an ophthalmic composition described herein, the ophthalmic composition further comprises an ophthalmically acceptable viscosity agent. In some embodiments of an ophthalmic composition described herein, the ophthalmically acceptable viscosity agent comprises hydroxyethyl cellulose, hydroxypropyl cellulose, or hydroxypropylmethyl-cellulose (HPMC). In some embodiments of an ophthalmic composition described herein, the ophthalmic composition further comprises 0.004 wt % to about 0.20 wt % citrate. In some embodiments of an ophthalmic composition described herein, the ophthalmic composition is a storage-stabilized composition.

Provided herein are ophthalmic compositions comprising about 0.25 wt % to about 2.0% wt % aceclidine or a pharmaceutically acceptable salt of aceclidine and water, wherein the ophthalmic composition further comprises a buffering agent to provide a pH of about 4.2 to about 7.9. In some embodiments of an ophthalmic composition described herein, the ophthalmic composition has a pH of about 4.8 to about 6.4. In some embodiments of an ophthalmic composition described herein, the ophthalmic composition comprises one of: at least about 80%, at least about 85%, at least about 90%, at least about 93%, at least about 95%, at least about 97%, at least about 98%, or at least about 99% of aceclidine or the pharmaceutically acceptable salt of aceclidine based on initial concentration after an extended period of time under a storage condition. In some embodiments of an ophthalmic composition described herein, the ophthalmic composition further has a potency of one of: at least 80%, at least 85%, at least 90%, at least 93%, at least 95%, at least 97%, at least 98%, or at least 99% after an extended period of time under a storage condition. In some embodiments of an ophthalmic composition described herein, the extended period of time is one of: about 1 week, about 2 weeks, about 3 weeks, about 1 month, about 2 months, about 3 months, about 4 months, about 5 months, about 6 months, about 8 months, about 10 months, about 12 months, about 18 months, about 24 months, about 36 months, about 4 years, or about 5 years. In some embodiments of an ophthalmic composition described herein, the storage condition has a storage temperature of about 0° C. to about 30° C., 2° C. to about 10° C., or about 16° C. to about 26° C. In some embodiments of an ophthalmic composition described herein, the ophthalmic composition further comprises an osmolarity adjusting agent. In some embodiments of an ophthalmic composition described herein, the osmolarity adjusting agent is sodium chloride. In some embodiments of an ophthalmic composition described herein, the sodium chloride is present in the ophthalmic composition at a concentration of one of: about 0.01 wt % to about 1.0 wt %, about 0.05 wt % to about 1.5 wt %, about 0.075 wt % to about 2.0 wt %, or about 0.1 wt % to about 3.0 wt %. In some embodiments of an ophthalmic composition described herein, the ophthalmic composition further comprises a buffering agent. In some embodiments of an ophthalmic composition described herein, the buffering agent is selected from borates, borate-polyol complexes, phosphate buffering agents, citrate buffering agents, acetate buffering agents, carbonate buffering agents, organic buffering agents, amino acid buffering agents, or combinations thereof. In some embodiments of an ophthalmic composition described herein, the ophthalmic composition has a dose-to-dose aceclidine concentration variation of one of: less than 50%, less than 40%, less than 30%, less than 20%, less than 10%, or less than 5%. In some embodiments of an ophthalmic composition described herein, the dose-to-dose aceclidine concentration variation is based on one of: 10 consecutive doses, 8 consecutive doses, 5 consecutive doses, 3 consecutive doses, or 2 consecutive doses. In some embodiments of an ophthalmic composition described herein, the ophthalmic composition further comprises a pH adjusting agent. In some embodiments of an ophthalmic composition described herein, the pH adjusting agent comprises DCl, HCl, NaOH, NaOD, CD₃COOD, C₆D₈O₇, CH₃COOH, C₆H₈O₇, or combinations thereof. In some embodiments of an ophthalmic composition described herein, the ophthalmic composition is not formulated as an injectable formulation. In some embodiments of an ophthalmic composition described herein, the ophthalmic composition further comprises one or more sodium phosphate buffers. In some embodiments of an ophthalmic composition described herein, a first sodium phosphate of the one or more sodium phosphate buffers is monosodium phosphate anhydrous. In some embodiments of an ophthalmic composition described herein, the monosodium phosphate anhydrous is present in the ophthalmic composition at a concentration of about 0.004 wt % to about 0.20 wt %. In some embodiments of an ophthalmic composition described herein, a second sodium phosphate buffer of the one or more sodium phosphate buffers is disodium phosphate anhydrous. In some embodiments of an ophthalmic composition described herein, the disodium phosphate anhydrous is present in the ophthalmic composition at a concentration of about 0.050 wt % to about 2.0 wt %. In some embodiments of an ophthalmic composition described herein, the ophthalmic composition comprises a preservative. In some embodiments of an ophthalmic composition described herein, the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof. In some embodiments of an ophthalmic composition described herein, the ophthalmic composition is free of a preservative selected from benzalkonium chloride, cetrimonium, sodium perborate, stabilized oxychloro complex, SofZia, polyquaternium-1, chlorobutanol, edetate disodium, polyhexamethylene biguanide, or combinations thereof. In some embodiments of an ophthalmic composition described herein, the ophthalmic composition is substantially free of a benzalkonium chloride preservative. In some embodiments of an ophthalmic composition described herein, the ophthalmic composition is substantially free of any preservative. In some embodiments of an ophthalmic composition described herein, the ophthalmic composition is essentially free of citrate and acetate buffering agents. In some embodiments of an ophthalmic composition described herein, the ophthalmic composition further comprises EDTA. In some embodiments of an ophthalmic composition described herein, the EDTA is present in the ophthalmic composition at a concentration of 0.01 wt % to about 0.50 wt %. In some embodiments of an ophthalmic composition described herein, the ophthalmic composition is essentially free of procaine and benactyzine, or pharmaceutically acceptable salts thereof. In some embodiments of an ophthalmic composition described herein, the ophthalmic composition further comprises a tonicity adjusting agent. In some embodiments of an ophthalmic composition described herein, the tonicity adjusting agent comprises a halide salt of a monovalent cation. In some embodiments of an ophthalmic composition described herein, the ophthalmic composition further comprises an ophthalmically acceptable viscosity agent. In some embodiments of an ophthalmic composition described herein, the ophthalmically acceptable viscosity agent comprises hydroxyethyl cellulose, hydroxypropyl cellulose, or hydroxypropylmethyl-cellulose (HPMC). In some embodiments of an ophthalmic composition described herein, the ophthalmic composition further comprises 0.004 wt % to about 0.20 wt % citrate. In some embodiments of an ophthalmic composition described herein, the ophthalmic composition is a storage-stabilized composition.

Provided herein are ophthalmic compositions for treating presbyopia comprising an ophthalmic agent and deuterated water. In some embodiments of an ophthalmic composition described herein, the ophthalmic agent is pilocarpine or a pharmaceutically acceptable salt of pilocarpine, aceclidine or a pharmaceutically acceptable salt of aceclidine, tropicamide or a pharmaceutically acceptable salt of tropicamide, or combinations thereof. In some embodiments of an ophthalmic composition described herein, the aceclidine or the pharmaceutically acceptable salt of aceclidine is present in the ophthalmic composition at a concentration of about 0.25 wt % to about 2.0% wt %. In some embodiments of an ophthalmic composition described herein, the aceclidine or the pharmaceutically acceptable salt of aceclidine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the pilocarpine or the pharmaceutically acceptable salt of pilocarpine is present in the ophthalmic composition at a concentration of about 0.5 wt % to about 2.5 wt %, about 0.01 wt % to 0.45 wt %, or about 0.001 wt % to about 2 wt %. In some embodiments of an ophthalmic composition described herein, the pilocarpine or the pharmaceutically acceptable salt of pilocarpine is present in the ophthalmic composition at a concentration of at least about 0.25 wt %, 0.5 wt %, or 1.0 wt %. In some embodiments of an ophthalmic composition described herein, the pilocarpine or the pharmaceutically acceptable salt of pilocarpine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the tropicamide or the pharmaceutically acceptable salt of tropicamide is present in the ophthalmic composition at a concentration about 0.010 wt % to about 0.1 wt % or about 0.025 wt % to about 0.1 wt %. In some embodiments of an ophthalmic composition described herein, the tropicamide or the pharmaceutically acceptable salt of tropicamide is present in the ophthalmic composition at a concentration of at least about 0.02 wt %, 0.5 wt %, or 1.0 wt %. In some embodiments of an ophthalmic composition described herein, the tropicamide or the pharmaceutically acceptable salt of tropicamide is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some embodiments of an ophthalmic composition described herein, the ophthalmic agent is atropine, atropine sulfate, noratropine, atropine-N-oxide, tropine, tropic acid, atropine methonitrate, diphenhydramine, dimenhydrinate, dicyclomine, flavoxate, oxybutynin, tiotropium, hyoscine, scopolamine (L-hyoscine), hydroxyzine, ipratropium, tropicamide, cyclopentolate, pirenzepine, homatropine, solifenacin, darifenacin, benzatropine, mebeverine, procyclidine, aclidinium bromide, trihexyphenidyl/benzhexol, tolterodine, anisodamine, or combinations thereof. In some embodiments of an ophthalmic composition described herein, the ophthalmic agent is aflibercept, ranibizumab, pegaptanib, cyclopentolate, phenylephrine, homatropine, scopolamine, cyclopentolate/phenylephrine, phenylephrine/scopolamine, tropicamide, ketorolac/phenylephrine, hydroxyamphetamine/tropicamide, cysteamine, ocriplasmin, mitomycin, dapiprazole, lidocaine, proparacaine, tetracaine, benoxinate, azithromycin, bacitracin, besifloxacin, boric acid, chloramphenicol, ciprofloxacin, erythromycin, ganciclovir, gatifloxacin, gentamicin, idoxuridine, levofloxacin, moxifloxacin, natamycin, norfloxacin, ofloxacin, bacitracin/polymyxin b, tobramycin, polymyxin b/trimethoprim, povidone iodine, trifluridine, gramicidin/neomycin/polymyxin b, sulfacetamide sodium, sulfisoxazole, bacitracin/neomycin/polymyxin b, oxytetracycline/polymyxin b, phenylephrine/sulfacetamide sodium, vidarabine, bromfenac, nepafenac, ketorolac, cyclosporine, flurbiprofen, suprofen, diclofenac, alcaftadine, azelastine, bepotastine, cromolyn, emedastine, epinastine, ketotifen, levocabastine, lodoxamide, nedocromil, naphazoline, naphazoline/pheniramine, naphazoline/zinc sulfate, olopatadine, oxymetazoline, pemirolast, phenylephrine/zinc sulfate, tetrahydrozoline, tetrahydrozoline/zinc sulfate, fluorescein, fluorescein/proparacaine, benoxinate/fluorescein, indocyanine green, trypan blue, acetylcholine, apraclonidine, betaxolol, bimatoprost, brimonidine, brinzolamide, brimonidine/brinzolamide, carbachol, carteolol, demecarium bromide, dipivefrin, dorzolamide, dorzolamide/timolol, echothiophate iodide, epinephrine, epinephrine/pilocarpine, latanoprost, levobunolol, levobetaxolol, metipranolol, physostigmine, pilocarpine, tafluprost, timolol, travoprost, unoprostone, artificial tear, dexamethasone, difluprednate, fluocinolone, fluorometholone, loteprednol, medrysone, prednisolone, rimexolone, triamcinolone, fluorometholone/sulfacetamide sodium, dexamethasone/neomycin, dexamethasone/tobramycin, dexamethasone/neomycin/polymyxin b, loteprednol/tobramycin, prednisolone/sulfacetamide sodium, bacitracin/hydrocortisone/neomycin/polymyxin b, hydrocortisone/neomycin/polymyxin b, chloramphenicol/hydrocortisone/polymyxin b, neomycin/polymyxin b/prednisolone, gentamicin/prednisolone, ketorolac/phenylephrine, diphenhydramine, dimenhydrinate, dicyclomine, flavoxate, oxybutynin, tiotropium, hyoscine, scopolamine (L-hyoscine), hydroxyzine, ipratropium, pirenzepine, solifenacin, darifenacin, benzatropine, mebeverine, procyclidine, aclidinium bromide, trihexyphenidyl/benzhexol, tolterodine, aceclidine, anisodamine, or combinations thereof. In some embodiments of an ophthalmic composition described herein, the ophthalmic agent is a miotic agent. In some embodiments of an ophthalmic composition described herein, the miotic agent is dapiprazole, thymoxamine, brimonidine, nicotine, apraclonidin, phentolamine, pharmaceutically acceptable salts thereof, or combinations thereof. In some embodiments of an ophthalmic composition described herein, the ophthalmic composition has a pH of about 4.2 to about 7.9. In some embodiments, the ophthalmic composition has a pH of about 4.5 to about 7.5. In some embodiments, the ophthalmic composition has a pH of about 5.5 to about 6.5. In some embodiments of an ophthalmic composition described herein, the ophthalmic composition has a pH of one of: less than about 7.3, less than about 7.2, less than about 7.1, less than about 7, less than about 6.8, less than about 6.5, less than about 6.4, less than about 6.3, less than about 6.2, less than about 6.1, less than about 6, less than about 5.9, less than about 5.8, less than about 5.2, less than about 4.8, or less than about 4.5 after an extended period of time under a storage condition. In some embodiments of an ophthalmic composition described herein, the ophthalmic composition comprises one of: at least about 80%, at least about 85%, at least about 90%, at least about 93%, at least about 95%, at least about 97%, at least about 98%, or at least about 99% of the pilocarpine or the pharmaceutically acceptable salt of pilocarpine based on initial concentration after an extended period of time under a storage condition. In some embodiments of an ophthalmic composition described herein, the ophthalmic composition comprises one of: at least about 80%, at least about 85%, at least about 90%, at least about 93%, at least about 95%, at least about 97%, at least about 98%, or at least about 99% of the aceclidine or the pharmaceutically acceptable salt of aceclidine based on initial concentration after an extended period of time under a storage condition. In some embodiments of an ophthalmic composition described herein, the ophthalmic composition comprises one of: at least about 80%, at least about 85%, wherein the ophthalmic composition comprises one of: at least about 80%, at least about 85%, at least about 90%, at least about 93%, at least about 95%, at least about 97%, at least about 98%, or at least about 99% of the tropicamide or the pharmaceutically acceptable salt of tropicamide based on initial concentration after an extended period of time under a storage condition. In some embodiments of an ophthalmic composition described herein, the ophthalmic composition further has a potency of one of: at least 80%, at least 85%, at least 90%, at least 93%, at least 95%, at least 97%, at least 98%, or at least 99% after an extended period of time under a storage condition. In some embodiments of an ophthalmic composition described herein, the extended period of time is one of: about 1 week, about 2 weeks, about 3 weeks, about 1 month, about 2 months, about 3 months, about 4 months, about 5 months, about 6 months, about 8 months, about 10 months, about 12 months, about 18 months, about 24 months, about 36 months, about 4 years, or about 5 years. In some embodiments of an ophthalmic composition described herein, the storage condition has a storage temperature of about 0° C. to about 30° C., 2° C. to about 10° C., or about 16° C. to about 26° C. In some embodiments of an ophthalmic composition described herein, the ophthalmic composition further comprises an osmolarity adjusting agent. In some embodiments of an ophthalmic composition described herein, the osmolarity adjusting agent is sodium chloride. In some embodiments of an ophthalmic composition described herein, the sodium chloride is present in the ophthalmic composition at a concentration of one of: about 0.01 wt % to about 1.0 wt %, about 0.05 wt % to about 1.5 wt %, about 0.075 wt % to about 2.0 wt %, or about 0.1 wt % to about 3.0 wt %. In some embodiments of an ophthalmic composition described herein, the ophthalmic composition further comprises a buffering agent. In some embodiments of an ophthalmic composition described herein, the buffering agent is selected from borates, borate-polyol complexes, phosphate buffering agents, citrate buffering agents, acetate buffering agents, carbonate buffering agents, organic buffering agents, amino acid buffering agents, or combinations thereof. In some embodiments of an ophthalmic composition described herein, the ophthalmic composition has a dose-to-dose aceclidine concentration variation of one of: less than 50%, less than 40%, less than 30%, less than 20%, less than 10%, or less than 5%. In some embodiments of an ophthalmic composition described herein, the dose-to-dose aceclidine concentration variation is based on one of: 10 consecutive doses, 8 consecutive doses, 5 consecutive doses, 3 consecutive doses, or 2 consecutive doses. In some embodiments of an ophthalmic composition described herein, the ophthalmic composition has a dose-to-dose pilocarpine concentration variation of one of: less than 50%, less than 40%, less than 30%, less than 20%, less than 10%, or less than 5%. In some embodiments of an ophthalmic composition described herein, the dose-to-dose pilocarpine concentration variation is based on one of: 10 consecutive doses, 8 consecutive doses, 5 consecutive doses, 3 consecutive doses, or 2 consecutive doses. In some embodiments of an ophthalmic composition described herein, the ophthalmic composition has a dose-to-dose tropicamide concentration variation of one of: less than 50%, less than 40%, less than 30%, less than 20%, less than 10%, or less than 5%. In some embodiments of an ophthalmic composition described herein, the dose-to-dose tropicamide concentration variation is based on one of: 10 consecutive doses, 8 consecutive doses, 5 consecutive doses, 3 consecutive doses, or 2 consecutive doses. In some embodiments of an ophthalmic composition described herein, the ophthalmic composition further comprises a pH adjusting agent. In some embodiments of an ophthalmic composition described herein, the pH adjusting agent comprises DCl, HCl, NaOH, NaOD, CD₃COOD, C₆D₈O₇, CH₃COOH, C₆H₈O₇, or combinations thereof. In some embodiments of an ophthalmic composition described herein, the ophthalmic composition comprises one of: less than 5% of water (H₂O), less than 4% of H₂O, less than 3% of H₂O, less than 2% of H₂O, less than 1% of H₂O, less than 0.5% of H₂O, less than 0.1% of H₂O, or 0% of H₂O. In some embodiments of an ophthalmic composition described herein, the ophthalmic composition is not formulated as an injectable formulation. In some embodiments of an ophthalmic composition described herein, the ophthalmic composition further comprises one or more sodium phosphate buffers. In some embodiments of an ophthalmic composition described herein, a first sodium phosphate of the one or more sodium phosphate buffers is monosodium phosphate anhydrous. In some embodiments of an ophthalmic composition described herein, the monosodium phosphate anhydrous is present in the ophthalmic composition at a concentration of about 0.004 wt % to about 0.20 wt %. In some embodiments of an ophthalmic composition described herein, a second sodium phosphate buffer of the one or more sodium phosphate buffers is disodium phosphate anhydrous. In some embodiments of an ophthalmic composition described herein, the disodium phosphate anhydrous is present in the ophthalmic composition at a concentration of about 0.050 wt % to about 2.0 wt %. In some embodiments of an ophthalmic composition described herein, the ophthalmic composition comprises a preservative. In some embodiments of an ophthalmic composition described herein, the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof. In some embodiments of an ophthalmic composition described herein, the ophthalmic composition is free of a preservative selected from benzalkonium chloride, cetrimonium, sodium perborate, stabilized oxychloro complex, SofZia, polyquaternium-1, chlorobutanol, edetate disodium, polyhexamethylene biguanide, or combinations thereof. In some embodiments of an ophthalmic composition described herein, the ophthalmic composition is substantially free of a benzalkonium chloride preservative. In some embodiments of an ophthalmic composition described herein, the ophthalmic composition is substantially free of any preservative. In some embodiments of an ophthalmic composition described herein, the ophthalmic composition is essentially free of citrate and acetate buffering agents. In some embodiments of an ophthalmic composition described herein, the ophthalmic composition further comprises EDTA. In some embodiments of an ophthalmic composition described herein, the EDTA is present in the ophthalmic composition at a concentration of 0.01 wt % to about 0.50 wt %. In some embodiments of an ophthalmic composition described herein, the ophthalmic composition is essentially free of procaine and benactyzine, or pharmaceutically acceptable salts thereof. In some embodiments of an ophthalmic composition described herein, the ophthalmic composition further comprises a tonicity adjusting agent. In some embodiments of an ophthalmic composition described herein, the tonicity adjusting agent comprises a halide salt of a monovalent cation. In some embodiments of an ophthalmic composition described herein, the ophthalmic composition further comprises an ophthalmically acceptable viscosity agent. In some embodiments of an ophthalmic composition described herein, the ophthalmically acceptable viscosity agent comprises hydroxyethyl cellulose, hydroxypropyl cellulose, or hydroxypropylmethyl-cellulose (HPMC). In some embodiments of an ophthalmic composition described herein, the ophthalmic composition further comprises 0.004 wt % to about 0.20 wt % citrate. In some embodiments of an ophthalmic composition described herein, the ophthalmic composition is a storage-stabilized composition.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized and the accompanying drawings of which:

FIG. 1 illustrates the relationship between pilocarpine concentration and the rate of pilocarpic acid formation in deuterated and non-deuterated water.

FIG. 2 illustrates the relationship between pH and the rate of pilocarpic acid formation in pilocarpine solutions of deuterated and non-deuterated water.

### DETAILED DESCRIPTION OF THE DISCLOSURE

The present disclosure recognizes that, in some cases, there is a need for a stabilized ophthalmic composition with extended shelf life upon storage. The present disclosure also recognizes that, in some cases, there is a need for stabilizing an ophthalmic composition through arresting or reducing hydrolysis of at least some of its active agents. The present disclosure further recognizes that, in some cases, there is a need for an ophthalmic composition that provides convenient and effective delivery of a muscarinic agent such as aceclidine, pilocarpine, or tropicamide in the eye of a patient.

Further, the present disclosure recognizes the need, in some cases, for an ophthalmic composition stabilized without the need for a preservative. The present disclosure recognizes that, in some cases, there is a need for an ophthalmic composition that is substantially free of a preservative.

The present disclosure recognizes that some muscarinic agents (e.g. aceclidine, pilocarpine, tropicamide, or pharmaceutically acceptable salts thereof) prevent or arrest the development of presbyopia or ameliorates, reduces, or treats presbyopia. Presbyopia is an age-related visual defect due to a decline in near focusing ability. Methods for treating presbyopia include use of corrective lens and surgical interventions. However, these methods have drawbacks including discomfort with the use of corrective lens and risks associated with surgical interventions. The present disclosure provides compositions and methods for treating presbyopia based therapeutics agents, such as muscarinic agents (e.g. aceclidine, pilocarpine, tropicamide, or pharmaceutically acceptable salts thereof). According to certain aspects of the present disclosure, the compositions and treatments for preventing or arresting the development of presbyopia allow convenient administration, reduce potential side effects, has suitable stability, and/or provide relatively consistent therapeutic effects.

Further, the present disclosure recognizes that some liquid muscarinic agent (e.g. aceclidine, pilocarpine, or tropicamide) compositions are formulated at a relatively lower pH range (e.g. less than 4.5) for stability of muscarinic agent (e.g. aceclidine, pilocarpine, tropicamide, or its pharmaceutically acceptable salts). For some individuals, the lower pH range in some instances causes discomfort or other side effects such as pain or burning sensation in the eye. For some individuals, the lower pH in some instances elicits a tear response which reduces the absorption of the drug in the eye and therefore the effectiveness.

Still further, the present disclosure recognizes that some muscarinic agent (e.g. aceclidine, pilocarpine, or tropicamide) liquid compositions formulated at lower concentrations present stability challenges that are less so in higher concentrations.

Finally, the present disclosure recognizes that, in some cases, deuterated water stabilizes ophthalmic compositions. In some cases, the deuterated water is a weak acid as compared to H₂O, as such deuterated water comprises a lower concentration of the reactive species (e.g., —OD) which in some instances leads to base catalyzed hydrolysis of an active agent in the ophthalmic composition. As such, in some instances, compositions comprising deuterated water leads to reduced base catalyzed hydrolysis when compared to compositions comprising H₂O. In some instances, deuterated water further lowers the buffering capacity of an ophthalmic composition, leading to less tear reflex in the eye.

Ophthalmic Compositions

Provided herein is an ophthalmic composition for treating presbyopia comprising an ophthalmic agent and deuterated water. Provided herein is an ophthalmic composition containing low concentrations of an ophthalmic agent. In some embodiments, the ophthalmic composition comprises about 0.25 wt % to about 2.0% wt %, about 0.5 wt % to about 2.5 wt %, about 0.01 wt % to 0.45 wt %, about 0.001 wt % to about 2 wt %, about 0.010 wt % to about 0.1 wt %, or about 0.025 wt % to about 0.1 wt % of an ophthalmic agent for treatment of presbyopia; and an ophthalmically acceptable carrier, wherein the ophthalmic agent is distributed with substantial uniformity throughout the ophthalmically acceptable carrier. In some embodiments, the ophthalmic composition comprises at least about 0.02 wt %, 0.25 wt %, 0.5 wt %, or 1.0 wt % of an ophthalmic agent for treatment of presbyopia; and an ophthalmically acceptable carrier, wherein the ophthalmic agent is distributed with substantial uniformity throughout the ophthalmically acceptable carrier. In some instances, the ophthalmic composition comprises aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some instances, the ophthalmic agent is atropine, atropine sulfate, noratropine, atropine-N-oxide, tropine, tropic acid, atropine methonitrate, diphenhydramine, dimenhydrinate, dicyclomine, flavoxate, oxybutynin, tiotropium, hyoscine, scopolamine (L-hyoscine), hydroxyzine, ipratropium, tropicamide, cyclopentolate, pirenzepine, homatropine, solifenacin, darifenacin, benzatropine, mebeverine, procyclidine, aclidinium bromide, trihexyphenidyl/benzhexol, tolterodine, anisodamine, or a combination thereof.

In some embodiments, the ophthalmic composition comprises one or more sympathetic agonists. In some embodiments, the sympathetic agonist is selected from phenylephrine or hydroxyamphetamine. In some embodiments, the ophthalmic composition comprises one or more of muscarinic antagonist: atropine, atropine sulfate, noratropine, atropine-N-oxide, tropine, tropic acid, atropine methonitrate, diphenhydramine, dimenhydrinate, dicyclomine, flavoxate, oxybutynin, tiotropium, hyoscine, scopolamine (L-hyoscine), hydroxyzine, ipratropium, tropicamide, cyclopentolate, pirenzepine, homatropine, solifenacin, darifenacin, benzatropine, mebeverine, procyclidine, aclidinium bromide, trihexyphenidyl/benzhexol, tolterodine, or anisodamine; in combination with one or more of sympathetic agonists: phenylephrine or hydroxyamphetamine.

In some embodiments, the ophthalmic agent for treating presbyopia is aflibercept, ranibizumab, pegaptanib, cyclopentolate, phenylephrine, homatropine, scopolamine, cyclopentolate/phenylephrine, phenylephrine/scopolamine, tropicamide, ketorolac/phenylephrine, hydroxyamphetamine/tropicamide, cysteamine, ocriplasmin, mitomycin, dapiprazole, lidocaine, proparacaine, tetracaine, benoxinate, azithromycin, bacitracin, besifloxacin, boric acid, chloramphenicol, ciprofloxacin, erythromycin, ganciclovir, gatifloxacin, gentamicin, idoxuridine, levofloxacin, moxifloxacin, natamycin, norfloxacin, ofloxacin, bacitracin/polymyxin b, tobramycin, polymyxin b/trimethoprim, povidone iodine, trifluridine, gramicidin/neomycin/polymyxin b, sulfacetamide sodium, sulfisoxazole, bacitracin/neomycin/polymyxin b, oxytetracycline/polymyxin b, phenylephrine/sulfacetamide sodium, vidarabine, bromfenac, nepafenac, ketorolac, cyclosporine, flurbiprofen, suprofen, diclofenac, alcaftadine, azelastine, bepotastine, cromolyn, emedastine, epinastine, ketotifen, levocabastine, lodoxamide, nedocromil, naphazoline, naphazoline/pheniramine, naphazoline/zinc sulfate, olopatadine, oxymetazoline, pemirolast, phenylephrine/zinc sulfate, tetrahydrozoline, tetrahydrozoline/zinc sulfate, fluorescein, fluorescein/proparacaine, benoxinate/fluorescein, indocyanine green, trypan blue, acetylcholine, apraclonidine, betaxolol, bimatoprost, brimonidine, brinzolamide, brimonidine/brinzolamide, carbachol, carteolol, demecarium bromide, dipivefrin, dorzolamide, dorzolamide/timolol, echothiophate iodide, epinephrine, epinephrine/pilocarpine, latanoprost, levobunolol, levobetaxolol, metipranolol, physostigmine, pilocarpine, tafluprost, timolol, travoprost, unoprostone, artificial tear, dexamethasone, difluprednate, fluocinolone, fluorometholone, loteprednol, medrysone, prednisolone, rimexolone, triamcinolone, fluorometholone/sulfacetamide sodium, dexamethasone/neomycin, dexamethasone/tobramycin, dexamethasone/neomycin/polymyxin b, loteprednol/tobramycin, prednisolone/sulfacetamide sodium, bacitracin/hydrocortisone/neomycin/polymyxin b, hydrocortisone/neomycin/polymyxin b, chloramphenicol/hydrocortisone/polymyxin b, neomycin/polymyxin b/prednisolone, gentamicin/prednisolone, ketorolac/phenylephrine, diphenhydramine, dimenhydrinate, dicyclomine, flavoxate, oxybutynin, tiotropium, hyoscine, scopolamine (L-hyoscine), hydroxyzine, ipratropium, pirenzepine, solifenacin, darifenacin, benzatropine, mebeverine, procyclidine, aclidinium bromide, trihexyphenidyl/benzhexol, tolterodine, aceclidine, anisodamine, or any combinations thereof. In some embodiments, the ophthalmic agent is aceclidine, tropicamide, pilocarpine, or combinations thereof.

In some embodiments, the ophthalmic agent for treating presbyopia is a miotic agent. In some instances, the miotic agent is dapiprazole, thymoxamine, brimonidine, nicotine, apraclonidin, phentolamine, pharmaceutically acceptable salts thereof, or combinations thereof.

In some embodiments, the ophthalmic agent for treating presbyopia is a muscarinic receptor agonist, muscarinic receptor antagonist, an alpha-1 adrenergic receptor antagonist, an alpha-2 adrenergic receptor agonist, a beta-adrenergic receptor antagonist, a nicotine receptor agonist, an antipsychotic, an antiemetic, a cannabinoid, a monoamine oxidase (MAO) inhibitor, an EP1 receptor agonist, an EP4 receptor agonist, an FP receptor agonist, a calcium channel modulator, an anticholinergic agent, or combinations thereof.

In some embodiments, the ophthalmic composition comprises an anti-inflammatory agent. In some instances, the anti-inflammatory agent is a nonsteroidal anti-inflammatory agent (NSAID). Exemplary NSAIDs include, but are not limited to, diclofenac, flurbiprofen, ketorolac, bromfenac, and nepafenac.

Described herein are ophthalmic composition for treating presbyopia comprising, in some embodiments, a cycloplegic agent. In some instances, the cycloplegic agent is present in the ophthalmic composition at a concentration of about 0.004 wt % to about 0.08 wt %. In some instances, the cycloplegic agent is present in the ophthalmic composition at a concentration of about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some instances, the cycloplegic agent is tropicamide or a pharmaceutically acceptable salt of tropicamide.

In some embodiments, the ophthalmic composition is essentially free of procaine and benactyzine, or pharmaceutically acceptable salts thereof. In some embodiments, the ophthalmic composition is substantially free of procaine and benactyzine, or pharmaceutically acceptable salts thereof. In some instances, the ophthalmic composition has no detectable amount of procaine and benactyzine, or pharmaceutically acceptable salts thereof.

Provided herein is an ophthalmic composition comprising a muscarinic agent. In some embodiments, the muscarinic agent is aceclidine or a pharmaceutically acceptable salt of aceclidine. In some embodiments, the muscarinic agent is pilocarpine or a pharmaceutically acceptable salt of pilocarpine. In some embodiments, the muscarinic agent is tropicamide or a pharmaceutically acceptable salt of tropicamide. In some embodiments, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

Provided herein is an ophthalmic composition comprising low concentrations of aceclidine or a pharmaceutically acceptable salt of aceclidine. In some embodiments, the ophthalmic composition comprises about 0.25 wt % to about 2.0% wt % of aceclidine or a pharmaceutically acceptable salt of aceclidine for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt % of aceclidine or a pharmaceutically acceptable salt of aceclidine for treatment of presbyopia; and an ophthalmically acceptable carrier, wherein the ophthalmic agent is distributed with substantial uniformity throughout the ophthalmically acceptable carrier.

Provided herein is an ophthalmic composition comprising low concentrations of pilocarpine or a pharmaceutically acceptable salt of pilocarpine. In some embodiments, the ophthalmic composition comprises about 0.5 wt % to about 2.5 wt %, about 0.01 wt % to 0.45 wt %, or about 0.001 wt % to about 2 wt % of pilocarpine or a pharmaceutically acceptable salt of pilocarpine for treatment of presbyopia; and an ophthalmically acceptable carrier, wherein the ophthalmic agent is distributed with substantial uniformity throughout the ophthalmically acceptable carrier. In some embodiments, the ophthalmic composition comprises at least about 0.25 wt %, 0.5 wt %, or 1.0 wt % of pilocarpine or a pharmaceutically acceptable salt of pilocarpine for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt % of pilocarpine or a pharmaceutically acceptable salt of pilocarpine for treatment of presbyopia; and an ophthalmically acceptable carrier, wherein the ophthalmic agent is distributed with substantial uniformity throughout the ophthalmically acceptable carrier.

Provided herein is an ophthalmic composition comprising low concentrations of tropicamide or a pharmaceutically acceptable salt of tropicamide. In some embodiments, the ophthalmic composition comprises about 0.010 wt % to about 0.1 wt % or about 0.025 wt % to about 0.1 wt % tropicamide or a pharmaceutically acceptable salt of tropicamide for treatment of presbyopia; and an ophthalmically acceptable carrier, wherein the ophthalmic agent is distributed with substantial uniformity throughout the ophthalmically acceptable carrier. In some embodiments, the ophthalmic composition comprises at least about 0.02 wt %, 0.5 wt %, or 1.0 wt % for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt % for treatment of presbyopia; and an ophthalmically acceptable carrier, wherein the ophthalmic agent is distributed with substantial uniformity throughout the ophthalmically acceptable carrier.

Provided herein is an ophthalmic composition comprising low concentrations of aceclidine or a pharmaceutically acceptable salt of aceclidine and pilocarpine or a pharmaceutically acceptable salt of pilocarpine. In some embodiments, the ophthalmic composition comprises about 0.25 wt % to about 2.0% wt % of aceclidine or a pharmaceutically acceptable salt of aceclidine for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt % of aceclidine or a pharmaceutically acceptable salt of aceclidine for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.5 wt % to about 2.5 wt %, about 0.01 wt % to 0.45 wt %, or about 0.001 wt % to about 2 wt % of pilocarpine or a pharmaceutically acceptable salt of pilocarpine for treatment of presbyopia; and an ophthalmically acceptable carrier, wherein the ophthalmic agent is distributed with substantial uniformity throughout the ophthalmically acceptable carrier. In some embodiments, the ophthalmic composition comprises at least about 0.25 wt %, 0.5 wt %, or 1.0 wt % of pilocarpine or a pharmaceutically acceptable salt of pilocarpine for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt % of pilocarpine or a pharmaceutically acceptable salt of pilocarpine for treatment of presbyopia; and an ophthalmically acceptable carrier, wherein the ophthalmic agent is distributed with substantial uniformity throughout the ophthalmically acceptable carrier.

Provided herein is an ophthalmic composition comprising low concentrations of aceclidine or a pharmaceutically acceptable salt of aceclidine and tropicamide or a pharmaceutically acceptable salt of tropicamide. In some embodiments, the ophthalmic composition comprises about 0.25 wt % to about 2.0% wt % of aceclidine or a pharmaceutically acceptable salt of aceclidine for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt % of aceclidine or a pharmaceutically acceptable salt of aceclidine for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.010 wt % to about 0.1 wt % or about 0.025 wt % to about 0.1 wt % tropicamide or a pharmaceutically acceptable salt of tropicamide for treatment of presbyopia; and an ophthalmically acceptable carrier, wherein the ophthalmic agent is distributed with substantial uniformity throughout the ophthalmically acceptable carrier. In some embodiments, the ophthalmic composition comprises at least about 0.02 wt %, 0.5 wt %, or 1.0 wt % for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt % for treatment of presbyopia; and an ophthalmically acceptable carrier, wherein the ophthalmic agent is distributed with substantial uniformity throughout the ophthalmically acceptable carrier.

Provided herein is an ophthalmic composition comprising low concentrations of aceclidine or a pharmaceutically acceptable salt of aceclidine, pilocarpine or a pharmaceutically acceptable salt of pilocarpine, and tropicamide or a pharmaceutically acceptable salt of tropicamide. In some embodiments, the ophthalmic composition comprises about 0.25 wt % to about 2.0% wt % of aceclidine or a pharmaceutically acceptable salt of aceclidine for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt % of aceclidine or a pharmaceutically acceptable salt of aceclidine for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.5 wt % to about 2.5 wt %, about 0.01 wt % to 0.45 wt %, or about 0.001 wt % to about 2 wt % of pilocarpine or a pharmaceutically acceptable salt of pilocarpine for treatment of presbyopia; and an ophthalmically acceptable carrier, wherein the ophthalmic agent is distributed with substantial uniformity throughout the ophthalmically acceptable carrier. In some embodiments, the ophthalmic composition comprises at least about 0.25 wt %, 0.5 wt %, or 1.0 wt % of pilocarpine or a pharmaceutically acceptable salt of pilocarpine for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt % of pilocarpine or a pharmaceutically acceptable salt of pilocarpine for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.010 wt % to about 0.1 wt % or about 0.025 wt % to about 0.1 wt % tropicamide or a pharmaceutically acceptable salt of tropicamide for treatment of presbyopia; and an ophthalmically acceptable carrier, wherein the ophthalmic agent is distributed with substantial uniformity throughout the ophthalmically acceptable carrier. In some embodiments, the ophthalmic composition comprises at least about 0.02 wt %, 0.5 wt %, or 1.0 wt % for treatment of presbyopia; and an ophthalmically acceptable carrier, wherein the ophthalmic agent is distributed with substantial uniformity throughout the ophthalmically acceptable carrier. In some embodiments, the ophthalmic composition comprises about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt % for treatment of presbyopia; and an ophthalmically acceptable carrier, wherein the ophthalmic agent is distributed with substantial uniformity throughout the ophthalmically acceptable carrier.

In some embodiments of an ophthalmic composition described herein, the aceclidine or a pharmaceutically acceptable salt of aceclidine, pilocarpine or a pharmaceutically acceptable salt of pilocarpine, and tropicamide or a pharmaceutically acceptable salt of tropicamide is present in the ophthalmic composition at a concentration of one of: from about 0.001 wt % to about 0.40 wt %, from about 0.001 wt % to about 0.30 wt %, from about 0.001 wt % to about 0.20 wt %, from about 0.001 wt % to about 0.10 wt %, from about 0.001 wt % to about 0.09 wt %, from about 0.001 wt % to about 0.08 wt %, from about 0.001 wt % to about 0.07 wt %, from about 0.001 wt % to about 0.06 wt %, from about 0.001 wt % to about 0.05 wt %, from about 0.001 wt % to about 0.04 wt %, from about 0.001 wt % to about 0.03 wt %, from about 0.001 wt % to about 0.025 wt %, from about 0.001 wt % to about 0.02 wt %, from about 0.001 wt % to about 0.01 wt %, from about 0.001 wt % to about 0.008 wt %, or from about 0.001 wt % to about 0.005 wt %. In some embodiments of an ophthalmic composition described herein, the aceclidine or a pharmaceutically acceptable salt of aceclidine, pilocarpine or a pharmaceutically acceptable salt of pilocarpine, and tropicamide or a pharmaceutically acceptable salt of tropicamide is present in the ophthalmic composition at a concentration from about 0.001 wt % to about 0.10 wt %.

In some embodiments of an ophthalmic composition described herein, the aceclidine or a pharmaceutically acceptable salt of aceclidine, pilocarpine or a pharmaceutically acceptable salt of pilocarpine, and tropicamide or a pharmaceutically acceptable salt of tropicamide is present in the ophthalmic composition at a concentration of one of: from about 0.001 mg/g to about 0.40 mg/g, from about 0.001 mg/g to about 0.30 mg/g, from about 0.001 mg/g to about 0.20 mg/g, from about 0.001 mg/g to about 0.10 mg/g, from about 0.001 mg/g to about 0.09 mg/g, from about 0.01 mg/g to about 0.40 mg/g, from about 0.01 mg/g to about 0.30 mg/g, from about 0.01 mg/g to about 0.20 mg/g, from about 0.01 mg/g to about 0.10 mg/g, from about 0.01 mg/g to about 0.09 mg/g, from about 0.01 mg/g to about 0.08 mg/g, from about 0.01 mg/g to about 0.07 mg/g, from about 0.01 mg/g to about 0.06 mg/g, from about 0.01 mg/g to about 0.05 mg/g, from about 0.01 mg/g to about 0.04 mg/g, from about 0.01 mg/g to about 0.03 mg/g, from about 0.01 mg/g to about 0.025 mg/g, from about 0.01 mg/g to about 0.02 mg/g, from about 0.01 mg/g to about 0.1 mg/g, from about 0.01 mg/g to about 0.25 mg/g, from about 0.01 mg/g to about 0.5 mg/g, from about 0.01 mg/g to about 0.75 mg/g, from about 0.01 mg/g to about 1.0 mg/g. In some embodiments of an ophthalmic composition described herein, the aceclidine or a pharmaceutically acceptable salt of aceclidine, pilocarpine or a pharmaceutically acceptable salt of pilocarpine, and tropicamide or a pharmaceutically acceptable salt of tropicamide is present in the ophthalmic composition at a concentration from about 0.01 mg/g to about 0.5 mg/g.

In some embodiments of an ophthalmic composition described herein, the aceclidine or a pharmaceutically acceptable salt of aceclidine, pilocarpine or a pharmaceutically acceptable salt of pilocarpine, and tropicamide or a pharmaceutically acceptable salt of tropicamide is present in the ophthalmic composition at a concentration of one of: from about 0.0001 mg to about 0.040 mg, from about 0.0001 mg to about 0.030 mg, from about 0.0001 mg to about 0.020 mg, from about 0.0001 mg to about 0.010 mg, from about 0.0001 mg to about 0.009 mg, from about 0.001 mg to about 0.040 mg, from about 0.001 mg to about 0.030 mg, from about 0.001 mg to about 0.020 mg, from about 0.001 mg to about 0.010 mg, from about 0.001 mg to about 0.009 mg, from about 0.001 mg to about 0.008 mg, from about 0.001 mg to about 0.007 mg, from about 0.001 mg to about 0.006 mg, from about 0.001 mg to about 0.005 mg, from about 0.001 mg to about 0.004 mg, from about 0.001 mg to about 0.003 mg, from about 0.001 mg to about 0.0025 mg, from about 0.001 mg to about 0.002 mg, from about 0.001 mg to about 0.01 mg, from about 0.001 mg to about 0.025 mg, from about 0.001 mg to about 0.05 mg, from about 0.001 mg to about 0.075 mg, from about 0.001 mg to about 1.0 mg. In some embodiments of an ophthalmic composition described herein, the aceclidine or a pharmaceutically acceptable salt of aceclidine, pilocarpine or a pharmaceutically acceptable salt of pilocarpine, and tropicamide or a pharmaceutically acceptable salt of tropicamide is present in the ophthalmic composition at a concentration from about 0.0003 mg to about 0.025 mg or 0.001 mg to about 0.05 mg.

The present disclosure further recognizes the challenges present in formulation of certain compositions that contain low concentrations, especially very low concentrations, of ophthalmic agents, such as muscarinic agents (e.g. aceclidine, pilocarpine, tropicamide, or its pharmaceutically acceptable salts). In particular, pharmaceutical compositions with ophthalmic agents at such low concentrations are difficult to maintain dose-to-dose uniformity in term of ophthalmic agent content and/or distribution.

In some aspects, described herein are formulations or solutions of muscarinic agents (e.g., aceclidine, pilocarpine, tropicamide, or its pharmaceutically acceptable salts) formulated in deuterated water. In some aspects, formulations or solutions of muscarinic agents (e.g., aceclidine, pilocarpine, tropicamide, or its pharmaceutically acceptable salts) formulated in deuterated water are stable at different temperatures, at different relative humidity, with an acidic pD, and with a potency of at least 80% relative to the ophthalmic agent. In additional aspects, formulations or solutions of muscarinic agents (e.g., aceclidine, pilocarpine, tropicamide, or its pharmaceutically acceptable salts) formulated in deuterated water has a lowered buffering capacity. In such instances, the lowered buffering capacity of the ophthalmic formulations or solutions when administered into the eye allows the ophthalmic formulation or solution to reach physiological pH at a faster rate than compared to an equivalent ophthalmic formulation or solution formulated in H₂O.

In some aspects, described herein are formulations of muscarinic agents (e.g. aceclidine, pilocarpine, tropicamide, or its pharmaceutically acceptable salts) at low concentrations that do not have a significant dose-to-dose variation. In some aspects, described herein are formulations of muscarinic agents (e.g. aceclidine, pilocarpine, tropicamide, or its pharmaceutically acceptable salts) at low concentrations that are stable at different temperatures, at different relative humidity, with an acidic pD, and with a potency of at least 80% relative to the ophthalmic agent.

In other aspects, described herein include formulating the ophthalmic composition as an ophthalmic gel or an ophthalmic ointment. For example, some ophthalmic gel or an ophthalmic ointment described herein allows desirable dose-to-dose uniformity, reduced or limited systemic exposure, or combinations thereof.

### Ophthalmic Solution Compositions

Disclosed herein, in certain embodiments, is an ophthalmic composition formulated as an aqueous solution. In some embodiments, the ophthalmic composition comprises about 0.25 wt % to about 2.0% wt % of aceclidine or a pharmaceutically acceptable salt of aceclidine and deuterated water. In some embodiments, the ophthalmic composition comprises about 0.5 wt % to about 2.5 wt %, about 0.01 wt % to 0.45 wt %, or about 0.001 wt % to about 2 wt % of pilocarpine or a pharmaceutically acceptable salt of pilocarpine. In some embodiments, the ophthalmic composition comprises about 0.010 wt % to about 0.1 wt % or about 0.025 wt % to about 0.1 wt % tropicamide or a pharmaceutically acceptable salt of tropicamide. In some embodiments, the ophthalmic composition comprises about 0.25 wt % to about 2.0% wt % of aceclidine or a pharmaceutically acceptable salt of aceclidine, about 0.5 wt % to about 2.5 wt %, about 0.01 wt % to 0.45 wt %, or about 0.001 wt % to about 2 wt % of pilocarpine or a pharmaceutically acceptable salt of pilocarpine, and deuterated water. In some embodiments, the ophthalmic composition comprises about 0.25 wt % to about 2.0% wt % of aceclidine or a pharmaceutically acceptable salt of aceclidine, about 0.010 wt % to about 0.1 wt % or about 0.025 wt % to about 0.1 wt % tropicamide or a pharmaceutically acceptable salt of tropicamide, and deuterated water. In some embodiments, the ophthalmic composition comprises about 0.25 wt % to about 2.0% wt % of aceclidine or a pharmaceutically acceptable salt of aceclidine, about 0.5 wt % to about 2.5 wt %, about 0.01 wt % to 0.45 wt %, or about 0.001 wt % to about 2 wt % of pilocarpine or a pharmaceutically acceptable salt of pilocarpine, about 0.010 wt % to about 0.1 wt % or about 0.025 wt % to about 0.1 wt % tropicamide or a pharmaceutically acceptable salt of tropicamide, and deuterated water. As used herein, deuterated water refers to D₂O, DHO, heavy water, and/or deuterium oxide. DHO comprises a mixture of H₂O and D₂O.

In some embodiments, the composition comprises at least about 80% of the ophthalmic agent (e.g. muscarinic agent) for an extended period of time under storage condition. In some embodiments, the composition comprises at least about 80% of the ophthalmic agent (e.g. muscarinic agent) for an extended period of time under a storage condition. In some embodiments, the composition comprises at least about 81% of the ophthalmic agent (e.g. muscarinic agent) for an extended period of time under a storage condition. In some embodiments, the composition comprises at least about 82% of the ophthalmic agent (e.g. muscarinic agent) for an extended period of time under a storage condition. In some embodiments, the composition comprises at least about 83% of the ophthalmic agent (e.g. muscarinic agent) for an extended period of time under a storage condition. In some embodiments, the composition comprises at least about 84% of the ophthalmic agent (e.g. muscarinic agent) for an extended period of time under a storage condition. In some embodiments, the composition comprises at least about 85% of the ophthalmic agent (e.g. muscarinic agent) for an extended period of time under a storage condition. In some embodiments, the composition comprises at least about 86% of the ophthalmic agent (e.g. muscarinic agent) for an extended period of time under a storage condition. In some embodiments, the composition comprises at least about 87% of the ophthalmic agent (e.g. muscarinic agent) for an extended period of time under a storage condition. In some embodiments, the composition comprises at least about 88% of the ophthalmic agent (e.g. muscarinic agent) for an extended period of time under a storage condition. In some embodiments, the composition comprises at least about 89% of the ophthalmic agent (e.g. muscarinic agent) for an extended period of time under a storage condition. In some embodiments, the composition comprises at least about 90% of the ophthalmic agent (e.g. muscarinic agent) for an extended period of time under a storage condition. In some embodiments, the composition comprises at least about 91% of the ophthalmic agent (e.g. muscarinic agent) for an extended period of time under a storage condition. In some embodiments, the composition comprises at least about 92% of the ophthalmic agent (e.g. muscarinic agent) for an extended period of time under a storage condition. In some embodiments, the composition comprises at least about 93% of the ophthalmic agent (e.g. muscarinic agent) for an extended period of time under a storage condition. In some embodiments, the composition comprises at least about 94% of the ophthalmic agent (e.g. muscarinic agent) for an extended period of time under a storage condition. In some embodiments, the composition comprises at least about 95% of the ophthalmic agent (e.g. muscarinic agent) for an extended period of time under a storage condition. In some embodiments, the composition comprises at least about 96% of the ophthalmic agent (e.g. muscarinic agent) for an extended period of time under a storage condition. In some embodiments, the composition comprises at least about 97% of the ophthalmic agent (e.g. muscarinic agent) for an extended period of time under a storage condition. In some embodiments, the composition comprises at least about 98% of the ophthalmic agent (e.g. muscarinic agent) for an extended period of time under a storage condition. In some embodiments, the composition comprises at least about 99% of the ophthalmic agent (e.g. muscarinic agent) for an extended period of time under a storage condition. In some embodiments, the concentration of the ophthalmic agent (e.g. muscarinic agent) is based on an initial concentration after an extended period of time under a storage condition. In some embodiments, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the composition has a potency of at least about 80% after extended period of time under a storage condition. In some embodiments, the composition has a potency of at least about 81% after extended period of time under a storage condition. In some embodiments, the composition has a potency of at least about 82% after extended period of time under a storage condition. In some embodiments, the composition has a potency of at least about 83% after extended period of time under a storage condition. In some embodiments, the composition has a potency of at least about 84% after extended period of time under a storage condition. In some embodiments, the composition has a potency of at least about 85% after extended period of time under a storage condition. In some embodiments, the composition has a potency of at least about 86% after extended period of time under a storage condition. In some embodiments, the composition has a potency of at least about 87% after extended period of time under a storage condition. In some embodiments, the composition has a potency of at least about 88% after extended period of time under a storage condition. In some embodiments, the composition has a potency of at least about 89% after extended period of time under a storage condition. In some embodiments, the composition has a potency of at least 90% after extended period of time under a storage condition. In some embodiments, the composition has a potency of at least 91% after extended period of time under a storage condition. In some embodiments, the composition has a potency of at least 92% after extended period of time under a storage condition. In some embodiments, the composition has a potency of at least 93% after extended period of time under a storage condition. In some embodiments, the composition has a potency of at least 94% after extended period of time under a storage condition. In some embodiments, the composition has a potency of at least 95% after extended period of time under a storage condition. In some embodiments, the composition has a potency of at least 96% after extended period of time under a storage condition. In some embodiments, the composition has a potency of at least 97% after extended period of time under a storage condition. In some embodiments, the composition has a potency of at least 98% after extended period of time under a storage condition. In some embodiments, the composition has a potency of at least 99% after extended period of time under a storage condition.

In some embodiments, the extended period of time is at least 1 week. In some embodiments, the extended period of time is at least 2 weeks. In some embodiments, the extended period of time is at least 3 weeks. In some embodiments, the extended period of time is at least 1 month. In some embodiments, the extended period of time is at least 2 months. In some embodiments, the extended period of time is at least 3 months. In some embodiments, the extended period of time is at least 4 months. In some embodiments, the extended period of time is at least 5 months. In some embodiments, the extended period of time is at least 6 months. In some embodiments, the extended period of time is at least 7 months. In some embodiments, the extended period of time is at least 8 months. In some embodiments, the extended period of time is at least 9 months. In some embodiments, the extended period of time is at least 10 months. In some embodiments, the extended period of time is at least 11 months. In some embodiments, the extended period of time is at least 12 months (i.e. 1 year). In some embodiments, the extended period of time is at least 18 months (i.e. 1.5 years). In some embodiments, the extended period of time is at least 24 months (i.e. 2 years). In some embodiments, the extended period of time is at least 36 months (i.e. 3 years). In some embodiments, the extended period of time is at least 3 years. In some embodiments, the extended period of time is at least 5 years, or more.

In some embodiments, the temperature of the storage condition is between about 20° C. and about 70° C. In some embodiments, the temperature of the storage condition is between about 25° C. and about 65° C., about 30° C. and about 60° C., about 35° C. and about 55° C., or about 40° C. and about 50° C. In some embodiments, the temperature of the storage condition is between about 0° C. to about 30° C., 2° C. to about 10° C., or from about 16° C. to about 26° C. In some embodiments, the temperature of the storage condition is about 25° C. In some embodiments, the temperature of the storage condition is about 40° C. In some embodiments, the temperature of the storage condition is about 60° C.

In some embodiments, the relative humidity of the storage condition is between about 50% and about 80%, or between about 60% and about 75%. In some embodiments, the relative humidity of the storage condition is about 60%. In some embodiments, the relative humidity of the storage condition is about 75%.

In some embodiments, the composition comprises less than 60% of H₂O. In some embodiments, the composition comprises less than 55% of H₂O. In some embodiments, the composition comprises less than 50% of H₂O. In some embodiments, the composition comprises less than 45% of H₂O. In some embodiments, the composition comprises less than 40% of H₂O. In some embodiments, the composition comprises less than 35% of H₂O. In some embodiments, the composition comprises less than 30% of H₂O. In some embodiments, the composition comprises less than 25% of H₂O. In some embodiments, the composition comprises less than 20% of H₂O. In some embodiments, the composition comprises less than 15% of H₂O. In some embodiments, the composition comprises less than 10% of H₂O.

In some embodiments, the composition comprises from less than 5% of H₂O to 0% of H₂O. In some embodiments, the composition comprises less than 5% of H₂O. In some embodiments, the composition comprises less than 4.5% of H₂O. In some embodiments, the composition comprises less than 4% of H₂O. In some embodiments, the composition comprises less than 3.5% of H₂O. In some embodiments, the composition comprises less than 3% of H₂O. In some embodiments, the composition comprises less than 2.5% of H₂O. In some embodiments, the composition comprises less than 2% of H₂O. In some embodiments, the composition comprises less than 1.5% of H₂O. In some embodiments, the composition comprises less than 1% of H₂O. In some embodiments, the composition comprises less than 0.5% of H₂O. In some embodiments, the composition comprises less than 0.4% of H₂O. In some embodiments, the composition comprises less than 0.3% of H₂O. In some embodiments, the composition comprises less than 0.2% of H₂O. In some embodiments, the composition comprises less than 0.1% of H₂O. In some embodiments, the composition comprises 0% of H₂O.

In some embodiments, the composition has a pH of between about 4 and about 8, about 4.2 to about 7.9, about 4.5 and about 7.8, about 5 and about 7.5, or about 5.5 and about 7. In some embodiments, the composition has a pH of about 8.0. In some embodiments, the composition has a pH of about 7.9. In some embodiments, the composition has a pH of about 7.8. In some embodiments, the composition has a pH of about 7.7. In some embodiments, the composition has a pH of about 7.6. In some embodiments, the composition has a pH of less than about 7.5. In some embodiments, the composition has a pH of less than about 7.4. In some embodiments, the composition has a pH of less than about 7.3. In some embodiments, the composition has a pH of less than about 7.2. In some embodiments, the composition has a pH of less than about 7.1. In some embodiments, the composition has a pH of less than about 7. In some embodiments, the composition has a pH of less than about 6.9. In some embodiments, the composition has a pH of less than about 6.8. In some embodiments, the composition has a pH of less than about 6.7. In some embodiments, the composition has a pH of less than about 6.6. In some embodiments, the composition has a pH of less than about 6.5. In some embodiments, the composition has a pH of less than about 6.4. In some embodiments, the composition has a pH of less than about 6.3. In some embodiments, the composition has a pH of less than about 6.2. In some embodiments, the composition has a pH of less than about 6.1. In some embodiments, the composition has a pH of less than about 6. In some embodiments, the composition has a pH of less than about 5.9. In some embodiments, the composition has a pH of less than about 5.8. In some embodiments, the composition has a pH of less than about 5.7. In some embodiments, the composition has a pH of less than about 5.6. In some embodiments, the composition has a pH of less than about 5.5. In some embodiments, the composition has a pH of less than about 5.4. In some embodiments, the composition has a pH of less than about 5.3. In some embodiments, the composition has a pH of less than about 5.2. In some embodiments, the composition has a pH of less than about 5.1. In some embodiments, the composition has a pH of less than about 5. In some embodiments, the composition has a pH of less than about 4.9. In some embodiments, the composition has a pH of less than about 4.8. In some embodiments, the composition has a pH of less than about 4.7. In some embodiments, the composition has a pH of less than about 4.6. In some embodiments, the composition has a pH of less than about 4.5. In some embodiments, the composition has a pH of less than about 4.4. In some embodiments, the composition has a pH of less than about 4.3. In some embodiments, the composition has a pH of less than about 4.2. In some embodiments, the composition has a pH of less than about 4.1. In some embodiments, the composition has a pH of less than about 4.

In some embodiments, the composition has a pD of between about 4 and about 8, about 4.2 to about 7.9, about 4.5 and about 7.8, about 5 and about 7.5, or about 5.5 and about 7. In some embodiments, the composition has a pD of about 8.0. In some embodiments, the composition has a pD of about 7.9. In some embodiments, the composition has a pD of about 7.8. In some embodiments, the composition has a pD of about 7.7. In some embodiments, the composition has a pD of about 7.6. In some embodiments, the composition has a pD of less than about 7.5. In some embodiments, the composition has a pD of less than about 7.4. In some embodiments, the composition has a pD of less than about 7.3. In some embodiments, the composition has a pD of less than about 7.2. In some embodiments, the composition has a pD of less than about 7.1. In some embodiments, the composition has a pD of less than about 7. In some embodiments, the composition has a pD of less than about 6.9. In some embodiments, the composition has a pD of less than about 6.8. In some embodiments, the composition has a pD of less than about 6.7. In some embodiments, the composition has a pD of less than about 6.6. In some embodiments, the composition has a pD of less than about 6.5. In some embodiments, the composition has a pD of less than about 6.4. In some embodiments, the composition has a pD of less than about 6.3. In some embodiments, the composition has a pD of less than about 6.2. In some embodiments, the composition has a pD of less than about 6.1. In some embodiments, the composition has a pD of less than about 6. In some embodiments, the composition has a pD of less than about 5.9. In some embodiments, the composition has a pD of less than about 5.8. In some embodiments, the composition has a pD of less than about 5.7. In some embodiments, the composition has a pD of less than about 5.6. In some embodiments, the composition has a pD of less than about 5.5. In some embodiments, the composition has a pD of less than about 5.4. In some embodiments, the composition has a pD of less than about 5.3. In some embodiments, the composition has a pD of less than about 5.2. In some embodiments, the composition has a pD of less than about 5.1. In some embodiments, the composition has a pD of less than about 5. In some embodiments, the composition has a pD of less than about 4.9. In some embodiments, the composition has a pD of less than about 4.8. In some embodiments, the composition has a pD of less than about 4.7. In some embodiments, the composition has a pD of less than about 4.6. In some embodiments, the composition has a pD of less than about 4.5. In some embodiments, the composition has a pD of less than about 4.4. In some embodiments, the composition has a pD of less than about 4.3. In some embodiments, the composition has a pD of less than about 4.2. In some embodiments, the composition has a pD of less than about 4.1. In some embodiments, the composition has a pD of less than about 4.

In some embodiments, the composition comprising deuterated water has a lowered buffering capacity than an equivalent composition comprising H₂O. As described elsewhere herein, in some embodiments, the lowered buffering capacity allows the composition comprising deuterated water to normalize to physiological pH at a faster rate than a composition comprising H₂O. In some embodiments, the lowered buffering capacity allows the composition to induce less tear reflex than an equivalent composition comprising H₂O.

In some instances, the composition comprising deuterated water stabilizes muscarinic agent (e.g., aceclidine, pilocarpine, or tropicamide). In some embodiments, this is due to a lower concentration of the reactive species (e.g., —OD) in the D₂O/aqueous system compared to the concentration of the reactive species (e.g., —OH) in an equivalent purely aqueous system. In some embodiments, the ophthalmic composition formulated with deuterated water allows for a more stable ophthalmic composition relative to the ophthalmic composition formulated with H₂O.

In some embodiments, the composition has a potency of at least 80% at a temperature of about 0° C., about 2° C., about 5° C., about 10° C., about 15° C., about 25° C., about 40° C., or about 60° C. In some embodiments, the composition has a potency of at least 85% at a temperature of about 0° C., about 2° C., about 5° C., about 10° C., about 15° C., about 25° C., about 40° C., or about 60° C. In some embodiments, the composition has a potency of at least 90% at a temperature of about 0° C., about 2° C., about 5° C., about 10° C., about 15° C., about 25° C., about 40° C., or about 60° C. In some embodiments, the composition has a potency of at least 93% at a temperature of about 0° C., about 2° C., about 5° C., about 10° C., about 15° C., about 25° C., about 40° C., or about 60° C. In some embodiments, the composition has a potency of at least 95% at a temperature of about 0° C., about 2° C., about 5° C., about 10° C., about 15° C., about 25° C., about 40° C., or about 60° C. In some embodiments, the composition has a potency of at least 97% at a temperature of about 0° C., about 2° C., about 5° C., about 10° C., about 15° C., about 25° C., about 40° C., or about 60° C. In some embodiments, the composition has a potency of at least 98% at a temperature of about 0° C., about 2° C., about 5° C., about 10° C., about 15° C., about 25° C., about 40° C., or about 60° C. In some embodiments, the composition has a potency of at least 99% at a temperature of about 0° C., about 2° C., about 5° C., about 10° C., about 15° C., about 25° C., about 40° C., or about 60° C. In some embodiments, the composition has a potency of at least 80%, at least 85%, at least 90%, at least 93%, at least 95%, at least 97%, at least 98%, or at least 99% at a temperature of from about 0° C. to about 30° C., 2° C. to about 10° C. or from about 16° C. to about 26° C.

In some embodiments, the composition has a potency of at least 80% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition has a potency of at least 85% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition has a potency of at least 90% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition has a potency of at least 93% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition has a potency of at least 95% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition has a potency of at least 97% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition has a potency of at least 98% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition has a potency of at least 99% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months.

In some embodiments, the composition comprises less than 20% of major degradant based on the concentration of the ophthalmic agent after extended period of time under a storage condition. In some embodiments, the composition comprises less than 15% of major degradant based on the concentration of the ophthalmic agent after extended period of time under a storage condition. In some embodiments, the composition comprises less than 10% of major degradant based on the concentration of the ophthalmic agent after extended period of time under a storage condition. In some embodiments, the composition comprises less than 5% of major degradant based on the concentration of the ophthalmic agent after extended period of time under a storage condition. In some embodiments, the composition comprises less than 2.0% of major degradant based on the concentration of the ophthalmic agent after extended period of time under a storage condition. In some embodiments, the composition comprises less than 1.5% of major degradant based on the concentration of the ophthalmic agent after extended period of time under a storage condition. In some embodiments, the composition comprises less than 1.0% of major degradant based on the concentration of the ophthalmic agent after extended period of time under a storage condition. In some embodiments, the composition comprises less than 0.5% of major degradant based on the concentration of the ophthalmic agent after extended period of time under a storage condition. In some embodiments, the composition comprises less than 0.4% of major degradant based on the concentration of the ophthalmic agent after extended period of time under a storage condition. In some embodiments, the composition comprises less than 0.3% of major degradant based on the concentration of the ophthalmic agent after extended period of time under a storage condition. In some embodiments, the composition comprises less than 0.2% of major degradant based on the concentration of the ophthalmic agent after extended period of time under a storage condition. In some embodiments, the composition comprises less than 0.1% of major degradant based on the concentration of the ophthalmic agent after extended period of time under a storage condition. In some instances, a storage condition comprises a temperature of about 25° C., about 40° C., or about 60° C. In some embodiments, a storage condition comprises a temperature is between about 0° C. to about 30° C., 2° C. to about 10° C., or from about 16° C. to about 26° C. In some instances, the extended period of time is at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the major degradant is pilocarpic acid, isopilocarpic acid, isopilocarpine, or combinations thereof. In some embodiments, the major degradant is pilocarpic acid. In some embodiments, the major degradant is isopilocarpic acid. In some embodiments, the major degradant is isopilocarpine.

In some embodiments, the composition comprises less than 20% of major degradant based on the concentration of the ophthalmic agent at a temperature of about 25° C., about 40° C., or about 60° C. In some embodiments, the composition comprises less than 15% of major degradant based on the concentration of the ophthalmic agent at a temperature of about 25° C., about 40° C., or about 60° C. In some embodiments, the composition comprises less than 10% of major degradant based on the concentration of the ophthalmic agent at a temperature of about 25° C., about 40° C., or about 60° C. In some embodiments, the composition comprises less than 5% of major degradant based on the concentration of the ophthalmic agent at a temperature of about 25° C., about 40° C., or about 60° C. In some embodiments, the major degradant is pilocarpic acid, isopilocarpic acid, isopilocarpine, or combinations thereof. In some embodiments, the major degradant is pilocarpic acid. In some embodiments, the major degradant is isopilocarpic acid. In some embodiments, the major degradant is isopilocarpine.

In some embodiments, the composition comprises from less than 2.5% of major degradant to less than 0.1% of major degradant based on the concentration of the ophthalmic agent at a temperature of about 25° C., about 40° C., or about 60° C. In some embodiments, the composition comprises less than 2.5% of major degradant based on the concentration of the ophthalmic agent at a temperature of about 25° C., about 40° C., or about 60° C. In some embodiments, the composition comprises less than 2.0% of major degradant based on the concentration of the ophthalmic agent at a temperature of about 25° C., about 40° C., or about 60° C. In some embodiments, the composition comprises less than 1.5% of major degradant based on the concentration of the ophthalmic agent at a temperature of about 25° C., about 40° C., or about 60° C. In some embodiments, the composition comprises less than 1.0% of major degradant based on the concentration of the ophthalmic agent at a temperature of about 25° C., about 40° C., or about 60° C. In some embodiments, the composition comprises less than 0.5% of major degradant based on the concentration of the ophthalmic agent at a temperature of about 25° C., about 40° C., or about 60° C. In some embodiments, the composition comprises less than 0.4% of major degradant based on the concentration of the ophthalmic agent at a temperature of about 25° C., about 40° C., or about 60° C. In some embodiments, the composition comprises less than 0.3% of major degradant based on the concentration of the ophthalmic agent at a temperature of about 25° C., about 40° C., or about 60° C. In some embodiments, the composition comprises less than 0.2% of major degradant based on the concentration of the ophthalmic agent at a temperature of about 25° C., about 40° C., or about 60° C. In some embodiments, the composition comprises less than 0.1% of major degradant based on the concentration of the ophthalmic agent at a temperature of about 25° C., about 40° C., or about 60° C. In some embodiments, the major degradant is pilocarpic acid, isopilocarpic acid, isopilocarpine, or combinations thereof. In some embodiments, the major degradant is pilocarpic acid. In some embodiments, the major degradant is isopilocarpic acid. In some embodiments, the major degradant is isopilocarpine.

According to another aspect of the disclosure, described herein is an ophthalmic composition that comprises about 0.001 wt % to about 3 wt % pilocarpine or a pharmaceutically acceptable salt of pilocarpine and water, wherein the ophthalmic composition further comprises a buffering agent to provide a pH of about 4.2 to about 7.9. In some embodiments, the pilocarpine or a pharmaceutically acceptable salt of pilocarpine is present in the ophthalmic composition at a concentration of about 0.5 wt % to about 2.5 wt %, about 0.01 wt % to 0.45 wt %, or about 0.001 wt % to about 2 wt %. In some embodiments, the pilocarpine or the pharmaceutically acceptable salt of pilocarpine is present in the ophthalmic composition at a concentration of at least about 0.25 wt %, 0.5 wt %, or 1.0 wt %.

In some embodiments, an ophthalmic composition comprises about 0.25 wt % to about 2.0% wt % of aceclidine or a pharmaceutically acceptable salt of aceclidine and water, wherein the ophthalmic composition further comprises a buffering agent to provide a pH of about 4.2 to about 7.9.

In some embodiments, an ophthalmic composition comprises about 0.010 wt % to about 0.1 wt % tropicamide or a pharmaceutically acceptable salt of tropicamide and water, wherein the ophthalmic composition further comprises a buffering agent to provide a pH of about 4.2 to about 7.9. In some embodiments, the tropicamide or the pharmaceutically acceptable salt of tropicamide is present in the ophthalmic composition at a concentration about 0.025 wt % to about 0.1 wt %. In some embodiments, the tropicamide or the pharmaceutically acceptable salt of tropicamide is present in the ophthalmic composition at a concentration of at least about 0.02 wt %.

Described herein, in some embodiments, is an ophthalmic composition comprising pilocarpine or a pharmaceutically acceptable salt of pilocarpine, tropicamide or a pharmaceutically acceptable salt of tropicamide, and water, wherein the ophthalmic composition further comprises a buffering agent to provide a pH of about 4.2 to about 7.9. In some embodiments, the ophthalmic composition comprises about 0.25 wt % to about 2.0% wt % of aceclidine or a pharmaceutically acceptable salt of aceclidine. In some embodiments, the ophthalmic composition comprises about 0.001 wt % to about 3 wt % pilocarpine or a pharmaceutically acceptable salt of pilocarpine. In some embodiments, the pilocarpine or a pharmaceutically acceptable salt of pilocarpine is present in the ophthalmic composition at a concentration of about 0.5 wt % to about 2.5 wt %, about 0.01 wt % to 0.45 wt %, or about 0.001 wt % to about 2 wt %. In some embodiments, the pilocarpine or the pharmaceutically acceptable salt of pilocarpine is present in the ophthalmic composition at a concentration of at least about 0.25 wt %, 0.5 wt %, or 1.0 wt %. In some embodiments, an ophthalmic composition comprises about 0.010 wt % to about 0.1 wt % tropicamide or a pharmaceutically acceptable salt of tropicamide. In some embodiments, the tropicamide or the pharmaceutically acceptable salt of tropicamide is present in the ophthalmic composition at a concentration about 0.025 wt % to about 0.1 wt %. In some embodiments, the tropicamide or the pharmaceutically acceptable salt of tropicamide is present in the ophthalmic composition at a concentration of at least about 0.02 wt %.

Described herein, in some embodiments, is an ophthalmic composition comprising aceclidine or a pharmaceutically acceptable salt of aceclidine, pilocarpine or a pharmaceutically acceptable salt of pilocarpine, tropicamide or a pharmaceutically acceptable salt of tropicamide, and water, wherein the ophthalmic composition further comprises a buffering agent to provide a pH of about 4.2 to about 7.9. In some embodiments, the ophthalmic composition comprises about 0.001 wt % to about 3 wt % pilocarpine or a pharmaceutically acceptable salt of pilocarpine. In some embodiments, the pilocarpine or a pharmaceutically acceptable salt of pilocarpine is present in the ophthalmic composition at a concentration of about 0.5 wt % to about 2.5 wt %, about 0.01 wt % to 0.45 wt %, or about 0.001 wt % to about 2 wt %. In some embodiments, the pilocarpine or the pharmaceutically acceptable salt of pilocarpine is present in the ophthalmic composition at a concentration of at least about 0.25 wt %, 0.5 wt %, or 1.0 wt %. In some embodiments, an ophthalmic composition comprises about 0.010 wt % to about 0.1 wt % tropicamide or a pharmaceutically acceptable salt of tropicamide. In some embodiments, the tropicamide or the pharmaceutically acceptable salt of tropicamide is present in the ophthalmic composition at a concentration about 0.025 wt % to about 0.1 wt %. In some embodiments, the tropicamide or the pharmaceutically acceptable salt of tropicamide is present in the ophthalmic composition at a concentration of at least about 0.02 wt %.

In some instances, the ophthalmic composition comprises one of: at least about 80%, at least about 85%, at least about 90%, at least about 93%, at least about 95%, at least about 97%, at least about 98%, or at least about 99% of the aceclidine or a pharmaceutically acceptable salt of aceclidine based on initial concentration after extended period of time under storage condition. In some instances, the ophthalmic composition comprises one of: at least about 80%, at least about 85%, at least about 90%, at least about 93%, at least about 95%, at least about 97%, at least about 98%, or at least about 99% of the pilocarpine or a pharmaceutically acceptable salt of pilocarpine based on initial concentration after extended period of time under storage condition. In some instances, the ophthalmic composition comprises one of: at least about 80%, at least about 85%, at least about 90%, at least about 93%, at least about 95%, at least about 97%, at least about 98%, or at least about 99% of the tropicamide or a pharmaceutically acceptable salt of tropicamide based on initial concentration after extended period of time under storage condition.

According to another aspect of the disclosure, described herein is an ophthalmic composition comprising one or more ophthalmic agents. In some instances, the one or more ophthalmic agents is aceclidine or a pharmaceutically acceptable salt of aceclidine. In some instances, the one or more ophthalmic agents is pilocarpine or a pharmaceutically acceptable salt of pilocarpine. In some instances, the one or more ophthalmic agents is tropicamide or a pharmaceutically acceptable salt of tropicamide. In some instances, the ophthalmic composition comprises aceclidine or a pharmaceutically acceptable salt of aceclidine and pilocarpine or a pharmaceutically acceptable salt of pilocarpine. In some instances, the ophthalmic composition comprises aceclidine or a pharmaceutically acceptable salt of aceclidine and tropicamide or a pharmaceutically acceptable salt of tropicamide. In some instances, the ophthalmic composition comprises aceclidine or a pharmaceutically acceptable salt of aceclidine, pilocarpine or a pharmaceutically acceptable salt of pilocarpine, and tropicamide or a pharmaceutically acceptable salt of tropicamide.

In some instances, an ophthalmic composition comprises an ophthalmic agent, wherein the ophthalmic agent is aflibercept, ranibizumab, pegaptanib, cyclopentolate, phenylephrine, homatropine, scopolamine, cyclopentolate/phenylephrine, phenylephrine/scopolamine, tropicamide, ketorolac/phenylephrine, hydroxyamphetamine/tropicamide, cysteamine, ocriplasmin, mitomycin, dapiprazole, lidocaine, proparacaine, tetracaine, benoxinate, azithromycin, bacitracin, besifloxacin, boric acid, chloramphenicol, ciprofloxacin, erythromycin, ganciclovir, gatifloxacin, gentamicin, idoxuridine, levofloxacin, moxifloxacin, natamycin, norfloxacin, ofloxacin, bacitracin/polymyxin b, tobramycin, polymyxin b/trimethoprim, povidone iodine, trifluridine, gramicidin/neomycin/polymyxin b, sulfacetamide sodium, sulfisoxazole, bacitracin/neomycin/polymyxin b, oxytetracycline/polymyxin b, phenylephrine/sulfacetamide sodium, vidarabine, bromfenac, nepafenac, ketorolac, cyclosporine, flurbiprofen, suprofen, diclofenac, alcaftadine, azelastine, bepotastine, cromolyn, emedastine, epinastine, ketotifen, levocabastine, lodoxamide, nedocromil, naphazoline, naphazoline/pheniramine, naphazoline/zinc sulfate, olopatadine, oxymetazoline, pemirolast, phenylephrine/zinc sulfate, tetrahydrozoline, tetrahydrozoline/zinc sulfate, fluorescein, fluorescein/proparacaine, benoxinate/fluorescein, indocyanine green, trypan blue, acetylcholine, apraclonidine, betaxolol, bimatoprost, brimonidine, brinzolamide, brimonidine/brinzolamide, carbachol, carteolol, demecarium bromide, dipivefrin, dorzolamide, dorzolamide/timolol, echothiophate iodide, epinephrine, epinephrine/pilocarpine, latanoprost, levobunolol, levobetaxolol, metipranolol, physostigmine, pilocarpine, tafluprost, timolol, travoprost, unoprostone, artificial tear, dexamethasone, difluprednate, fluocinolone, fluorometholone, loteprednol, medrysone, prednisolone, rimexolone, triamcinolone, fluorometholone/sulfacetamide sodium, dexamethasone/neomycin, dexamethasone/tobramycin, dexamethasone/neomycin/polymyxin b, loteprednol/tobramycin, prednisolone/sulfacetamide sodium, bacitracin/hydrocortisone/neomycin/polymyxin b, hydrocortisone/neomycin/polymyxin b, chloramphenicol/hydrocortisone/polymyxin b, neomycin/polymyxin b/prednisolone, gentamicin/prednisolone, ketorolac/phenylephrine, diphenhydramine, dimenhydrinate, dicyclomine, flavoxate, oxybutynin, tiotropium, hyoscine, scopolamine (L-hyoscine), hydroxyzine, ipratropium, pirenzepine, solifenacin, darifenacin, benzatropine, mebeverine, procyclidine, aclidinium bromide, trihexyphenidyl/benzhexol, tolterodine, aceclidine, anisodamine, or any combinations thereof. In some embodiments, the ophthalmic agent is aceclidine, tropicamide, pilocarpine, or combinations thereof.

In some instances, an ophthalmic composition comprises an ophthalmic agent, wherein the ophthalmic agent is a miotic agent. In some instances, the miotic agent is dapiprazole, thymoxamine, brimonidine, nicotine, apraclonidin, phentolamine, pharmaceutically acceptable salts thereof, or combinations thereof.

In some instances, an ophthalmic composition comprises an ophthalmic agent, wherein the ophthalmic agent is a muscarinic receptor agonist, muscarinic receptor antagonist, an alpha-1 adrenergic receptor antagonist, an alpha-2 adrenergic receptor agonist, a beta-adrenergic receptor antagonist, a nicotine receptor agonist, an antipsychotic, an antiemetic, a cannabinoid, a monoamine oxidase (MAO) inhibitor, an EP1 receptor agonist, an EP4 receptor agonist, an FP receptor agonist, a calcium channel modulator, an anticholinergic agent, or combinations thereof.

In some instances, the ophthalmic composition has a pH of one of: less than about 7.3, less than about 7.2, less than about 7.1, less than about 7, less than about 6.8, less than about 6.5, less than about 6.4, less than about 6.3, less than about 6.2, less than about 6.1, less than about 6, less than about 5.9, less than about 5.8, less than about 5.2, less than about 4.8, or less than about 4.2 after extended period of time under storage condition.

In some instances, the ophthalmic composition further has a potency of one of: at least 80%, at least 85%, at least 90%, at least 93%, at least 95%, at least 97%, at least 98%, or at least 99% after extended period of time under storage condition.

In some instances, the extended period of time is one of: about 1 week, about 2 weeks, about 3 weeks, about 1 month, about 2 months, about 3 months, about 4 months, about 5 months, about 6 months, about 8 months, about 10 months, about 12 months, about 18 months, about 24 months, about 36 months, about 4 years, or about 5 years.

In some instances, the storage condition has a storage temperature of one of: about 25° C., about 40° C., or about 60° C. In some cases, the storage condition has a storage temperature of from about 2° C. to about 10° C., or from about 16° C. to about 26° C. In some cases, the storage condition has a relative humidity of about 60% or about 75%.

In some instances, the ophthalmic composition is in the form of an aqueous solution. In some cases, the muscarinic agent is present in the composition at a concentration of one of: about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %. In some cases, the muscarinic agent is present in the composition at a concentration of about 0.25 wt % to about 2.0% wt %, about 0.5 wt % to about 2.5 wt %, about 0.01 wt % to 0.45 wt %, about 0.001 wt % to about 2 wt %, about 0.010 wt % to about 0.1 wt %, or about 0.025 wt % to about 0.1 wt %. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some instances, the ophthalmic composition further comprises an osmolarity adjusting agent. In some cases, the osmolarity adjusting agent is sodium chloride.

In some instances, the ophthalmic composition further comprises a preservative. In some cases, the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof.

Described herein, in some embodiments, is an ophthalmic composition substantially free of a preservative. In some instances, composition substantially free of benzalkonium chloride preservative. In some instances, the composition has no detectable amount of a benzalkonium chloride preservative. In some instances, the composition has no detectable amount of a benzalkonium chloride. In some instances, the composition is substantially free of a preservative selected from cetrimonium, sodium perborate, stabilized oxychloro complex, SofZia, polyquaternium-1, chlorobutanol, edetate disodium, polyhexamethylene biguanide, or combinations thereof. In some instances, the composition has no detectable amount of a preservative. In some instances, the composition is substantially free of any preservative. In some instances, the ophthalmic composition further comprises a buffer agent. In some cases, the buffer agent is selected from borates, borate-polyol complexes, phosphate buffering agents, citrate buffering agents, acetate buffering agents, carbonate buffering agents, organic buffering agents, amino acid buffering agents, or combinations thereof.

In some instances, the ophthalmic composition further comprises a tonicity adjusting agent. In some cases, the tonicity adjusting agent is selected from sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, dextrose, mannitol, sorbitol, dextrose, sucrose, urea, propylene glycol, glycerin, or a combination thereof.

In some instances, the ophthalmic composition further comprises a penetration agent. In some instances, the penetration agent is benzalkonium chloride.

In some instances, the ophthalmic composition is stored in a plastic container. In some cases, the material of the plastic container comprises low-density polyethylene (LDPE).

In some instances, the ophthalmic composition has a dose-to-dose muscarinic agent concentration variation of one of: less than 50%, less than 40%, less than 30%, less than 20%, less than 10%, or less than 5%. In some cases, the dose-to-dose muscarinic agent concentration variation is based on one of: 10 consecutive doses, 8 consecutive doses, 5 consecutive doses, 3 consecutive doses, or 2 consecutive doses.

In some instances, the ophthalmic composition has a pH of one of: from about 3.8 to about 7.5, from about 4.2 to about 7.5, from about 4.8 to about 7.3, from about 5.2 to about 7.2, from about 5.8 to about 7.1, from about 6.0 to about 7.0, or from about 6.2 to about 6.8.

In some instances, the ophthalmic composition further comprises a pH adjusting agent. I In some embodiments of an ophthalmic composition described herein, the pH adjusting agent comprises DCl, HCl, NaOH, NaOD, CD 3 COOD, C₆D₈O₇, CH₃COOH, C₆H₈O₇, or combinations thereof.

In some instances, the ophthalmic composition comprises one of: less than 5% of D₂O, less than 4% of D₂O, less than 3% of D₂O, less than 2% of D₂O, less than 1% of D₂O, less than 0.5% of D₂O, less than 0.1% of D₂O, or 0% D₂O. In some cases, the ophthalmic composition is essentially free of D₂O.

In some instances, the ophthalmic composition further comprises a pharmaceutically acceptable carrier.

In some instances, the ophthalmic composition is formulated as an ophthalmic solution for treatment of an ophthalmic disorder. In some cases, the ophthalmic disorder or condition is presbyopia.

In some instances, the ophthalmic composition is not formulated as an injectable formulation.

Ophthalmic Agent Concentration

In some embodiments, the compositions described herein have a concentration of ophthalmic agent between about 0.001% to about 2.5%, between about 0.005% to about 2.5%, between about 0.010% to about 2.5%, between about 0.015% to about 2.5%, between about 0.020% to about 2.5%, between about 0.025% to about 2.5%, between about 0.030% to about 2.5%, between about 0.040% to about 2.5%, between about 0.045% to about 2.5%, between about 0.05% to about 2.5%, between about 0.060% to about 2.5%, between about 0.07% to about 2.5%, between about 0.08% to about 2.5%, between about 0.090% to about 2.5%, between about 0.1% to about 2.5%, between about 0.2% to about 2.5%, between about 0.3% to about 2.5%, between about 0.4% to about 2.5%, between about 0.5% to about 2.5%, between about 0.6% to about 2.5%, between about 0.7% to about 2.5%, between about 0.8% to about 2.5%, between about 0.9% to about 2.5%, between about 1.0% to about 2.5%, or between about 1.5% to about 2.5% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 2.0%, between about 0.005% to about 2.0%, between about 0.010% to about 2.0%, between about 0.015% to about 2.0%, between about 0.020% to about 2.0%, between about 0.025% to about 2.0%, between about 0.030% to about 2.0%, between about 0.040% to about 2.0%, between about 0.045% to about 2.0%, between about 0.05% to about 2.0%, between about 0.060% to about 2.0%, between about 0.07% to about 2.0%, between about 0.08% to about 2.0%, between about 0.090% to about 2.0%, between about 0.1% to about 2.0%, between about 0.2% to about 2.0%, between about 0.3% to about 2.0%, between about 0.4% to about 2.0%, between about 0.5% to about 2.0%, between about 0.6% to about 2.0%, between about 0.7% to about 2.0%, between about 0.8% to about 2.0%, between about 0.9% to about 2.0%, between about 1.0% to about 2.0%, or between about 1.5% to about 2.0% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 1.5%, between about 0.005% to about 1.5%, between about 0.010% to about 1.5%, between about 0.015% to about 1.5%, between about 0.020% to about 1.5%, between about 0.025% to about 1.5%, between about 0.030% to about 1.5%, between about 0.040% to about 1.5%, between about 0.045% to about 1.5%, between about 0.05% to about 1.5%, between about 0.060% to about 1.5%, between about 0.07% to about 1.5%, between about 0.08% to about 1.5%, between about 0.090% to about 1.5%, between about 0.1% to about 1.5%, between about 0.2% to about 1.5%, between about 0.3% to about 1.5%, between about 0.4% to about 1.5%, between about 0.5% to about 1.5%, between about 0.6% to about 1.5%, between about 0.7% to about 1.5%, between about 0.8% to about 1.5%, between about 0.9% to about 1.5%, or between about 1.0% to about 1.5% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 1.0%, between about 0.005% to about 1.0%, between about 0.010% to about 1.0%, between about 0.015% to about 1.0%, between about 0.020% to about 1.0%, between about 0.025% to about 1.0%, between about 0.030% to about 1.0%, between about 0.040% to about 1.0%, between about 0.045% to about 1.0%, between about 0.05% to about 1.0%, between about 0.060% to about 1.0%, between about 0.07% to about 1.0%, between about 0.08% to about 1.0%, between about 0.090% to about 1.0%, between about 0.1% to about 1.0%, between about 0.2% to about 1.0%, between about 0.3% to about 1.0%, between about 0.4% to about 1.0%, between about 0.5% to about 1.0%, between about 0.6% to about 1.0%, between about 0.7% to about 1.0%, between about 0.8% to about 1.0%, or between about 0.9% to about 1.0% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.9%, between about 0.005% to about 0.9%, between about 0.010% to about 0.9%, between about 0.015% to about 0.9%, between about 0.020% to about 0.9%, between about 0.025% to about 0.9%, between about 0.030% to about 0.9%, between about 0.040% to about 0.9%, between about 0.045% to about 0.9%, between about 0.05% to about 0.9%, between about 0.060% to about 0.9%, between about 0.07% to about 0.9%, between about 0.08% to about 0.9%, between about 0.090% to about 0.9%, between about 0.1% to about 0.9%, between about 0.2% to about 0.9%, between about 0.3% to about 0.9%, between about 0.4% to about 0.9%, between about 0.5% to about 0.9%, between about 0.6% to about 0.9%, between about 0.7% to about 0.9%, or between about 0.8% to about 0.9% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.8%, between about 0.005% to about 0.8%, between about 0.010% to about 0.8%, between about 0.015% to about 0.8%, between about 0.020% to about 0.8%, between about 0.025% to about 0.8%, between about 0.030% to about 0.8%, between about 0.040% to about 0.8%, between about 0.045% to about 0.8%, between about 0.05% to about 0.8%, between about 0.060% to about 0.8%, between about 0.07% to about 0.8%, between about 0.08% to about 0.8%, between about 0.090% to about 0.8%, between about 0.1% to about 0.8%, between about 0.2% to about 0.8%, between about 0.3% to about 0.8%, between about 0.4% to about 0.8%, between about 0.5% to about 0.8%, between about 0.6% to about 0.8%, or between about 0.7% to about 0.8% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.7%, between about 0.005% to about 0.7%, between about 0.010% to about 0.7%, between about 0.015% to about 0.7%, between about 0.020% to about 0.7%, between about 0.025% to about 0.7%, between about 0.030% to about 0.7%, between about 0.040% to about 0.7%, between about 0.045% to about 0.7%, between about 0.05% to about 0.7%, between about 0.060% to about 0.7%, between about 0.07% to about 0.7%, between about 0.08% to about 0.7%, between about 0.090% to about 0.7%, between about 0.1% to about 0.7%, between about 0.2% to about 0.7%, between about 0.3% to about 0.7%, between about 0.4% to about 0.7%, between about 0.5% to about 0.7%, or between about 0.6% to about 0.7% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.6%, between about 0.005% to about 0.6%, between about 0.010% to about 0.6%, between about 0.015% to about 0.6%, between about 0.020% to about 0.6%, between about 0.025% to about 0.6%, between about 0.030% to about 0.6%, between about 0.040% to about 0.6%, between about 0.045% to about 0.6%, between about 0.05% to about 0.6%, between about 0.060% to about 0.6%, between about 0.07% to about 0.6%, between about 0.08% to about 0.6%, between about 0.090% to about 0.6%, between about 0.1% to about 0.6%, between about 0.2% to about 0.6%, between about 0.3% to about 0.6%, between about 0.4% to about 0.6%, or between about 0.5% to about 0.6% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.5%, between about 0.005% to about 0.5%, between about 0.010% to about 0.5%, between about 0.015% to about 0.5%, between about 0.020% to about 0.5%, between about 0.025% to about 0.5%, between about 0.030% to about 0.5%, between about 0.040% to about 0.5%, between about 0.045% to about 0.5%, between about 0.05% to about 0.5%, between about 0.060% to about 0.5%, between about 0.07% to about 0.5%, between about 0.08% to about 0.5%, between about 0.090% to about 0.5%, between about 0.1% to about 0.5%, between about 0.2% to about 0.5%, between about 0.3% to about 0.5%, or between about 0.4% to about 0.5% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.4%, between about 0.005% to about 0.4%, between about 0.010% to about 0.4%, between about 0.015% to about 0.4%, between about 0.020% to about 0.4%, between about 0.025% to about 0.4%, between about 0.030% to about 0.4%, between about 0.040% to about 0.4%, between about 0.045% to about 0.4%, between about 0.05% to about 0.4%, between about 0.060% to about 0.4%, between about 0.07% to about 0.4%, between about 0.08% to about 0.4%, between about 0.090% to about 0.4%, between about 0.1% to about 0.4%, between about 0.2% to about 0.4%, or between about 0.3% to about 0.4% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.3%, between about 0.005% to about 0.3%, between about 0.010% to about 0.3%, between about 0.015% to about 0.3%, between about 0.020% to about 0.3%, between about 0.025% to about 0.3%, between about 0.030% to about 0.3%, between about 0.040% to about 0.3%, between about 0.045% to about 0.3%, between about 0.05% to about 0.3%, between about 0.060% to about 0.3%, between about 0.07% to about 0.3%, between about 0.08% to about 0.3%, between about 0.090% to about 0.3%, between about 0.1% to about 0.3%, or between about 0.2% to about 0.3% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.2%, between about 0.005% to about 0.2%, between about 0.010% to about 0.2%, between about 0.015% to about 0.2%, between about 0.020% to about 0.2%, between about 0.025% to about 0.2%, between about 0.030% to about 0.2%, between about 0.040% to about 0.2%, between about 0.045% to about 0.2%, between about 0.05% to about 0.2%, between about 0.060% to about 0.2%, between about 0.07% to about 0.2%, between about 0.08% to about 0.2%, between about 0.090% to about 0.2%, or between about 0.1% to about 0.2% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.1%, between about 0.005% to about 0.1%, between about 0.010% to about 0.1%, between about 0.015% to about 0.1%, between about 0.020% to about 0.1%, between about 0.025% to about 0.1%, between about 0.030% to about 0.1%, between about 0.040% to about 0.1%, between about 0.045% to about 0.1%, between about 0.05% to about 0.1%, between about 0.060% to about 0.1%, between about 0.07% to about 0.1%, between about 0.08% to about 0.1%, or between about 0.090% to about 0.1% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.09%, between about 0.005% to about 0.09%, between about 0.010% to about 0.09%, between about 0.015% to about 0.09%, between about 0.020% to about 0.09%, between about 0.025% to about 0.09%, between about 0.030% to about 0.09%, between about 0.040% to about 0.09%, between about 0.045% to about 0.09%, between about 0.05% to about 0.09%, between about 0.060% to about 0.09%, between about 0.07% to about 0.09%, or between about 0.08% to about 0.09% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.08%, between about 0.005% to about 0.08%, between about 0.010% to about 0.08%, between about 0.015% to about 0.08%, between about 0.020% to about 0.08%, between about 0.025% to about 0.08%, between about 0.030% to about 0.08%, between about 0.040% to about 0.08%, between about 0.045% to about 0.08%, between about 0.05% to about 0.08%, between about 0.060% to about 0.08%, or between about 0.07% to about 0.08% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.07%, between about 0.005% to about 0.07%, between about 0.010% to about 0.07%, between about 0.015% to about 0.07%, between about 0.020% to about 0.07%, between about 0.025% to about 0.07%, between about 0.030% to about 0.07%, between about 0.040% to about 0.07%, between about 0.045% to about 0.07%, between about 0.05% to about 0.07%, or between about 0.060% to about 0.07% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.06%, between about 0.005% to about 0.06%, between about 0.010% to about 0.06%, between about 0.015% to about 0.06%, between about 0.020% to about 0.06%, between about 0.025% to about 0.06%, between about 0.030% to about 0.06%, between about 0.040% to about 0.06%, between about 0.045% to about 0.06%, or between about 0.05% to about 0.06% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.050%, between about 0.005% to about 0.050%, between about 0.010% to about 0.050%, between about 0.015% to about 0.050%, between about 0.020% to about 0.050%, between about 0.025% to about 0.050%, between about 0.030% to about 0.050%, between about 0.035% to about 0.050%, between about 0.040% to about 0.050%, or between about 0.045% to about 0.050% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.045%, between about 0.005% to about 0.045%, between about 0.010% to about 0.045%, between about 0.015% to about 0.045%, between about 0.020% to about 0.045%, between about 0.025% to about 0.045%, between about 0.030% to about 0.045%, between about 0.035% to about 0.045%, or between about 0.040% to about 0.045% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.040%, between about 0.005% to about 0.040%, between about 0.010% to about 0.040%, between about 0.015% to about 0.040%, between about 0.020% to about 0.040%, between about 0.025% to about 0.040%, between about 0.030% to about 0.040%, between about 0.035% to about 0.040% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.035%, between about 0.005% to about 0.035%, between about 0.010% to about 0.035%, between about 0.015% to about 0.035%, between about 0.020% to about 0.035%, between about 0.025% to about 0.035%, or between about 0.030% to about 0.035% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.030%, between about 0.005% to about 0.030%, between about 0.010% to about 0.030%, between about 0.015% to about 0.030%, between about 0.020% to about 0.030%, or between about 0.025% to about 0.030% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.025%, between about 0.005% to about 0.025%, between about 0.010% to about 0.025%, between about 0.015% to about 0.025%, or between about 0.020% to about 0.025% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.020%, between about 0.005% to about 0.020%, between about 0.010% to about 0.020%, or between about 0.015% to about 0.020% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.015%, between about 0.005% to about 0.015%, or between about 0.010% to about 0.015% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent between about 0.001% to about 0.010%, between about 0.005% to about 0.010%, or between about 0.008% to about 0.010% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some embodiments, the compositions described herein have a concentration of ophthalmic agent at least about 0.001%, 0.005%, 0.010%, 0.015%, 0.020%, 0.025%, 0.030%, 0.035%, 0.040%, 0.045%, 0.050%, 0.055%, 0.060%, 0.065%, 0.070%, 0.075%, 0.080%, 0.085%, 0.090%, 0.095%, 0.1%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.0%, 1.5%, 2.0%, 2.5%, 3.0%, 3.5%, or 4.0% of the ophthalmic agent, or pharmaceutically acceptable prodrug or salt thereof, by weight of the composition. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some instances, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

Without wishing to be bound by any particular theory, it is contemplated herein that the low concentration of the ophthalmic agent (e.g. muscarinic agent such as aceclidine, pilocarpine, or tropicamide) in the disclosed ophthalmic composition provides sufficient and consistent therapeutic benefits to an individual in need thereof, while reducing or avoiding the ocular side effects including glare from pupillary dilation and blurred vision due to loss of accommodation that are associated with ophthalmic formulations containing higher concentrations of the ophthalmic agent (e e.g. muscarinic agent such as aceclidine, pilocarpine, or tropicamide).

Solution Stability

In some embodiments, the composition described herein comprises a buffering agent. In some embodiments, a buffering agent is selected from borates, borate-polyol complexes, phosphate buffering agents, citrate buffering agents, acetate buffering agents, carbonate buffering agents, organic buffering agents, amino acid buffering agents, or combinations thereof. In some embodiments, the composition described herein comprises buffer comprising deuterated water. In some embodiments, a deuterated buffer is selected from borates, borate-polyol complexes, phosphate buffering agents, citrate buffering agents, acetate buffering agents, carbonate buffering agents, organic buffering agents, amino acid buffering agents, or combinations thereof, formulated in deuterated water.

In some instances, borates include boric acid, salts of boric acid, other pharmaceutically acceptable borates, and combinations thereof. In some cases, borates include boric acid, sodium borate, potassium borate, calcium borate, magnesium borate, manganese borate, and other such borate salts.

As used herein, the term polyol comprises any compound having at least one hydroxyl group on each of two adjacent carbon atoms that are not in trans configuration relative to each other. In some embodiments, the polyols is linear or cyclic, substituted or unsubstituted, or mixtures thereof, so long as the resultant complex is water soluble and pharmaceutically acceptable. In some instances, examples of polyol include: sugars, sugar alcohols, sugar acids and uronic acids. In some cases, polyols include, but are not limited to: mannitol, glycerin, xylitol and sorbitol.

In some embodiments, phosphate buffering agents include phosphoric acid; alkali metal phosphates such as disodium hydrogen phosphate, sodium dihydrogen phosphate, trisodium phosphate, dipotassium hydrogen phosphate, potassium dihydrogen phosphate, and tripotassium phosphate; alkaline earth metal phosphates such as calcium phosphate, calcium hydrogen phosphate, calcium dihydrogen phosphate, monomagnesium phosphate, dimagnesium phosphate (magnesium hydrogen phosphate), and trimagnesium phosphate; ammonium phosphates such as diammonium hydrogen phosphate and ammonium dihydrogen phosphate; or a combination thereof. In some instances, the phosphate buffering agent is an anhydride. In some instances, the phosphate buffering agent is a hydrate.

In some embodiments, borate-polyol complexes include those described in U.S. Pat. No. 6,503,497. In some instances, the borate-polyol complexes comprise borates in an amount of from about 0.01 to about 2.0% w/v, and one or more polyols in an amount of from about 0.01% to about 5.0% w/v.

In some instances, the buffering agent has a concentration of at least about 5 mM, 10 mM, 20 mM, 30 mM, 40 mM, 50 mM, 60 mM, 80 mM, 90 mM, or 100 mM. In some instances, the buffering agent has a concentration of no more than about 75 mM. In some instances, the buffering agent has a concentration in a range of about 5 mM to about 100 mM, about 10 mM to about 90 mM, about 20 mM to about 80 mM, or about 40 mM to about 60 mM.

In some cases, citrate buffering agents include citric acid and sodium citrate. In some instances, the citrate buffering agent comprises citrate. In some instances, the citrate is present in the ophthalmic composition at a concentration of between about 0.001% to about 0.20%, between about 0.004% to about 0.20%, between about 0.005% to about 0.20%, between about 0.010% to about 0.20%, between about 0.015% to about 0.20%, between about 0.020% to about 0.20%, between about 0.025% to about 0.20%, between about 0.030% to about 0.20%, between about 0.035% to about 0.20%, between about 0.040% to about 0.20%, or between about 0.045% to about 0.20% by weight of the composition. In some instances, the citrate is present in the ophthalmic composition at a concentration of at least or about 0.001%, 0.002%, 0.003%, 0.004%, 0.005%, 0.006%, 0.007%, 0.008%, 0.009%, 0.010%, 0.020%, 0.030%, 0.040%, 0.050%, 0.060%, 0.070%, 0.080%, 0.090%, 0.10%, 0.20%, 0.30%, or 0.40% by weight of the composition.

In some instances, acetate buffering agents include acetic acid, potassium acetate, and sodium acetate.

In some instances, carbonate buffering agents include sodium bicarbonate and sodium carbonate.

In some cases, organic buffering agents include Good's Buffer, such as for example 2-(N-morpholino)ethanesulfonic acid (IVIES), N-(2-Acetamido)iminodiacetic acid, N-(Carbamoylmethyl)iminodiacetic acid (ADA), piperazine-N,N′-bis(2-ethanesulfonic acid (PIPES), N-(2-acetamido)-2-aminoethanesulfonic acid (ACES), β-Hydroxy-4-morpholinepropanesulfonic acid, 3-Morpholino-2-hydroxypropanesulfonic acid (MOPSO), cholamine chloride, 3-(N-morpholino)propansulfonic acid (MOPS), N,N-bis(2-hydroxyethyl)-2-aminoethanesulfonic acid (BES), 2-[(2-Hydroxy-1,1-bis(hydroxymethyl)ethyl)amino]ethanesulfonic acid (TES), 4-(2-hydroxyethyl)-1-piperazineethanesulfonic acid (HEPES), 3-(N,N-Bis[2-hydroxyethyl]amino)-2-hydroxypropanesulfonic acid (DIPSO), acetamidoglycine, 3-{[1,3-Dihydroxy-2-(hydroxymethyl)-2-propanyl]amino}-2-hydroxy-1-propanesulfonic acid (TAPSO), piperazine-1,4,-bis(2-hydroxypropanesulphonic acid) (POPSO), 4-(2-hydroxyethyl)piperazine-1-(2-hydroxypropanesulfonic acid) hydrate (HEPPSO), 3-[4-(2-hydroxyethyl)-1-piperazinyl]propanesulfonic acid (HEPPS), tricine, glycinamide, bicine or N-tris(hydroxymethyl)methyl-3-aminopropanesulfonic acid sodium (TAPS); glycine; and diethanolamine (DEA).

In some cases, amino acid buffering agents include taurine, aspartic acid and its salts (e.g., potassium salts, etc), E-aminocaproic acid, and the like.

Described herein, in some embodiments, is a composition essentially free of a citrate buffering agent, an acetate buffering agent, or a combination thereof. In some embodiments, the composition is substantially free of a citrate buffering agent, an acetate buffering agent, or a combination thereof. In some instances, the composition has no detectable amount of a citrate buffering agent, an acetate buffering agent, or a combination thereof.

In some instances, the composition described herein further comprises a tonicity adjusting agent. Tonicity adjusting agent is an agent introduced into a preparation such as an ophthalmic composition to reduce local irritation by preventing osmotic shock at the site of application. In some instances, buffer solution and/or a pH adjusting agent that broadly maintains the ophthalmic solution at a particular ion concentration and pH are considered as tonicity adjusting agents. In some cases, tonicity adjusting agents include various salts, such as halide salts of a monovalent cation. In some cases, tonicity adjusting agents include mannitol, sorbitol, dextrose, sucrose, urea, and glycerin. In some instances, suitable tonicity adjustors comprise sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, dextrose, mannitol, sorbitol, dextrose, sucrose, urea, propylene glycol, glycerin, or a combination thereof.

In some instances, the concentration of the tonicity adjusting agent in a composition described herein is between about 0.5% and about 2.0%. In some instances, the concentration of the tonicity adjusting agent in a composition described herein is between about 0.7% and about 1.8%, about 0.8% and about 1.5%, or about 1% and about 1.3%. In some instances, the concentration of the tonicity adjusting agent is about 0.6%, 0.7%, 0.8%, 0.9%, 1.0%, 1.1%, 1.2%, 1.3%, 1.4%, 1.5%, 1.6%, 1.7%, 1.8%, or 1.9%. In some cases, the percentage is a weight percentage.

In some cases, the composition described herein further comprises a pH adjusting agent. In some embodiments, the pH adjusting agent used is an acid or a base. In some embodiments, the base is oxides, hydroxides, carbonates, bicarbonates and the likes. In some instances, the oxides are metal oxides such as calcium oxide, magnesium oxide and the likes; hydroxides are of alkali metals and alkaline earth metals such as sodium hydroxide, potassium hydroxide, calcium hydroxide and the likes or their deuterated equivalents, and carbonates are sodium carbonate, sodium bicarbonates, potassium bicarbonates and the likes. In some instances, the acid is mineral acid and organic acids such as hydrochloric acid, nitric acid, phosphoric acid, acetic acid, citric acid, fumaric acid, malic acid tartaric acid and the likes or their deuterated equivalents. In some instances, the pH adjusting agent comprises, but is not limited to, acetate, bicarbonate, ammonium chloride, citrate, phosphate, pharmaceutically acceptable salts thereof and combinations or mixtures thereof. In some embodiments, the pH adjusting agent comprises DCl and NaOD. In some embodiments, the pH adjusting agent comprises DCl, HCl, NaOH, NaOD, CD₃COOD, C₆D₈O₇, CH₃COOH, C₆H₈O₇, or combinations thereof.

In some instances, the pH adjusting agent is present in the ophthalmic composition at a concentration of between about 0.001% to about 0.20%, between about 0.004% to about 0.20%, between about 0.005% to about 0.20%, between about 0.010% to about 0.20%, between about 0.015% to about 0.20%, between about 0.020% to about 0.20%, between about 0.025% to about 0.20%, between about 0.030% to about 0.20%, between about 0.035% to about 0.20%, between about 0.040% to about 0.20%, or between about 0.045% to about 0.20% by weight of the composition. In some instances, the pH adjusting agent is present in the ophthalmic composition at a concentration of at least or about 0.001%, 0.002%, 0.003%, 0.004%, 0.005%, 0.006%, 0.007%, 0.008%, 0.009%, 0.010%, 0.020%, 0.030%, 0.040%, 0.050%, 0.060%, 0.070%, 0.080%, 0.090%, 0.10%, 0.20%, 0.30%, or 0.40% by weight of the composition. In some instances, the pH adjusting agent comprises, but is not limited to, acetate, bicarbonate, ammonium chloride, citrate, phosphate, pharmaceutically acceptable salts thereof and combinations or mixtures thereof. In some embodiments, the pH adjusting agent comprises DCl and NaOD.

In some instances, the pH adjusting agent is citrate. In some instances, the citrate is present in the ophthalmic composition at a concentration of between about 0.001% to about 0.20%, between about 0.004% to about 0.20%, between about 0.005% to about 0.20%, between about 0.010% to about 0.20%, between about 0.015% to about 0.20%, between about 0.020% to about 0.20%, between about 0.025% to about 0.20%, between about 0.030% to about 0.20%, between about 0.035% to about 0.20%, between about 0.040% to about 0.20%, or between about 0.045% to about 0.20% by weight of the composition. In some instances, the citrate is present in the ophthalmic composition at a concentration of at least or about 0.001%, 0.002%, 0.003%, 0.004%, 0.005%, 0.006%, 0.007%, 0.008%, 0.009%, 0.010%, 0.020%, 0.030%, 0.040%, 0.050%, 0.060%, 0.070%, 0.080%, 0.090%, 0.10%, 0.20%, 0.30%, or 0.40% by weight of the composition.

In some cases, the composition described herein further comprises one or more sodium phosphate buffers. Exemplary sodium phosphate buffers include, but are not limited to, monosodium phosphate (sodium dihydrogen phosphate), disodium phosphate, trisodium phosphate, monosodium phosphate anhydrous, disodium phosphate anhydrous, and trisodium phosphate anhydrous. In some instances, a sodium phosphate of the one or more sodium phosphate buffers is monosodium phosphate. In some instances, a sodium phosphate of the one or more sodium phosphate buffers is disodium phosphate. In some instances, a sodium phosphate of the one or more sodium phosphate buffers is anhydrous. In some instances, a sodium phosphate of the one or more sodium phosphate buffers is monosodium phosphate anhydrous. In some instances, a sodium phosphate of the one or more sodium phosphate buffers is disodium phosphate anhydrous.

The concentration of the sodium phosphate, in some embodiments, is between about 0.001% to about 0.20%, between about 0.004% to about 0.20%, between about 0.005% to about 0.20%, between about 0.010% to about 0.20%, between about 0.015% to about 0.20%, between about 0.020% to about 0.20%, between about 0.025% to about 0.20%, between about 0.030% to about 0.20%, between about 0.035% to about 0.20%, between about 0.040% to about 0.20%, or between about 0.045% to about 0.20% by weight of the composition. In some embodiments, the sodium phosphate is between about 0.01% to about 2.0%, between about 0.04% to about 2.0%, between about 0.05% to about 2.0%, between about 0.010% to about 2.0%, between about 0.015% to about 2.0%, between about 0.020% to about 2.0%, between about 0.025% to about 2.0%, between about 0.030% to about 2.0%, between about 0.035% to about 2.0%, between about 0.040% to about 2.0%, or between about 0.045% to about 2.0% by weight of the composition. In some instances, the sodium phosphate is present in the ophthalmic composition at a concentration of at least or about 0.001%, 0.002%, 0.003%, 0.004%, 0.005%, 0.006%, 0.007%, 0.008%, 0.009%, 0.010%, 0.020%, 0.030%, 0.040%, 0.050%, 0.060%, 0.070%, 0.080%, 0.090%, 0.10%, 0.20%, 0.30%, 0.40%, 0.50%, 0.60%, 0.70%, 0.80%, 0.90%, 1.0%, 2.0%, 3.0%, 4.0%, or more than 4.0% by weight of the composition.

The concentration of the monosodium phosphate anhydrous, in some embodiments, is between about 0.001% to about 0.20%, between about 0.004% to about 0.20%, between about 0.005% to about 0.20%, between about 0.010% to about 0.20%, between about 0.015% to about 0.20%, between about 0.020% to about 0.20%, between about 0.025% to about 0.20%, between about 0.030% to about 0.20%, between about 0.035% to about 0.20%, between about 0.040% to about 0.20%, or between about 0.045% to about 0.20% by weight of the composition. In some embodiments, the monosodium phosphate anhydrous is between about 0.01% to about 2.0%, between about 0.04% to about 2.0%, between about 0.05% to about 2.0%, between about 0.010% to about 2.0%, between about 0.015% to about 2.0%, between about 0.020% to about 2.0%, between about 0.025% to about 2.0%, between about 0.030% to about 2.0%, between about 0.035% to about 2.0%, between about 0.040% to about 2.0%, or between about 0.045% to about 2.0% by weight of the composition. In some instances, the monosodium phosphate anhydrous is present in the ophthalmic composition at a concentration of at least or about 0.001%, 0.002%, 0.003%, 0.004%, 0.005%, 0.006%, 0.007%, 0.008%, 0.009%, 0.010%, 0.020%, 0.030%, 0.040%, 0.050%, 0.060%, 0.070%, 0.080%, 0.090%, 0.10%, 0.20%, 0.30%, 0.40%, 0.50%, 0.60%, 0.70%, 0.80%, 0.90%, 1.0%, 2.0%, 3.0%, 4.0%, or more than 4.0% by weight of the composition.

The concentration of the disodium phosphate anhydrous, in some embodiments, is between about 0.001% to about 0.20%, between about 0.004% to about 0.20%, between about 0.005% to about 0.20%, between about 0.010% to about 0.20%, between about 0.015% to about 0.20%, between about 0.020% to about 0.20%, between about 0.025% to about 0.20%, between about 0.030% to about 0.20%, between about 0.035% to about 0.20%, between about 0.040% to about 0.20%, or between about 0.045% to about 0.20% by weight of the composition. In some embodiments, the disodium phosphate anhydrous is between about 0.01% to about 2.0%, between about 0.04% to about 2.0%, between about 0.05% to about 2.0%, between about 0.010% to about 2.0%, between about 0.015% to about 2.0%, between about 0.020% to about 2.0%, between about 0.025% to about 2.0%, between about 0.030% to about 2.0%, between about 0.035% to about 2.0%, between about 0.040% to about 2.0%, or between about 0.045% to about 2.0% by weight of the composition. In some instances, the disodium phosphate anhydrous is present in the ophthalmic composition at a concentration of at least or about 0.001%, 0.002%, 0.003%, 0.004%, 0.005%, 0.006%, 0.007%, 0.008%, 0.009%, 0.010%, 0.020%, 0.030%, 0.040%, 0.050%, 0.060%, 0.070%, 0.080%, 0.090%, 0.10%, 0.20%, 0.30%, 0.40%, 0.50%, 0.60%, 0.70%, 0.80%, 0.90%, 1.0%, 2.0%, 3.0%, 4.0%, or more than 4.0% by weight of the composition.

Described herein is an ophthalmic composition comprising, in some embodiments, a chelator. In some embodiments, the chelator is a bicarboxylic acid, a tricarboxylic acid, or an aminopolycarboxylic acid. In some instances, the chelator is ethylenediaminetetraacetic acid (EDTA), ethylene glycol-bis((3-aminoethyl ether)-N,N,N′,N′-tetraacetic acid (EGTA), and penta(carboxymethyl)diethylenetriamine (DTPA), or salts and hydrates thereof. In some instances, the chelating agent include monomeric polyacids such as EDTA, cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccmic acid (DMSA), aminotrimethylene phosphonic acid (ATPA), citric acid, ophthalmologically acceptable salts thereof, and combinations thereof. In some instances, the chelating agents include pyrophosphates, tripolyphosphates, and, hexametaphosphates, chelating antibiotics such as chloroquine and tetracycline, nitrogen-containing chelating agent containing two or more chelating nitrogen atoms within an imino group or in an aromatic ring (e.g., diimines, 2,2′-bipyridines, etc.), and various polyamines such as cyclam (1,4,7,11-tetraazacyclotetradecane), N(C₁-C₃₀ alkyl)-substituted cyclams (e.g., hexadecyclam, tetramethylhexadecylcyclam), diethylenetriamine (DETA), spermine, diethylnorspermine (DENSPM), diethylhomo-spermine (DEHOP), and deferoxamine (N′-[5-[[4-[[5-(acetylhydroxyamino)pentyl]amino]-1,4-dioxobutyl]hydroxy-a-mino]pentyl]-N′-(5-aminopentyl)-N-hydroxybutanediamide; also known as desferrioxamine B and DFO).

The concentration of the chelator, in some embodiments, is between about 0.001% to about 0.20%, between about 0.004% to about 0.20%, between about 0.005% to about 0.20%, between about 0.010% to about 0.20%, between about 0.015% to about 0.20%, between about 0.020% to about 0.20%, between about 0.025% to about 0.20%, between about 0.030% to about 0.20%, between about 0.035% to about 0.20%, between about 0.040% to about 0.20%, or between about 0.045% to about 0.20% by weight of the composition. In some embodiments, the chelator is between about 0.01% to about 2.0%, between about 0.04% to about 2.0%, between about 0.05% to about 2.0%, between about 0.010% to about 2.0%, between about 0.015% to about 2.0%, between about 0.020% to about 2.0%, between about 0.025% to about 2.0%, between about 0.030% to about 2.0%, between about 0.035% to about 2.0%, between about 0.040% to about 2.0%, or between about 0.045% to about 2.0% by weight of the composition. In some instances, the chelator is present in the ophthalmic composition at a concentration of at least or about 0.001%, 0.002%, 0.003%, 0.004%, 0.005%, 0.006%, 0.007%, 0.008%, 0.009%, 0.010%, 0.020%, 0.030%, 0.040%, 0.050%, 0.060%, 0.070%, 0.080%, 0.090%, 0.10%, 0.20%, 0.30%, 0.40%, 0.50%, 0.60%, 0.70%, 0.80%, 0.90%, 1.0%, 2.0%, 3.0%, 4.0%, or more than 4.0% by weight of the composition. In some instances, the chelator is EDTA.

In some instances, the composition has a pH of between about 4 and about 8, about 4.2 to about 7.9, about 4.5 and about 7.8, about 5 and about 7.5, or about 5.5 and about 7. In some embodiments, the composition has a pH of about 8.0. In some embodiments, the composition has a pH of about 7.9. In some embodiments, the composition has a pH of about 7.8. In some embodiments, the composition has a pH of about 7.7. In some embodiments, the composition has a pH of about 7.6. In some embodiments, the composition has a pH of less than about 7.5. In some embodiments, the composition has a pH of less than about 7.4. In some embodiments, the composition has a pH of less than about 7.3. In some embodiments, the composition has a pH of less than about 7.2. In some embodiments, the composition has a pH of less than about 7.1. In some embodiments, the composition has a pH of less than about 7. In some embodiments, the composition has a pH of less than about 6.9. In some embodiments, the composition has a pH of less than about 6.8. In some embodiments, the composition has a pH of less than about 6.7. In some embodiments, the composition has a pH of less than about 6.6. In some embodiments, the composition has a pH of less than about 6.5. In some embodiments, the composition has a pH of less than about 6.4. In some embodiments, the composition has a pH of less than about 6.3. In some embodiments, the composition has a pH of less than about 6.2. In some embodiments, the composition has a pH of less than about 6.1. In some embodiments, the composition has a pH of less than about 6. In some embodiments, the composition has a pH of less than about 5.9. In some embodiments, the composition has a pH of less than about 5.8. In some embodiments, the composition has a pH of less than about 5.7. In some embodiments, the composition has a pH of less than about 5.6. In some embodiments, the composition has a pH of less than about 5.5. In some embodiments, the composition has a pH of less than about 5.4. In some embodiments, the composition has a pH of less than about 5.3. In some embodiments, the composition has a pH of less than about 5.2. In some embodiments, the composition has a pH of less than about 5.1. In some embodiments, the composition has a pH of less than about 5. In some embodiments, the composition has a pH of less than about 4.9. In some embodiments, the composition has a pH of less than about 4.8. In some embodiments, the composition has a pH of less than about 4.7. In some embodiments, the composition has a pH of less than about 4.6. In some embodiments, the composition has a pH of less than about 4.5. In some embodiments, the composition has a pH of less than about 4.4. In some embodiments, the composition has a pH of less than about 4.3. In some embodiments, the composition has a pH of less than about 4.2. In some embodiments, the composition has a pH of less than about 4.1. In some embodiments, the composition has a pH of less than about 4. In some embodiments, the pH is the pH of the composition after extended period of time under a storage condition.

In some instances, the composition has a pD of between about 4 and about 8, about 4.2 to about 7.9, about 4.5 and about 7.8, about 5 and about 7.5, or about 5.5 and about 7. In some embodiments, the composition has a pD of about 8.0. In some embodiments, the composition has a pD of about 7.9. In some embodiments, the composition has a pD of about 7.8. In some embodiments, the composition has a pD of about 7.7. In some embodiments, the composition has a pD of about 7.6. In some embodiments, the composition has a pD of less than about 7.5. In some embodiments, the composition has a pD of less than about 7.4. In some embodiments, the composition has a pD of less than about 7.3. In some embodiments, the composition has a pD of less than about 7.2. In some embodiments, the composition has a pD of less than about 7.1. In some embodiments, the composition has a pD of less than about 7. In some embodiments, the composition has a pD of less than about 6.9. In some embodiments, the composition has a pD of less than about 6.8. In some embodiments, the composition has a pD of less than about 6.7. In some embodiments, the composition has a pD of less than about 6.6. In some embodiments, the composition has a pD of less than about 6.5. In some embodiments, the composition has a pD of less than about 6.4. In some embodiments, the composition has a pD of less than about 6.3. In some embodiments, the composition has a pD of less than about 6.2. In some embodiments, the composition has a pD of less than about 6.1. In some embodiments, the composition has a pD of less than about 6. In some embodiments, the composition has a pD of less than about 5.9. In some embodiments, the composition has a pD of less than about 5.8. In some embodiments, the composition has a pD of less than about 5.7. In some embodiments, the composition has a pD of less than about 5.6. In some embodiments, the composition has a pD of less than about 5.5. In some embodiments, the composition has a pD of less than about 5.4. In some embodiments, the composition has a pD of less than about 5.3. In some embodiments, the composition has a pD of less than about 5.2. In some embodiments, the composition has a pD of less than about 5.1. In some embodiments, the composition has a pD of less than about 5. In some embodiments, the composition has a pD of less than about 4.9. In some embodiments, the composition has a pD of less than about 4.8. In some embodiments, the composition has a pD of less than about 4.7. In some embodiments, the composition has a pD of less than about 4.6. In some embodiments, the composition has a pD of less than about 4.5. In some embodiments, the composition has a pD of less than about 4.4. In some embodiments, the composition has a pD of less than about 4.3. In some embodiments, the composition has a pD of less than about 4.2. In some embodiments, the composition has a pD of less than about 4.1. In some embodiments, the composition has a pD of less than about 4. In some embodiments, the pD is the pD of the composition after extended period of time under a storage condition.

In some instances, the composition has an initial pH of between about 4 and about 8, about 4.2 to about 7.9, about 4.5 and about 7.8, about 5 and about 7.5, or about 5.5 and about 7. In some embodiments, the composition has an initial pH of about 8.0. In some embodiments, the composition has an initial pH of about 7.9. In some embodiments, the composition has an initial pH of about 7.8. In some embodiments, the composition has an initial pH of about 7.7. In some embodiments, the composition has an initial pH of about 7.6. In some embodiments, the composition has an initial pH of about 7.5. In some embodiments, the composition has an initial pH of about 7.4. In some embodiments, the composition has an initial pH of about 7.3. In some embodiments, the composition has an initial pH of about 7.2. In some embodiments, the composition has an initial pH of about 7.1. In some embodiments, the composition has an initial pH of about 7. In some embodiments, the composition has an initial pH of about 6.9. In some embodiments, the composition has an initial pH of about 6.8. In some embodiments, the composition has an initial pH of about 6.7. In some embodiments, the composition has an initial pH of about 6.6. In some embodiments, the composition has an initial pH of about 6.5. In some embodiments, the composition has an initial pH of about 6.4. In some embodiments, the composition has an initial pH of about 6.3. In some embodiments, the composition has an initial pH of about 6.2. In some embodiments, the composition has an initial pH of about 6.1. In some embodiments, the composition has an initial pH of about 6. In some embodiments, the composition has an initial pH of about 5.9. In some embodiments, the composition has an initial pH of about 5.8. In some embodiments, the composition has an initial pH of about 5.7. In some embodiments, the composition has an initial pH of about 5.6. In some embodiments, the composition has an initial pH of about 5.5. In some embodiments, the composition has an initial pH of about 5.4. In some embodiments, the composition has an initial pH of about 5.3. In some embodiments, the composition has an initial pH of about 5.2. In some embodiments, the composition has an initial pH of about 5.1. In some embodiments, the composition has an initial pH of about 5. In some embodiments, the composition has an initial pH of about 4.9. In some embodiments, the composition has an initial pH of about 4.8. In some embodiments, the composition has an initial pH of about 4.7. In some embodiments, the composition has an initial pH of about 4.6. In some embodiments, the composition has an initial pH of about 4.5. In some embodiments, the composition has an initial pH of about 4.4. In some embodiments, the composition has an initial pH of about 4.3. In some embodiments, the composition has an initial pH of about 4.2. In some embodiments, the composition has an initial pH of about 4.1. In some embodiments, the composition has an initial pH of about 4.

In some instances, the composition has an initial pD of between about 4 and about 8, about 4.2 to about 7.9, about 4.5 and about 7.8, about 5 and about 7.5, or about 5.5 and about 7. In some embodiments, the composition has an initial pD of about 8.0. In some embodiments, the composition has an initial pD of about 7.9. In some embodiments, the composition has an initial pD of about 7.8. In some embodiments, the composition has an initial pD of about 7.7. In some embodiments, the composition has an initial pD of about 7.6. In some embodiments, the composition has an initial pD of about 7.5. In some embodiments, the composition has an initial pD of about 7.4. In some embodiments, the composition has an initial pD of about 7.3. In some embodiments, the composition has an initial pD of about 7.2. In some embodiments, the composition has an initial pD of about 7.1. In some embodiments, the composition has an initial pD of about 7. In some embodiments, the composition has an initial pD of about 6.9. In some embodiments, the composition has an initial pD of about 6.8. In some embodiments, the composition has an initial pD of about 6.7. In some embodiments, the composition has an initial pD of about 6.6. In some embodiments, the composition has an initial pD of about 6.5. In some embodiments, the composition has an initial pD of about 6.4. In some embodiments, the composition has an initial pD of about 6.3. In some embodiments, the composition has an initial pD of about 6.2. In some embodiments, the composition has an initial pD of about 6.1. In some embodiments, the composition has an initial pD of about 6. In some embodiments, the composition has an initial pD of about 5.9. In some embodiments, the composition has an initial pD of about 5.8. In some embodiments, the composition has an initial pD of about 5.7. In some embodiments, the composition has an initial pD of about 5.6. In some embodiments, the composition has an initial pD of about 5.5. In some embodiments, the composition has an initial pD of about 5.4. In some embodiments, the composition has an initial pD of about 5.3. In some embodiments, the composition has an initial pD of about 5.2. In some embodiments, the composition has an initial pD of about 5.1. In some embodiments, the composition has an initial pD of about 5. In some embodiments, the composition has an initial pD of about 4.9. In some embodiments, the composition has an initial pD of about 4.8. In some embodiments, the composition has an initial pD of about 4.7. In some embodiments, the composition has an initial pD of about 4.6. In some embodiments, the composition has an initial pD of about 4.5. In some embodiments, the composition has an initial pD of about 4.4. In some embodiments, the composition has an initial pD of about 4.3. In some embodiments, the composition has an initial pD of about 4.2. In some embodiments, the composition has an initial pD of about 4.1. In some embodiments, the composition has an initial pD of about 4.

In some embodiments, the pH of the composition described herein is associated with the stability of the composition. In some embodiments, a stable composition comprises a pH of between about 4 and about 8, about 4.2 to about 7.9, about 4.5 and about 7.8, about 5 and about 7.5, or about 5.5 and about 7. In some embodiments, a stable composition comprises a pH of about 8.0. In some embodiments, a stable composition comprises a pH of about 7.9. In some embodiments, a stable composition comprises a pH of about 7.8. In some embodiments, a stable composition comprises a pH of about 7.7. In some embodiments, a stable composition comprises a pH of about 7.6. In some embodiments, a stable composition comprises a pH of less than about 7.5. In some embodiments, a stable composition comprises a pH of less than about 7.4. In some embodiments, a stable composition comprises a pH of less than about 7.3. In some embodiments, a stable composition comprises a pH of less than about 7.2. In some embodiments, a stable composition comprises a pH of less than about 7.1. In some embodiments, a stable composition comprises a pH of less than about 7. In some embodiments, a stable composition comprises a pH of less than about 6.9. In some embodiments, a stable composition comprises a pH of less than about 6.8. In some embodiments, a stable composition comprises a pH of less than about 6.7. In some embodiments, a stable composition comprises a pH of less than about 6.6. In some embodiments, a stable composition comprises a pH of less than about 6.5. In some embodiments, a stable composition comprises a pH of less than about 6.4. In some embodiments, a stable composition comprises a pH of less than about 6.3. In some embodiments, a stable composition comprises a pH of less than about 6.2. In some embodiments, a stable composition comprises a pH of less than about 6.1. In some embodiments, a stable composition comprises a pH of less than about 6. In some embodiments, a stable composition comprises a pH of less than about 5.9. In some embodiments, a stable composition comprises a pH of less than about 5.8. In some embodiments, a stable composition comprises a pH of less than about 5.7. In some embodiments, a stable composition comprises a pH of less than about 5.6. In some embodiments, a stable composition comprises a pH of less than about 5.5. In some embodiments, a stable composition comprises a pH of less than about 5.4. In some embodiments, a stable composition comprises a pH of less than about 5.3. In some embodiments, a stable composition comprises a pH of less than about 5.2. In some embodiments, a stable composition comprises a pH of less than about 5.1. In some embodiments, a stable composition comprises a pH of less than about 5. In some embodiments, a stable composition comprises a pH of less than about 4.9. In some embodiments, a stable composition comprises a pH of less than about 4.8. In some embodiments, a stable composition comprises a pH of less than about 4.7. In some embodiments, a stable composition comprises a pH of less than about 4.6. In some embodiments, a stable composition comprises a pH of less than about 4.5. In some embodiments, a stable composition comprises a pH of less than about 4.4. In some embodiments, a stable composition comprises a pH of less than about 4.3. In some embodiments, a stable composition comprises a pH of less than about 4.2. In some embodiments, a stable composition comprises a pH of less than about 4.1. In some embodiments, a stable composition comprises a pH of less than about 4.

In some embodiments, the pD of the composition described herein is associated with the stability of the composition. In some embodiments, a stable composition comprises a pD of between about 4 and about 8, about 4.2 to about 7.9, about 4.5 and about 7.8, about 5 and about 7.5, or about 5.5 and about 7. In some embodiments, a stable composition comprises a pD of about 8.0. In some embodiments, a stable composition comprises a pD of about 7.9. In some embodiments, a stable composition comprises a pD of about 7.8. In some embodiments, a stable composition comprises a pD of about 7.7. In some embodiments, a stable composition comprises a pD of about 7.6. In some embodiments, a stable composition comprises a pD of less than about 7.5. In some embodiments, a stable composition comprises a pD of less than about 7.4. In some embodiments, a stable composition comprises a pD of less than about 7.3. In some embodiments, a stable composition comprises a pD of less than about 7.2. In some embodiments, a stable composition comprises a pD of less than about 7.1. In some embodiments, a stable composition comprises a pD of less than about 7. In some embodiments, a stable composition comprises a pD of less than about 6.9. In some embodiments, a stable composition comprises a pD of less than about 6.8. In some embodiments, a stable composition comprises a pD of less than about 6.7. In some embodiments, a stable composition comprises a pD of less than about 6.6. In some embodiments, a stable composition comprises a pD of less than about 6.5. In some embodiments, a stable composition comprises a pD of less than about 6.4. In some embodiments, a stable composition comprises a pD of less than about 6.3. In some embodiments, a stable composition comprises a pD of less than about 6.2. In some embodiments, a stable composition comprises a pD of less than about 6.1. In some embodiments, a stable composition comprises a pD of less than about 6. In some embodiments, a stable composition comprises a pD of less than about 5.9. In some embodiments, a stable composition comprises a pD of less than about 5.8. In some embodiments, a stable composition comprises a pD of less than about 5.7. In some embodiments, a stable composition comprises a pD of less than about 5.6. In some embodiments, a stable composition comprises a pD of less than about 5.5. In some embodiments, a stable composition comprises a pD of less than about 5.4. In some embodiments, a stable composition comprises a pD of less than about 5.3. In some embodiments, a stable composition comprises a pD of less than about 5.2. In some embodiments, a stable composition comprises a pD of less than about 5.1. In some embodiments, a stable composition comprises a pD of less than about 5. In some embodiments, a stable composition comprises a pD of less than about 4.9. In some embodiments, a stable composition comprises a pD of less than about 4.8. In some embodiments, a stable composition comprises a pD of less than about 4.7. In some embodiments, a stable composition comprises a pD of less than about 4.6. In some embodiments, a stable composition comprises a pD of less than about 4.5. In some embodiments, a stable composition comprises a pD of less than about 4.4. In some embodiments, a stable composition comprises a pD of less than about 4.3. In some embodiments, a stable composition comprises a pD of less than about 4.2. In some embodiments, a stable composition comprises a pD of less than about 4.1. In some embodiments, a stable composition comprises a pD of less than about 4.

As described elsewhere herein, in some instances, the D₂O/aqueous system stabilizes a muscarinic agent (e.g. aceclidine, pilocarpine, or tropicamide). In some embodiments, this is due to a lower concentration of the reactive species (e.g., —OD) in the D₂O aqueous system compared to the concentration of the reactive species (e.g., —OH) in an equivalent purely aqueous system. In some instances, the concentration of the reactive species (e.g., —OD) in the D₂O/aqueous system is about one third less than the concentration of the reactive species (e.g., —OH) in the equivalent purely aqueous system. In some cases, this is due to a lower or smaller dissociation constant of D₂O than H₂O. For example, the Kₐ(H₂O) is 1×10¹⁴, whereas the Kₐ(D₂O) is 1×10¹⁵. As such, D₂O is a weaker acid than H₂O. In some embodiments, the ophthalmic composition formulated with deuterated water allows for a more stable ophthalmic composition relative to the ophthalmic composition formulated with H₂O.

In some embodiments, the presence of deuterated water shifts the pKa of the buffer. In some embodiments, the presence of deuterated water allows for the ophthalmic composition to simulate the stability of a lower pH system. In some instances, the buffer capacity of the ophthalmic composition is lowered, thereby allowing a faster shift in pH. In some instances, the lowered buffering capacity of the ophthalmic composition when administered into the eye allows the ophthalmic composition to reach physiological pH at a faster rate than compared to an ophthalmic composition formulated in H₂O. In some instances, the ophthalmic composition formulated with deuterated water allows for a lower tear production, or less tear reflex in the eye, in comparison with an ophthalmic composition formulated with H₂O.

In some instances, the composition described herein further comprises a disinfecting agent. In some cases, disinfecting agents include polymeric biguanides, polymeric quaternary ammonium compounds, chlorites, bisbiguanides, chlorite compounds (e.g. potassium chlorite, sodium chlorite, calcium chlorite, magnesium chlorite, or mixtures thereof), and a combination thereof.

In some instances, the composition described herein further comprises a preservative. In some cases, a preservative is added at a concentration to a composition described herein to prevent the growth of or to destroy a microorganism introduced into the composition. In some instances, microorganisms refer to bacteria (e.g. Proteus mirabilis, Serratia marcesens), virus (e.g. Herpes simplex virus, herpes zoster virus), fungus (e.g. fungi from the genus Fusarium), yeast (e.g. Candida albicans), parasites (e.g. Plasmodium spp., Gnathostoma spp.), protozoan (e.g. Giardia lamblia), nematodes (e.g. Onchocercus volvulus), worm (e.g. Dirofilaria immitis), and/or amoeba (e.g. Acanthameoba).

In some instances, the concentration of the preservative is between about 0.0001% and about 1%, about 0.001% and about 0.8%, about 0.004% and about 0.5%, about 0.008% and about 0.1%, and about 0.01% and about 0.08%. In some cases, the concentration of the preservatives is about 0.001%, 0.002%, 0.003%, 0.004%, 0.005%, 0.006%, 0.008%, 0.009%, 0.009%, 0.01%, 0.015%, 0.02%, 0.025%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% or 1.0%. In some instances, the concentration of the preservative is by weight of the composition.

In some embodiments, the preservative is selected from benzalkonium chloride, cetrimonium, sodium perborate, stabilized oxychloro complex, SofZia (Alcon), polyquaternium-1, chlorobutanol, edetate disodium, and polyhexamethylene biguanide.

The ophthalmic composition as described herein, in some embodiments, is substantially free of a preservative. In some instances, the ophthalmic composition is substantially free of a benzalkonium chloride preservative. In some instances, the composition has no detectable amount of a benzalkonium chloride preservative. In some instances, the composition has no detectable amount of a benzalkonium chloride. In some instances, the composition is substantially free of a preservative selected from cetrimonium, sodium perborate, stabilized oxychloro complex, SofZia, polyquaternium-1, chlorobutanol, edetate disodium, polyhexamethylene biguanide, or combinations thereof. In some instances, the composition has no detectable amount of a preservative. In some instances, the composition is substantially free of any preservative.

In some embodiments, the composition described herein is stored in a plastic container. In some embodiments, the material of the plastic container comprises high density polyethylene (HDPE), low density polyethylene (LDPE), polyethylene terephthalate (PET), polyvinyl chloride (PVC), polypropylene (PP), polystyrene (PS), fluorine treated HDPE, post-consumer resin (PCR), K-resin (SBC), or bioplastic. In some embodiments, the material of the plastic container comprises LDPE.

In some embodiments, the composition described herein is stored in a plastic container. In some embodiments, the composition stored in a plastic container has a pH of between about 4 and about 8, about 4.2 to about 7.9, about 4.5 and about 7.9, or about 4.9 and about 7.5. In some embodiments, the composition stored in a plastic container has a pH of about 7.9. In some embodiments, the composition stored in a plastic container has a pH of about 7.8. In some embodiments, the composition stored in a plastic container has a pH of about 7.7. In some embodiments, the composition stored in a plastic container has a pH of about 7.6. In some embodiments, the composition stored in a plastic container has a pH of less than about 7.5. In some embodiments, the composition stored in a plastic container has a pH of less than about 7.4. In some embodiments, the composition stored in a plastic container has a pH of less than about 7.3. In some embodiments, the composition stored in a plastic container has a pH of less than about 7.2. In some embodiments, the composition stored in a plastic container has a pH of less than about 7.1. In some embodiments, the composition stored in a plastic container has a pH of less than about 7. In some embodiments, the composition stored in a plastic container has a pH of less than about 6.9. In some embodiments, the composition stored in a plastic container has a pH of less than about 6.8. In some embodiments, the composition stored in a plastic container has a pH of less than about 6.7. In some embodiments, the composition stored in a plastic container has a pH of less than about 6.6. In some embodiments, the composition stored in a plastic container has a pH of less than about 6.5. In some embodiments, the composition stored in a plastic container has a pH of less than about 6.4. In some embodiments, the composition stored in a plastic container has a pH of less than about 6.3. In some embodiments, the composition stored in a plastic container has a pH of less than about 6.2. In some embodiments, the composition stored in a plastic container has a pH of less than about 6.1. In some embodiments, the composition stored in a plastic container has a pH of less than about 6. In some embodiments, the composition stored in a plastic container has a pH of less than about 5.9. In some embodiments, the composition stored in a plastic container has a pH of less than about 5.8. In some embodiments, the composition stored in a plastic container has a pH of less than about 5.7. In some embodiments, the composition stored in a plastic container has a pH of less than about 5.6. In some embodiments, the composition stored in a plastic container has a pH of less than about 5.5. In some embodiments, the composition stored in a plastic container has a pH of less than about 5.4. In some embodiments, the composition stored in a plastic container has a pH of less than about 5.3. In some embodiments, the composition stored in a plastic container has a pH of less than about 5.2. In some embodiments, the composition stored in a plastic container has a pH of less than about 5.1. In some embodiments, the composition stored in a plastic container has a pH of less than about 5. In some embodiments, the composition stored in a plastic container has a pH of less than about 4.9. In some embodiments, the composition stored in a plastic container has a pH of less than about 4.8. In some embodiments, the composition stored in a plastic container has a pH of less than about 4.7. In some embodiments, the composition stored in a plastic container has a pH of less than about 4.6. In some embodiments, the composition stored in a plastic container has a pH of less than about 4.5. In some embodiments, the composition stored in a plastic container has a pH of less than about 4.4. In some embodiments, the composition stored in a plastic container has a pH of less than about 4.3. In some embodiments, the composition stored in a plastic container has a pH of less than about 4.2. In some embodiments, the composition stored in a plastic container has a pH of less than about 4.1. In some embodiments, the composition stored in a plastic container has a pH of less than about 4.

In some embodiments, the composition described herein is stored in a plastic container. In some embodiments, the composition stored in a plastic container has a pD of between about 4 and about 8, about 4.2 to about 7.9, about 4.5 and about 7.9, or about 4.9 and about 7.5. In some embodiments, the composition stored in a plastic container has a pD of about 7.9. In some embodiments, the composition stored in a plastic container has a pD of about 7.8. In some embodiments, the composition stored in a plastic container has a pD of about 7.7. In some embodiments, the composition stored in a plastic container has a pD of about 7.6. In some embodiments, the composition stored in a plastic container has a pD of less than about 7.5. In some embodiments, the composition stored in a plastic container has a pD of less than about 7.4. In some embodiments, the composition stored in a plastic container has a pD of less than about 7.3. In some embodiments, the composition stored in a plastic container has a pD of less than about 7.2. In some embodiments, the composition stored in a plastic container has a pD of less than about 7.1. In some embodiments, the composition stored in a plastic container has a pD of less than about 7. In some embodiments, the composition stored in a plastic container has a pD of less than about 6.9. In some embodiments, the composition stored in a plastic container has a pD of less than about 6.8. In some embodiments, the composition stored in a plastic container has a pD of less than about 6.7. In some embodiments, the composition stored in a plastic container has a pD of less than about 6.6. In some embodiments, the composition stored in a plastic container has a pD of less than about 6.5. In some embodiments, the composition stored in a plastic container has a pD of less than about 6.4. In some embodiments, the composition stored in a plastic container has a pD of less than about 6.3. In some embodiments, the composition stored in a plastic container has a pD of less than about 6.2. In some embodiments, the composition stored in a plastic container has a pD of less than about 6.1. In some embodiments, the composition stored in a plastic container has a pD of less than about 6. In some embodiments, the composition stored in a plastic container has a pD of less than about 5.9. In some embodiments, the composition stored in a plastic container has a pD of less than about 5.8. In some embodiments, the composition stored in a plastic container has a pD of less than about 5.7. In some embodiments, the composition stored in a plastic container has a pD of less than about 5.6. In some embodiments, the composition stored in a plastic container has a pD of less than about 5.5. In some embodiments, the composition stored in a plastic container has a pD of less than about 5.4. In some embodiments, the composition stored in a plastic container has a pD of less than about 5.3. In some embodiments, the composition stored in a plastic container has a pD of less than about 5.2. In some embodiments, the composition stored in a plastic container has a pD of less than about 5.1. In some embodiments, the composition stored in a plastic container has a pD of less than about 5. In some embodiments, the composition stored in a plastic container has a pD of less than about 4.9. In some embodiments, the composition stored in a plastic container has a pD of less than about 4.8. In some embodiments, the composition stored in a plastic container has a pD of less than about 4.7. In some embodiments, the composition stored in a plastic container has a pD of less than about 4.6. In some embodiments, the composition stored in a plastic container has a pD of less than about 4.5. In some embodiments, the composition stored in a plastic container has a pD of less than about 4.4. In some embodiments, the composition stored in a plastic container has a pD of less than about 4.3. In some embodiments, the composition stored in a plastic container has a pD of less than about 4.2. In some embodiments, the composition stored in a plastic container has a pD of less than about 4.1. In some embodiments, the composition stored in a plastic container has a pD of less than about 4.

In some embodiments, the composition stored in a plastic container has a potency of at least 80% after extended period of time under a storage condition. In some embodiments, the composition stored in a plastic container has a potency of at least 85% after extended period of time under a storage condition. In some embodiments, the composition stored in a plastic container has a potency of at least 90% after extended period of time under a storage condition. In some embodiments, the composition stored in a plastic container has a potency of at least 93% after extended period of time under a storage condition. In some embodiments, the composition stored in a plastic container has a potency of at least 95% after extended period of time under a storage condition. In some embodiments, the composition stored in a plastic container has a potency of at least 97% after extended period of time under a storage condition. In some embodiments, the composition stored in a plastic container has a potency of at least 98% after extended period of time under a storage condition. In some embodiments, the composition stored in a plastic container has a potency of at least 99% after extended period of time under a storage condition. In some instances, the storage condition comprises a temperature of about 25° C., about 40° C., or about 60° C. In some instances, the extended period of time is at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months.

In some embodiments, the composition stored in a plastic container has a potency of at least 80% at a temperature of about 0° C., about 2° C., about 5° C., about 10° C., about 15° C., about 25° C., about 40° C., or about 60° C. In some embodiments, the composition stored in a plastic container has a potency of at least 85% at a temperature of about 0° C., about 2° C., about 5° C., about 10° C., about 15° C., about 25° C., about 40° C., or about 60° C. In some embodiments, the composition stored in a plastic container has a potency of at least 90% at a temperature of about 0° C., about 2° C., about 5° C., about 10° C., about 15° C., about 25° C., about 40° C., or about 60° C. In some embodiments, the composition stored in a plastic container has a potency of at least 93% at a temperature of about 0° C., about 2° C., about 5° C., about 10° C., about 15° C., about 25° C., about 40° C., or about 60° C. In some embodiments, the composition stored in a plastic container has a potency of at least 95% at a temperature of about 0° C., about 2° C., about 5° C., about 10° C., about 15° C., about 25° C., about 40° C., or about 60° C. In some embodiments, the composition stored in a plastic container has a potency of at least 97% at a temperature of about 0° C., about 2° C., about 5° C., about 10° C., about 15° C., about 25° C., about 40° C., or about 60° C. In some embodiments, the composition stored in a plastic container has a potency of at least 98% at a temperature of about 0° C., about 2° C., about 5° C., about 10° C., about 15° C., about 25° C., about 40° C., or about 60° C. In some embodiments, the composition stored in a plastic container has a potency of at least 99% at a temperature of about 0° C., about 2° C., about 5° C., about 10° C., about 15° C., about 25° C., about 40° C., or about 60° C. In some embodiments, the composition stored in a plastic container has a potency of at least 80%, at least 85%, at least 90%, at least 93%, at least 95%, at least 97%, at least 98%, or at least 99% at a temperature of from about 0° C. to about 30° C., 2° C. to about 10° C. or from about 16° C. to about 26° C.

In some embodiments, the composition stored in a plastic container has a potency of at least 80% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container has a potency of at least 85% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container has a potency of at least 90% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container has a potency of at least 93% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container has a potency of at least 95% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container has a potency of at least 97% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container has a potency of at least 98% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container has a potency of at least 99% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months.

In some embodiments, the composition stored in a plastic container comprises less than 20% of major degradant based on the concentration of the ophthalmic agent after extended period of time under a storage condition. In some embodiments, the composition stored in a plastic container comprises less than 15% of major degradant based on the concentration of the ophthalmic agent after extended period of time under a storage condition. In some embodiments, the composition stored in a plastic container comprises less than 10% of major degradant based on the concentration of the ophthalmic agent after extended period of time under a storage condition. In some embodiments, the composition stored in a plastic container comprises less than 5% of major degradant based on the concentration of the ophthalmic agent after extended period of time under a storage condition. In some embodiments, the major degradant is pilocarpic acid, isopilocarpic acid, isopilocarpine, or combinations thereof. In some embodiments, the major degradant is pilocarpic acid. In some embodiments, the major degradant is isopilocarpic acid. In some embodiments, the major degradant is isopilocarpine.

In some embodiments, the composition stored in a plastic container comprises from less than 2.5% of major degradant to less than 0.1% of major degradant based on the concentration of the ophthalmic agent after extended period of time under a storage condition. In some embodiments, the composition stored in a plastic container comprises less than 2.5% of major degradant based on the concentration of the ophthalmic agent after extended period of time under a storage condition. In some embodiments, the composition stored in a plastic container comprises less than 2.0% of major degradant based on the concentration of the ophthalmic agent after extended period of time under a storage condition. In some embodiments, the composition stored in a plastic container comprises less than 1.5% of major degradant based on the concentration of the ophthalmic agent after extended period of time under a storage condition. In some embodiments, the composition stored in a plastic container comprises less than 1.0% of major degradant based on the concentration of the ophthalmic agent after extended period of time under a storage condition. In some embodiments, the composition stored in a plastic container comprises less than 0.5% of major degradant based on the concentration of the ophthalmic agent after extended period of time under a storage condition. In some embodiments, the composition stored in a plastic container comprises less than 0.4% of major degradant based on the concentration of the ophthalmic agent after extended period of time under a storage condition. In some embodiments, the composition stored in a plastic container comprises less than 0.3% of major degradant based on the concentration of the ophthalmic agent after extended period of time under a storage condition. In some embodiments, the composition stored in a plastic container comprises less than 0.2% of major degradant based on the concentration of the ophthalmic agent after extended period of time under a storage condition. In some embodiments, the composition stored in a plastic container comprises less than 0.1% of major degradant based on the concentration of the ophthalmic agent after extended period of time under a storage condition. In some instances, a storage condition comprises a temperature of about 25° C., about 40° C., or about 60° C. In some embodiments, a storage condition comprises a temperature is between about 0° C. to about 30° C., 2° C. to about 10° C., or from about 16° C. to about 26° C. In some instances, the extended period of time is at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the major degradant is pilocarpic acid, isopilocarpic acid, isopilocarpine, or combinations thereof. In some embodiments, the major degradant is pilocarpic acid. In some embodiments, the major degradant is isopilocarpic acid. In some embodiments, the major degradant is isopilocarpine.

In some embodiments, the composition stored in a plastic container comprises less than 20% of major degradant based on the concentration of the ophthalmic agent at a temperature of about 25° C., about 40° C., or about 60° C. In some embodiments, the composition stored in a plastic container comprises less than 15% of major degradant based on the concentration of the ophthalmic agent at a temperature of about 25° C., about 40° C., or about 60° C. In some embodiments, the composition stored in a plastic container comprises less than 10% of major degradant based on the concentration of the ophthalmic agent at a temperature of about 25° C., about 40° C., or about 60° C. In some embodiments, the composition stored in a plastic container comprises less than 5% of major degradant based on the concentration of the ophthalmic agent at a temperature of about 25° C., about 40° C., or about 60° C. In some embodiments, the major degradant is pilocarpic acid, isopilocarpic acid, isopilocarpine, or combinations thereof. In some embodiments, the major degradant is pilocarpic acid. In some embodiments, the major degradant is isopilocarpic acid. In some embodiments, the major degradant is isopilocarpine.

In some embodiments, the composition stored in a plastic container comprises from less than 2.5% of major degradant to less than 0.1% of major degradant based on the concentration of the ophthalmic agent at a temperature of about 25° C., about 40° C., or about 60° C. In some embodiments, the composition stored in a plastic container comprises less than 2.5% of major degradant based on the concentration of the ophthalmic agent at a temperature of about 25° C., about 40° C., or about 60° C. In some embodiments, the composition stored in a plastic container comprises less than 2.0% of major degradant based on the concentration of the ophthalmic agent at a temperature of about 25° C., about 40° C., or about 60° C. In some embodiments, the composition stored in a plastic container comprises less than 1.5% of major degradant based on the concentration of the ophthalmic agent at a temperature of about 25° C., about 40° C., or about 60° C. In some embodiments, the composition stored in a plastic container comprises less than 1.0% of major degradant based on the concentration of the ophthalmic agent at a temperature of about 25° C., about 40° C., or about 60° C. In some embodiments, the composition stored in a plastic container comprises less than 0.5% of major degradant based on the concentration of the ophthalmic agent at a temperature of about 25° C., about 40° C., or about 60° C. In some embodiments, the composition stored in a plastic container comprises less than 0.4% of major degradant based on the concentration of the ophthalmic agent at a temperature of about 25° C., about 40° C., or about 60° C. In some embodiments, the composition stored in a plastic container comprises less than 0.3% of major degradant based on the concentration of the ophthalmic agent at a temperature of about 25° C., about 40° C., or about 60° C. In some embodiments, the composition stored in a plastic container comprises less than 0.2% of major degradant based on the concentration of the ophthalmic agent at a temperature of about 25° C., about 40° C., or about 60° C. In some embodiments, the composition stored in a plastic container comprises less than 0.1% of major degradant based on the concentration of the ophthalmic agent at a temperature of about 25° C., about 40° C., or about 60° C. In some embodiments, the major degradant is pilocarpic acid, isopilocarpic acid, isopilocarpine, or combinations thereof. In some embodiments, the major degradant is pilocarpic acid. In some embodiments, the major degradant is isopilocarpic acid. In some embodiments, the major degradant is isopilocarpine.

In some embodiments, the composition stored in a plastic container comprises less than 20% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container comprises less than 15% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container comprises less than 10% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container comprises less than 5% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the major degradant is pilocarpic acid, isopilocarpic acid, isopilocarpine, or combinations thereof. In some embodiments, the major degradant is pilocarpic acid. In some embodiments, the major degradant is isopilocarpic acid. In some embodiments, the major degradant is isopilocarpine.

In some embodiments, the composition stored in a plastic container comprises from less than 2.5% of major degradant to less than 0.1% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container comprises less than 2.5% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container comprises less than 2.0% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container comprises less than 1.5% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container comprises less than 1.0% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container comprises less than 0.5% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container comprises less than 0.4% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container comprises less than 0.3% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container comprises less than 0.2% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the composition stored in a plastic container comprises less than 0.1% of major degradant based on the concentration of the ophthalmic agent for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the major degradant is pilocarpic acid, isopilocarpic acid, isopilocarpine, or combinations thereof. In some embodiments, the major degradant is pilocarpic acid. In some embodiments, the major degradant is isopilocarpic acid. In some embodiments, the major degradant is isopilocarpine.

In some embodiments, the composition described herein is stored in a glass container. In some embodiments, the glass container is a glass vial, such as for example, a type I, type II or type III glass vial. In some embodiments, the glass container is a type I glass vial. In some embodiments, the type I glass vial is a borosilicate glass vial.

In some embodiments, the composition stored in a glass container has a pH of higher than about 7. In some embodiments, the composition stored in a glass container has a pH of higher than about 7.5. In some embodiments, the composition stored in a glass container has a pH of higher than about 8. In some embodiments, the composition stored in a glass container has a pH of higher than about 8.5. In some embodiments, the composition stored in a glass container has a pH of higher than about 9.

In some embodiments, the composition stored in a glass container has a pD of higher than about 7. In some embodiments, the composition stored in a glass container has a pD of higher than about 7.5. In some embodiments, the composition stored in a glass container has a pD of higher than about 8. In some embodiments, the composition stored in a glass container has a pD of higher than about 8.5. In some embodiments, the composition stored in a glass container has a pD of higher than about 9.

In some embodiments, the composition stored in a glass container has a potency of less than 60% at a temperature of about 25° C., about 40° C., or about 60° C. In some embodiments, the composition stored in a glass container has a potency of less than 60% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months.

In some embodiments, the composition stored in a glass container is less stable than a composition stored in a plastic container.

In some embodiments, the composition is stored under in the dark. In some instances, the composition is stored in the presence of light. In some instances, the light is indoor light, room light, or sun light. In some instances, the composition is stable while stored in the presence of light.

In some embodiments, the composition described herein is formulated as an aqueous solution. In some embodiments, the aqueous solution is a stable aqueous solution. In some instances, the aqueous solution is stored in a plastic container as described above. In some instances, the aqueous solution is not stored in a glass container. In some instances, the aqueous solution is stored in the dark. In some instances, the aqueous solution is stored in the presence of light. In some instances, the aqueous solution is stable in the presence of light.

In a specific embodiment, the ophthalmically acceptable formulations alternatively comprise a cyclodextrin. Cyclodextrins are cyclic oligosaccharides containing 6, 7, or 8 glucopyranose units, referred to as α-cyclodextrin, β-cyclodextrin, or γ-cyclodextrin respectively. Cyclodextrins have a hydrophilic exterior, which enhances water-soluble, and a hydrophobic interior which forms a cavity. In an aqueous environment, hydrophobic portions of other molecules often enter the hydrophobic cavity of cyclodextrin to form inclusion compounds. Additionally, cyclodextrins are also capable of other types of nonbonding interactions with molecules that are not inside the hydrophobic cavity. Cyclodextrins have three free hydroxyl groups for each glucopyranose unit, or 18 hydroxyl groups on α-cyclodextrin, 21 hydroxyl groups on β-cyclodextrin, and 24 hydroxyl groups on γ-cyclodextrin. In some embodiments, one or more of these hydroxyl groups are reacted with any of a number of reagents to form a large variety of cyclodextrin derivatives, including hydroxypropyl ethers, sulfonates, and sulfoalkylethers. Shown below is the structure of β-cyclodextrin and the hydroxypropyl-β-cyclodextrin (HPβCD).

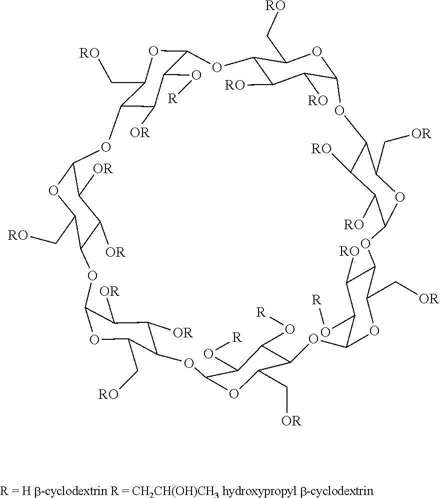

In some embodiments, the use of cyclodextrins in the pharmaceutical compositions described herein improves the solubility of the drug. Inclusion compounds are involved in many cases of enhanced solubility; however other interactions between cyclodextrins and insoluble compounds also improves solubility. Hydroxypropyl-β-cyclodextrin (HPβCD) is commercially available as a pyrogen free product. It is a nonhygroscopic white powder that readily dissolves in water. HPβCD is thermally stable and does not degrade at neutral pH. Thus, cyclodextrins improve the solubility of a therapeutic agent in a composition or formulation. Accordingly, in some embodiments, cyclodextrins are included to increase the solubility of the ophthalmically acceptable ophthalmic agents within the formulations described herein. In other embodiments, cyclodextrins in addition serve as controlled release excipients within the formulations described herein.

By way of example only, cyclodextrin derivatives for use include α-cyclodextrin, β-cyclodextrin, γ-cyclodextrin, hydroxyethyl-β-cyclodextrin, hydroxypropyl-γ-cyclodextrin, sulfated β-cyclodextrin, sulfated α-cyclodextrin, sulfobutyl ether β-cyclodextrin.

The concentration of the cyclodextrin used in the compositions and methods disclosed herein varies according to the physiochemical properties, pharmacokinetic properties, side effect or adverse events, formulation considerations, or other factors associated with the therapeutically ophthalmic agent, or a salt or prodrug thereof, or with the properties of other excipients in the composition. Thus, in certain circumstances, the concentration or amount of cyclodextrin used in accordance with the compositions and methods disclosed herein will vary, depending on the need. When used, the amount of cyclodextrins needed to increase solubility of the ophthalmic agent and/or function as a controlled release excipient in any of the formulations described herein is selected using the principles, examples, and teachings described herein.

Other stabilizers that are useful in the ophthalmically acceptable formulations disclosed herein include, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In some embodiments, amide analogues of stabilizers are also used. In further embodiments, the chosen stabilizer changes the hydrophobicity of the formulation, improves the mixing of various components in the formulation, controls the moisture level in the formula, or controls the mobility of the phase.

In other embodiments, stabilizers are present in sufficient amounts to inhibit the degradation of the ophthalmic agent. Examples of such stabilizing agents include, but are not limited to: glycerol, methionine, monothioglycerol, EDTA, ascorbic acid, polysorbate 80, polysorbate 20, arginine, heparin, dextran sulfate, cyclodextrins, pentosan polysulfate and other heparinoids, divalent cations such as magnesium and zinc, or combinations thereof. In some embodiments, the stabilizer is EDTA.

Stabilizing agents, in some embodiments, are present in the composition at about 0.001%, 0.005%, 0.010%, 0.015%, 0.020%, 0.025%, 0.030%, 0.035%, 0.040%, 0.045%, 0.050%, 0.055%, 0.060%, 0.065%, 0.070%, 0.075%, 0.080%, 0.085%, 0.090%, 0.095%, 0.1%, 0.2%, 0.3%, 0.4%, 0.5%, 0.6%, 0.7%, 0.8%, 0.9%, 1.0%, 1.5%, 2.0%, 2.5%, or 3.0%. In some embodiments, the stabilizing agent is present in the composition from about 0.001% to about 0.05%, from about 0.001% to about 0.04%, from about 0.001% to about 0.03%, from about 0.001% to about 0.025%, from about 0.001% to about 0.02%, from about 0.001% to about 0.01%, from about 0.001% to about 0.008%, or from about 0.001% to about 0.005%. In some cases, the percentage is a weight percentage.

In some embodiments, EDTA is present in the composition at about 0.001%, 0.005%, 0.010%, 0.015%, 0.020%, 0.025%, 0.030%, 0.035%, 0.040%, 0.045%, 0.050%, 0.055%, 0.060%, 0.065%, 0.070%, 0.075%, 0.080%, 0.085%, 0.090%, 0.095%, 0.1%, 0.2%, 0.3%, 0.4%, 0.5%, 0.6%, 0.7%, 0.8%, 0.9%, 1.0%, 1.5%, 2.0%, 2.5%, or 3.0%. In some embodiments, EDTA is present in the composition from about 0.01% to about 0.05%, from about 0.01% to about 0.04%, from about 0.01% to about 0.03%, from about 0.01% to about 0.025%, from about 0.01% to about 0.02%, from about 0.001% to about 0.01%, from about 0.001% to about 0.008%, or from about 0.001% to about 0.005%. In some cases, the percentage is a weight percentage.

Additional useful stabilization agents for ophthalmically acceptable formulations include one or more anti-aggregation additives to enhance stability of ophthalmic formulations by reducing the rate of protein aggregation. The anti-aggregation additive selected depends upon the nature of the conditions to which the ophthalmic agents, for example a muscarinic agent (e.g. aceclidine, pilocarpine, tropicamide, or its pharmaceutically acceptable salts), are exposed. For example, certain formulations undergoing agitation and thermal stress require a different anti-aggregation additive than a formulation undergoing lyophilization and reconstitution. Useful anti-aggregation additives include, by way of example only, urea, guanidinium chloride, simple amino acids such as glycine or arginine, sugars, polyalcohols, polysorbates, polymers such as polyethylene glycol and dextrans, alkyl saccharides, such as alkyl glycoside, and surfactants.

Other useful formulations optionally include one or more ophthalmically acceptable antioxidants to enhance chemical stability where required. Suitable antioxidants include, by way of example only, ascorbic acid, methionine, sodium thiosulfate and sodium metabisulfite. In one embodiment, antioxidants are selected from metal chelating agents, thiol containing compounds and other general stabilizing agents.

Still other useful compositions include one or more ophthalmically acceptable surfactants to enhance physical stability or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In some embodiments, the ophthalmically acceptable pharmaceutical formulations described herein are stable with respect to compound degradation (e.g. less than 30% degradation, less than 25% degradation, less than 20% degradation, less than 15% degradation, less than 10% degradation, less than 8% degradation, less than 5% degradation, less than 3% degradation, less than 2% degradation, or less than 5% degradation) over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, or at least about 6 months under storage conditions (e.g. room temperature). In other embodiments, the formulations described herein are stable with respect to compound degradation over a period of at least about 1 week. Also described herein are formulations that are stable with respect to compound degradation over a period of at least about 1 month.

In other embodiments, an additional surfactant (co-surfactant) and/or buffering agent is combined with one or more of the pharmaceutically acceptable vehicles previously described herein so that the surfactant and/or buffering agent maintains the product at an optimal pH for stability. Suitable co-surfactants include, but are not limited to: a) natural and synthetic lipophilic agents, e.g., phospholipids, cholesterol, and cholesterol fatty acid esters and derivatives thereof; b) nonionic surfactants, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL) and other Cremophors, sulfosuccinates, alkyl sulphates (SLS); PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate; Brij® 700, ascorbyl-6-palmitate, stearylamine, sodium lauryl sulfate, polyoxethyleneglycerol triiricinoleate, and any combinations or mixtures thereof; c) anionic surfactants include, but are not limited to, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and d) cationic surfactants such as cetyltrimethylammonium bromide, and lauryldimethylbenzyl-ammonium chloride.

In a further embodiment, when one or more co-surfactants are utilized in the ophthalmically acceptable formulations of the present disclosure, they are combined, e.g., with a pharmaceutically acceptable vehicle and is present in the final formulation, e.g., in an amount ranging from about 0.1% to about 20%, from about 0.5% to about 10%.

In one embodiment, the surfactant has an HLB value of 0 to 20. In additional embodiments, the surfactant has an HLB value of 0 to 3, of 4 to 6, of 7 to 9, of 8 to 18, of 13 to 15, of 10 to 18.

pH

In some embodiments, the pH of a composition described herein is adjusted (e.g., by use of a buffer and/or a pH adjusting agent) to an ophthalmically compatible pH range of from about 4 to about 8, about 4.2 to about 7.9, about 4.5 to about 7.5, or about 5 to about 7. In some embodiments, the ophthalmic composition has a pH of from about 5.0 to about 7.0. In some embodiments, the ophthalmic composition has a pH of from about 5.5 to about 7.0. In some embodiments, the ophthalmic composition has a pH of from about 6.0 to about 7.0.

In some embodiments, useful formulations include one or more pH adjusting agents or buffering agents. Suitable pH adjusting agents or buffers include, but are not limited to acetate, bicarbonate, ammonium chloride, citrate, phosphate, deuterated forms of acetate, bicarbonate, ammonium chloride, citrate, phosphate, pharmaceutically acceptable salts thereof and combinations or mixtures thereof. In some embodiments, the pH adjusting agents or buffers include deuterated hydrochloric acid (DCl), deuterated sodium hydroxide (NaOD), deuterated acetic acid (CD₃COOD), or deuterated citric acid (C₆D₈O₇).

In one embodiment, when one or more buffers are utilized in the formulations of the present disclosure, they are combined, e.g., with a pharmaceutically acceptable vehicle and are present in the final formulation, e.g., in an amount ranging from about 0.1% to about 20%, from about 0.5% to about 10%. In certain embodiments of the present disclosure, the amount of buffer included in the gel formulations are an amount such that the pH of the gel formulation does not interfere with the body's natural buffering system.

In one embodiment, diluents are also used to stabilize compounds because they provide a more stable environment. In some instances, salts dissolved in buffered solutions (which also provides pH control or maintenance) are utilized as diluents in the art, including, but not limited to a phosphate buffered saline solution. In some embodiments, pH and pD of the present disclosure are based on the apparent (measured) pH of a system, using an electrode calibrated with aqueous buffers. In the case of a 100% D₂O system the apparent pH will be less than the pD (−log₁₀[molar deuteron concentration]) of the system by approximately 0.44 units. In the case of a 100% H₂O system, the apparent pH is the pH (−log₁₀[molar proton concentration]) of the system. In the case of a mixed H₂O/D₂O system, the apparent pH is less than the pH of the system by approximately 0-0.44 units depending on the ratio between H₂O and D₂O.

In some embodiments, the pD is calculated according to the formula disclosed in Glasoe et al., “Use of glass electrodes to measure acidities in deuterium oxide,” J. Physical Chem. 64(1): 188-190 (1960). In some embodiments, the pD is calculated as pD=pH+0.4, in which pH is the measured or observed pH of the ophthalmic composition formulated in a solution comprising deuterated water (e.g., D₂O).

In some embodiments, the ophthalmic aqueous, gel, or ointment composition described herein has a pH of between about 4 and about 8, between about 4.5 and about 8, between about 4.9 and about 7.9, between about 5.4 and about 7.9, between about 5.9 and about 7.9, between about 6.4 and about 7.9, or between about 7.4 and about 7.9. In some embodiments, the ophthalmic aqueous, gel, or ointment composition described herein has a pH of between about 4.5-7.5, between about 5.0 and about 7.5, between about 5.5 and about 7.5, between about 6.0 and about 7.5, or between about 7.0 and about 7.5. In some embodiments, the ophthalmic aqueous, gel, or ointment composition described herein has a pH of between about 4.5-7.0, between about 5.0 and about 7.0, between about 5.5 and about 7.0, between about 6.0 and about 7.0, or between about 6.5 and about 7.0. In some embodiments, the ophthalmic aqueous, gel, or ointment composition described herein has a pH of between about 4.9-7.4, between about 5.4 and about 7.4, between about 5.9 and about 7.4, between about 6.4 and about 7.4, or between about 6.9 and about 7.4. In some embodiments, the ophthalmic aqueous, gel, or ointment composition described herein has a pH of between about 4.5-6.5, between about 5.0 and about 6.5, between about 5.5 and about 6.5, or between about 6.0 and about 6.5. In some embodiments, the ophthalmic aqueous, gel, or ointment composition described herein has a pH of between about 4.9-6.9, between about 5.4 and about 6.9, between about 5.9 and about 6.9, or between about 6.4 and about 6.9. In some embodiments, the ophthalmic aqueous, gel, or ointment composition described herein has a pH of between about 4.5-6.0, between about 5.0 and about 6.0, or between about 5.5 and about 6.0. In some embodiments, the ophthalmic aqueous, gel, or ointment composition described herein has a pH of between about 4.9-6.4, between about 5.4 and about 6.4, or between about 5.9 and about 6.4. In some embodiments, the ophthalmic aqueous, gel, or ointment composition described herein has a pH of between about 4.5-5.5, or between about 5.0 and about 5.5. In some embodiments, the ophthalmic aqueous, gel, or ointment composition described herein has a pH of between about 4.9-5.9, or between about 5.4 and about 5.9. In some embodiments, the ophthalmic aqueous, gel, or ointment composition described herein has a pH of between about 4.5-5.0. In some embodiments, the ophthalmic aqueous, gel, or ointment composition described herein has a pH of between about 4.9-5.4.

In some embodiments, the ophthalmic composition is an ophthalmic aqueous composition. In some instances, the ophthalmic aqueous composition has a pH of between about 4 and about 8, about 4.2 to about 7.9, about 4.5 and about 7.8, about 5 and about 7.5, or about 5.5 and about 7. In some embodiments, the ophthalmic aqueous composition has a pH of about 8.0. In some embodiments, the ophthalmic aqueous composition has a pH of about 7.9. In some embodiments, the ophthalmic aqueous composition has a pH of about 7.8. In some embodiments, the ophthalmic aqueous composition has a pH of about 7.7. In some embodiments, the ophthalmic aqueous composition has a pH of about 7.6. In some embodiments, the ophthalmic aqueous composition has a pH of about 7.5. In some embodiments, the ophthalmic aqueous composition has a pH of about 7.4. In some embodiments, the ophthalmic aqueous composition has a pH of about 7.3. In some embodiments, the ophthalmic aqueous composition has a pH of about 7.2. In some embodiments, the ophthalmic aqueous composition has a pH of about 7.1. In some embodiments, the ophthalmic aqueous composition has a pH of about 7. In some embodiments, the ophthalmic aqueous composition has a pH of about 6.9. In some embodiments, the ophthalmic aqueous composition has a pH of about 6.8. In some embodiments, the ophthalmic aqueous composition has a pH of about 6.7. In some embodiments, the ophthalmic aqueous composition has a pH of about 6.6. In some embodiments, the ophthalmic aqueous composition has a pH of about 6.5. In some embodiments, the ophthalmic aqueous composition has a pH of about 6.4. In some embodiments, the ophthalmic aqueous composition has a pH of about 6.3. In some embodiments, the ophthalmic aqueous composition has a pH of about 6.2. In some embodiments, the ophthalmic aqueous composition has a pH of about 6.1. In some embodiments, the ophthalmic aqueous composition has a pH of about 6. In some embodiments, the ophthalmic aqueous composition has a pH of about 5.9. In some embodiments, the ophthalmic aqueous composition has a pH of about 5.8. In some embodiments, the ophthalmic aqueous composition has a pH of about 5.7. In some embodiments, the ophthalmic aqueous composition has a pH of about 5.6. In some embodiments, the ophthalmic aqueous composition has a pH of about 5.5. In some embodiments, the ophthalmic aqueous composition has a pH of about 5.4. In some embodiments, the ophthalmic aqueous composition has a pH of about 5.3. In some embodiments, the ophthalmic aqueous composition has a pH of about 5.2. In some embodiments, the ophthalmic aqueous composition has a pH of about 5.1. In some embodiments, the ophthalmic aqueous composition has a pH of about 5. In some embodiments, the ophthalmic aqueous composition has a pH of about 4.9. In some embodiments, the ophthalmic aqueous composition has a pH of about 4.8. In some embodiments, the ophthalmic aqueous composition has a pH of about 4.7. In some embodiments, the ophthalmic aqueous composition has a pH of about 4.6. In some embodiments, the ophthalmic aqueous composition has a pH of about 4.5. In some embodiments, the ophthalmic aqueous composition has a pH of about 4.4. In some embodiments, the ophthalmic aqueous composition has a pH of about 4.3. In some embodiments, the ophthalmic aqueous composition has a pH of about 4.2. In some embodiments, the ophthalmic aqueous composition has a pH of about 4.1. In some embodiments, the ophthalmic aqueous composition has a pH of about 4. In some embodiments, the pH is an initial pH of the ophthalmic aqueous composition. In some embodiments, the pH is the pH of the ophthalmic aqueous composition after extended period of time under a storage condition.

In some instances, the ophthalmic aqueous composition has an initial pH of between about 4 and about 8, about 4.2 to about 7.9, about 4.5 and about 7.8, about 5 and about 7.5, or about 5.5 and about 7. In some embodiments, the ophthalmic aqueous composition has an initial pH of about 8.0. In some embodiments, the ophthalmic aqueous composition has an initial pH of about 7.9. In some embodiments, the ophthalmic aqueous composition has an initial pH of about 7.8. In some embodiments, the ophthalmic aqueous composition has an initial pH of about 7.7. In some embodiments, the ophthalmic aqueous composition has an initial pH of about 7.6. In some embodiments, the ophthalmic aqueous composition has an initial pH of about 7.5. In some embodiments, the ophthalmic aqueous composition has an initial pH of about 7.4. In some embodiments, the ophthalmic aqueous composition has an initial pH of about 7.3. In some embodiments, the ophthalmic aqueous composition has an initial pH of about 7.2. In some embodiments, the ophthalmic aqueous composition has an initial pH of about 7.1. In some embodiments, the ophthalmic aqueous composition has an initial pH of about 7. In some embodiments, the ophthalmic aqueous composition has an initial pH of about 6.9. In some embodiments, the ophthalmic aqueous composition has an initial pH of about 6.8. In some embodiments, the ophthalmic aqueous composition has an initial pH of about 6.7. In some embodiments, the ophthalmic aqueous composition has an initial pH of about 6.6. In some embodiments, the ophthalmic aqueous composition has an initial pH of about 6.5. In some embodiments, the ophthalmic aqueous composition has an initial pH of about 6.4. In some embodiments, the ophthalmic aqueous composition has an initial pH of about 6.3. In some embodiments, the ophthalmic aqueous composition has an initial pH of about 6.2. In some embodiments, the ophthalmic aqueous composition has an initial pH of about 6.1. In some embodiments, the ophthalmic aqueous composition has an initial pH of about 6. In some embodiments, the ophthalmic aqueous composition has an initial pH of about 5.9. In some embodiments, the ophthalmic aqueous composition has an initial pH of about 5.8. In some embodiments, the ophthalmic aqueous composition has an initial pH of about 5.7. In some embodiments, the ophthalmic aqueous composition has an initial pH of about 5.6. In some embodiments, the ophthalmic aqueous composition has an initial pH of about 5.5. In some embodiments, the ophthalmic aqueous composition has an initial pH of about 5.4. In some embodiments, the ophthalmic aqueous composition has an initial pH of about 5.3. In some embodiments, the ophthalmic aqueous composition has an initial pH of about 5.2. In some embodiments, the ophthalmic aqueous composition has an initial pH of about 5.1. In some embodiments, the ophthalmic aqueous composition has an initial pH of about 5. In some embodiments, the ophthalmic aqueous composition has an initial pH of about 4.9. In some embodiments, the ophthalmic aqueous composition has an initial pH of about 4.8. In some embodiments, the ophthalmic aqueous composition has an initial pH of about 4.7. In some embodiments, the ophthalmic aqueous composition has an initial pH of about 4.6. In some embodiments, the ophthalmic aqueous composition has an initial pH of about 4.5. In some embodiments, the ophthalmic aqueous composition has an initial pH of about 4.4. In some embodiments, the ophthalmic aqueous composition has an initial pH of about 4.3. In some embodiments, the ophthalmic aqueous composition has an initial pH of about 4.2. In some embodiments, the ophthalmic aqueous composition has an initial pH of about 4.1. In some embodiments, the ophthalmic aqueous composition has an initial pH of about 4.

In some instances, the ophthalmic aqueous composition has a pH of between about 4 and about 8, about 4.9 to about 7.2, about 4.5 and about 7.8, about 5 and about 7.5, or about 5.5 and about 7. In some embodiments, the ophthalmic aqueous composition has a pH of about 8.0. In some embodiments, the ophthalmic aqueous composition has a pH of about 7.9. In some embodiments, the ophthalmic aqueous composition has a pH of about 7.8. In some embodiments, the ophthalmic aqueous composition has a pH of about 7.7. In some embodiments, the ophthalmic aqueous composition has a pH of about 7.6. In some embodiments, the ophthalmic aqueous composition has a pH of less than about 7.5. In some embodiments, the ophthalmic aqueous composition has a pH of less than about 7.4. In some embodiments, the ophthalmic aqueous composition has a pH of less than about 7.3. In some embodiments, the ophthalmic aqueous composition has a pH of less than about 7.2. In some embodiments, the ophthalmic aqueous composition has a pH of less than about 7.1. In some embodiments, the ophthalmic aqueous composition has a pH of less than about 7. In some embodiments, the ophthalmic aqueous composition has a pH of less than about 6.9. In some embodiments, the ophthalmic aqueous composition has a pH of less than about 6.8. In some embodiments, the ophthalmic aqueous composition has a pH of less than about 6.7. In some embodiments, the ophthalmic aqueous composition has a pH of less than about 6.6. In some embodiments, the ophthalmic aqueous composition has a pH of less than about 6.5. In some embodiments, the ophthalmic aqueous composition has a pH of less than about 6.4. In some embodiments, the ophthalmic aqueous composition has a pH of less than about 6.3. In some embodiments, the ophthalmic aqueous composition has a pH of less than about 6.2. In some embodiments, the ophthalmic aqueous composition has a pH of less than about 6.1. In some embodiments, the ophthalmic aqueous composition has a pH of less than about 6. In some embodiments, the ophthalmic aqueous composition has a pH of less than about 5.9. In some embodiments, the ophthalmic aqueous composition has a pH of less than about 5.8. In some embodiments, the ophthalmic aqueous composition has a pH of less than about 5.7. In some embodiments, the ophthalmic aqueous composition has a pH of less than about 5.6. In some embodiments, the ophthalmic aqueous composition has a pH of less than about 5.5. In some embodiments, the ophthalmic aqueous composition has a pH of less than about 5.4. In some embodiments, the ophthalmic aqueous composition has a pH of less than about 5.3. In some embodiments, the ophthalmic aqueous composition has a pH of less than about 5.2. In some embodiments, the ophthalmic aqueous composition has a pH of less than about 5.1. In some embodiments, the ophthalmic aqueous composition has a pH of less than about 5. In some embodiments, the ophthalmic aqueous composition has a pH of less than about 4.9. In some embodiments, the ophthalmic aqueous composition has a pH of less than about 4.8. In some embodiments, the ophthalmic aqueous composition has a pH of less than about 4.7. In some embodiments, the ophthalmic aqueous composition has a pH of less than about 4.6. In some embodiments, the ophthalmic aqueous composition has a pH of less than about 4.5. In some embodiments, the ophthalmic aqueous composition has a pH of less than about 4.4. In some embodiments, the ophthalmic aqueous composition has a pH of less than about 4.3. In some embodiments, the ophthalmic aqueous composition has a pH of less than about 4.2. In some embodiments, the ophthalmic aqueous composition has a pH of less than about 4.1. In some embodiments, the ophthalmic aqueous composition has a pH of less than about 4. In some embodiments, the pH is the pH of the ophthalmic aqueous composition after extended period of time under a storage condition.

In some embodiments, the pH of the ophthalmic aqueous composition described herein is associated with the stability of the ophthalmic aqueous composition. In some embodiments, a stable composition comprises a pH of between about 4 and about 8, about 4.2 to about 7.9, about 4.5 and about 7.8, about 5 and about 7.5, or about 5.5 and about 7. In some embodiments, a stable composition comprises a pH of about 8.0. In some embodiments, a stable composition comprises a pH of about 7.9. In some embodiments, a stable composition comprises a pH of about 7.8. In some embodiments, a stable composition comprises a pH of about 7.7. In some embodiments, a stable composition comprises a pH of about 7.6. In some embodiments, a stable composition comprises a pH of less than about 7.5. In some embodiments, a stable composition comprises a pH of less than about 7.4. In some embodiments, a stable composition comprises a pH of less than about 7.3. In some embodiments, a stable composition comprises a pH of less than about 7.2. In some embodiments, a stable composition comprises a pH of less than about 7.1. In some embodiments, a stable composition comprises a pH of less than about 7. In some embodiments, a stable composition comprises a pH of less than about 6.9. In some embodiments, a stable composition comprises a pH of less than about 6.8. In some embodiments, a stable composition comprises a pH of less than about 6.7. In some embodiments, a stable composition comprises a pH of less than about 6.6. In some embodiments, a stable composition comprises a pH of less than about 6.5. In some embodiments, a stable composition comprises a pH of less than about 6.4. In some embodiments, a stable composition comprises a pH of less than about 6.3. In some embodiments, a stable composition comprises a pH of less than about 6.2. In some embodiments, a stable composition comprises a pH of less than about 6.1. In some embodiments, a stable composition comprises a pH of less than about 6. In some embodiments, a stable composition comprises a pH of less than about 5.9. In some embodiments, a stable composition comprises a pH of less than about 5.8. In some embodiments, a stable composition comprises a pH of less than about 5.7. In some embodiments, a stable composition comprises a pH of less than about 5.6. In some embodiments, a stable composition comprises a pH of less than about 5.5. In some embodiments, a stable composition comprises a pH of less than about 5.4. In some embodiments, a stable composition comprises a pH of less than about 5.3. In some embodiments, a stable composition comprises a pH of less than about 5.2. In some embodiments, a stable composition comprises a pH of less than about 5.1. In some embodiments, a stable composition comprises a pH of less than about 5. In some embodiments, a stable composition comprises a pH of less than about 4.9. In some embodiments, a stable composition comprises a pH of less than about 4.8. In some embodiments, a stable composition comprises a pH of less than about 4.7. In some embodiments, a stable composition comprises a pH of less than about 4.6. In some embodiments, a stable composition comprises a pH of less than about 4.5. In some embodiments, a stable composition comprises a pH of less than about 4.4. In some embodiments, a stable composition comprises a pH of less than about 4.3. In some embodiments, a stable composition comprises a pH of less than about 4.2. In some embodiments, a stable composition comprises a pH of less than about 4.1. In some embodiments, a stable composition comprises a pH of less than about 4.

In some embodiments, the D₂O/aqueous system stabilizes a muscarinic agent (e.g. aceclidine, pilocarpine, or tropicamide). In some embodiments, this is due to a lower concentration of the reactive species (e.g., —OD) in the D₂O/aqueous system compared to the concentration of the reactive species (e.g., —OH) in an equivalent purely aqueous system. In some instances, the concentration of the reactive species (e.g., —OD) in the D₂O/aqueous system is about one third less than the concentration of the reactive species (e.g., —OH) in the equivalent purely aqueous system. In some cases, this is due to a lower or smaller dissociation constant of D₂O than H₂O. For example, the Kₐ(H₂O) is 1×10¹⁴, whereas the Kₐ(D₂O) is 1×10¹⁵. As such, D₂O is a weaker acid than H₂O. In some embodiments, the ophthalmic composition formulated with deuterated water allows for a more stable ophthalmic composition relative to the ophthalmic composition formulated with H₂O.

In some embodiments, the presence of deuterated water shifts the pKa of the buffer. In some embodiments, the presence of deuterated water allows for the ophthalmic composition to simulate the stability of a lower pH system. In some instances, the buffer capacity of the ophthalmic composition is lowered, thereby allowing a faster shift in pH. In some instances, the lowered buffering capacity of the ophthalmic composition when administered into the eye allows the ophthalmic composition to reach physiological pH at a faster rate than compared to an ophthalmic composition formulated in H₂O. In some instances, the ophthalmic composition formulated with deuterated water allows for a lower tear production, or less tear reflex in the eye, in comparison with an ophthalmic composition formulated with H₂O.

In some embodiment, the ophthalmic gel or ointment composition described herein has a pH of about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, or about 7.9.

In some embodiment, the pH of the ophthalmic aqueous, gel, or ointment composition described herein is suitable for sterilization (e.g., by filtration or aseptic mixing or heat treatment and/or autoclaving (e.g., terminal sterilization) of ophthalmic formulations described herein. As used in in the present disclosure, the term “aqueous composition” comprises compositions that are based on D₂O.

In some embodiments, the pharmaceutical formulations described herein are stable with respect to pH over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 18 months, at least about 24 months, at least about 3 years, at least about 4 years, at least about 5 years, at least about 6 years, at least about 7 years, at least about 8 years, at least about 9 years, at least about 10 years, or more. In other embodiments, the formulations described herein are stable with respect to pH over a period of at least about 1 week. In other embodiments, the formulations described herein are stable with respect to pH over a period of at least about 2 weeks. In other embodiments, the formulations described herein are stable with respect to pH over a period of at least about 3 weeks. In other embodiments, the formulations described herein are stable with respect to pH over a period of at least about 1 month. Also described herein are formulations that are stable with respect to pH over a period of at least about 2 months, at least about 3 months, at least about 4 months, at least about 5 months, at least about 6 months, at least about 12 months, at least about 18 months, at least about 2 years, or more.

### Aqueous Solution Dose-to-Dose Uniformity

Typical ophthalmic aqueous solutions are packaged in eye drop bottles and administered as drops. For example, a single administration (i.e. a single dose) of an ophthalmic aqueous solution comprises a single drop, two drops, three drops or more into the eyes of the patient. In some embodiments, one dose of the ophthalmic aqueous solution described herein is one drop of the aqueous solution composition from the eye drop bottle.

In some embodiments, a drop comprises at least or about 10 microliters (μL), 15 μL, 20 μL, 25 μL, 30 μL, 35 μL, 40 μL, 45 μL, 50 μL, 75 μL, 100 μL, 125 μL, 150 μL, or more than 150 μL. In some embodiments, a drop comprises about 10 μL to about 100 μL, about 10 μL to about 75 μL, about 10 μL to about 50 μL, about 20 μL to about 100 μL, about 25 μL to about 75 μL, about 50 μL to about 75 μL, or about 50 μL to about 100 μL.

In some cases, described herein include ophthalmic aqueous compositions which provide a dose-to-dose uniform concentrations. In some instances, the dose-to-dose uniform concentration does not present significant variations of drug content from one dose to another. In some instances, the dose-to-dose uniform concentration does provide consistent drug content from one dose to another.

In some embodiments, the composition has a dose-to-dose ophthalmic agent concentration variation of less than 50%. In some embodiments, the composition has a dose-to-dose ophthalmic agent concentration variation of less than 40%. In some embodiments, the composition has a dose-to-dose ophthalmic agent concentration variation of less than 30%. In some embodiments, the composition has a dose-to-dose ophthalmic agent concentration variation of less than 20%. In some embodiments, the composition has a dose-to-dose ophthalmic agent concentration variation of less than 10%. In some embodiments, the composition has a dose-to-dose ophthalmic agent concentration variation of less than 5%.

In some embodiments, the dose-to-dose ophthalmic agent concentration variation is based on 10 consecutive doses. In some embodiments, the dose-to-dose ophthalmic agent concentration variation is based on 8 consecutive doses. In some embodiments, the dose-to-dose ophthalmic agent concentration variation is based on 5 consecutive doses. In some embodiments, the dose-to-dose ophthalmic agent concentration variation is based on 3 consecutive doses. In some embodiments, the dose-to-dose ophthalmic agent concentration variation is based on 2 consecutive doses.

A nonsettling formulation should not require shaking to disperse drug uniformly. A “no-shake” formulation is potentially advantageous over formulations that require shaking for the simple reason that patients' shaking behavior is a major source of variability in the amount of drug dosed. It has been reported that patients often times do not or forget to shake their ophthalmic compositions that requires shaking before administering a dose, despite the instructions to shake that were clearly marked on the label. On the other hand, even for those patients who do shake the product, it is normally not possible to determine whether the shaking is adequate in intensity and/or duration to render the product uniform. In some embodiments, the ophthalmic gel compositions and ophthalmic ointment compositions described herein are “no-shake” formulations that maintained the dose-to-dose uniformity described herein.

To evaluate the dose-to-dose uniformity, drop bottles or tubes containing the ophthalmic aqueous compositions, the ophthalmic gel compositions, or ophthalmic ointment compositions are stored upright for a minimum of 12 hours prior to the start of the test. To simulate the recommended dosing of these products, predetermined number of drops or strips are dispensed from each commercial bottles or tubes at predetermined time intervals for an extended period of time or until no product was left in the bottle or tube. All drops and strips are dispensed into tared glass vials, capped, and stored at room temperature until analysis. Concentrations of. a muscarinic agent such as aceclidine, pilocarpine, or tropicamide in the expressed drops are determined using a reverse-phase HPLC method.

### Aqueous Solution Viscosity

In some embodiments, the composition has a Brookfield RVDV viscosity of from about 10 to about 50,000 cps at about 20° C. and sheer rate of 1 s⁻¹. In some embodiments, the composition has a Brookfield RVDV viscosity of from about 100 to about 40,000 cps at about 20° C. and sheer rate of 1 s⁻¹. In some embodiments, the composition has a Brookfield RVDV viscosity of from about 500 to about 30,000 cps at about 20° C. and sheer rate of 1 s⁻¹. In some embodiments, the composition has a Brookfield RVDV viscosity of from about 1000 to about 20,000 cps at about 20° C. and sheer rate of 1 s⁻¹. In some embodiments, the composition has a Brookfield RVDV viscosity of from about 2000 to about 10,000 cps at about 20° C. and sheer rate of 1 s⁻¹. In some embodiments, the composition has a Brookfield RVDV viscosity of from about 4000 to about 8000 cps at about 20° C. and sheer rate of 1 s⁻¹.

In some embodiments, the ophthalmic aqueous formulation contains a viscosity enhancing agent sufficient to provide a viscosity of between about 500 and 50,000 centipoise, between about 750 and 50,000 centipoise; between about 1000 and 50,000 centipoise; between about 1000 and 40,000 centipoise; between about 2000 and 30,000 centipoise; between about 3000 and 20,000 centipoise; between about 4000 and 10,000 centipoise, or between about 5000 and 8000 centipoise.

A viscosity enhancing agent, in some embodiments, is present in the composition at about 0.001%, 0.005%, 0.010%, 0.015%, 0.020%, 0.025%, 0.030%, 0.035%, 0.040%, 0.045%, 0.050%, 0.055%, 0.060%, 0.065%, 0.070%, 0.075%, 0.080%, 0.085%, 0.090%, 0.095%, 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, 5.5%, or 6%. In some embodiments, the viscosity enhancing agent is present in the composition from about 0.001% to about 0.05%, from about 0.001% to about 0.04%, from about 0.001% to about 0.03%, from about 0.001% to about 0.025%, from about 0.001% to about 0.02%, from about 0.001% to about 0.01%, from about 0.001% to about 0.008%, or from about 0.001% to about 0.005%. In some cases, the percentage is a weight percentage.

In some embodiments, the compositions described herein are low viscosity compositions at body temperature. In some embodiments, low viscosity compositions contain from about 1% to about 10% of a viscosity enhancing agent (e.g., gelling components such as polyoxyethylene-polyoxypropylene copolymers). In some embodiments, low viscosity compositions contain from about 2% to about 10% of a viscosity enhancing agent (e.g., gelling components such as polyoxyethylene-polyoxypropylene copolymers). In some embodiments, low viscosity compositions contain from about 5% to about 10% of a viscosity enhancing agent (e.g., gelling components such as polyoxyethylene-polyoxypropylene copolymers). In some embodiments, low viscosity compositions are substantially free of a viscosity enhancing agent (e.g., gelling components such as polyoxyethylene-polyoxypropylene copolymers). In some embodiments, a low viscosity ophthalmic agent composition described herein provides an apparent viscosity of from about 100 cP to about 10,000 cP. In some embodiments, a low viscosity ophthalmic agent composition described herein provides an apparent viscosity of from about 500 cP to about 10,000 cP. In some embodiments, a low viscosity ophthalmic agent composition described herein provides an apparent viscosity of from about 1000 cP to about 10,000 cP.

### Osmolarity

In some embodiments, a composition disclosed herein is formulated in order to not disrupt the ionic balance of the eye. In some embodiments, a composition disclosed herein has an ionic balance that is the same as or substantially the same as the eye. In some embodiments, a composition disclosed herein does not disrupt the ionic balance of the eye.

As used herein, “practical osmolarity/osmolality” or “deliverable osmolarity/osmolality” means the osmolarity/osmolality of a composition as determined by measuring the osmolarity/osmolality of the ophthalmic agent and all excipients except the gelling and/or the thickening agent (e.g., polyoxyethylene-polyoxypropylene copolymers, carboxymethylcellulose or the like). The practical osmolarity of a composition disclosed herein is measured by a suitable method, e.g., a freezing point depression method as described in Viegas et. al., Int. J. Pharm., 1998, 160, 157-162. In some instances, the practical osmolarity of a composition disclosed herein is measured by vapor pressure osmometry (e.g., vapor pressure depression method) that allows for determination of the osmolarity of a composition at higher temperatures. In some instances, vapor pressure depression method allows for determination of the osmolarity of a composition comprising a gelling agent (e.g., a thermoreversible polymer) at a higher temperature wherein the gelling agent is in the form of a gel.

In some embodiments, the osmolarity at a target site of action (e.g., the eye) is about the same as the delivered osmolarity of a composition described herein. In some embodiments, a composition described herein has a deliverable osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L, or about 250 mOsm/L to about 320 mOsm/L.

The practical osmolality of an ophthalmic composition disclosed herein is from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg, or from about 280 mOsm/kg to about 320 mOsm/kg. In some embodiments, a composition described herein has a practical osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L.

In some embodiments, suitable osmolarity adjusting agents include, but are not limited to any pharmaceutically acceptable sugar, salt or any combinations or mixtures thereof, such as, but not limited to dextrose, glycerin, mannitol, sorbitol, sodium chloride, and other electrolytes. In some instances, the tonicity adjusting agent is selected from sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, dextrose, mannitol, sorbitol, dextrose, sucrose, urea, propylene glycol, glycerin, or a combination thereof.

In some instances, the osmolarity adjusting agent is present in the composition between about 0.01% and about 3.0%. In some instances, the osmolarity adjusting agent is present in the composition is between about 0.7% and about 1.8%, about 0.8% and about 1.5%, or about 1% and about 1.3%. In some instances, the osmolarity adjusting agent is present in the composition from about 0.01 wt % to about 1.0 wt %, from about 0.05 wt % to about 1.5 wt %, from about 0.075 wt % to about 2.0 wt %, or from about 0.1 wt % to about 3.0 wt %. In some instances, the osmolarity adjusting agent is present in the composition is about 0.6%, 0.7%, 0.8%, 0.9%, 1.0%, 1.1%, 1.2%, 1.3%, 1.4%, 1.5%, 1.6%, 1.7%, 1.8%, or 1.9%. In some instances, the osmolarity adjusting agent is present in the composition is about 0.001%, 0.002%, 0.003%, 0.004%, 0.005%, 0.006%, 0.008%, 0.009%, 0.009%, 0.01%, 0.015%, 0.02%, 0.025%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 2.0%, 3.0%, 4.0%, or more than 4.0%. In some cases, the percentage is a weight percentage.

In some embodiments, the osmolarity adjusting agent is sodium chloride. In some instances, the sodium chloride is present in the composition between about 0.01% and about 3.0%. In some instances, the sodium chloride is present in the composition is between about 0.7% and about 1.8%, about 0.8% and about 1.5%, or about 1% and about 1.3%. In some instances, the sodium chloride is present in the composition from about 0.01 wt % to about 1.0 wt %, from about 0.05 wt % to about 1.5 wt %, from about 0.075 wt % to about 2.0 wt %, or from about 0.1 wt % to about 3.0 wt %. In some instances, the sodium chloride is present in the composition is about 0.6%, 0.7%, 0.8%, 0.9%, 1.0%, 1.1%, 1.2%, 1.3%, 1.4%, 1.5%, 1.6%, 1.7%, 1.8%, or 1.9%. In some instances, the sodium chloride is present in the composition is about 0.001%, 0.002%, 0.003%, 0.004%, 0.005%, 0.006%, 0.008%, 0.009%, 0.009%, 0.01%, 0.015%, 0.02%, 0.025%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 2.0%, 3.0%, 4.0%, or more than 4.0%. In some cases, the percentage is a weight percentage.

In some embodiment, the ophthalmic compositions described herein include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

### Sterility

In some embodiments, the compositions are sterilized. Included within the embodiments disclosed herein are means and processes for sterilization of a pharmaceutical composition disclosed herein for use in humans. The goal is to provide a safe pharmaceutical product, relatively free of infection causing micro-organisms. The U. S. Food and Drug Administration has provided regulatory guidance in the publication “Guidance for Industry: Sterile Drug Products Produced by Aseptic Processing” available at: http://www.fda.gov/cder/guidance/5882fnl.htm, which is incorporated herein by reference in its entirety.

As used herein, sterilization means a process used to destroy or remove microorganisms that are present in a product or packaging. Any suitable method available for sterilization of objects and compositions is used. Available methods for the inactivation of microorganisms include, but are not limited to, the application of extreme heat, lethal chemicals, or gamma radiation. In some embodiments, a process for the preparation of an ophthalmic formulation comprises subjecting the formulation to a sterilization method selected from heat sterilization, chemical sterilization, radiation sterilization or filtration sterilization. The method used depends largely upon the nature of the device or composition to be sterilized. Detailed descriptions of many methods of sterilization are given in Chapter 40 of Remington: The Science and Practice of Pharmacy published by Lippincott, Williams & Wilkins, and is incorporated by reference with respect to this subject matter.

### Filtration

Filtration sterilization is a method used to remove but not destroy microorganisms from solutions. Membrane filters are used to filter heat-sensitive solutions. Such filters are thin, strong, homogenous polymers of mixed cellulosic esters (MCE), polyvinylidene fluoride (PVF; also known as PVDF), or polytetrafluoroethylene (PTFE) and have pore sizes ranging from 0.1 to 0.22 μm. Solutions of various characteristics are optionally filtered using different filter membranes. For example, PVF and PTFE membranes are well suited to filtering organic solvents while aqueous solutions are filtered through PVF or MCE membranes. Filter apparatus are available for use on many scales ranging from the single point-of-use disposable filter attached to a syringe up to commercial scale filters for use in manufacturing plants. The membrane filters are sterilized by autoclave or chemical sterilization. Validation of membrane filtration systems is performed following standardized protocols (Microbiological Evaluation of Filters for Sterilizing Liquids, Vol 4, No. 3. Washington, D.C.: Health Industry Manufacturers Association, 1981) and involve challenging the membrane filter with a known quantity (ca. 10⁷/cm²) of unusually small microorganisms, such as Brevundimonas diminuta (ATCC 19146).

Pharmaceutical compositions are optionally sterilized by passing through membrane filters. Formulations comprising nanoparticles (U.S. Pat. No. 6,139,870) or multilamellar vesicles (Richard et al., International Journal of Pharmaceutics (2006), 312(1-2):144-50) are amenable to sterilization by filtration through 0.22 μm filters without destroying their organized structure.

In some embodiments, the methods disclosed herein comprise sterilizing the formulation (or components thereof) by means of filtration sterilization. In ophthalmic gel compositions that comprises thermosetting polymers, filtration is carried out below (e.g. about 5° C.) the gel temperature (Tgel) of a formulation described herein and with viscosity that allows for filtration in a reasonable time using a peristaltic pump (e.g. below a theoretical value of 100 cP).

Accordingly, provided herein are methods for sterilization of ophthalmic formulations that prevent degradation of polymeric components (e.g., thermosetting and/or other viscosity enhancing agents) and/or the ophthalmic agent during the process of sterilization. In some embodiments, degradation of the ophthalmic agent (e.g. a muscarinic agent such as aceclidine, pilocarpine, or tropicamide) is reduced or eliminated through the use of specific pH ranges for buffer components and specific proportions of viscosity enhancing agents in the formulations. In some embodiments, the choice of an appropriate viscosity enhancing agents or thermosetting polymer allows for sterilization of formulations described herein by filtration. In some embodiments, the use of an appropriate thermosetting polymer or other viscosity enhancing agents in combination with a specific pH range for the formulation allows for high temperature sterilization of formulations described with substantially no degradation of the therapeutic agent or the polymeric excipients. An advantage of the methods of sterilization provided herein is that, in certain instances, the formulations are subjected to terminal sterilization via autoclaving without any loss of the ophthalmic agent and/or excipients and/or viscosity enhancing agents during the sterilization step and are rendered substantially free of microbes and/or pyrogens.

### Radiation Sterilization

One advantage of radiation sterilization is the ability to sterilize many types of products without heat degradation or other damage. The radiation commonly employed is beta radiation or alternatively, gamma radiation from a ⁶⁰Co source. The penetrating ability of gamma radiation allows its use in the sterilization of many product types, including solutions, compositions and heterogeneous mixtures. The germicidal effects of irradiation arise from the interaction of gamma radiation with biological macromolecules. This interaction generates charged species and free-radicals. Subsequent chemical reactions, such as rearrangements and cross-linking processes, result in the loss of normal function for these biological macromolecules. The formulations described herein are also optionally sterilized using beta irradiation.

### Sterilization by Heat

Many methods are available for sterilization by the application of high heat. One method is through the use of a saturated steam autoclave. In this method, saturated steam at a temperature of at least 121° C. is allowed to contact the object to be sterilized. The transfer of heat is either directly to the microorganism, in the case of an object to be sterilized, or indirectly to the microorganism by heating the bulk of an aqueous solution to be sterilized. This method is widely practiced as it allows flexibility, safety and economy in the sterilization process.

### Sterilization by Ethylene Oxide

In some embodiments, the methods disclosed herein comprise sterilizing the formulation (or components thereof) using ethylene oxide (EtO) sterilization. In some instances, the method for ethylene oxide sterilization comprises injecting a chamber or sterilizing unit using a sterilant or sterilizing agent. In some instances, the sterilant or sterilizing agent is a gas sterilant. In some instances, the sterilant or sterilizing agent is ethylene oxide. In some instances, the gas sterilant is ethylene oxide.

### Microorganisms

In some embodiments, the compositions are substantially free of microorganisms. Acceptable bioburden or sterility levels are based on applicable standards that define therapeutically acceptable compositions, including but not limited to United States Pharmacopeia Chapters <1111> et seq. For example, acceptable sterility (e.g., bioburden) levels include about 10 colony forming units (cfu) per gram of formulation, about 50 cfu per gram of formulation, about 100 cfu per gram of formulation, about 500 cfu per gram of formulation or about 1000 cfu per gram of formulation. In some embodiments, acceptable bioburden levels or sterility for formulations include less than 10 cfu/mL, less than 50 cfu/mL, less than 500 cfu/mL or less than 1000 cfu/mL microbial agents. In addition, acceptable bioburden levels or sterility include the exclusion of specified objectionable microbiological agents. By way of example, specified objectionable microbiological agents include but are not limited to Escherichia coli (E. coli), Salmonella sp., Pseudomonas aeruginosa (P. aeruginosa) and/or other specific microbial agents.

An important component of the sterility assurance quality control, quality assurance and validation process is the method of sterility testing. Sterility testing, by way of example only, is performed by two methods. The first is direct inoculation wherein a sample of the composition to be tested is added to growth medium and incubated for a period of time up to 21 days. Turbidity of the growth medium indicates contamination. Drawbacks to this method include the small sampling size of bulk materials which reduces sensitivity, and detection of microorganism growth based on a visual observation. An alternative method is membrane filtration sterility testing. In this method, a volume of product is passed through a small membrane filter paper. The filter paper is then placed into media to promote the growth of microorganisms. This method has the advantage of greater sensitivity as the entire bulk product is sampled. The commercially available Millipore Steritest sterility testing system is optionally used for determinations by membrane filtration sterility testing. For the filtration testing of creams or ointments Steritest filter system No. TLHVSL210 are used. For the filtration testing of emulsions or viscous products Steritest filter system No. TLAREM210 or TDAREM210 are used. For the filtration testing of pre-filled syringes Steritest filter system No. TTHASY210 are used. For the filtration testing of material dispensed as an aerosol or foam Steritest filter system No. TTHVA210 are used. For the filtration testing of soluble powders in ampoules or vials Steritest filter system No. TTHADA210 or TTHADV210 are used.

Testing for E. coli and Salmonella comprises the use of lactose broths incubated at 30-35° C. for 24-72 hours, incubation in MacConkey and/or EMB agars for 18-24 hours, and/or the use of Rappaport medium. Testing for the detection of P. aeruginosa comprises the use of NAC agar. United States Pharmacopeia Chapter <62> further enumerates testing procedures for specified objectionable microorganisms.

In certain embodiments, the ophthalmic formulation described herein has less than about 60 colony forming units (CFU), less than about 50 colony forming units, less than about 40 colony forming units, or less than about 30 colony forming units of microbial agents per gram of formulation. In certain embodiments, the ophthalmic formulations described herein are formulated to be isotonic with the eye.

### Endotoxins

An additional aspect of the sterilization process is the removal of by-products from the killing of microorganisms (hereinafter, “Product”). The process of depyrogenation removes pyrogens from the sample. Pyrogens are endotoxins or exotoxins which induce an immune response. An example of an endotoxin is the lipopolysaccharide (LPS) molecule found in the cell wall of gram-negative bacteria. While sterilization procedures such as autoclaving or treatment with ethylene oxide kill the bacteria, the LPS residue induces a proinflammatory immune response, such as septic shock. Because the molecular size of endotoxins varies widely, the presence of endotoxins is expressed in “endotoxin units” (EU). One EU is equivalent to 100 picograms of E. coli LPS. In some cases, humans develop a response to as little as 5 EU/kg of body weight. The bioburden (e.g., microbial limit) and/or sterility (e.g., endotoxin level) is expressed in any units as recognized in the art. In certain embodiments, ophthalmic compositions described herein contain lower endotoxin levels (e.g. <4 EU/kg of body weight of a subject) when compared to conventionally acceptable endotoxin levels (e.g., 5 EU/kg of body weight of a subject). In some embodiments, the ophthalmic formulation has less than about 5 EU/kg of body weight of a subject. In other embodiments, the ophthalmic formulation has less than about 4 EU/kg of body weight of a subject. In additional embodiments, the ophthalmic formulation has less than about 3 EU/kg of body weight of a subject. In additional embodiments, the ophthalmic formulation has less than about 2 EU/kg of body weight of a subject.

In some embodiments, the ophthalmic formulation has less than about 5 EU/kg of formulation. In other embodiments, the ophthalmic formulation has less than about 4 EU/kg of formulation. In additional embodiments, the ophthalmic formulation has less than about 3 EU/kg of formulation. In some embodiments, the ophthalmic formulation has less than about 2 EU/kg Product. In other embodiments, the ophthalmic formulation has less than about 1 EU/kg Product. In additional embodiments, the ophthalmic formulation has less than about 0.2 EU/kg Product. In some embodiments, the ophthalmic formulation has less than about 5 EU/g of unit or Product. In other embodiments, the ophthalmic formulation has less than about 4 EU/g of unit or Product. In additional embodiments, the ophthalmic formulation has less than about 3 EU/g of unit or Product. In some embodiments, the ophthalmic formulation has less than about 5 EU/mg of unit or Product. In other embodiments, the ophthalmic formulation has less than about 4 EU/mg of unit or Product. In additional embodiments, the ophthalmic formulation has less than about 3 EU/mg of unit or Product. In certain embodiments, ophthalmic formulations described herein contain from about 1 to about 5 EU/mL of formulation. In certain embodiments, ophthalmic formulations described herein contain from about 2 to about 5 EU/mL of formulation, from about 3 to about 5 EU/mL of formulation, or from about 4 to about 5 EU/mL of formulation.

In certain embodiments, ophthalmic compositions described herein contain lower endotoxin levels (e.g. <0.5 EU/mL of formulation) when compared to conventionally acceptable endotoxin levels (e.g., 0.5 EU/mL of formulation). In some embodiments, the ophthalmic formulation has less than about 0.5 EU/mL of formulation. In other embodiments, the ophthalmic formulation has less than about 0.4 EU/mL of formulation. In additional embodiments, the ophthalmic formulation has less than about 0.2 EU/mL of formulation.

Pyrogen detection, by way of example only, is performed by several methods. Suitable tests for sterility include tests described in United States Pharmacopoeia (USP)<71> Sterility Tests (23rd edition, 1995). The rabbit pyrogen test and the Limulus amebocyte lysate test are both specified in the United States Pharmacopeia Chapters <85> and <151> (USP23/NF 18, Biological Tests, The United States Pharmacopeial Convention, Rockville, MD, 1995). Alternative pyrogen assays have been developed based upon the monocyte activation-cytokine assay. Uniform cell lines suitable for quality control applications have been developed and have demonstrated the ability to detect pyrogenicity in samples that have passed the rabbit pyrogen test and the Limulus amebocyte lysate test (Taktak et al, J. Pharm. Pharmacol. (1990), 43:578-82). In an additional embodiment, the ophthalmic formulation is subject to depyrogenation. In a further embodiment, the process for the manufacture of the ophthalmic formulation comprises testing the formulation for pyrogenicity. In certain embodiments, the formulations described herein are substantially free of pyrogens.

### Ophthalmic Mucus Penetrating Particle (MPP) Compositions

Mucus-penetrating particles (MPPs) are particles that rapidly traverse mucus (e.g. human mucus). In some cases, MPPs comprise of a nanoparticle with a particle size of between about 200 nm and 500 nm. In some instances, the nanoparticle is further coated with a mucus penetrating agent. In some instances, a composition described herein is formulated with MPPs for mucus penetration. In some instances, an ophthalmic agent composition described herein is formulated with MPPs for mucus penetration. In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some embodiments, the ophthalmic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

In some instances, the ophthalmic agent is atropine, atropine sulfate, noratropine, atropine-N-oxide, tropine, tropic acid, atropine methonitrate, diphenhydramine, dimenhydrinate, dicyclomine, flavoxate, oxybutynin, tiotropium, hyoscine, scopolamine (L-hyoscine), hydroxyzine, ipratropium, tropicamide, cyclopentolate, pirenzepine, homatropine, solifenacin, darifenacin, benzatropine, mebeverine, procyclidine, aclidinium bromide, trihexyphenidyl/benzhexol, tolterodine, anisodamine, or a combination thereof.

In some embodiments, the ophthalmic agent for treating presbyopia is aflibercept, ranibizumab, pegaptanib, cyclopentolate, phenylephrine, homatropine, scopolamine, cyclopentolate/phenylephrine, phenylephrine/scopolamine, tropicamide, ketorolac/phenylephrine, hydroxyamphetamine/tropicamide, cysteamine, ocriplasmin, mitomycin, dapiprazole, lidocaine, proparacaine, tetracaine, benoxinate, azithromycin, bacitracin, besifloxacin, boric acid, chloramphenicol, ciprofloxacin, erythromycin, ganciclovir, gatifloxacin, gentamicin, idoxuridine, levofloxacin, moxifloxacin, natamycin, norfloxacin, ofloxacin, bacitracin/polymyxin b, tobramycin, polymyxin b/trimethoprim, povidone iodine, trifluridine, gramicidin/neomycin/polymyxin b, sulfacetamide sodium, sulfisoxazole, bacitracin/neomycin/polymyxin b, oxytetracycline/polymyxin b, phenylephrine/sulfacetamide sodium, vidarabine, bromfenac, nepafenac, ketorolac, cyclosporine, flurbiprofen, suprofen, diclofenac, alcaftadine, azelastine, bepotastine, cromolyn, emedastine, epinastine, ketotifen, levocabastine, lodoxamide, nedocromil, naphazoline, naphazoline/pheniramine, naphazoline/zinc sulfate, olopatadine, oxymetazoline, pemirolast, phenylephrine/zinc sulfate, tetrahydrozoline, tetrahydrozoline/zinc sulfate, fluorescein, fluorescein/proparacaine, benoxinate/fluorescein, indocyanine green, trypan blue, acetylcholine, apraclonidine, betaxolol, bimatoprost, brimonidine, brinzolamide, brimonidine/brinzolamide, carbachol, carteolol, demecarium bromide, dipivefrin, dorzolamide, dorzolamide/timolol, echothiophate iodide, epinephrine, epinephrine/pilocarpine, latanoprost, levobunolol, levobetaxolol, metipranolol, physostigmine, pilocarpine, tafluprost, timolol, travoprost, unoprostone, artificial tear, dexamethasone, difluprednate, fluocinolone, fluorometholone, loteprednol, medrysone, prednisolone, rimexolone, triamcinolone, fluorometholone/sulfacetamide sodium, dexamethasone/neomycin, dexamethasone/tobramycin, dexamethasone/neomycin/polymyxin b, loteprednol/tobramycin, prednisolone/sulfacetamide sodium, bacitracin/hydrocortisone/neomycin/polymyxin b, hydrocortisone/neomycin/polymyxin b, chloramphenicol/hydrocortisone/polymyxin b, neomycin/polymyxin b/prednisolone, gentamicin/prednisolone, ketorolac/phenylephrine, diphenhydramine, dimenhydrinate, dicyclomine, flavoxate, oxybutynin, tiotropium, hyoscine, scopolamine (L-hyoscine), hydroxyzine, ipratropium, pirenzepine, solifenacin, darifenacin, benzatropine, mebeverine, procyclidine, aclidinium bromide, trihexyphenidyl/benzhexol, tolterodine, aceclidine, anisodamine, or any combinations thereof. In some embodiments, the ophthalmic agent is aceclidine, tropicamide, pilocarpine, or combinations thereof.

In some embodiments, the ophthalmic agent for treating presbyopia is a miotic agent. In some instances, the miotic agent is dapiprazole, thymoxamine, brimonidine, nicotine, apraclonidin, phentolamine, pharmaceutically acceptable salts thereof, or combinations thereof.

In some embodiments, the ophthalmic agent for treating presbyopia is a muscarinic receptor agonist, muscarinic receptor antagonist, an alpha-1 adrenergic receptor antagonist, an alpha-2 adrenergic receptor agonist, a beta-adrenergic receptor antagonist, a nicotine receptor agonist, an antipsychotic, an antiemetic, a cannabinoid, a monoamine oxidase (MAO) inhibitor, an EP1 receptor agonist, an EP4 receptor agonist, an FP receptor agonist, a calcium channel modulator, an anticholinergic agent, or combinations thereof.

In some instances, a muscarinic agent composition described herein is formulated with MPPs for mucus penetration. In some embodiments, the muscarinic agent is aceclidine or a pharmaceutically acceptable salt of aceclidine. In some embodiments, the muscarinic agent is pilocarpine or a pharmaceutically acceptable salt of pilocarpine. In some embodiments, the muscarinic agent is tropicamide or a pharmaceutically acceptable salt of tropicamide. In some embodiments, the muscarinic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof. In some instances, the ophthalmic composition described herein is formulated with MPPs for mucus penetration. In some instances, the ophthalmic composition described herein is formulated with MPPs for mucus penetration. In a non-limiting example, the MMPs for use in the disclosed composition is obtained from Kala Pharmaceuticals, Inc. (100 Beaver Street #201, Waltham, MA 02453).

In some embodiments, the nanoparticle comprises of any suitable material, such as an organic material, an inorganic material, a polymer, or combinations thereof. In some instances, the nanoparticle comprises of inorganic material, such as for example, a metal (e.g., Ag, Au, Pt, Fe, Cr, Co, Ni, Cu, Zn, and other transition metals), a semiconductor (e.g., silicon, silicon compounds and alloys, cadmium selenide, cadmium sulfide, indium arsenide, and indium phosphide), or an insulator (e.g., ceramics such as silicon oxide). In some instances, the nanoparticle comprises organic materials such as a synthetic polymer and/or a natural polymer. Examples of synthetic polymers include non-degradable polymers such as polymethacrylate and degradable polymers such as polylactic acid, polyglycolic acid and copolymers thereof. Examples of natural polymers include hyaluronic acid, chitosan, and collagen.

In some embodiments, the nanoparticle is coated with a mucus penetrating agent. In some instances, the mucus penetrating agent comprises any suitable material, such as a hydrophobic material, a hydrophilic material, and/or an amphiphilic material. In some instances, the mucus penetrating agent is a polymer. In some instances, the polymer a synthetic polymer (i.e., a polymer not produced in nature). In other embodiments, the polymer is a natural polymer (e.g., a protein, polysaccharide, rubber). In certain embodiments, the polymer is a surface active polymer. In certain embodiments, the polymer is a non-ionic polymer. In certain embodiments, the polymer is a non-ionic block copolymer. In some embodiments, the polymer is a diblock copolymer, a triblock copolymer, e.g., e.g., where one block is a hydrophobic polymer and another block is a hydrophilic polymer. In some embodiments, the polymer is charged or uncharged.

Additional examples of suitable polymers include, but are not limited to, polyamines, polyethers, polyamides, polyesters, polycarbamates, polyureas, polycarbonates, polystyrenes, polyimides, polysulfones, polyurethanes, polyacetylenes, polyethylenes, polyethyeneimines, polyisocyanates, polyacrylates, polymethacrylates, polyacrylonitriles, and polyarylates. Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), poly(ethylene glycol), poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) (jointly referred to herein as “polyacrylic acids”), and copolymers and mixtures thereof, polydioxanone and its copolymers, polyhydroxyalkanoates, polypropylene fumarate), polyoxymethylene, poloxamers, poly(ortho)esters, poly(butyric acid), poly(valeric acid), poly(lactide-co-caprolactone), and trimethylene carbonate, polyvinylpyrrolidone.

In some cases, an ophthalmic agent (e.g. a muscarinic agent such as aceclidine, pilocarpine, or tropicamide) is present in the MPP formulation at a concentration of between about 0.001 wt % and about 0.05 wt %, between about 0.005% to about 0.050%, between about 0.010% to about 0.050%, between about 0.015% to about 0.050%, between about 0.020% to about 0.050%, between about 0.025% to about 0.050%, between about 0.030% to about 0.050%, between about 0.035% to about 0.050%, between about 0.040% to about 0.050%, or between about 0.045% to about 0.050% of the ophthalmic agent, or pharmaceutically acceptable prodrug or salt thereof, by weight of the composition. In some instances, additional agents such as buffers, pH adjusting agents, and/or preservatives are formulated in the MPP formulation.

In some instances, ophthalmic agent-MPP composition is formulated using any suitable method. In some embodiments, a milling process is used to reduce the size of a solid material to form particles in the micrometer to nanometer size range. In some cases, dry and wet milling processes such as jet milling, cryo-milling, ball milling, media milling, and homogenization are known and are used in methods described herein. Generally, in a wet milling process, a suspension of the material to be used as the nanoparticle is mixed with milling media with or without excipients to reduce particle size. Dry milling is a process wherein the material to be used as the nanoparticle is mixed with milling media with or without excipients to reduce particle size. In a cryo-milling process, a suspension of the material to be used as the nanoparticle is mixed with milling media with or without excipients under cooled temperatures.

In some embodiments, any suitable grinding medium is used for milling. In some embodiments, a ceramic and/or polymeric material and/or a metal is used. Examples of suitable materials include zirconium oxide, silicon carbide, silicon oxide, silicon nitride, zirconium silicate, yttrium oxide, glass, alumina, alpha-alumina, aluminum oxide, polystyrene, poly(methyl methacrylate), titanium, steel. In some cases, a grinding medium has any suitable size. For example, the grinding medium has an average diameter of at least about 0.1 mm, at least about 0.2 mm, at least about 0.5 mm, at least about 0.8 mm, at least about 1 mm, at least about 2 mm, or at least about 5 mm. In some cases, the grinding medium has an average diameter of less than or equal to about 5 mm, less than or equal to about 2 mm, less than or equal to about 1 mm, less than or equal to about 0.8, less than or equal to about 0.5 mm, or less than or equal to about 0.2 mm. Combinations of the above-referenced ranges are also possible (e.g., an average diameter of at least about 0.5 millimeters and less than or equal to about 1 mm). Other ranges are also possible.

In some embodiments, any suitable solvent are used for milling. In some cases, the choice of solvent is dependent on factors such as the solid material (e.g. a muscarinic agent such as aceclidine, pilocarpine, or tropicamide) being milled, the particular type of stabilizer/mucus penetrating agent being used (e.g., one that renders the particle mucus penetrating), the grinding material be used, among other factors. In some cases, suitable solvents are ones that do not substantially dissolve the solid material or the grinding material, but dissolve the stabilizer/mucus penetrating agent to a suitable degree. Non-limiting examples of solvents include, but are not limited to, water, buffered solutions, other aqueous solutions, alcohols (e.g., ethanol, methanol, butanol), and mixtures thereof that optionally include other components such as pharmaceutical excipients, polymers, pharmaceutical agents, salts, preservative agents, viscosity modifiers, tonicity modifier, taste masking agents, antioxidants, pH modifier, and other pharmaceutical excipients. In other embodiments, an organic solvent is used. In some cases, a pharmaceutical agent (e.g. a muscarinic agent such as aceclidine, pilocarpine, or tropicamide) has any suitable solubility in these or other solvents, such as a solubility in one or more of the ranges described above for aqueous solubility or for solubility in a coating solution.

In some instances, a MPP is a MPP as described in WO2013/166385. In some instances, a MPP is a MPP as described in Lai et al., “Rapid transport of large polymeric nanoparticles in fresh undiluted human mucus,” PNAS 104(5):1482-1487 (2007). In some instances, an ophthalmic agent-MPP composition is formulated using a method as described in WO2013/166385. In some instances, an ophthalmic agent-MPP composition is formulated using a method as described in Lai et al., “Rapid transport of large polymeric nanoparticles in fresh undiluted human mucus,” PNAS 104(5):1482-1487 (2007). In some embodiments, the ophthalmic agent is aceclidine or a pharmaceutically acceptable salt of aceclidine. In some embodiments, the ophthalmic agent is pilocarpine or a pharmaceutically acceptable salt of pilocarpine. In some embodiments, the ophthalmic agent is tropicamide or a pharmaceutically acceptable salt of tropicamide. In some embodiments, the ophthalmic agent is aceclidine or a pharmaceutically acceptable salt of aceclidine, pilocarpine or a pharmaceutically acceptable salt of pilocarpine, tropicamide or a pharmaceutically acceptable salt of tropicamide, or combinations thereof.

Ophthalmic Gel Compositions

Gels have been defined in various ways. For example, the United States Pharmacopoeia defines gels as semisolid systems consisting of either suspensions made up of small inorganic particles or large organic molecules interpenetrated by a liquid. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles.

In some embodiments, gels are also classified as being hydrophobic or hydrophilic. In certain embodiments, the base of a non-limiting example of a hydrophobic gel comprises a liquid paraffin with polyethylene or fatty oils gelled with colloidal silica, or aluminum or zinc soaps. In contrast, the base of a non-limiting example of a hydrophilic gel comprises water, glycerol, or propylene glycol gelled with a suitable gelling agent (e.g., tragacanth, starch, cellulose derivatives, carboxyvinylpolymers, and magnesium-aluminum silicates). In certain embodiments, the rheology of the compositions disclosed herein is pseudo plastic, plastic, thixotropic, or dilatant.

In some embodiments, the ophthalmic composition is an ophthalmic gel, and wherein the ophthalmically acceptable carrier comprises water and at least one viscosity-enhancing agent. In some embodiments, the viscosity-enhancing agent is selected from cellulose-based polymers, polyoxyethylene-polyoxypropylene triblock copolymers, dextran-based polymers, polyvinyl alcohol, dextrin, polyvinylpyrrolidone, polyalkylene glycols, chitosan, collagen, gelatin, hyaluronic acid, or combinations thereof.

In some embodiment, the ophthalmic gel composition described herein is a semi-solid or id in a gelled state before it is topically administered (e.g. at room temperature). For example, suitable viscosity-enhancing agents for such gels include by way of example only, gelling agents and suspending agents. In one embodiment, the enhanced viscosity formulation does not include a buffer. In other embodiments, the enhanced viscosity formulation comprises a pharmaceutically acceptable buffer. Sodium chloride or other tonicity agents are optionally used to adjust tonicity, if necessary.

By way of example only, the ophthalmically acceptable viscosity agent comprises hydroxypropyl methylcellulose, hydroxyethyl cellulose, polyvinylpyrrolidone, carboxymethyl cellulose, polyvinyl alcohol, sodium chondroitin sulfate, sodium hyaluronate. Other viscosity enhancing agents compatible with the targeted ocular site include, but are not limited to, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose) or combinations thereof. In specific embodiments, the viscosity-enhancing excipient is a combination of MCC and CMC. In another embodiment, the viscosity-enhancing agent is a combination of carboxymethylated chitosan, or chitin, and alginate. The combination of chitin and alginate with the ophthalmic agents disclosed herein acts as a controlled release formulation, restricting the diffusion of the ophthalmic agents from the formulation. Moreover, the combination of carboxymethylated chitosan and alginate is optionally used to assist in increasing the permeability of the ophthalmic agents in the eye.

In some embodiments, the viscosity agent is present in the composition at about 0.001%, 0.005%, 0.010%, 0.015%, 0.020%, 0.025%, 0.030%, 0.035%, 0.040%, 0.045%, 0.050%, 0.055%, 0.060%, 0.065%, 0.070%, 0.075%, 0.080%, 0.085%, 0.090%, 0.095%, 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, 5.5%, or 6%. In some embodiments, the viscosity agent is present in the composition from about 0.001% to about 0.05%, from about 0.001% to about 0.04%, from about 0.001% to about 0.03%, from about 0.001% to about 0.025%, from about 0.001% to about 0.02%, from about 0.001% to about 0.01%, from about 0.001% to about 0.008%, or from about 0.001% to about 0.005%. In some cases, the percentage is a weight percentage.

In some embodiments is an enhanced viscosity formulation, comprising from about 0.1 mM and about 100 mM of an ophthalmic agent, a pharmaceutically acceptable viscosity agent, and water for injection, the concentration of the viscosity agent in the water being sufficient to provide an enhanced viscosity formulation with a final viscosity from about 100 to about 100,000 cP. In certain embodiments, the viscosity of the gel is in the range from about 100 to about 50,000 cP, about 100 cP to about 1,000 cP, about 500 cP to about 1500 cP, about 1000 cP to about 3000 cP, about 2000 cP to about 8,000 cP, about 4,000 cP to about 50,000 cP, about 10,000 cP to about 500,000 cP, about 15,000 cP to about 1,000,000 cP. In other embodiments, when an even more viscous medium is desired, the biocompatible gel comprises at least about 35%, at least about 45%, at least about 55%, at least about 65%, at least about 70%, at least about 75%, or even at least about 80% or so by weight of the ophthalmic agent. In highly concentrated samples, the biocompatible enhanced viscosity formulation comprises at least about 25%, at least about 35%, at least about 45%, at least about 55%, at least about 65%, at least about 75%, at least about 85%, at least about 90% or at least about 95% or more by weight of the ophthalmic agent.

In one embodiment, the pharmaceutically acceptable enhanced viscosity ophthalmically acceptable formulation comprises at least one ophthalmic agent and at least one gelling agent. Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum and any combinations or mixtures thereof. In some other embodiments, hydroxypropylmethylcellulose (Methocel®) is utilized as the gelling agent. In certain embodiments, the viscosity enhancing agents described herein are also utilized as the gelling agent for the gel formulations presented herein.

In some embodiments, the ophthalmic gel composition described herein is an in situ gel formulation. In some instances, the in situ gel formation is based on increased pre-corneal residence time of the ophthalmic composition which improves ocular bioavailability, corneal mucoadhesion, lysosomal interaction and ionic gelation, improved corneal absorption, thermal gelation, or a combination thereof. In some instances, the in situ gel formulation is activated by pH, temperature, ion, UV, or solvent exchange.

In some instances, the ophthalmic gel composition comprises an ophthalmic agent such as a muscarinic agent (e.g., aceclidine, pilocarpine, tropicamide, or pharmaceutically acceptable salts thereof) and one or more gelling agents. In some instances, the gelling agent comprises, but is not limited to, poloxamer (e.g. Poloxamer 407), tetronics, ethyl (hydroxyethyl) cellulose, cellulose acetate phthalate (CAP), carbopol (e.g. Carbopol 1342P NF, Carbopol 980 NF), alginates (e.g. low acetyl gellan gum (Gelrite®)), gellan, hyaluronic acid, pluronics (e.g. Pluronic F-127), chitosan, polyvinyl alcohol (PVA), polyvinylpyrrolidone (PVP), dextran, hydroxy propyl methyl cellulose (HPMC), hydroxyethylcellulose (HEC), methylcellulose (MC), thiolated xyloglucan, polymethacrilic acid (PMMA), polyethylene glycol (PEG), pseudolatexes, xyloglucans, or combinations thereof.

In some instances, the in situ gel formation further comprises a permeation enhancer. In some instances, the permeation enhancer comprises surfactants (e.g. non-ionic surfactants), benzalkonium chloride, EDTA, surface-active heteroglycosides, calcium chelators, hydroxyl propyl beta cyclodextrin (HP beta CD), bile salts, and the like. In some instances, the permeation enhancer is EDTA. In some embodiments, EDTA is present in the composition at about 0.001%, 0.005%, 0.010%, 0.015%, 0.020%, 0.025%, 0.030%, 0.035%, 0.040%, 0.045%, 0.050%, 0.055%, 0.060%, 0.065%, 0.070%, 0.075%, 0.080%, 0.085%, 0.090%, 0.095%, 0.1%, 0.2%, 0.3%, 0.4%, 0.5%, 0.6%, 0.7%, 0.8%, 0.9%, 1.0%, 1.5%, 2.0%, 2.5%, or 3.0%. In some embodiments, EDTA is present in the composition from about 0.001% to about 0.05%, from about 0.001% to about 0.04%, from about 0.001% to about 0.03%, from about 0.001% to about 0.025%, from about 0.001% to about 0.02%, from about 0.001% to about 0.01%, from about 0.001% to about 0.008%, or from about 0.001% to about 0.005%. In some cases, the percentage is a weight percentage.

In some embodiments, other gel formulations are useful depending upon the particular ophthalmic agent, other pharmaceutical agent or excipients/additives used, and as such are considered to fall within the scope of the present disclosure. For example, other commercially-available glycerin-based gels, glycerin-derived compounds, conjugated, or crosslinked gels, matrices, hydrogels, and polymers, as well as gelatins and their derivatives, alginates, and alginate-based gels, and even various native and synthetic hydrogel and hydrogel-derived compounds are all expected to be useful in the ophthalmic agent formulations described herein. In some embodiments, ophthalmically acceptable gels include, but are not limited to, alginate hydrogels SAF®-Gel (ConvaTec, Princeton, N.J.), Duoderm® Hydroactive Gel (ConvaTec), Nu-gel® (Johnson & Johnson Medical, Arlington, Tex.); Carrasyn® (V) Acemannan Hydrogel (Carrington Laboratories, Inc., Irving, Tex.); glycerin gels Elta® Hydrogel (Swiss-American Products, Inc., Dallas, Tex.) and K-Y® Sterile (Johnson & Johnson). In further embodiments, biodegradable biocompatible gels also represent compounds present in ophthalmically acceptable formulations disclosed and described herein.

In some embodiments, the viscosity-enhancing agent is a cellulose-based polymer selected from cellulose gum, alkylcellulose, hydroxyl-alkyl cellulose, hydroxyl-alkyl alkylcellulose, carboxy-alkyl cellulose, or combinations thereof. In some embodiments, the viscosity-enhancing agent is hydroxyl-alkyl alkylcellulose. In some embodiment, the viscosity-enhancing agent is hydroxypropyl methylcellulose.

In certain embodiments, the enhanced viscosity formulation is characterized by a phase transition between room temperature and body temperature (including an individual with a serious fever, e.g., up to about 42° C.). In some embodiments, the phase transition occurs at 1° C. below body temperature, at 2° C. below body temperature, at 3° C. below body temperature, at 4° C. below body temperature, at 6° C. below body temperature, at 8° C. below body temperature, or at 10° C. below body temperature. In some embodiments, the phase transition occurs at about 15° C. below body temperature, at about 20° C. below body temperature, or at about 25° C. below body temperature. In specific embodiments, the gelation temperature (Tgel) of a formulation described herein is about 20° C., about 25° C., or about 30° C. In certain embodiments, the gelation temperature (Tgel) of a formulation described herein is about 35° C., or about 40° C. Included within the definition of body temperature is the body temperature of a healthy individual, or an unhealthy individual, including an individual with a fever (up to ˜42° C.). In some embodiments, the pharmaceutical compositions described herein are liquids at about room temperature and are administered at or about room temperature.

Copolymers polyoxypropylene and polyoxyethylene (e.g. polyoxyethylene-polyoxypropylene triblock copolymers) form thermosetting gels when incorporated into aqueous solutions. These polymers have the ability to change from the liquid state to the gel state at temperatures close to body temperature, therefore allowing useful formulations that are applied to the targeted ocular site. The liquid state-to-gel state phase transition is dependent on the polymer concentration and the ingredients in the solution.

In some embodiments, the amount of thermosetting polymer in any formulation described herein is about 10%, about 15%, about 20%, about 25%, about 30%, about 35%, or about 40% of the total weight of the formulation. In some embodiments, the amount of thermosetting polymer in any formulation described herein is about 10%, about 11%, about 12%, about 13%, about 14%, about 15%, about 16%, about 17%, about 18%, about 19%, about 20%, about 21%, about 22%, about 23%, about 24%, or about 25% of the total weight of the formulation. In some embodiments, the amount of thermosetting polymer (e.g., Poloxamer 407) in any formulation described herein is about 7.5% of the total weight of the formulation. In some embodiments, the amount of thermosetting polymer (e.g., Poloxamer 407) in any formulation described herein is about 10% of the total weight of the formulation. In some embodiments, the amount of thermosetting polymer (e.g., Poloxamer 407) in any formulation described herein is about 11% of the total weight of the formulation. In some embodiments, the amount of thermosetting polymer (e.g., Poloxamer 407) in any formulation described herein is about 12% of the total weight of the formulation. In some embodiments, the amount of thermosetting polymer (e.g., Poloxamer 407) in any formulation described herein is about 13% of the total weight of the formulation. In some embodiments, the amount of thermosetting polymer (e.g., Poloxamer 407) in any formulation described herein is about 14% of the total weight of the formulation. In some embodiments, the amount of thermosetting polymer (e.g., Poloxamer 407) in any formulation described herein is about 15% of the total weight of the formulation. In some embodiments, the amount of thermosetting polymer (e.g., Poloxamer 407) in any formulation described herein is about 16% of the total weight of the formulation. In some embodiments, the amount of thermosetting polymer (e.g., Poloxamer 407) in any formulation described herein is about 17% of the total weight of the formulation. In some embodiments, the amount of thermosetting polymer (e.g., Poloxamer 407) in any formulation described herein is about 18% of the total weight of the formulation. In some embodiments, the amount of thermosetting polymer (e.g., Poloxamer 407) in any formulation described herein is about 19% of the total weight of the formulation. In some embodiments, the amount of thermosetting polymer (e.g., Poloxamer 407) in any formulation described herein is about 20% of the total weight of the formulation. In some embodiments, the amount of thermosetting polymer (e.g., Poloxamer 407) in any formulation described herein is about 21% of the total weight of the formulation. In some embodiments, the amount of thermosetting polymer (e.g., Poloxamer 407) in any formulation described herein is about 23% of the total weight of the formulation. In some embodiments, the amount of thermosetting polymer (e.g., Poloxamer 407) in any formulation described herein is about 25% of the total weight of the formulation. In some embodiments, the amount of thickening agent (e.g., a gelling agent) in any formulation described herein is about 1%, about 5%, about 10%, or about 15% of the total weight of the formulation. In some embodiments, the amount of thickening agent (e.g., a gelling agent) in any formulation described herein is about 0.5%, about 1%, about 1.5%, about 2%, about 2.5%, about 3%, about 3.5%, about 4%, about 4.5%, or about 5% of the total weight of the formulation.

In an alternative embodiment, the thermogel is a PEG-PLGA-PEG triblock copolymer (Jeong et al, Nature (1997), 388:860-2; Jeong et al, J. Control. Release (2000), 63:155-63; Jeong et al, Adv. Drug Delivery Rev. (2002), 54:37-51). The polymer exhibits sol-gel behavior over a concentration of about 5% w/w to about 40% w/w. Depending on the properties desired, the lactide/glycolide molar ratio in the PLGA copolymer ranges from about 1:1 to about 20:1. The resulting copolymers are soluble in water and form a free-flowing liquid at room temperature, but form a hydrogel at body temperature. A commercially available PEG-PLGA-PEG triblock copolymer is RESOMER RGP t50106 manufactured by Boehringer Ingelheim. This material is composed of a PLGA copolymer of 50:50 poly(DL-lactide-co-glycolide) and is 10% w/w of PEG and has a molecular weight of about 6000.

Additional biodegradable thermoplastic polyesters include AtriGel® (provided by Atrix Laboratories, Inc.) and/or those disclosed, e.g., in U.S. Pat. Nos. 5,324,519; 4,938,763; 5,702,716; 5,744,153; and 5,990,194; wherein the suitable biodegradable thermoplastic polyester is disclosed as a thermoplastic polymer. Examples of suitable biodegradable thermoplastic polyesters include polylactides, polyglycolides, polycaprolactones, copolymers thereof, terpolymers thereof, and any combinations thereof. In some such embodiments, the suitable biodegradable thermoplastic polyester is a polylactide, a polyglycolide, a copolymer thereof, a terpolymer thereof, or a combination thereof. In one embodiment, the biodegradable thermoplastic polyester is 50/50 poly(DL-lactide-co-glycolide) having a carboxy terminal group; is present in about 30 wt. % to about 40 wt. % of the composition; and has an average molecular weight of about 23,000 to about 45,000. Alternatively, in another embodiment, the biodegradable thermoplastic polyester is 75/25 poly (DL-lactide-co-glycolide) without a carboxy terminal group; is present in about 40 wt. % to about 50 wt. % of the composition; and has an average molecular weight of about 15,000 to about 24,000. In further or alternative embodiments, the terminal groups of the poly(DL-lactide-co-glycolide) are either hydroxyl, carboxyl, or ester depending upon the method of polymerization. Polycondensation of lactic or glycolic acid provides a polymer with terminal hydroxyl and carboxyl groups. Ring-opening polymerization of the cyclic lactide or glycolide monomers with water, lactic acid, or glycolic acid provides polymers with the same terminal groups. However, ring-opening of the cyclic monomers with a monofunctional alcohol such as methanol, ethanol, or 1-dodecanol provides a polymer with one hydroxyl group and one ester terminal groups. Ring-opening polymerization of the cyclic monomers with a diol such as 1,6-hexanediol or polyethylene glycol provides a polymer with only hydroxyl terminal groups.

Since the polymer systems of thermosetting gels dissolve more completely at reduced temperatures, methods of solubilization include adding the required amount of polymer to the amount of water to be used at reduced temperatures. Generally after wetting the polymer by shaking, the mixture is capped and placed in a cold chamber or in a thermostatic container at about 0-10° C. in order to dissolve the polymer. The mixture is stirred or shaken to bring about a more rapid dissolution of the thermosetting gel polymer. The ophthalmic agent and various additives such as buffers, salts, and preservatives are subsequently added and dissolved. In some instances the pharmaceutically agent is suspended if it is insoluble in water. The pH is modulated by the addition of appropriate buffering agents.

Ophthalmic Ointment Compositions

An ointment is a homogeneous, viscous, semi-solid preparation, most commonly a greasy, thick oil (e.g. oil 80%-water 20%) with a high viscosity, intended for external application to the skin or mucous membranes. Ointments have a water number that defines the maximum amount of water that it contains. They are used as emollients or for the application of active ingredients to the skin for protective, therapeutic, or prophylactic purposes and where a degree of occlusion is desired. Ointments are used topically on a variety of body surfaces. These include the skin and the mucous membranes of the eye (an eye ointment), vulva, anus, and nose.

The vehicle of an ointment is known as the ointment base. The choice of a base depends upon the clinical indication for the ointment. The different types of ointment bases are: hydrocarbon bases, e.g. hard paraffin, soft paraffin, microcrystalline wax and ceresine; absorption bases, e.g. wool fat, beeswax; water soluble bases, e.g. macrogols 200, 300, 400; emulsifying bases, e.g. emulsifying wax, cetrimide; vegetable oils, e.g. olive oil, coconut oil, sesame oil, almond oil and peanut oil.

Ointments are formulated using hydrophobic, hydrophilic, or water-emulsifying bases to provide preparations that are immiscible, miscible, or emulsifiable with skin secretions. In some embodiments, they are also derived from hydrocarbon (fatty), absorption, water-removable, or water-soluble bases. The active agents are dispersed in the base, and later they get divided after the drug penetration into the target sites (e.g. membranes, skins, etc.).

The present disclosure recognizes that it is sometimes difficult to incorporate into the ointment a drug of low concentration with sufficient dose-to-dose uniformity for effectively treating a disorder or disease. In some embodiments, poly(ethylene-glycols), polyethoxylated castor oils (Cremophor® EL), alcohols having 12 to 20 carbon atoms or a mixture of two or more of said components are effective excipients for dispersing and/or dissolving effective amounts of ophthalmic drugs, in particular of ascomycins and staurosporine derivatives, in an ointment base, in particular in an ointment base substantially comprising oleaginous and hydrocarbon components, and that the resulting ointments are excellently tolerated by the skin and by ocular tissue.

The present disclosure further recognizes that ophthalmic drugs, such as a muscarinic agents (e.g. aceclidine, pilocarpine, tropicamide, or its pharmaceutically acceptable salts), incorporated in the ointment compositions describes herein target the choroid and/or retina in a patient when the compositions are topically administered to the ocular surface, in particular to the sclera of said patient. In some embodiments, an ophthalmic ointment composition comprises an ophthalmic drug, an ointment base and an agent for dispersing and/or dissolving said drug in the ointment base, selected from a poly(ethylene-glycol), a polyethoxylated castor oil, an alcohol having 12 to 20 carbon atoms and a mixture of two or more of said components.

In some embodiments, the ointment bases include ophthalmically acceptable oil and fat bases, such as natural wax e.g. white and yellow bees wax, carnauba wax, wool wax (wool fat), purified lanolin, anhydrous lanolin; petroleum wax e.g. hard paraffin, microcrystalline wax; hydrocarbons e.g. liquid paraffin, white and yellow soft paraffin, white petrolatum, yellow petrolatum; or combinations thereof.

The above mentioned oil and fat bases are described in more detail, for instance, in the British Pharmacopoeia, Edition 2001, or the European Pharmacopoeia, 3rd Edition.

In some embodiments, the ointment base is present in amounts of about 50 to about 95, preferably of 70 to 90% by weight based on the total weight of the composition.

A preferred ointment base comprises a combination of one or more of one or more natural waxes like those indicated above, preferably wool wax (wool fat), and one or more hydrocarbons like those indicated above, preferably a soft paraffin or a petrolatum, more preferably in combination with liquid paraffin.

A special embodiment of the aforementioned ointment base comprises e.g. 5 to 17 parts by weight of wool fat, and 50 to 65 parts by weight of white petrolatum as well as 20 to 30 parts by weight of liquid paraffin.

In some embodiments, the agent for dispersing and/or dissolving the ophthalmic drug in the ointment base is selected from a poly(ethylene-glycol), a polyethoxylated castor oil, an alcohol having 12 to 20 carbon atoms and a mixture of two or more of said components. The agent is preferably used in amounts of 1 to 20 percent, more preferably 1 to 10 percent by weight of the entire semisolid ophthalmic composition.

Alcohols having 12 to 20 carbon atoms include particularly stearyl alcohol (C₁₈H₃₇OH), cetyl alcohol (C16H33OH) and mixtures thereof. Preferred are so-called cetostearyl alcohols, mixtures of solid alcohols substantially consisting of stearyl and cetyl alcohol and preferably comprising not less than 40 percent by weight of stearyl alcohol and a sum of stearyl alcohol and cetyl alcohol amounting to at least 90 percent by weight, and compositions comprising not less than 80 percent by weight of cetylstearyl alcohol and an emulsifier, in particular sodium cetostearyl sulfate and/or sodium lauryl sulfate, preferably in amounts not less than 7 percent by weight of emulsifier.

Polyethoxylated castor oils are reaction products of natural or hydrogenated castor oils and ethylene glycol. In some instances, such products are obtained in known manner, e.g. by reaction of a natural or hydrogenated castor oil or fractions thereof with ethylene oxide, e.g. in a molar ratio of from about 1:30 to about 1:60, with optional removal of free polyethylene glycol components from the product, e.g. in accordance with the methods disclosed in German Auslegeschriften 1,182,388 and 1,518,819. Especially suitable and preferred is a product commercially available under the trade name Cremophor® EL having a molecular weight (by steam osmometry)=ca. 1630, a saponification no.=ca. 65-70, an acid no.=ca. 2, an iodine no.=ca. 28-32 and an nD 25=ca.1.471. Also suitable for use in this category is, for instance, Nikkol® HCO-60, a reaction product of hydrogenated castor oil and ethylene oxide exhibiting the following characteristics: acid no.=ca. 0.3; saponification no.=ca. 47.4; hydroxy value=ca. 42.5. pH (5%)=ca. 4.6; Color APHA=ca. 40; m.p.=ca. 36.0° C.; Freezing point=ca. 32.4° C.; H₂O content (%, KF)=ca. 0.03.

Poly(ethylene-glycols) are used in some embodiments as the agent for dispersing and/or dissolving the ophthalmic drug in the ointment base according to the present disclosure. Suitable poly(ethylene-glycol)s are typically mixtures of polymeric compounds of the general formula H—(OCH2-CH2)nOH, wherein the index n typically range from 4 to 230 and the mean molecular weight from about 200 to about 10000. Preferably n is a number from about 6 to about 22 and the mean molecular weight between about 300 and about 1000, more preferably n ranges from about 6 to about 13 and the mean molecular weight from about 300 to about 600, most preferably n has a value of about 8.5 to about 9 and the relative molecular weight is about 400. Suitable poly(ethylene-glycols) are readily available commercially, for example poly(ethylene-glycols) having a mean molecular weight of about 200, 300, 400, 600, 1000, 1500, 2000, 3000, 4000, 6000, 8000 and 10000.

The poly(ethylene-glycols), in particular the preferred types described in the foregoing paragraph, are preferably used in amounts of 1 to 10, more preferably 1 to 5 percent by weight of the entire semisolid ophthalmic composition.

An especially preferred embodiment of the compositions according to the instant disclosure comprises an agent for dispersing and/or dissolving of the drug in the ointment base which is selected from a poly(ethylene-glycol), a polyethoxylated castor oil and preferably a mixture of said components.

### Gel/Ointment Viscosity

In some embodiments, the composition has a Brookfield RVDV viscosity of from about 10,000 to about 300,000 cps at about 20° C. and sheer rate of 1 s⁻¹. In some embodiments, the composition has a Brookfield RVDV viscosity of from about 15,000 to about 200,000 cps at about 20° C. and sheer rate of 1 s⁻¹. In some embodiments, the composition has a Brookfield RVDV viscosity of from about 50,000 to about 150,000 cps at about 20° C. and sheer rate of 1 s⁻¹. In some embodiments, the composition has a Brookfield RVDV viscosity of from about 70,000 to about 130,000 cps at about 20° C. and sheer rate of 1 s⁻¹. In some embodiments, the composition has a Brookfield RVDV viscosity of from about 90,000 to about 110,000 cps at about 20° C. and sheer rate of 1 s⁻¹.

In some embodiments, the ophthalmic gel formulation contains a viscosity enhancing agent sufficient to provide a viscosity of between about 500 and 1,000,000 centipoise, between about 750 and 1,000,000 centipoise; between about 1000 and 1,000,000 centipoise; between about 1000 and 400,000 centipoise; between about 2000 and 100,000 centipoise; between about 3000 and 50,000 centipoise; between about 4000 and 25,000 centipoise; between about 5000 and 20,000 centipoise; or between about 6000 and 15,000 centipoise. In some embodiments, the ophthalmic gel formulation contains a viscosity enhancing agent sufficient to provide a viscosity of between about 50,0000 and 1,000,000 centipoise.

In some embodiments, the compositions described herein are low viscosity compositions at body temperature. In some embodiments, low viscosity compositions contain from about 1% to about 10% of a viscosity enhancing agent (e.g., gelling components such as polyoxyethylene-polyoxypropylene copolymers). In some embodiments, low viscosity compositions contain from about 2% to about 10% of a viscosity enhancing agent (e.g., gelling components such as polyoxyethylene-polyoxypropylene copolymers). In some embodiments, low viscosity compositions contain from about 5% to about 10% of a viscosity enhancing agent (e.g., gelling components such as polyoxyethylene-polyoxypropylene copolymers). In some embodiments, low viscosity compositions are substantially free of a viscosity enhancing agent (e.g., gelling components such as polyoxyethylene-polyoxypropylene copolymers). In some embodiments, a low viscosity ophthalmic agent composition described herein provides an apparent viscosity of from about 100 cP to about 10,000 cP. In some embodiments, a low viscosity ophthalmic agent composition described herein provides an apparent viscosity of from about 500 cP to about 10,000 cP. In some embodiments, a low viscosity ophthalmic agent composition described herein provides an apparent viscosity of from about 1000 cP to about 10,000 cP.

In some embodiments, the compositions described herein are viscous compositions at body temperature. In some embodiments, viscous compositions contain from about 10% to about 25% of a viscosity enhancing agent (e.g., gelling components such as polyoxyethylene-polyoxypropylene copolymers). In some embodiments, the viscous compositions contain from about 14% to about 22% of a viscosity enhancing agent (e.g., gelling components such as polyoxyethylene-polyoxypropylene copolymers). In some embodiments, the viscous compositions contain from about 15% to about 21% of a viscosity enhancing agent (e.g., gelling components such as polyoxyethylene-polyoxypropylene copolymers). In some embodiments, a viscous ophthalmic composition described herein provides an apparent viscosity of from about 100,000 cP to about 1,000,000 cP. In some embodiments, a viscous ophthalmic composition described herein provides an apparent viscosity of from about 150,000 cP to about 500,000 cP. In some embodiments, a viscous ophthalmic composition described herein provides an apparent viscosity of from about 250,000 cP to about 500,000 cP. In some of such embodiments, a viscous ophthalmic composition is a liquid at room temperature and gels at about between room temperature and body temperature (including an individual with a serious fever, e.g., up to about 42° C.). In some embodiments, a viscous ophthalmic composition is administered as monotherapy for treatment of an ophthalmic disease or condition described herein.

In some embodiments, the viscosity of the gel formulations presented herein is measured by any means described. For example, in some embodiments, an LVDV-II+CP Cone Plate Viscometer and a Cone Spindle CPE-40 is used to calculate the viscosity of the gel formulation described herein. In other embodiments, a Brookfield (spindle and cup) viscometer is used to calculate the viscosity of the gel formulation described herein. In some embodiments, the viscosity ranges referred to herein are measured at room temperature. In other embodiments, the viscosity ranges referred to herein are measured at body temperature (e.g., at the average body temperature of a healthy human).

### Gel/Ointment Dose-to-Dose Uniformity

Typical ophthalmic gels are packaged in eye drop bottles and administered as drops. For example, a single administration (i.e. a single dose) of an ophthalmic gel comprises a single drop, two drops, three drops or more into the eyes of the patient. Furthermore, typical ophthalmic ointments are packaged in tubes or other squeezable containers with a dispensing nozzle through which strips of the ointment are delivered. For example, a single administration (i.e. a single dose) of an ophthalmic ointment comprises a single strip, or multiple strips into the eyes of the patient. In some embodiments, one dose of the ophthalmic gel described herein is one drop of the gel composition from the eye drop bottle. In some embodiments, one dose of the ophthalmic ointment is one strip of the ointment composition dispensed through the nozzle of a dispersing tube.

In some cases, described herein include ophthalmic gel compositions which provide a dose-to-dose uniform concentrations. In some instances, the dose-to-dose uniform concentration does not present significant variations of drug content from one dose to another. In some instances, the dose-to-dose uniform concentration does provide consistent drug content from one dose to another.

In some cases, described herein include ophthalmic ointment compositions which provide a dose-to-dose uniform concentrations. In some instances, the dose-to-dose uniform concentration does not present significant variations of drug content from one dose to another. In some instances, the dose-to-dose uniform concentration does provide consistent drug content from one dose to another.

In some embodiments, the composition has a dose-to-dose ophthalmic agent concentration variation of less than 50%. In some embodiments, the composition has a dose-to-dose ophthalmic agent concentration variation of less than 40%. In some embodiments, the composition has a dose-to-dose ophthalmic agent concentration variation of less than 30%. In some embodiments, the composition has a dose-to-dose ophthalmic agent concentration variation of less than 20%. In some embodiments, the composition has a dose-to-dose ophthalmic agent concentration variation of less than 10%. In some embodiments, the composition has a dose-to-dose ophthalmic agent concentration variation of less than 5%.

In some embodiments, the dose-to-dose ophthalmic agent concentration variation is based on 10 consecutive doses. In some embodiments, the dose-to-dose ophthalmic agent concentration variation is based on 8 consecutive doses. In some embodiments, the dose-to-dose ophthalmic agent concentration variation is based on 5 consecutive doses. In some embodiments, the dose-to-dose ophthalmic agent concentration variation is based on 3 consecutive doses. In some embodiments, the dose-to-dose ophthalmic agent concentration variation is based on 2 consecutive doses.

A nonsettling formulation should not require shaking to disperse drug uniformly. A “no-shake” formulation is potentially advantageous over formulations that require shaking for the simple reason that patients' shaking behavior is a major source of variability in the amount of drug dosed. It has been reported that patients often times do not or forget to shake their ophthalmic compositions that requires shaking before administering a dose, despite the instructions to shake that were clearly marked on the label. On the other hand, even for those patients who do shake the product, it is normally not possible to determine whether the shaking is adequate in intensity and/or duration to render the product uniform. In some embodiments, the ophthalmic gel compositions and ophthalmic ointment compositions described herein are “no-shake” formulations that maintained the dose-to-dose uniformity described herein.

To evaluate the dose-to-dose uniformity, drop bottles or tubes containing the ophthalmic aqueous compositions, the ophthalmic gel compositions, or ophthalmic ointment compositions are stored upright for a minimum of 12 hours prior to the start of the test. To simulate the recommended dosing of these products, predetermined number of drops or strips are dispensed from each commercial bottles or tubes at predetermined time intervals for an extended period of time or until no product was left in the bottle or tube. All drops and strips are dispensed into tared glass vials, capped, and stored at room temperature until analysis. Concentrations of a muscarinic agent such as aceclidine, pilocarpine, or tropicamide in the expressed drops were determined using a reverse-phase HPLC method.

### Methods of Treatment

Disclosed herein are methods of arresting presbyopia development or slowing progression of presbyopia by administering to an eye of an individual in need thereof an effective amount of an ophthalmic composition as described above. Also disclosed herein are methods of preventing presbyopia development by administering to an eye of an individual in need thereof an effective amount of an ophthalmic composition as described above.

In some embodiments, the ophthalmic aqueous formulations described herein are packaged in eye drop bottles and administered as drops. For example, a single administration (i.e. a single dose) of an ophthalmic aqueous formulation comprises a single drop, two drops, three drops or more into the eyes of the patient. In some embodiments, the ophthalmic gel formulations described herein are packaged in eye drop bottles and administered as drops. For example, a single administration (i.e. a single dose) of an ophthalmic gel comprises a single drop, two drops, three drops or more into the eyes of the patient. In some embodiments, the ophthalmic ointment formulations described herein are packaged in tubes or other squeezable containers with a dispensing nozzle through which strips of the ointment are delivered. For example, a single administration (i.e. a single dose) of an ophthalmic ointment comprises a single strip, or multiple strips into the eyes of the patient. In some embodiments, one dose of the ophthalmic aqueous formulation described herein is one drop of the aqueous composition from the eye drop bottle. In some embodiments, one dose of the ophthalmic gel described herein is one drop of the gel composition from the eye drop bottle. In some embodiments, one dose of the ophthalmic ointment is one strip of the ointment composition dispensed through the nozzle of a dispersing tube. In some embodiments, the ophthalmic composition is not formulated as an injectable formulation.

In some embodiments, the ophthalmic composition is formulated as an ophthalmic solution for treatment of presbyopia, progression of presbyopia, or slowing progression of presbyopia.

In some embodiments of the disclosed method, the ophthalmic composition is stored below room temperature prior to first use. In some embodiments of the disclosed method, the ophthalmic composition is stored at between about 2° C. to about 10° C. prior to first use. In some embodiments of the disclosed method, the ophthalmic composition is stored at about 2° C., about 3° C., about 4° C., about 5° C., about 6° C., about 7° C., about 8° C., about 9° C., or about 10° C. prior to first use. In some embodiments of the disclosed method, the ophthalmic composition is stored at between about 4° C. to about 8° C. prior to first use.

In some embodiments of the disclosed method, the ophthalmic composition is stored at room temperature after first use. In some embodiments of the disclosed method, the ophthalmic composition is stored at between about 16° C. to about 26° C. after to first use. In some embodiments of the disclosed method, the ophthalmic composition is stored at about 16° C., about 17° C., about 18° C., about 19° C., about 20° C., about 21° C., about 22° C., about 23° C., about 24° C., about 25° C., or about 26° C. after first use.

In some embodiments, the ophthalmic aqueous formulations are administered as follows: the lower lid of the eye to be administered was pulled down and a predetermined amount of the aqueous formulation (e.g. 1-3 drops) is applied to the inside of the eyelid. The ophthalmic tip of the dispensing mechanism does not touch any surface to avoid contamination and/or injury.

In some embodiments, the ophthalmic gel formulations are administered as follows: the lower lid of the eye to be administered was pulled down and a predetermined amount of gel (e.g. 1-3 drops) is applied to the inside of the eyelid. The ophthalmic tip of the dispensing mechanism does not touch any surface to avoid contamination and/or injury.

In some embodiments, the ophthalmic ointment formulations are administered as follows: the lower lid of the eye to be administered was pulled down and a small amount of ointment (approximately 0.25 inches) was applied to the inside of the eyelid. The ophthalmic tip of the dispensing mechanism does not touch any surface to avoid contamination and/or injury.

In some embodiments, the ophthalmic composition is administered at predetermined time intervals over an extended period of time. In some embodiments, the ophthalmic composition is administered once every day. In some embodiments, the ophthalmic composition is administered every other day. In some embodiments, the ophthalmic composition is administered over 1 week, 2 weeks, 1 month, 2 months, 3 months, 6 moths, 1 year, 2 years, 3 years, 4 years, 5 years, 6 years, 7 years, 8 years, 9 years, 10 years, 11 years, or 12-15 years.

In some embodiments, the ophthalmic composition is administered in doses having a dose-to-dose ophthalmic agent concentration variation of less than 50%, less than 40%, less than 30%, less than 20%, less than 10%, or less than 5%.

The number of times a composition is administered to an individual in need thereof depends on the discretion of a medical professional, the disorder, the severity of the disorder, and the individual's response to the formulation. In some embodiments, a composition disclosed herein is administered once to an individual in need thereof with a mild acute condition. In some embodiments, a composition disclosed herein is administered more than once to an individual in need thereof with a moderate or severe acute condition. In the case wherein the patient's condition does not improve, upon the doctor's discretion the administration of an ophthalmic agent is administered chronically, that is, for an extended period of time, including throughout the duration of the patient's life in order to ameliorate or otherwise control or limit the symptoms of the patient's disease or condition.

In the case wherein the patient's condition does not improve, upon the doctor's discretion the administration of the ophthalmic agent is administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the ophthalmic agent is given continuously; alternatively, the dose of drug being administered is temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday is from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's ophthalmic conditions has occurred, a maintenance ophthalmic agent dose is administered if necessary. Subsequently, the dosage or the frequency of administration, or both, is optionally reduced, as a function of the symptoms, to a level at which the improved disease, disorder or condition is retained. In certain embodiments, patients require intermittent treatment on a long-term basis upon any recurrence of symptoms.

The amount of ophthalmic agent that will correspond to such an amount will vary depending upon factors such as the particular compound, disease condition and its severity, according to the particular circumstances surrounding the case, including, e.g., the specific ophthalmic agent being administered, the route of administration, the condition being treated, the target area being treated, and the subject or host being treated. The desired dose is presented in a single dose or as divided doses administered simultaneously (or over a short period of time) or at appropriate intervals.

In some embodiments, the initial administration is a particular ophthalmic agent and the subsequent administration a different formulation or ophthalmic agent.

### Fluid-Dispensing Device

In certain embodiments, described herein include an ophthalmic product, which comprises a fluid-dispensing device comprising a reservoir and a dispensing tip fitted onto the reservoir, and the composition described herein, wherein the composition is dispensed from the dispensing tip into an eye of an individual in need thereof. In some instances, the composition in the reservoir is substantially preservative-free. In other instances, the composition in the reservoir comprises a preservative, but is filtered prior to dispensing from the dispensing tip, and the dispensed composition is substantially preservative-free.

In some embodiments, the ophthalmic composition comprises a muscarinic agent. In some cases, the ophthalmic product comprises a fluid-dispensing device comprising a reservoir and a dispensing tip fitted onto the reservoir; and an ophthalmic composition comprising from about 0.001 wt % to about 0.05 wt % of a muscarinic agent and deuterated water, at a pH of from about 4.2 to about 7.9, in the reservoir; wherein the ophthalmic composition is dispensed from the dispensing tip into an eye of an individual in need thereof, and wherein the dispensed ophthalmic composition is substantially preservative-free.

In some embodiments, the ophthalmic composition comprises an ophthalmic agent. In some cases, the ophthalmic product comprises a fluid-dispensing device comprising a reservoir and a dispensing tip fitted onto the reservoir; and an ophthalmic composition comprising an ophthalmic agent and deuterated water, at a pH of from about 4 to about 8, in the reservoir; wherein the ophthalmic agent is not a muscarinic agent and does not extend singlet oxygen lifetime, wherein the ophthalmic composition is dispensed from the dispensing tip into an eye of an individual in need thereof, and wherein the dispensed ophthalmic composition is substantially preservative-free.

As used herein, the term “substantially preservative-free” or “substantially free of a preservative” refers to the composition as having one of: less than about 1%, less than about 0.5%, less than about 0.4%, less than about 0.3%, less than about 0.2%, less than about 0.1%, less than about 0.01%, or less than about 0.001% of a preservative. In some instances, the term refers to the composition as having 0% of a preservative, or preservative-free.

In some embodiments, the reservoir comprises of a polymeric material, for example, polyvinyl chloride (PVC) plastics or non-PVC plastics. In some instances, the material of the reservoir comprises high-density polyethylene (HDPE), low-density polyethylene (LDPE), polyethylene terephthalate (PET), polyvinyl chloride (PVC), polypropylene (PP), polystyrene (PS), fluorine treated HDPE, post-consumer resin (PCR), K-resin (SBC), or bioplastic. In some embodiments, the material of the reservoir comprises ethylene vinyl acetate (EVA) and block copolymers such as Kraton®. In some cases, the material of the reservoir comprises high-density polyethylene (HDPE). In some cases, the material of the reservoir comprises low-density polyethylene (LDPE). In some cases, the material of the reservoir comprises polyethylene terephthalate (PET). In some cases, the material of the reservoir comprises polypropylene (PP). In some cases, the material of the reservoir comprises polystyrene (PS). In some cases, the material of the reservoir comprises ethylene vinyl acetate (EVA).

In some instances, the reservoir further comprises a plasticizer. Exemplary plasticizer comprises families of phthalate esters such as di-2-ethylhexylphthalate (DEHP), mono-(2-ethylhexyl) phthalate (MEHP), and triethylhexyltrimellitate (TEHTM); citrate esters such as acetyltri-n-hexyl citrate, acetyltri-n-(hexyl/octyl/decyl) citrate, acetyltri-n-(octyl/decyl) citrate, and n-butyryltri-n-hexyl citrate; and non-phthalate plasticizers such as TEHTM, di(isononyl) cyclohexane-1,2-dicarboxylate (DINCH), or n-butyryltri-n-hexyl citrate.

In some embodiments, the reservoir is at least partially elastically deformable so as to dispense the ophthalmic composition by pressing on the reservoir.

In some embodiments, the reservoir comprises glass.

In some embodiments, the reservoir stores multiple unit doses of the composition described herein.

In some embodiments, the fluid-dispensing device described herein is a multi-dose fluid-dispensing device.

In some embodiments, the fluid-dispensing device described herein enables storage of a preservative-free or substantially preservative-free composition. In some cases, the fluid-dispensing device is a multi-dose preservative-free device.

In some instances, a fluid-dispensing device from Aptar Pharma (AptarGroup) is utilized for delivery of a composition described herein. In some cases, the composition is preservative-free.

In some cases, a fluid-dispensing device from Nemera La Verpilliére S.A.S. is utilized for delivery of a composition described herein. In some cases, a fluid-dispensing device as described in U.S. Pat. Nos. 8,986,266 and/or 8,863,998 is utilized for delivery of a composition described herein. In some cases, the composition is preservative-free.

In some cases, a fluid-dispensing device from CIS Pharma is utilized for delivery of a composition described herein. In some cases, the composition is preservative-free.

In some embodiments, the fluid-dispensing device described herein optionally comprises an atomizer, a pump, or a mister. In such cases, a mechanical system such as a pump, a mister, or an atomizer is incorporated into the fluid-dispensing device to facilitate delivery of the composition described herein and optionally to facilitate dose uniformity (e.g., between each administration, minimize excessive drug volume, and/or enhance droplet uniformity). In additional cases, a mechanical system such as a pump, a mister, or an atomizer is incorporated into the fluid-dispensing device to enhance and/or optimize the amount of drug delivered to the eye.

In some instances, an atomizer and/or pump system from Aero Pump GMBH (Adelphi Healthcare Packaging) is utilized with the fluid-dispensing device and the composition described herein. In some instances, a multiple-dosage fluid-dispensing device from Aero Pump GMBH is utilized for delivery of the composition described herein. In some cases, a fluid-dispensing device as described in U.S. Patent Publication 2016/279663 and/or 2015/076174 (Aero Pump GMBH) is utilized with the fluid-dispensing device and the composition described herein.

In some embodiments, a fluid-dispensing device from Eyenovia, Inc. is utilized for delivery of the composition described herein. In some cases, a fluid-dispensing device comprising one or more of a delivery system and/or component described in U.S. patents and Patent Publications U.S. Pat. Nos. 9,539,604, 9,087,145, 9,463,486, or 2012/143152 are utilized for delivery of the composition described herein.

In some cases, a fluid-dispensing device comprising one or more of a delivery system and/or component from Kedalion Therapeutics is utilized for delivery of the composition described herein.

In some cases, a fluid-dispensing device comprising one or more of a delivery system and/or component from Aptar Pharma (e.g., a pump dispensing system) is utilized for delivery of the composition described herein.

In some embodiments, the fluid-dispensing device optionally comprises an internal filter or membrane. In some instances, the internal filter or membrane is located within the fluid-dispensing device at a position capable of removing a preservative from the ophthalmic composition prior to dispensing the ophthalmic composition into the eye of the individual. In some instances, the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof. In some instances, the internal filter or membrane is located within the fluid-dispensing device at a position capable of removing a preservative selected from benzalkonium chloride, cetrimonium, sodium perborate, stabilized oxychloro complex, SofZia, polyquaternium-1, chlorobutanol, edetate disodium, polyhexamethylene biguanide, or combinations thereof, from the ophthalmic composition prior to dispensing the ophthalmic composition into the eye of the individual. In some instances, the internal filter or membrane is located within the fluid-dispensing device at a position capable of removing a preservative selected from benzalkonium chloride (BAK, BAC, or BKC) from the ophthalmic composition prior to dispensing the ophthalmic composition into the eye of the individual. In some cases, the internal filter or membrane is located at the junction connecting the dispensing tip to the reservoir. In other cases, the internal filter or membrane is located within the dispensing tip.

In some instances, the internal filter or membrane is located within the fluid-dispensing device at a position capable of removing a microorganism and/or an endotoxin from the ophthalmic composition prior to dispensing the ophthalmic composition into the eye of the individual. In some cases, the internal filter or membrane is located at the junction connecting the dispensing tip to the reservoir. In other cases, the internal filter or membrane is located within the dispensing tip. In some cases, the ophthalmic composition is a preservative-free composition.

In some cases, the internal filter or membrane comprises cellulose acetate, cellulose nitrate, nylon, polyether sulfone (PES), polypropylene (PP), polyvinyl difluoride (PVDF), silicone, polycarbonate, or a combination thereof.

In some embodiments, a filter system from TearClear is utilized with a fluid-dispensing device and composition described herein. In some cases, a filter system from TearClear removes a preservative from the composition described herein in-situ, e.g., the filter system is within the fluid-dispensing device which removes a preservative from the composition as the composition is passed from the filter and dispensed into the eye of an individual.

In some cases, the dispensed composition comprises one of: less than about 1%, less than about 0.5%, less than about 0.4%, less than about 0.3%, less than about 0.2%, less than about 0.1%, less than about 0.01%, less than about 0.001%, or less than about 0.0001% of a preservative. In some cases, the dispensed composition is preservative-free.

In some instances, the droplet volume dispensed from the fluid-dispensing device described herein is from about 0.1 μL to about 50 μL. In some instances, the droplet volume is one of: about 0.1 μL to about 40 μL, about 0.5 μL to about 30 μL, about 1 μL to about 30 μL, about 5 μL to about 20 μL, about 10 μL to about 20 μL, about 5 μL to about 40 μL, about 5 μL to about 30 μL, about 6 μL to about 8 μL, about 6 μL to about 7 μL, about 7 μL to about 8 μL, about 10 μL to about 40 μL, or about 10 μL to about 30 μL. In some cases, the droplet volume dispensed from the fluid-dispensing device described herein is about 0.1 μL, about 0.2 μL, about 0.3 μL, about 0.4 μL, about 0.5 μL, about 1 μL, about 5 μL, about 6 μL, about 7 μL, about 8 μL, about 9 μL, about 10 μL, about 20 μL, about 30 μL, about 40 μL, or about 50 μL.

In some embodiments, the linear size or diameter of the droplet when spherical is about 1 up to less than 100 microns. In some cases, the linear size or diameter of the droplet is about 20 to 100 microns, about 1 to 20 microns, 1-15 microns, 1-10 microns, 8-20 microns, 8-15 microns, 8-12 microns, or 1-5 microns. In the context of an aerosol or mist, the size of the droplet is, for example, 1-5 microns, 1-10 microns, less than 10 microns, greater than 10 microns, or up to 100 microns.

In some cases, the diameter of the droplet is calculated using the equation V=4πr³ where the diameter=2r.

In some instances, the fluid-dispensing device is suitable for dispensing the composition described herein having a viscosity described herein. In some cases, the composition has a viscosity of up to 500 cP, up to 600 cP, up to 1000 cP, up to 10,000 cP, or up to 50,000 cP.

In some instances, the fluid-dispensing device described herein facilitates at least 60%, 70%, 80%, 85%, 90%, 95%, or 99% of the ejected mass of a droplet deposited on the eye of an individual. In some cases, the fluid-dispensing device described herein facilitates at least 70% of the ejected mass of a droplet to be deposited on the eye of an individual. In some cases, the fluid-dispensing device described herein facilitates at least 80% of the ejected mass of a droplet to be deposited on the eye of an individual. In some cases, the fluid-dispensing device described herein facilitates at least 90% of the ejected mass of a droplet to be deposited on the eye of an individual. In some cases, the fluid-dispensing device described herein facilitates at least 95% of the ejected mass of a droplet to be deposited on the eye of an individual. In some cases, the fluid-dispensing device described herein facilitates at least 99% of the ejected mass of a droplet to be deposited on the eye of an individual.

### Kits/Articles of Manufacture

The disclosure also provides kits for preventing or arresting presbyopia development. Such kits generally will comprise one or more of the ophthalmic compositions disclosed herein, and instructions for using the kit. The disclosure also contemplates the use of one or more of the ophthalmic compositions, in the manufacture of medicaments for treating, abating, reducing, or ameliorating the symptoms of a disease, dysfunction, or disorder in a mammal, such as a human that has, is suspected of having, or at risk for developing presbyopia.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

The articles of manufacture provided herein contain packaging materials. Packaging materials for use in packaging pharmaceutical products are also presented herein. See, e.g., U.S. Pat. Nos. 5,323,907, 5,052,558 and 5,033,252. Examples of pharmaceutical packaging materials include, but are not limited to, drop bottles, tubes, pumps, bags, vials, containers, syringes, bottles, and any packaging material suitable for a selected formulation and intended mode of administration and treatment. A wide array of ophthalmic compositions provided herein are contemplated as are a variety of treatments for any disease, disorder, or condition that benefits by controlled release administration of an ophthalmic agent to the eye.

In some embodiments, a kit comprises one or more additional containers, each with one or more of various materials (such as rinses, wipes, and/or devices) desirable from a commercial and user standpoint for use of a formulation described herein. Such materials also include labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

In certain embodiments, the ophthalmic compositions are presented in a dispenser device which contains one or more unit dosage forms containing a compound provided herein. In a further embodiment, the dispenser device is accompanied by instructions for administration. In yet a further embodiment, the dispenser is also accompanied with a notice associated with the container in form prescribed by a governmental agency regulating the manufacture, use, or sale of pharmaceuticals, which notice is reflective of approval by the agency of the form of the drug for human or veterinary administration. In another embodiment, such notice, for example, is the labeling approved by the U.S. Food and Drug Administration for prescription drugs, or the approved product insert. In yet another embodiment, compositions containing a compound provided herein formulated in a compatible pharmaceutical carrier are also prepared, placed in an appropriate container, and labeled for treatment of an indicated condition.

### EXAMPLES

### Example 1—Ophthalmic Formulations

Exemplary compositions for preparation of ophthalmic formulations are described in Tables 1-24.

TABLE 1

| Ingredient | Concentration (wt %) |
|---|---|
| Aceclidine or pharmaceutically acceptable salt of aceclidine | 0.25-2.0 (wt %) |
| Buffer agent and/or pH adjusting agent (e.g., borates, citrate | q.s. for pH = 4.2-7.9 and q.s. |
| and/or DCl, citrate) | for 0.004 wt %-0.20 wt % |
| Preservative (e.g. benzalkonium chloride, cetrimonium sodium | 0.001-0.10 (wt %) |
| perborate, etc.) | |
| Tonicity and/or Osmolarity adjustor (e.g. NaCl, mannitol, etc) | q.s. to 0.5-2.0 wt % |
| Deuterated Water | q.s. to 100 wt % |

TABLE 2

| Ingredient | Concentration (wt %) |
|---|---|
| Aceclidine or pharmaceutically acceptable salt of aceclidine | 0.25-2.0 (wt %) |
| Buffer agent and/or pH adjusting agent (e.g., borates, citrate | q.s. for pH = 4.2-7.9 and q.s. |
| and/or DCl, citrate) | for 0.004 wt %-0.20 wt % |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Deuterated Water | q.s. to 100 wt % |

TABLE 3

| Ingredient | Concentration (wt %) |
|---|---|
| Aceclidine or pharmaceutically acceptable salt of aceclidine | 0.25-2.0 (wt %) |
| Buffer agent (monosodium phosphate anhydrous, disodium | q.s. for pH = 4.2-7.9 .s. for |
| phosphate anhydrous) and/or pH adjusting agent (e.g., borates | 0.004 wt %-0.20 wt % |
| and/or DCl) | |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Deuterated Water | q.s. to 100 wt % |

TABLE 4

| Ingredient | Concentration (wt %) |
|---|---|
| Aceclidine or pharmaceutically acceptable salt of aceclidine | 0.25-2.0 (wt %) |
| Pilocarpine or pharmaceutically acceptable salt of pilocarpine | 0.001-2.5 (wt %) |
| Buffer agent and/or pH adjusting agent (e.g., borates, citrate | q.s. for pH = 4.2-7.9 and q.s. |
| and/or DCl, citrate) | for 0.004 wt %-0.20 wt % |
| Preservative (e.g. benzalkonium chloride, cetrimonium sodium | 0.001-0.10 (wt %) |
| perborate, etc.) | |
| Tonicity and/or Osmolarity adjustor (e.g. NaCl, mannitol, etc) | q.s. to 0.5-2.0 wt % |
| Deuterated Water | q.s. to 100 wt % |

TABLE 5

| Ingredient | Concentration (wt %) |
|---|---|
| Aceclidine or pharmaceutically acceptable salt of aceclidine | 0.25-2.0 (wt %) |
| Pilocarpine or pharmaceutically acceptable salt of pilocarpine | 0.001-2.5 (wt %) |
| Buffer agent and/or pH adjusting agent (e.g., borates, citrate | q.s. for pH = 4.2-7.9 and q.s. |
| and/or DCl, citrate) | for 0.004 wt %-0.20 wt % |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Deuterated Water | q.s. to 100 wt % |

TABLE 6

| Ingredient | Concentration (wt %) |
|---|---|
| Aceclidine or pharmaceutically acceptable salt of aceclidine | 0.25-2.0 (wt %) |
| Pilocarpine or pharmaceutically acceptable salt of pilocarpine | 0.001-2.5 (wt %) |
| Buffer agent (monosodium phosphate anhydrous, disodium | q.s. for pH = 4.2-7.9 .s. for |
| phosphate anhydrous) and/or pH adjusting agent (e.g., borates | 0.004 wt %-0.20 wt % |
| and/or DCl) | |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Deuterated Water | q.s. to 100 wt % |

TABLE 7

| Ingredient | Concentration (wt %) |
|---|---|
| Aceclidine or pharmaceutically acceptable salt of aceclidine | 0.25-2.0 (wt %) |
| Tropicamide or pharmaceutically acceptable salt of tropicamide | 0.01-.025 (wt %) |
| Buffer agent and/or pH adjusting agent (e.g., borates, citrate | q.s. for pH = 4.2-7.9 and q.s. |
| and/or DCl, citrate) | for 0.004 wt %-0.20 wt % |
| Preservative (e.g. benzalkonium chloride, cetrimonium sodium | 0.001-0.10 (wt %) |
| perborate, etc.) | |
| Tonicity and/or Osmolarity adjustor (e.g. NaCl, mannitol, etc) | q.s. to 0.5-2.0 wt % |
| Deuterated Water | q.s. to 100 wt % |

TABLE 8

| Ingredient | Concentration (wt %) |
|---|---|
| Aceclidine or pharmaceutically acceptable salt of aceclidine | 0.25-2.0 (wt %) |
| Tropicamide or pharmaceutically acceptable salt of tropicamide | 0.01-.025 (wt %) |
| Buffer agent and/or pH adjusting agent (e.g., borates, citrate | q.s. for pH-4.2-7.9 and q.s. |
| and/or DCl, citrate) | for 0.004 wt %-0.20 wt % |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Deuterated Water | q.s. to 100 wt % |

TABLE 9

| Ingredient | Concentration (wt %) |
|---|---|
| Aceclidine or pharmaceutically acceptable salt of aceclidine | 0.25-2.0 (wt %) |
| Tropicamide or pharmaceutically acceptable salt of tropicamide | 0.01-.025 (wt %) |
| Buffer agent (monosodium phosphate anhydrous, disodium | q.s. for pH = 4.2-7.9 .s. for |
| phosphate anhydrous) and/or pH adjusting agent (e.g., borates | 0.004 wt %-0.20 wt % |
| and/or DCl) | |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Deuterated Water | q.s. to 100 wt % |

TABLE 10

| Ingredient | Concentration (wt %) |
|---|---|
| Aceclidine or pharmaceutically acceptable salt of aceclidine | 0.25-2.0 (wt %) |
| Pilocarpine or pharmaceutically acceptable salt of pilocarpine | 0.001-2.5 (wt %) |
| Tropicamide or pharmaceutically acceptable salt of tropicamide | 0.01-.025 (wt %) |
| Buffer agent and/or pH adjusting agent (e.g., borates, citrate | q.s. for pH = 4.2-7.9 and q.s. |
| and/or DCl, citrate) | for 0.004 wt %-0.20 wt % |
| Preservative (e.g. benzalkonium chloride, cetrimonium sodium | 0.001-0.10 (wt %) |
| perborate, etc.) | |
| Tonicity and/or Osmolarity adjustor (e.g. NaCl, mannitol, etc) | q.s. to 0.5-2.0 wt % |
| Deuterated Water | q.s. to 100 wt % |

TABLE 11

| Ingredient | Concentration (wt %) |
|---|---|
| Aceclidine or pharmaceutically acceptable salt of aceclidine | 0.25-2.0 (wt %) |
| Pilocarpine or pharmaceutically acceptable salt of pilocarpine | 0.001-2.5 (wt %) |
| Tropicamide or pharmaceutically acceptable salt of tropicamide | 0.01-.025 (wt %) |
| Buffer agent and/or pH adjusting agent (e.g., borates, citrate | q.s. for pH = 4.2-7.9 and q.s. |
| and/or DCl, citrate) | for 0.004 wt %-0.20 wt % |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Deuterated Water | q.s. to 100 wt % |

TABLE 12

| Ingredient | Concentration (wt %) |
|---|---|
| Aceclidine or pharmaceutically acceptable salt of aceclidine | 0.25-2.0 (wt %) |
| Pilocarpine or pharmaceutically acceptable salt of pilocarpine | 0.001-2.5 (wt %) |
| Tropicamide or pharmaceutically acceptable salt of tropicamide | 0.01-.025 (wt %) |
| Buffer agent (monosodium phosphate anhydrous, disodium | q.s. for pH = 4.2-7.9 .s. for |
| phosphate anhydrous) and/or pH adjusting agent (e.g., borates | 0.004 wt %-0.20 wt % |
| and/or DCl) | |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Deuterated Water | q.s. to 100 wt % |

TABLE 13

| Ingredient | Concentration (wt %) |
|---|---|
| Aceclidine or pharmaceutically acceptable salt of aceclidine | 0.25-2.0 (wt %) |
| Buffer agent and/or pH adjusting agent (e.g., borates, citrate | q.s. for pH = 4.2-7.9 and q.s. |
| and/or DCl, citrate) | for 0.004 wt %-0.20 wt % |
| Preservative (e.g. benzalkonium chloride, cetrimonium sodium | 0.001-0.10 (wt %) |
| perborate, etc.) | |
| Tonicity and/or Osmolarity adjustor (e.g. NaCl, mannitol, etc) | q.s. to 0.5-2.0 wt % |
| Water | q.s. to 100 wt % |

TABLE 14

| Ingredient | Concentration (wt %) |
|---|---|
| Aceclidine or pharmaceutically acceptable salt of aceclidine | 0.25-2.0 (wt %) |
| Buffer agent and/or pH adjusting agent (e.g., borates, citrate | q.s. for pH = 4.2-7.9 and q.s. |
| and/or DCI, citrate) | for 0.004 wt %-0.20 wt % |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Water | q.s. to 100 wt % |

TABLE 15

| Ingredient | Concentration (wt %) |
|---|---|
| Aceclidine or pharmaceutically acceptable salt of aceclidine | 0.25-2.0 (wt %) |
| Buffer agent (monosodium phosphate anhydrous, disodium | q.s. for pH = 4.2-7.9 .s. for |
| phosphate anhydrous) and/or pH adjusting agent (e.g., borates | 0.004 wt %-0.20 wt % |
| and/or DC1) | |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Water | q.s. to 100 wt % |

TABLE 16

| Ingredient | Concentration (wt %) |
|---|---|
| Aceclidine or pharmaceutically acceptable salt of aceclidine | 0.25-2.0 (wt %) |
| Pilocarpine or pharmaceutically acceptable salt of pilocarpine | 0.001-2.5 (wt %) |
| Buffer agent and/or pH adjusting agent (e.g., borates, citrate | q.s. for pH = 4.2-7.9 and q.s. |
| and/or DCI, citrate) | for 0.004 wt %-0.20 wt % |
| Preservative (e.g. benzalkonium chloride, cetrimonium sodium | 0.001-0.10 (wt %) |
| perborate, etc.) | |
| Tonicity and/or Osmolarity adjustor (e.g. NaCl, mannitol, etc) | q.s. to 0.5-2.0 wt % |
| Water | q.s. to 100 wt % |

TABLE 17

| Ingredient | Concentration (wt %) |
|---|---|
| Aceclidine or pharmaceutically acceptable salt of aceclidine | 0.25-2.0 (wt %) |
| Pilocarpine or pharmaceutically acceptable salt of pilocarpine | 0.001-2.5 (wt %) |
| Buffer agent and/or pH adjusting agent (e.g., borates, citrate | q.s. for pH = 4.2-7.9 and q.s. |
| and/or DCI, citrate) | for 0.004 wt %-0.20 wt % |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Water | q.s. to 100 wt % |

TABLE 18

| Ingredient | Concentration (wt %) |
|---|---|
| Aceclidine or pharmaceutically acceptable salt of aceclidine | 0.25-2.0 (wt %) |
| Pilocarpine or pharmaceutically acceptable salt of pilocarpine | 0.001-2.5 (wt %) |
| Buffer agent (monosodium phosphate anhydrous, disodium | q.s. for pH = 4.2-7.9 .s. for |
| phosphate anhydrous) and/or pH adjusting agent (e.g., borates | 0.004 wt %-0.20 wt % |
| and/or DCI) | |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Water | q.s. to 100 wt % |

TABLE 19

| Ingredient | Concentration (wt %) |
|---|---|
| Aceclidine or pharmaceutically acceptable salt of aceclidine | 0.25-2.0 (wt %) |
| Tropicamide or pharmaceutically acceptable salt of tropicamide | 0.01-.025 (wt %) |
| Buffer agent and/or pH adjusting agent (e.g., borates, citrate | q.s. for pH = 4.2-7.9 and q.s. |
| and/or DCI, citrate) | for 0.004 wt %-0.20 wt % |
| Preservative (e.g. benzalkonium chloride, cetrimonium sodium | 0.001-0.10 (wt %) |
| perborate, etc.) | |
| Tonicity and/or Osmolarity adjustor (e.g. NaCl, mannitol, etc) | q.s. to 0.5-2.0 wt % |
| Water | q.s. to 100 wt % |

TABLE 20

| Ingredient | Concentration (wt %) |
|---|---|
| Aceclidine or pharmaceutically acceptable salt of aceclidine | 0.25-2.0 (wt %) |
| Tropicamide or pharmaceutically acceptable salt of tropicamide | 0.01-.025 (wt %) |
| Buffer agent and/or pH adjusting agent (e.g., borates, citrate | q.s. for pH = 4.2-7.9 and q.s. |
| and/or DCI, citrate) | for 0.004 wt %-0.20 wt % |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Water | q.s. to 100 wt % |

TABLE 21

| Ingredient | Concentration (wt %) |
|---|---|
| Aceclidine or pharmaceutically acceptable salt of aceclidine | 0.25-2.0 (wt %) |
| Tropicamide or pharmaceutically acceptable salt of tropicamide | 0.01-.025 (wt %) |
| Buffer agent (monosodium phosphate anhydrous, disodium | q.s. for pH = 4.2-7.9 .s. for |
| phosphate anhydrous) and/or pH adjusting agent (e.g., borates | 0.004 wt %-0.20 wt % |
| and/or DC1) | |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Water | q.s. to 100 wt % |

TABLE 22

| Ingredient | Concentration (wt %) |
|---|---|
| Aceclidine or pharmaceutically acceptable salt of aceclidine | 0.25-2.0 (wt %) |
| Pilocarpine or pharmaceutically acceptable salt of pilocarpine | 0.001-2.5 (wt %) |
| Tropicamide or pharmaceutically acceptable salt of tropicamide | 0.01-.025 (wt %) |
| Buffer agent and/or pH adjusting agent (e.g., borates, citrate | q.s. for pH = 4.2-7.9 and q.s. |
| and/or DCI, citrate) | for 0.004 wt %-0.20 wt % |
| Preservative (e.g. benzalkonium chloride, cetrimonium sodium | 0.001-0.10 (wt %) |
| perborate, etc.) | |
| Tonicity and/or Osmolarity adjustor (e.g. NaCl, mannitol, etc) | q.s. to 0.5-2.0 wt % |
| Water | q.s. to 100 wt % |

TABLE 23

| Ingredient | Concentration (wt %) |
|---|---|
| Aceclidine or pharmaceutically acceptable salt of aceclidine | 0.25-2.0 (wt %) |
| Pilocarpine or pharmaceutically acceptable salt of pilocarpine | 0.001-2.5 (wt %) |
| Tropicamide or pharmaceutically acceptable salt of tropicamide | 0.01-.025 (wt %) |
| Buffer agent and/or pH adjusting agent (e.g., borates, citrate | q.s. for pH = 4.2-7.9 and q.s. |
| and/or DCI, citrate) | for 0.004 wt %-0.20 wt % |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Water | q.s. to 100 wt % |

TABLE 24

| Ingredient | Concentration (wt %) |
|---|---|
| Aceclidine or pharmaceutically acceptable salt of aceclidine | 0.25-2.0 (wt %) |
| Pilocarpine or pharmaceutically acceptable salt of pilocarpine | 0.001-2.5 (wt %) |
| Tropicamide or pharmaceutically acceptable salt of tropicamide | 0.01-.025 (wt %) |
| Buffer agent (monosodium phosphate anhydrous, disodium | q.s. for pH = 4.2-7.9 .s. for |
| phosphate anhydrous) and/or pH adjusting agent (e.g., borates | 0.004 wt %-0.20 wt % |
| and/or DCI) | |
| Preservative (e.g. benzalkonium chloride, cetrimonium sodium | 0% |
| perborate, etc.) | |
| Tonicity and/or Osmolarity adjustor (e.g. NaCl, mannitol, etc) | q.s. to 0.5-2.0 wt % |
| Water | q.s. to 100 wt % |

### Example 2—Stability Analysis

Five aceclidine solutions, five tropicamide, five pilocarpine, five aceclidine and pilocarpine, five aceclidine and tropicamide, and five aceclidine, pilocarpine, and tropicamide solutions are prepared. The pH of the five solutions is determined. Each solution is thoroughly mixed. A 0.22 micron filter is placed on the tip of the syringe and the solution is aliquoted into separate sterile containers according to Table 25.

TABLE 25
Container Filling Outline

| | | Total |
|---|---|---|
| | Volume of Drug | Containers |
| Type of Container | Product in Container | Filled |
| Sterile Eyedroppers | 5-mL | 72 |
| Sterile Glass Vials | 5-mL | 72 |

The samples are then stored at different conditions for stability analysis. The samples are analyzed at different time points up to 2 months. The storage conditions include: 40° C. with 75% relative humidity (RH) (samples are transferred from 2-8° C. condition after 3 days), 25° C. with 60% RH, and 60° C. The time points are 1 week, 2 weeks, 1 month, and 2 months. At each of the time point, one plastic eyedropper (LDPE plastic) and one glass vial from each of the stored condition are removed and allowed to equilibrate to ambient conditions. Once equilibrated, both the plastic eyedropper and the glass vials are inverted 3 times. The solution in the eyedroppers is transferred to an HPLC vial in a drop wise fashion through the dropper. The solution in the glass vial is aliquoted into an HPLC vial using a glass Pasteur pipette. The samples are then tested for purity and potency using the UPLC method listed in Table 26.

TABLE 26
UPLC Method Parameters

| Parameter | Condition | |
|---|---|---|
| Column | EMD, Hiber HR PurospherSTAR C-18, | |
| | 100 × 2.1 mm, 2 μm | |
| Mobile Phase/Diluent | 87:13, 50 mM Potassium | |
| | Phosphate:Acetonitrile, pH 3.5 | |
| Flow | Isocratic | |
| Flow Rate | 0.5 | mL/min |
| Detection Wavelength | 210 | nm |
| Column Temperature | 30 ± 3° | C. |
| Autosampler Temperature | 5 ± 3° | C. |
| Run Time | 6.0 | minutes |
| Injection Volume | 10 | μL* |

| | |
|---|---|
| Needle Wash Solution | 90/10 Water:Acetonitrile |

*Modified from original method to maintain sensitivity at 100 μg/mL nominal.

Stability data for the various solutions and Arrhenius based shelf life predictions are determined.

### Example 3—Dose Uniformity (10-Dose)

To evaluate the dose-to-dose uniformity, drop bottles containing the ophthalmic aqueous composition are stored upright for a predetermined period of time (e.g. 12 hours) prior to the start of the test. To simulate the recommended dosing of the product, 10 drops of the aqueous composition are dispensed from each bottle at predetermined time intervals (e.g. consecutively, every 1 minute, every 10 minutes, every hour or every 24 hours). All drops are dispensed into tared glass vials, capped, and stored at room temperature until analysis. Concentrations of aceclidine, pilocarpine, and tropicamide in the expressed drops are determined using a reverse-phase HPLC method.

### Example 4—Dose Uniformity (5-Dose)

To evaluate the dose-to-dose uniformity, drop bottles containing the ophthalmic aqueous composition are stored upright for a predetermined period of time (e.g. 12 hours) prior to the start of the test. To simulate the recommended dosing of the product, 5 drops of the aqueous composition are dispensed from each bottle at predetermined time intervals (e.g. consecutively, every 1 minute, every 10 minutes, every hour or every 24 hours). All drops are dispensed into tared glass vials, capped, and stored at room temperature until analysis. Concentrations of aceclidine, pilocarpine, and tropicamide in the expressed drops are determined using a reverse-phase HPLC method.

### Example 5—Dose Uniformity (2-Dose)

To evaluate the dose-to-dose uniformity, drop bottles containing the ophthalmic aqueous composition are stored upright for a predetermined period of time (e.g. 12 hours) prior to the start of the test. To simulate the recommended dosing of the product, 2 drops of the aqueous composition are dispensed from each bottle at predetermined time intervals (e.g. consecutively, every 1 minute, every 10 minutes, every hour or every 24 hours). All drops are dispensed into tared glass vials, capped, and stored at room temperature until analysis. Concentrations of aceclidine, pilocarpine, and tropicamide in the expressed drops are determined using a reverse-phase HPLC method.

### Example 6—Formulation Stability Comparison and Determination of Shelf Life and Activation Energy

Aceclidine, pilocarpine, and tropicamide are used for this experiment. Various formulations as described in Example 1 are analyzed at t=0, 2 weeks, and 4 weeks. The conditions that are tested include 40° C. with 75% relative humidity (RH), 25° C. with 60% RH, and 60° C. A RP-HPLC method is used to carry out the analysis.

Purity, potency, and degradation of aceclidine, pilocarpine, and tropicamide are determined as well pH and pD stability. Data is also used to determine shelf life and activation energy.

### Example 7—Effect of pH on Ophthalmic Acceptance in Guinea Pits

A cohort of guinea pigs is administered 50 μL of ophthalmic formulations having different pH values described herein. For example, ophthalmic formulations comprising H₂O or deuterated water (e.g., D₂O) are administered to the animals. Animal behavior is recorded at predetermined time intervals to evaluate the acceptance of the ophthalmic formulations.

### Example 8—In Vivo Rabbit Eye Irritation Test

The exemplary compositions disclosed herein are subjected to rabbit eye irritation test to evalaute their safety profile. The test composition are tested for eye irritation test in New Zealand Rabbits (see for example Abraham M H, et al., Draize rabbit eye test compatibility with eye irritation thresholds in humans: a quantitative structure-activity relationship analysis. Toxicol Sci. 2003 December; 76(2):384-91. Epub 2003 Sep. 26; see also Gettings S D et al., A comparison of low volume, Draize and in vitro eye irritation test data. III. Surfactant-based formulations. Food Chem Toxicol. 1998 March; 36(3):209-31). The study involves single ocular administration into the right eye and the same volume of its placebo in the left eye of each of the three rabbits. Rabbits are examined immediately and after instillation of the compositions for 4, 24, 48 and 72 hours post instillation to note the signs/symptoms of eye irritation, if any. The test compositions show no sign of irritancy in cornea, iris and conjunctivae of the rabbit eyes.

### Example 9: Stability of Pilocarpine in Deuterated Water

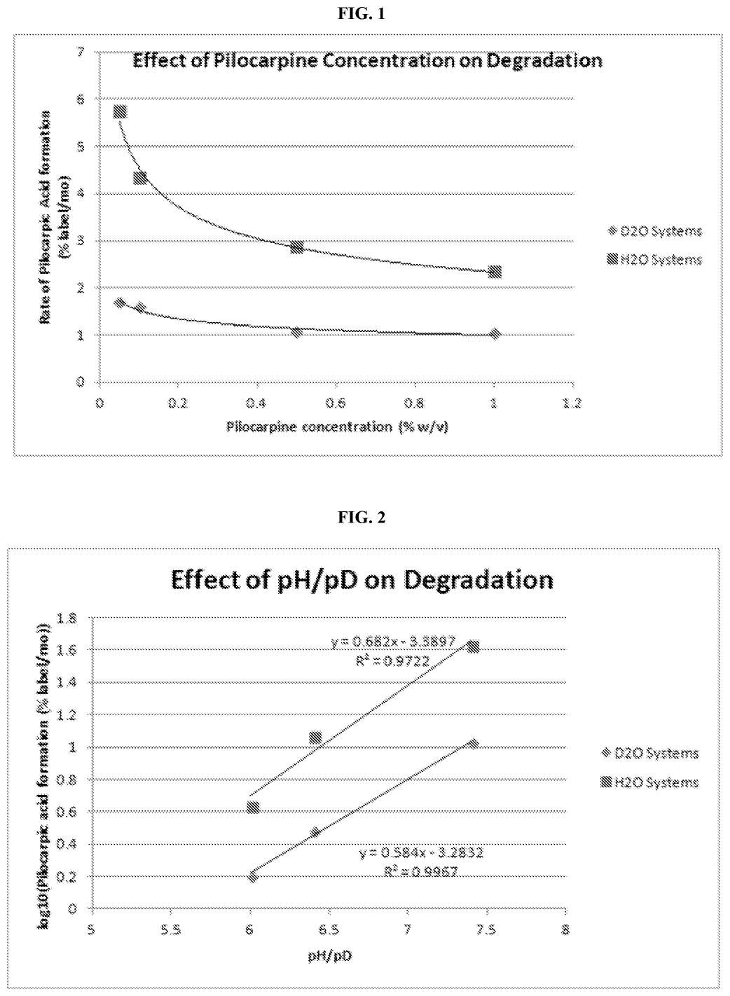

The stability of pilocarpine was tested over a range of concentrations. Pilocarpic acid formation, a major degradant of pilocarpine, was measured. As seen in FIG. 1, there was improved stability at higher concentrations of pilocarpine. There was also improved stability in deuterated water as compared to non-deuterated water. This stabilizing factor increases as pilocarpine concentration decreases.

The stability of pilocarpine, as measured by the percent of pilocarpic acid formation, was measured at different pH/pD values, as depicted in FIG. 2. A linear log/log relationship was identified between the log(rate of pilocarpic acid formation) and pH.

### Example 10: Pilocarpine Stability at 6 Months

A non-GMP stability study was performed on the water-based and deuterated-water based pilocarpine formulations listed in Table 27.

TABLE 27
Pilocarpine Formulations

| # | Formulation Composition | Lot No. | Conditions |
|---|---|---|---|
| 1 | 1.00% Pilocarpine, 100% D₂O, 0.01% BAK, | 1293-12- | 25/60 |
| | 0.04% Citrate, 0.9% NaCl, pH* 5.60 | 20-1 | 40/75 |
| 2 | 0.50% Pilocarpine, 100% D₂O, 0.01% BAK, | 1293-12- | 25/60 |
| | 0.04% Citrate, 0.9% NaCl, pH* 5.60 | 21-1 | 40/75 |
| 3 | 0.10% Pilocarpine, 100% D₂O, 0.01% BAK, | 1293-12- | 25/60 |
| | 0.04% Citrate, 0.9% NaCl, pH* 5.60 | 22-1 | 40/75 |
| 4 | 0.05% Pilocarpine, 100% D₂O, 0.01% BAK, | 1293-12- | 25/60 |
| | 0.04% Citrate, 0.9% NaCl, pH* 5.60 | 23-1 | 40/75 |
| 5 | 0.10% Pilocarpine, 100% D₂O, 0.01% BAK, | 1293-12- | 25/60 |
| | 0.04% Phosphate, 0.9% NaCl, pH* 7.00 | 24-1 | only |
| 6 | 0.10% Pilocarpine, 100% D₂O, 0.01% BAK, | 1293-12- | 25/60 |
| | 0.04% Citrate, 0.9% NaCl, pH* 6.00 | 25-1 | 40/75 |
| 7 | 1.00% Pilocarpine, 100% H₂O, 0.01% BAK, | 1293-12- | 25/60 |
| | 0.04% Citrate, 0.9% NaCl, pH 6.00 | 26-1 | 40/75 |
| 8 | 0.50% Pilocarpine, 100% H₂O, 0.01% BAK, | 1293-12- | 25/60 |
| | 0.04% Citrate, 0.9% NaCl, pH 6.00 | 27-1 | 40/75 |
| 9 | 0.10% Pilocarpine, 100% H₂O, 0.01% BAK, | 1293-12- | 25/60 |
| | 0.04% Phosphate, 0.9% NaCl, pH 6.00 | 28-1 | 40/75 |
| 10 | 0.05% Pilocarpine, 100% H₂O, 0.01% BAK, | 1293-12- | 25/60 |
| | 0.04% Citrate, 0.9% NaCl, pH 6.00 | 29-1 | 40/75 |
| 11 | 0.10% Pilocarpine, 100% H₂O, 0.01% BAK, | 1293-12- | 25/60 |
| | 0.04% Phosphate, 0.9% NaCl, pH 7.40 | 30-1 | only |
| 12 | 0.10% Pilocarpine, 100% H₂O, 0.01% BAK, | 1293-12- | 25/60 |
| | 0.04% Citrate, 0.9% NaCl, pH 6.40 | 31-1 | 40/75 |

Each formulation was packaged as a 5 mL Rexam bottle containing 5 mL of pilocarpine formulations. The pilocarpine was stored at the conditions listed in Table 27. The samples were then stored at different conditions for stability analysis. The samples were analyzed at different time points up to 2 months. Prior to testing, the samples were stored at 5° C. with ambient relative humidity (RH). The storage conditions included 25° C. with 60% RH and 40° C. with 75% RH. The time points were 1 month, 3 months, and 6 months of storage. Prior to testing, 3 bottles were removed for each formulated at each storage condition and equilibrated to ambient conditions prior to testing. The results are listed in Tables 28-39.

TABLE 28
Stability results for Formulation 1
Storage Condition: 5° C. ± 3° C./Ambient RH

| | Time Point |
|---|---|
| Parameter | Initial |
| Visual Appearance | Clear solution, free of visible particulates |
| Clear solution, free of visible | |
| particulates | |
| pH | 5.55 |
| Pilocarpine Hydrochloride | 98.5% |
| Content | |
| (HPLC Assay) | |
| Pilocarpine Hydrochloride | RRT 1.29 = 0.06% |
| Related Substances (% Area) | |
| Pilocarpic Acid | 0.05% |
| Isopilocarpic Acid | ND1 |
| Isopilocarpine | 0.05% |
| Pilocarpine Hydrochloride | 0.16% |
| Total Related Substances | |

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 5.55 | 5.67 | 5.53 | 5.26 |
| Pilocarpine Hydrochloride | 98.5% | 97.6% | 99.9% | 97.5% |
| Content | | | | |
| (HPLC Assay) | | | | |
| Pilocarpine Hydrochloride | RRT 1.29 = 0.06% | RRT 1.05 = 0.07% | RRT 1.05 = 0.15% | RRT 1.05 = 0.20% |
| Related Substances (% Area) | | | | |
| Pilocarpic Acid | 0.05% | 0.36% | 0.91% | 1.76% |
| Isopilocarpic Acid | ND1 | ND1 | ND1 | ND1 |
| Isopilocarpine | 0.05% | 0.20% | 0.39% | 0.71% |
| Pilocarpine Hydrochloride | 0.16% | 0.63% | 1.45% | 2.68% |
| Total Related Substances | | | | |

Storage Condition: 40° C. ± 2° C./75% RH ± 5% RH

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 5.55 | 5.34 | 5.01 | 4.69 |
| Pilocarpine Hydrochloride | 98.5% | 98.5% | 96.8% | 92.8% |
| Content | | | | |
| (HPLC Assay) | | | | |
| Pilocarpine Hydrochloride | RRT 1.29 = 0.06% | RRT 1.05 = 0.07% | RRT 1.05 = 0.19% | RRT 1.05 = 0.14% |
| Related Substances (% Area) | | | | |
| Pilocarpic Acid | 0.05% | 1.45% | 3.24% | 5.03% |
| Isopilocarpic Acid | ND1 | ND1 | ND1 | ND1 |
| Isopilocarpine | 0.05% | 0.75% | 1.57% | 2.40% |
| Pilocarpine Hydrochloride | 0.16% | 2.27% | 5.00% | 7.56% |
| Total Related Substances | | | | |

TABLE 29
Stability data for Formulation 2
Storage Condition: 5° C. ± 3° C./Ambient RH

| | Time Point |
|---|---|
| Parameter | Initial |
| Visual Appearance | Clear solution, free of visible particulates |
| pH | 5.57 |
| Pilocarpine | 100.6% |
| Hydrochloride | |
| Content | |
| (HPLC Assay) | |
| Pilocarpine | RRT 1.29 = 0.05% |
| Hydrochloride | |
| Related | |
| Substances | |
| (% Area) | |
| Pilocarpic Acid | ND¹ |
| Isopilocarpic Acid | ND¹ |
| Isopilocarpine | 0.06% |
| Pilocarpine | 0.11% |
| Hydrochloride | |
| Total Related | |
| Substances | |

Storage Condition: 25° C. ± 2° C./60% RH ± 5% RH

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 5.57 | 5.51 | 5.40 | 5.35 |
| Pilocarpine | 100.6% | 97.3% | 99.6% | 95.9% |
| Hydrochloride | | | | |
| Content | | | | |
| (HPLC Assay) | | | | |
| Pilocarpine | RRT 1.29 = 0.05% | RRT 1.05 = 0.06% | RRT 1.05 = 0.14% | RRT 1.05 = 0.20% |
| Hydrochloride | | | | |
| Related | | | | |
| Substances | | | | |
| (% Area) | | | | |
| Pilocarpic Acid | ND¹ | 0.30% | 0.81% | 1.61% |
| Isopilocarpic Acid | ND¹ | ND¹ | ND¹ | ND¹ |
| Isopilocarpine | 0.06% | 0.17% | 0.34% | 0.58% |
| Pilocarpine | 0.11% | 0.53% | 1.29% | 2.39% |
| Hydrochloride | | | | |
| Total Related | | | | |
| Substances | | | | |
| | Time Point | | | |
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 5.57 | 5.36 | 5.02 | 4.88 |
| Pilocarpine | 100.6% | 98.9% | 97.8% | 91.3% |
| Hydrochloride | | | | |
| Content | | | | |
| (HPLC Assay) | | | | |
| Pilocarpine | RRT 1.29 = 0.05% | RRT 1.05 = 0.05% | RRT 1.05 = 0.12% | RRT 1.05 = 0.14% |
| Hydrochloride | | | | |
| Related | | | | |
| Substances | | | | |
| (% Area) | | | | |
| Pilocarpic Acid | ND¹ | 1.49% | 3.25% | 5.62% |
| Isopilocarpic Acid | ND¹ | ND¹ | ND¹ | ND¹ |
| Isopilocarpine | 0.06% | 0.71% | 1.44% | 2.56% |
| Pilocarpine | 0.11% | 2.25% | 4.81% | 8.32% |
| Hydrochloride | | | | |
| Total Related | | | | |
| Substances | | | | |

TABLE 30
Stability data for Formulation 3
Storage Condition: 5° C. ± 3° C./Ambient RH

| | Time Point |
|---|---|
| Parameter | Initial |
| Visual Appearance | Clear solution, free of visible particulates |
| pH | 5.59 |
| Pilocarpine | 99.1% |
| Hydrochloride | |
| Content | |
| (HPLC Assay) | |
| Pilocarpine | RRT 1.29 = 0.05% |
| Hydrochloride | |
| Related | |
| Substances | |
| (% Area) | |
| Pilocarpic Acid | ND¹ |
| Isopilocarpic Acid | ND¹ |
| Isopilocarpine | 0.05% |
| Pilocarpine | 0.10% |
| Hydrochloride | |
| Total Related | |
| Substances | |

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 5.59 | 5.52 | 5.41 | 5.51 |
| Pilocarpine | 99.1% | 101.4% | 98.5% | 96.1% |
| Hydrochloride | | | | |
| Content | | | | |
| (HPLC Assay) | | | | |
| Pilocarpine | RRT 1.29 = 0.05% | ND¹ | ND¹ | RRT 1.05 = 0.18% |
| Hydrochloride | | | | |
| Related | | | | |
| Substances | | | | |
| (% Area) | | | | |
| Pilocarpic Acid | ND¹ | 0.36% | 0.98% | 2.07% |
| Isopilocarpic Acid | ND¹ | ND¹ | ND¹ | ND¹ |
| Isopilocarpine | 0.05% | 0.17% | 0.38% | 0.72% |
| Pilocarpine | 0.10% | 0.53% | 1.36% | 2.96% |
| Hydrochloride | | | | |
| Total Related | | | | |
| Substances | | | | |
| | Time Point | | | |
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 5.59 | 5.50 | 5.17 | 5.22 |
| Pilocarpine | 99.1% | 98.5% | 94.0% | 87.9% |
| Hydrochloride | | | | |
| Content | | | | |
| (HPLC Assay) | | | | |
| Pilocarpine | RRT 1.29 = 0.05% | ND¹ | ND¹ | RRT 1.05 = 0.13% |
| Hydrochloride | | | | |
| Related | | | | |
| Substances | | | | |
| (% Area) | | | | |
| Pilocarpic Acid | ND¹ | 2.09% | 4.87% | 8.17% |
| Isopilocarpic Acid | ND¹ | ND¹ | 0.07% | 0.08% |
| Isopilocarpine | 0.05% | 0.94% | 2.15% | 3.75% |
| Pilocarpine | 0.10% | 3.03% | 7.09% | 12.13% |
| Hydrochloride | | | | |
| Total Related | | | | |
| Substances | | | | |

TABLE 31
Stability data for Formulation 4
Storage Condition: 5° C. ± 3° C./Ambient RH

| | Time Point |
|---|---|
| Parameter | Initial |
| Visual | Clear solution, free of visible particulates |
| Appearance | |
| pH | 5.61 |
| Pilocarpine | 100.4% |
| Hydrochloride | |
| Content | |
| (HPLC Assay) | |
| Pilocarpine | RRT 0.97 = 0.08% |
| Hydrochloride | RRT 1.28 = 0.06% |
| Related | |
| Substances | |
| (% Area) | |
| Pilocarpic Acid | ND¹ |
| Isopilocarpic | ND¹ |
| Acid | |
| Isopilocarpine | 0.05% |
| Pilocarpine | 0.19% |
| Hydrochloride | |
| Total Related | |
| Substances | |

Storage Condition: 25° C. ± 2° C./60% RH ± 5% RH

| | Time Point | | | |
|---|---|---|---|---|
| | Initial | T = 1 Month | T = 3 Months | T = 6 Months |
| Parameter | 26 May 2020 | 29 Jun. 2020 | 27 Aug. 2020 | 11 Dec. 2020 |
| Visual | Clear solution, | Clear solution, | Clear solution, | Clear solution, |
| Appearance | free of visible | free of visible | free of visible | free of visible |
| | particulates | particulates | particulates | particulates |
| pH | 5.61 | 5.57 | 5.53 | 5.54 |
| Pilocarpine | 100.4% | 102.6% | 99.3% | 96.6% |
| Hydrochloride | | | | |
| Content | | | | |
| (HPLC Assay) | | | | |
| Pilocarpine | RRT 0.97 = 0.08% | RRT 0.97 = 0.07% | RRT 0.97 = 0.06% | RRT 1.05 = 0.17% |
| Hydrochloride | RRT 1.28 = 0.06% | | | |
| Related | | | | |
| Substances | | | | |
| (% Area) | | | | |
| Pilocarpic Acid | ND¹ | 0.37% | 1.00% | 2.07% |
| Isopilocarpic | ND¹ | ND¹ | ND¹ | ND¹ |
| Acid | | | | |
| Isopilocarpine | 0.05% | 0.18% | 0.37% | 0.66% |
| Pilocarpine | 0.19% | 0.62% | 1.43% | 2.89% |
| Hydrochloride | | | | |
| Total Related | | | | |
| Substances | | | | |

| | Time Point | | | |
|---|---|---|---|---|
| Parameter | Initial | T = 1 Month | T = 3 Months | T = 6 Months |
| Visual | Clear solution, | Clear solution, | Clear solution, | Clear solution, |
| Appearance | free of visible | free of visible | free of visible | free of visible |
| | particulates | particulates | particulates | particulates |
| pH | 5.61 | 5.48 | 5.16 | 5.30 |
| Pilocarpine | 100.4% | 98.9% | 94.6% | 85.9% |
| Hydrochloride | | | | |
| Content | | | | |
| (HPLC Assay) | | | | |
| Pilocarpine | RRT 0.97 = 0.08% | ND¹ | ND¹ | RRT 1.05 = 0.12% |
| Hydrochloride | RRT 1.28 = 0.06% | | | |
| Related | | | | |
| Substances | | | | |
| (% Area) | | | | |
| Pilocarpic Acid | ND¹ | 2.33% | 5.21% | 9.53% |
| Isopilocarpic | ND¹ | ND¹ | 0.13% | 0.12% |
| Acid | | | | |
| Isopilocarpine | 0.05% | 1.03% | 2.27% | 4.42% |
| Pilocarpine | 0.19% | 3.36% | 7.61% | 14.19% |
| Hydrochloride | | | | |
| Total Related | | | | |
| Substances | | | | |

TABLE 32
Stability data for Formulation 5
Storage Condition: 25° C. ± 2° C./60% RH ± 5% RH

| | Time Point | | | |
|---|---|---|---|---|
| Parameter | Initial | T = 1 Month | T = 3 Months | T = 6 Months |
| Visual | Clear solution, | Clear solution, | Clear solution, | Clear solution, |
| Appearance | free of visible | free of visible | free of visible | free of visible |
| | particulates | particulates | particulates | particulates |
| pH | 6.96 | 6.89 | 6.82 | 6.66 |
| Pilocarpine | 99.4% | 95.1% | 83.9% | 69.6% |
| Hydrochloride | | | | |
| Content | | | | |
| (HPLC Assay) | | | | |
| Pilocarpine | RRT 1.05 = 0.07% | RRT 1.05 = 0.09% | RRT 0.23 = 0.09% | RRT 1.05 = 0.16% |
| Hydrochloride | RRT 1.29 = 0.07% | | RRT 0.37 = 0.07% | |
| Related | | | RRT 1.05 = 0.19% | |
| Substances | | | | |
| (% Area) | | | | |
| Pilocarpic Acid | 0.18% | 4.90% | 11.88% | 21.27% |
| Isopilocarpic | ND¹ | 0.08% | 0.37% | 1.08% |
| Acid | | | | |
| Isopilocarpine | 0.09% | 1.68% | 3.83% | 6.56% |
| Pilocarpine | 0.41% | 6.75% | 16.43% | 29.06% |
| Hydrochloride | | | | |
| Total Related | | | | |
| Substances | | | | |

Storage Condition: 40° C. ± 2° C./75% RH ± 5% RH

| | Time Point | | | |
|---|---|---|---|---|
| Parameter | Initial | T = 1 Month | T = 3 Months | T = 6 Months |
| Visual | Clear solution, | Clear solution, | Clear solution, | |
| Appearance | free of visible | free of visible | free of visible | |
| | particulates | particulates | particulates | |
| pH | 6.96 | 6.71 | 6.44 | |
| Pilocarpine | 99.4% | 73.6% | 50.9% | |
| Hydrochloride | | | | |
| Content | | | | |
| (HPLC Assay) | | | | |
| Pilocarpine | RRT 1.05 = 0.07% | RRT 1.05 = 0.06% | ND¹ | |
| Hydrochloride | RRT 1.29 = 0.07% | | | |
| Related | | | | |
| Substances | | | | |
| (% Area) | | | | |
| Pilocarpic Acid | 0.18% | 18.14% | 33.82% | |
| Isopilocarpic | ND¹ | 0.93% | 2.62% | |
| Acid | | | | |
| Isopilocarpine | 0.09% | 7.08% | 12.54% | |
| Pilocarpine | 0.41% | 26.21% | 48.98% | |
| Hydrochloride | | | | |
| Total Related | | | | |
| Substances | | | | |

TABLE 33
Stability data for Formulation 6
Storage Condition: 5° C. ± 3° C./Ambient RH

| | Time Point |
|---|---|
| Parameter | Initial |
| Visual Appearance | Clear solution, free of visible particulates |
| pH | 6.02 |
| Pilocarpine | 99.6% |
| Hydrochloride | |
| Content | |
| (HPLC Assay) | |
| Pilocarpine | RRT 1.29 = 0.06% |
| Hydrochloride | |
| Related | |
| Substances | |
| (% Area) | |
| Pilocarpic Acid | ND¹ |
| Isopilocarpic Acid | ND¹ |
| Isopilocarpine | 0.05% |
| Pilocarpine | 0.11% |
| Hydrochloride | |
| Total Related | |
| Substances | |

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 6.02 | 6.05 | 5.84 | 5.88 |
| Pilocarpine | 99.6% | 100.2% | 96.0% | 93.9% |
| Hydrochloride | | | | |
| Content | | | | |
| (HPLC Assay) | | | | |
| Pilocarpine | RRT 1.29 = 0.06% | ND1 | RRT 1.05 = 0.06% | RRT 1.05 = 0.18% |
| Hydrochloride | | | | |
| Related | | | | |
| Substances | | | | |
| (% Area) | | | | |
| Pilocarpic Acid | ND¹ | 0.81% | 2.20% | 4.11% |
| Isopilocarpic Acid | ND¹ | ND¹ | ND¹ | ND¹ |
| Isopilocarpine | 0.05% | 0.32% | 0.80% | 1.45% |
| Pilocarpine | 0.11% | 1.13% | 3.06% | 5.74% |
| Hydrochloride | | | | |
| Total Related | | | | |
| Substances | | | | |

Storage Condition: 40° C. ± 2° C./75% RH ± 5% RH

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 6.02 | 5.78 | 5.63 | 5.49 |
| Pilocarpine | 99.6% | 95.4% | 86.3% | 77.2% |
| Hydrochloride | | | | |
| Content | | | | |
| (HPLC Assay) | | | | |
| Pilocarpine | RRT 1.29 = 0.06% | ND¹ | ND¹ | RRT 1.05 = 0.11% |
| Hydrochloride | | | | |
| Related | | | | |
| Substances | | | | |
| (% Area) | | | | |
| Pilocarpic Acid | ND¹ | 4.12% | 9.32% | 14.56% |
| Isopilocarpic Acid | ND¹ | 0.06% | 0.22% | 0.29% |
| Isopilocarpine | 0.05% | 1.84% | 4.17% | 7.15% |
| Pilocarpine | 0.11% | 6.02% | 13.71% | 22.10% |
| Hydrochloride | | | | |
| Total Related | | | | |
| Substances | | | | |

TABLE 34
Stability data for Formulation 7
Storage Condition: 5° C. ± 3° C./Ambient RH

| | Time Point |
|---|---|
| Parameter | Initial |
| Visual Appearance | Clear solution, free of visible particulates |
| pH | 5.94 |
| Pilocarpine | 98.6% |
| Hydrochloride | |
| Content | |
| (HPLC Assay) | |
| Pilocarpine | RRT 1.05 = 0.06% |
| Hydrochloride | RRT 1.29 = 0.23% |
| Related | |
| Substances | |
| (% Area) | |
| Pilocarpic Acid | 0.08% |
| Isopilocarpic Acid | ND¹ |
| Isopilocarpine | 0.07% |
| Pilocarpine | 0.44% |
| Hydrochloride | |
| Total Related | |
| Substances | |

Storage Condition: 25° C. ± 2° C./60% RH ± 5% RH

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 5.94 | 5.80 | 5.60 | 5.28 |
| Pilocarpine | 98.6% | 99.9% | 98.2% | 92.2% |
| Hydrochloride | | | | |
| Content | | | | |
| (HPLC Assay) | | | | |
| Pilocarpine | RRT 1.05 = 0.06% | RRT 1.05 = 0.09% | RRT 1.05 = 0.22% | RRT 1.05 = 0.23% |
| Hydrochloride | RRT 1.29 = 0.23% | | | |
| Related | | | | |
| Substances | | | | |
| (% Area) | | | | |
| Pilocarpic Acid | 0.08% | 1.41% | 3.22% | 5.24% |
| Isopilocarpic Acid | ND¹ | ND¹ | ND¹ | ND¹ |
| Isopilocarpine | 0.07% | 0.50% | 1.07% | 1.68% |
| Pilocarpine | 0.44% | 2.00% | 4.51% | 7.15% |
| Hydrochloride | | | | |
| Total Related | | | | |
| Substances | | | | |

Storage Condition: 40° C. ± 2° C./75% RH ± 5% RH

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 5.94 | 5.43 | 4.78 | 4.67 |
| Pilocarpine | 98.6% | 97.5% | 92.5% | 87.4% |
| Hydrochloride | | | | |
| Content | | | | |
| (HPLC Assay) | | | | |
| Pilocarpine | RRT 1.05 = 0.06% | RRT 1.05 = 0.09% | RRT 1.05 = 0.20% | RRT 1.05 = 0.14% |
| Hydrochloride | | | | |
| Related | | | | |
| Substances | RRT 1.29 = 0.23% | | | |
| (% Area) | | | | |
| Pilocarpic Acid | 0.08% | 4.22% | 7.49% | 9.45% |
| Isopilocarpic Acid | ND¹ | ND¹ | 0.06% | ND1 |
| Isopilocarpine | 0.07% | 1.74% | 3.15% | 4.63% |
| Pilocarpine | 0.44% | 6.05% | 10.90% | 14.22% |
| Hydrochloride | | | | |
| Total Related | | | | |
| Substances | | | | |

TABLE 35
Stability data for Formulation 8
Storage Condition: 5° C. ± 3° C./Ambient RH

| | Time Point |
|---|---|
| Parameter | Initial |
| Visual Appearance | Clear solution, free of visible particulates |
| pH | 5.94 |
| Pilocarpine | 98.9% |
| Hydrochloride | |
| Content | |
| (HPLC Assay) | |
| Pilocarpine | RRT 1.29 = 0.30% |
| Hydrochloride | |
| Related | |
| Substances | |
| (% Area) | |
| Pilocarpic Acid | 0.07% |
| Isopilocarpic Acid | ND¹ |
| Isopilocarpine | 0.06% |
| Pilocarpine | 0.43% |
| Hydrochloride | |
| Total Related | |
| Substances | |

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 5.94 | 5.82 | 5.72 | 5.45 |
| Pilocarpine | 98.9% | 99.7% | 98.8% | 94.2% |
| Hydrochloride | | | | |
| Content | | | | |
| (HPLC Assay) | | | | |
| Pilocarpine | RRT 1.29 = 0.30% | RRT 1.05 = 0.08% | RRT 1.05 = 0.15% | RRT 1.05 = 0.23% |
| Hydrochloride | | | | |
| Related | | | | |
| Substances | | | | |
| (% Area) | | | | |
| Pilocarpic Acid | 0.07% | 1.45% | 3.42% | 5.80% |
| Isopilocarpic Acid | ND¹ | ND¹ | ND¹ | 0.05% |
| Isopilocarpine | 0.06% | 0.50% | 1.07% | 1.78% |
| Pilocarpine | 0.43% | 2.03% | 4.64% | 7.86% |
| Hydrochloride | | | | |
| Total Related | | | | |
| Substances | | | | |

Storage Condition: 40° C. ± 2° C./75% RH ± 5% RH

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 5.94 | 5.51 | 5.09 | 4.88 |
| Pilocarpine | 98.9% | 94.5% | 90.2% | 83.8. % |
| Hydrochloride | | | | |
| Content | | | | |
| (HPLC Assay) | | | | |
| Pilocarpine | RRT 1.29 = 0.30% | RRT 1.05 = 0.06% | RRT 1.05 = 0.17% | RRT 1.05 = 0.14% |
| Hydrochloride | | | | |
| Related | | | | |
| Substances | | | | |
| (% Area) | | | | |
| Pilocarpic Acid | 0.07% | 4.93% | 9.06% | 11.54% |
| Isopilocarpic Acid | ND¹ | 0.05% | 0.10% | 0.08% |
| Isopilocarpine | 0.06% | 1.93% | 3.65% | 5.37% |
| Pilocarpine | 0.43% | 6.97% | 12.98% | 17.13% |
| Hydrochloride | | | | |
| Total Related | | | | |
| Substances | | | | |

TABLE 36
Stability data for Formulation 9
Storage Condition: 5° C. ± 3° C./Ambient RH

| | Time Point |
|---|---|
| Parameter | Initial |
| Visual Appearance | Clear solution, free of visible particulates |
| pH | 5.99 |
| Pilocarpine | 99.9% |
| Hydrochloride | |
| Content | |
| (HPLC Assay) | |
| Pilocarpine | RRT 1.29 = 0.32% |
| Hydrochloride | |
| Related | |
| Substances (% | |
| Area) | |
| Pilocarpic Acid | 0.07% |
| Isopilocarpic Acid | ND¹ |
| Isopilocarpine | 0.06% |
| Pilocarpine | 0.45% |
| Hydrochloride | |
| Total Related | |
| Substances | |

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, free |
| | free of visible | free of visible | free of visible | of visible |
| | particulates | particulates | particulates | particulates |
| pH | 5.99 | 5.97 | 5.91 | 5.72 |
| Pilocarpine | 99.9% | 99.3% | 94.8% | 88.1% |
| Hydrochloride | | | | |
| Content | | | | |
| (HPLC Assay) | | | | |
| Pilocarpine | RRT 1.29 = 0.32% | ND1 | RRT 1.05 = 0.08% | RRT 1.05 = 0.20% |
| Hydrochloride | | | | |
| Related | | | | |
| Substances (% | | | | |
| Area) | | | | |
| Pilocarpic Acid | 0.07% | 1.87% | 4.49% | 8.12% |
| Isopilocarpic Acid | ND¹ | ND¹ | 0.06% | 0.10% |
| Isopilocarpine | 0.06% | 0.57% | 1.26% | 2.22% |
| Pilocarpine | 0.45% | 2.44% | 5.89% | 10.64% |
| Hydrochloride | | | | |
| Total Related | | | | |
| Substances | | | | |

Storage Condition: 40° C. ± 2° C./75% RH ± 5% RH

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, free |
| | free of visible | free of visible | free of visible | of visible |
| | particulates | particulates | particulates | particulates |
| pH | 5.99 | 5.74 | 5.36 | 5.17 |
| Pilocarpine | 99.9% | 90.4% | 82.4% | 78.7% |
| Hydrochloride | | | | |
| Content | | | | |
| (HPLC Assay) | | | | |
| Pilocarpine | RRT 1.29 = 0.32% | ND¹ | RRT 1.05 = 0.06% | RRT 1.05 = 0.13% |
| Hydrochloride | | | | |
| Related | | | | |
| Substances (% | | | | |
| Area) | | | | |
| Pilocarpic Acid | 0.07% | 7.54% | 13.65% | 14.75% |
| Isopilocarpic Acid | ND1 | 0.12% | 0.23% | 0.11% |
| Isopilocarpine | 0.06% | 2.73% | 5.16% | 6.61% |
| Pilocarpine | 0.45% | 10.39% | 19.10% | 21.59% |
| Hydrochloride | | | | |
| Total Related | | | | |
| Substances | | | | |

TABLE 37
Stability data for Formulation 10
Storage Condition: 5° C. ± 3° C./Ambient RH

| | Time Point |
|---|---|
| Parameter | Initial |
| Visual Appearance | Clear solution, free of visible particulates |
| pH | 6.00 |
| Pilocarpine | 100.5% |
| Hydrochloride Content | |
| (HPLC Assay) | |
| Pilocarpine | RRT 0.97 = 0.07% |
| Hydrochloride Related | RRT 1.29 = 0.28% |
| Substances (% Area) | |
| Pilocarpic Acid | 0.06% |
| Isopilocarpic Acid | ND¹ |
| Isopilocarpine | 0.06% |
| Pilocarpine | 0.47% |
| Hydrochloride Total | |
| Related Substances | |

Storage Condition: 25° C. ± 2° C./60% RH ± 5% RH

| | Time Point | | | |
|---|---|---|---|---|
| Parameter | Initial | T = 1 month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 6.00 | 5.97 | 5.76 | 5.85 |
| Pilocarpine | 100.5% | 99.5% | 95.8% | 88.6% |
| Hydrochloride Content | | | | |
| (HPLC Assay) | | | | |
| Pilocarpine | RRT 0.97 = 0.07% | RRT 0.97 = 0.07% | RRT 0.97 = 0.07% | RRT 1.06 = 0.15% |
| Hydrochloride Related | RRT 1.29 = 0.28% | | | |
| Substances (% Area) | | | | |
| Pilocarpic Acid | 0.06% | 1.76% | 4.27% | 7.98% |
| Isopilocarpic Acid | ND¹ | ND¹ | 0.07% | 0.10% |
| Isopilocarpine | 0.06% | 0.54% | 1.23% | 2.28% |
| Pilocarpine | 0.47% | 2.37% | 5.64% | 10.51% |
| Hydrochloride Total | | | | |
| Related Substances | | | | |

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 6.00 | 5.76 | 5.57 | 5.47 |
| Pilocarpine | 100.5% | 89.2% | 76.1% | 63.0% |
| Hydrochloride Content | | | | |
| (HPLC Assay) | | | | |
| Pilocarpine | RRT 0.97 = 0.07% | ND¹ | ND¹ | RRT 1.05 = 0.07% |
| Hydrochloride Related | RRT 1.29 = 0.28% | | | RRT 1.29 = 0.05% |
| Substances (% Area) | | | | |
| Pilocarpic Acid | 0.06% | 9.06% | 17.97% | 25.13% |
| Isopilocarpic Acid | ND¹ | 0.19% | 0.49% | 0.62% |
| Isopilocarpine | 0.06% | 3.33% | 6.84% | 10.98% |
| Pilocarpine | 0.47% | 12.58% | 25.30% | 36.85% |
| Hydrochloride Total | | | | |
| Related Substances | | | | |

TABLE 38
Stability data for Formulation 11
Storage Condition: 5° C. ± 3° C./Ambient RH

| | Time Point |
|---|---|
| Parameter | Initial |
| Visual | Clear solution, free of visible particulates |
| Appearance | |
| pH | 7.37 |
| Pilocarpine | 99.2% |
| Hydrochloride | |
| Content | |
| (HPLC Assay) | |
| Pilocarpine | RRT 1.05 = 0.20% |
| Hydrochloride | RRT 1.29 = 0.26% |
| Related | |
| Substances (% | |
| Area) | |
| Pilocarpic Acid | 0.64% |
| Isopilocarpic | ND¹ |
| Acid | |
| Isopilocarpine | 0.15% |
| Pilocarpine | 1.25% |
| Hydrochloride | |
| Total Related | |
| Substances | |

Storage Condition: 25° C. ± 2° C./60% RH ± 5% RH

| | Time Point | | | |
|---|---|---|---|---|
| Parameter | Initial | T = 1 Month | T = 3 Months | T = 6 Months |
| Visual | Clear solution, | Clear solution, | Clear solution, | Clear solution, |
| Appearance | free of visible | free of visible | free of visible | free of visible |
| | particulates | particulates | particulates | particulates |
| pH | 7.37 | 7.17 | 6.86 | 6.81 |
| Pilocarpine | 99.2% | 79.3% | 56.3% | 36.4% |
| Hydrochloride | | | | |
| Content | | | | |
| (HPLC Assay) | | | | |
| Pilocarpine | RRT 1.05 = 0.20% | RRT 1.05 = 0.17% | RRT 1.05 = 0.20% | RRT 1.05 = 0.09% |
| Hydrochloride | RRT 1.29 = 0.26% | | | |
| Related | | | | |
| Substances | | | | |
| (% Area) | | | | |
| Pilocarpic Acid | 0.64% | 16.89% | 33.22% | 47.83% |
| Isopilocarpic | ND¹ | 0.59% | 2.31% | 4.71% |
| Acid | | | | |
| Isopilocarpine | 0.15% | 4.29% | 7.34% | 9.75% |
| Pilocarpine | 1.25% | 21.94% | 43.07% | 62.39% |
| Hydrochloride | | | | |
| Total Related | | | | |
| Substances | | | | |

Storage Condition: 40° C. ± 2° C./75% RH ± 5% RH

| | Time Point | | | |
|---|---|---|---|---|
| Parameter | Initial | T = 1 Month | T = 3 Months | T = 6 Months |
| Visual | Clear solution, | Clear solution, | Clear solution, | |
| Appearance | free of visible | free of visible | free of visible | |
| | particulates | particulates | particulates | |
| pH | 7.37 | 6.82 | 6.63 | |
| Pilocarpine | 99.2% | 40.1% | 18.2% | |
| Hydrochloride | | | | |
| Content | | | | |
| (HPLC Assay) | | | | |
| Pilocarpine | RRT 1.05 = 0.20% | RRT 1.05 = 0.07% | RRT 1.05 = 0.05% | |
| Hydrochloride | RRT 1.29 = 0.26% | | | |
| Related | | | | |
| Substances | | | | |
| (% Area) | | | | |
| Pilocarpic Acid | 0.64% | 43.22% | 57.64% | |
| Isopilocarpic | ND¹ | 4.28% | 6.85% | |
| Acid | | | | |
| Isopilocarpine | 0.15% | 11.95% | 16.79% | |
| Pilocarpine | 1.25% | 59.52% | 81.33% | |
| Hydrochloride | | | | |
| Total Related | | | | |
| Substances | | | | |

TABLE 39
Stability data for Formulation 12
Storage Condition: 5° C. ± 3° C./Ambient RH

| | Time Point |
|---|---|
| Parameter | Initial |
| Visual Appearance | Clear solution, free of visible particulates |
| pH | 6.38 |
| Pilocarpine | 67.4% |
| Hydrochloride | |
| Content | |
| (HPLC Assay) | |
| Pilocarpine | RRT 1.05 = 0.06% |
| Hydrochloride | RRT 1.28 = 0.33% |
| Related | |
| Substances | |
| (% Area) | |
| Pilocarpic Acid | 0.13% |
| Isopilocarpic Acid | ND¹ |
| Isopilocarpine | 0.05% |
| Pilocarpine | 0.57% |
| Hydrochloride | |
| Total Related | |
| Substances | |

Storage Condition: 25° C. ± 2° C./60% RH ± 5% RH

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 6.38 | 6.24 | 6.13 | 5.98 |
| Pilocarpine | 67.4% | 96.9% | 91.0% | 81.9% |
| Hydrochloride | | | | |
| Content | | | | |
| (HPLC Assay) | | | | |
| Pilocarpine | RRT 1.05 = 0.06% | RRT 1.05 = 0.07% | RRT 1.05 = 0.13% | RRT 1.05 = 0.19% |
| Hydrochloride | RRT 1.28 = 0.33% | | | |
| Related | | | | |
| Substances | | | | |
| (% Area) | | | | |
| Pilocarpic Acid | 0.13% | 3.37% | 7.57% | 13.22% |
| Isopilocarpic Acid | ND¹ | ND¹ | 0.13% | 0.30% |
| Isopilocarpine | 0.05% | 1.03% | 2.19% | 3.80% |
| Pilocarpine | 0.57% | 4.47% | 10.02% | 17.51% |
| Hydrochloride | | | | |
| Total Related | | | | |
| Substances | | | | |

Storage Condition: 40° C. ± 2° C./75% RH ± 5% RH

| | Time Point | | | |
|---|---|---|---|---|
| Parameter | Initial | T = 1 Month | T = 3 Months | T = 6 Months |
| Visual Appearance | Clear solution, | Clear solution, | Clear solution, | Clear solution, |
| | free of visible | free of visible | free of visible | free of visible |
| | particulates | particulates | particulates | particulates |
| pH | 6.38 | 5.95 | 5.65 | 5.52 |
| Pilocarpine | 67.4% | 84.4% | 69.8% | 58.0% |
| Hydrochloride | | | | |
| Content | | | | |
| (HPLC Assay) | | | | |
| Pilocarpine | RRT 1.05 = 0.06% | ND¹ | RRT 1.05 = 0.06% | RRT 1.05 = 0.08% |
| Hydrochloride | RRT 1.28 = 0.33% | | | |
| Related | | | | |
| Substances | | | | |
| (% Area) | | | | |
| Pilocarpic Acid | 0.13% | 11.83% | 21.49% | 27.94% |
| Isopilocarpic Acid | ND1 | 0.30% | 0.67% | 0.77% |
| Isopilocarpine | 0.05% | 4.37% | 8.33% | 12.70% |
| Pilocarpine | 0.57% | 16.50% | 30.55% | 41.49% |
| Hydrochloride | | | | |
| Total Related | | | | |
| Substances | | | | |

### Example 10—Presbyopia Clinical Trial

The effects of the ophthalmic compositions as described in Example 1 are evaluated in subjects for treatment of presbyopia. 300 subjects both male and female between the ages of 40-55 are chosen to participate in the trial. Exclusion and inclusion criteria are listed in Table 40.

TABLE 40

| | |
|---|---|
| Inclusion Criteria | Subjective complaints of poor near vision that |
| | impact activities of daily living |
| Exclusion Criteria | History of cataract surgery, phakic intraocular |
| | lens surgery, corneal inlay surgery, radial |
| | keratotomy, or any intraocular surgery |
| | Use of any topical ophthalmic medications, |
| | including artificial tears other than the study |
| | medications during the study |
| | Use of temporary or permanent punctal plugs |
| | or history of punctal cautery in one or both |
| | eyes |
| | Corneal abnormalities (including keratoconus, |
| | corneal scar, Fuchs' endothelial dystrophy, |
| | guttata, or edema) in either eye that are likely |
| | to interfere with visual acuity |
| | Narrow iridocorneal angles (Shaffer grade ≤ 2 |
| | or lower on gonioscopy examination), history |
| | of angle-closure glaucoma, or previous |
| | iridotomy |
| | Diagnosis of any type of glaucoma or ocular |
| | hypertension |

The experimental group will administer one drop bilaterally of an ophthalmic composition as described herein once a day for 30 days. The placebo comparator group will administer one drop bilaterally of a vehicle control as described herein once a day for 30 days. Visual acuity will be measured.

While preferred embodiments of the present disclosure have been shown and described herein, such embodiments are provided by way of example only. Various alternatives to the embodiments described herein are optionally employed in practicing the disclosure. It is intended that the following claims define the scope of the disclosure and that methods and structures within the scope of these claims and their equivalents be covered thereby.

What is claimed is:

1. An ophthalmic composition for treating presbyopia comprising aceclidine or a pharmaceutically acceptable salt of aceclidine and deuterated water.

2. The ophthalmic composition of claim 1, wherein the aceclidine or the pharmaceutically acceptable salt of aceclidine is present in the ophthalmic composition at a concentration of about 0.25 wt % to about 2.0% wt %.

3. The ophthalmic composition of claim 1, wherein the aceclidine or the pharmaceutically acceptable salt of aceclidine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

4. The ophthalmic composition of any one of claims 1-3, wherein the ophthalmic composition further comprises pilocarpine or a pharmaceutically acceptable salt of pilocarpine.

5. The ophthalmic composition of claim 4, wherein the pilocarpine or the pharmaceutically acceptable salt of pilocarpine is present in the ophthalmic composition at a concentration of about 0.5 wt % to about 2.5 wt %, about 0.01 wt % to 0.45 wt %, or about 0.001 wt % to about 2 wt %.

6. The ophthalmic composition of claim 4, wherein the pilocarpine or the pharmaceutically acceptable salt of pilocarpine is present in the ophthalmic composition at a concentration of at least about 0.25 wt %, 0.5 wt %, or 1.0 wt %.

7. The ophthalmic composition of claim 4, wherein the pilocarpine or the pharmaceutically acceptable salt of pilocarpine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

8. The ophthalmic composition of any one of claims 1-3, wherein the ophthalmic composition further comprises tropicamide or a pharmaceutically acceptable salt of tropicamide.

9. The ophthalmic composition of claim 8, wherein the tropicamide or the pharmaceutically acceptable salt of tropicamide is present in the ophthalmic composition at a concentration about 0.010 wt % to about 0.1 wt % or about 0.025 wt % to about 0.1 wt %.

10. The ophthalmic composition of claim 8, wherein the tropicamide or the pharmaceutically acceptable salt of tropicamide is present in the ophthalmic composition at a concentration of at least about 0.02 wt %, 0.5 wt %, or 1.0 wt %.

11. The ophthalmic composition of claim 8, wherein the tropicamide or the pharmaceutically acceptable salt of tropicamide is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

12. The ophthalmic composition of any one of claims 1-3, wherein the ophthalmic composition further comprises pilocarpine or a pharmaceutically acceptable salt of pilocarpine and tropicamide or a pharmaceutically acceptable salt of tropicamide.

13. The ophthalmic composition of claim 12, wherein the pilocarpine or the pharmaceutically acceptable salt of pilocarpine is present in the ophthalmic composition at a concentration of about 0.5 wt % to about 2.5 wt %, about 0.01 wt % to 0.45 wt %, or about 0.001 wt % to about 2 wt %.

14. The ophthalmic composition of claim 12, wherein the pilocarpine or the pharmaceutically acceptable salt of pilocarpine is present in the ophthalmic composition at a concentration of at least about 0.25 wt %, 0.5 wt %, or 1.0 wt %.

15. The ophthalmic composition of claim 12, wherein the pilocarpine or the pharmaceutically acceptable salt of pilocarpine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

16. The ophthalmic composition of claim 12, wherein the tropicamide or the pharmaceutically acceptable salt of tropicamide is present in the ophthalmic composition at a concentration about 0.010 wt % to about 0.1 wt % or about 0.025 wt % to about 0.1 wt %.

17. The ophthalmic composition of claim 12, wherein the tropicamide or the pharmaceutically acceptable salt of tropicamide is present in the ophthalmic composition at a concentration of at least about 0.02 wt %, 0.5 wt %, or 1.0 wt %.

18. The ophthalmic composition of claim 12, wherein the tropicamide or the pharmaceutically acceptable salt of tropicamide is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

19. The ophthalmic composition of any one of claims 1-18, wherein the ophthalmic composition has a pH of about 4.2 to about 7.9.

20. The ophthalmic composition of any one of claims 1-18, wherein the ophthalmic composition has a pH of about 4.5 to about 7.5.

21. The ophthalmic composition of any one of claims 1-18, wherein the ophthalmic composition has a pH of about 5.5 to about 6.5.

22. The ophthalmic composition of any one of claims 1-18, wherein the ophthalmic composition has a pH of one of: less than about 7.3, less than about 7.2, less than about 7.1, less than about 7, less than about 6.8, less than about 6.5, less than about 6.4, less than about 6.3, less than about 6.2, less than about 6.1, less than about 6, less than about 5.9, less than about 5.8, less than about 5.2, less than about 4.8, or less than about 4.5 after an extended period of time under a storage condition.

23. The ophthalmic composition of any of claims 1-22, wherein the ophthalmic composition comprises one of: at least about 80%, at least about 85%, at least about 90%, at least about 93%, at least about 95%, at least about 97%, at least about 98%, or at least about 99% of the aceclidine or the pharmaceutically acceptable salt of aceclidine based on initial concentration after an extended period of time under a storage condition.

24. The ophthalmic composition of any of claim 4-7 or 12-15, wherein the ophthalmic composition comprises one of: at least about 80%, at least about 85%, at least about 90%, at least about 93%, at least about 95%, at least about 97%, at least about 98%, or at least about 99% of the pilocarpine or the pharmaceutically acceptable salt of pilocarpine based on initial concentration after an extended period of time under a storage condition.

25. The ophthalmic composition of any of claim 8-12 or 16-18, wherein the ophthalmic composition comprises one of: at least about 80%, at least about 85%, at least about 90%, at least about 93%, at least about 95%, at least about 97%, at least about 98%, or at least about 99% of the tropicamide or the pharmaceutically acceptable salt of tropicamide based on initial concentration after an extended period of time under a storage condition.

26. The ophthalmic composition of claim 1-25, wherein the ophthalmic composition further has a potency of one of: at least 80%, at least 85%, at least 90%, at least 93%, at least 95%, at least 97%, at least 98%, or at least 99% after an extended period of time under a storage condition.

27. The ophthalmic composition of any one of claims 22-26, wherein the extended period of time is one of: about 1 week, about 2 weeks, about 3 weeks, about 1 month, about 2 months, about 3 months, about 4 months, about 5 months, about 6 months, about 8 months, about 10 months, about 12 months, about 18 months, about 24 months, about 36 months, about 4 years, or about 5 years.

28. The ophthalmic composition of any one of claims 22-26, wherein the storage condition has a storage temperature of about 0° C. to about 30° C., 2° C. to about 10° C., or about 16° C. to about 26° C.

29. The ophthalmic composition of any of claims 1-28, wherein the ophthalmic composition further comprises an osmolarity adjusting agent.

30. The ophthalmic composition of claim 29, wherein the osmolarity adjusting agent is sodium chloride.

31. The ophthalmic composition of claim 30, wherein the sodium chloride is present in the ophthalmic composition at a concentration of one of: about 0.01 wt % to about 1.0 wt %, about 0.05 wt % to about 1.5 wt %, about 0.075 wt % to about 2.0 wt %, or about 0.1 wt % to about 3.0 wt %.

32. The ophthalmic composition of any of claims 1-31, wherein the ophthalmic composition further comprises a buffering agent.

33. The ophthalmic composition of claim 32, wherein the buffering agent is selected from borates, borate-polyol complexes, phosphate buffering agents, citrate buffering agents, acetate buffering agents, carbonate buffering agents, organic buffering agents, amino acid buffering agents, or combinations thereof.

34. The ophthalmic composition of any of claims 1-33, wherein the ophthalmic composition has a dose-to-dose aceclidine concentration variation of one of: less than 50%, less than 40%, less than 30%, less than 20%, less than 10%, or less than 5%.

35. The ophthalmic composition of claim 34, wherein the dose-to-dose aceclidine concentration variation is based on one of: 10 consecutive doses, 8 consecutive doses, 5 consecutive doses, 3 consecutive doses, or 2 consecutive doses.

36. The ophthalmic composition of any of claim 4-7 or 12-15, wherein the ophthalmic composition has a dose-to-dose pilocarpine concentration variation of one of: less than 50%, less than 40%, less than 30%, less than 20%, less than 10%, or less than 5%.

37. The ophthalmic composition of claim 36, wherein the dose-to-dose pilocarpine concentration variation is based on one of: 10 consecutive doses, 8 consecutive doses, 5 consecutive doses, 3 consecutive doses, or 2 consecutive doses.

38. The ophthalmic composition of any of claim 8-12 or 16-18, wherein the ophthalmic composition has a dose-to-dose tropicamide concentration variation of one of: less than 50%, less than 40%, less than 30%, less than 20%, less than 10%, or less than 5%.

39. The ophthalmic composition of claim 38, wherein the dose-to-dose tropicamide concentration variation is based on one of: 10 consecutive doses, 8 consecutive doses, 5 consecutive doses, 3 consecutive doses, or 2 consecutive doses.

40. The ophthalmic composition of any of claims 1-39, wherein the ophthalmic composition further comprises a pH adjusting agent.

41. The ophthalmic composition of claim 40, wherein the pH adjusting agent comprises DCI, HCl, NaOH, NaOD, CD₃COOD, C₆D₈O₇, CH₃COOH, C₆H₈O₇, or combinations thereof.

42. The ophthalmic composition of any of claims 1-41, wherein the ophthalmic composition comprises one of: less than 5% of water (H₂O), less than 4% of H₂O, less than 3% of H₂O, less than 2% of H₂O, less than 1% of H₂O, less than 0.5% of H₂O, less than 0.1% of H₂O, or 0% of H₂O.

43. The ophthalmic composition of any of claims 1-42, wherein the ophthalmic composition is not formulated as an injectable formulation.

44. The ophthalmic composition of any of claims 1-43, wherein the ophthalmic composition further comprises one or more sodium phosphate buffers.

45. The ophthalmic composition of claim 44, wherein a first sodium phosphate of the one or more sodium phosphate buffers is monosodium phosphate anhydrous.

46. The ophthalmic composition of claim 45, wherein the monosodium phosphate anhydrous is present in the ophthalmic composition at a concentration of about 0.004 wt % to about 0.20 wt %.

47. The ophthalmic composition of any of claims 44-46, wherein a second sodium phosphate buffer of the one or more sodium phosphate buffers is disodium phosphate anhydrous.

48. The ophthalmic composition of claim 47, wherein the disodium phosphate anhydrous is present in the ophthalmic composition at a concentration of about 0.050 wt % to about 2.0 wt %.

49. The ophthalmic composition of any one of claims 1-48, wherein the ophthalmic composition comprises a preservative.

50. The ophthalmic composition of claim 49, wherein the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof.

51. The ophthalmic composition of any one of claims 1-48, wherein the ophthalmic composition is free of a preservative selected from benzalkonium chloride, cetrimonium, sodium perborate, stabilized oxychloro complex, SofZia, polyquaternium-1, chlorobutanol, edetate disodium, polyhexamethylene biguanide, or combinations thereof.

52. The ophthalmic composition of any one of claims 1-48, wherein the ophthalmic composition is substantially free of a benzalkonium chloride preservative.

53. The ophthalmic composition of any one of claims 1-48, wherein the ophthalmic composition is substantially free of any preservative.

54. The ophthalmic composition of any of claims 1-53, wherein the ophthalmic composition is essentially free of citrate and acetate buffering agents.

55. The ophthalmic composition of any of claims 1-54, wherein the ophthalmic composition further comprises EDTA.

56. The ophthalmic composition of claim 55, wherein the EDTA is present in the ophthalmic composition at a concentration of 0.01 wt % to about 0.50 wt %.

57. The ophthalmic composition of any of claims 1-56, wherein the ophthalmic composition is essentially free of procaine and benactyzine, or pharmaceutically acceptable salts thereof.

58. The ophthalmic composition of any of claims 1-57, wherein the ophthalmic composition further comprises a tonicity adjusting agent.

59. The ophthalmic composition of claim 58, wherein the tonicity adjusting agent comprises a halide salt of a monovalent cation.

60. The ophthalmic composition of any of claims 1-59, wherein the ophthalmic composition further comprises an ophthalmically acceptable viscosity agent.

61. The ophthalmic composition of claim 60, wherein the ophthalmically acceptable viscosity agent comprises hydroxyethyl cellulose, hydroxypropyl cellulose, or hydroxypropylmethyl-cellulose (HPMC).

62. The ophthalmic composition of any of claims 1-61, wherein the ophthalmic composition further comprises 0.004 wt % to about 0.20 wt % citrate.

63. The ophthalmic composition of any of claims 1-62, wherein the ophthalmic composition is a storage-stabilized composition.

64. An ophthalmic composition comprising about 0.001 wt % to about 3 wt % pilocarpine or a pharmaceutically acceptable salt of pilocarpine and water, wherein the ophthalmic composition further comprises a buffering agent to provide a pH of about 4.2 to about 7.9.

65. The ophthalmic composition of claim 64, wherein the pilocarpine or the pharmaceutically acceptable salt of pilocarpine is present in the ophthalmic composition at a concentration of about 0.5 wt % to about 2.5 wt %, about 0.01 wt % to 0.45 wt %, or about 0.001 wt % to about 2 wt %.

66. The ophthalmic composition of claim 64, wherein the pilocarpine or the pharmaceutically acceptable salt of pilocarpine is present in the ophthalmic composition at a concentration of at least about 0.25 wt %, 0.5 wt %, or 1.0 wt %.

67. The ophthalmic composition of claim 64, wherein the pilocarpine or the pharmaceutically acceptable salt of pilocarpine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

68. The ophthalmic composition of any one of claims 64-67, wherein the ophthalmic composition further comprises aceclidine or a pharmaceutically acceptable salt of aceclidine.

69. The ophthalmic composition of claim 68, wherein the aceclidine or the pharmaceutically acceptable salt of aceclidine is present in the ophthalmic composition at a concentration of about 0.25 wt % to about 2.0% wt %.

70. The ophthalmic composition of claim 68, wherein the aceclidine or the pharmaceutically acceptable salt of aceclidine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

71. The ophthalmic composition of any one of claims 64-67, wherein the ophthalmic composition further comprises tropicamide or a pharmaceutically acceptable salt of tropicamide.

72. The ophthalmic composition of claim 71, wherein the tropicamide or the pharmaceutically acceptable salt of tropicamide is present in the ophthalmic composition at a concentration about 0.010 wt % to about 0.1 wt % or about 0.025 wt % to about 0.1 wt %.

73. The ophthalmic composition of claim 71, wherein the tropicamide or the pharmaceutically acceptable salt of tropicamide is present in the ophthalmic composition at a concentration of at least about 0.02 wt %, 0.5 wt %, or 1.0 wt %.

74. The ophthalmic composition of claim 71, wherein the tropicamide or the pharmaceutically acceptable salt of tropicamide is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

75. The ophthalmic composition of any one of claims 64-67, wherein the ophthalmic composition further comprises aceclidine or a pharmaceutically acceptable salt of aceclidine and tropicamide or a pharmaceutically acceptable salt of tropicamide.

76. The ophthalmic composition of claim 75, wherein the aceclidine or the pharmaceutically acceptable salt of aceclidine is present in the ophthalmic composition at a concentration of about 0.25 wt % to about 2.0% wt %.

77. The ophthalmic composition of claim 75, wherein the aceclidine or the pharmaceutically acceptable salt of aceclidine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

78. The ophthalmic composition of claim 75, wherein the tropicamide or the pharmaceutically acceptable salt of tropicamide is present in the ophthalmic composition at a concentration about 0.010 wt % to about 0.1 wt % or about 0.025 wt % to about 0.1 wt %.

79. The ophthalmic composition of claim 75, wherein the tropicamide or the pharmaceutically acceptable salt of tropicamide is present in the ophthalmic composition at a concentration of at least about 0.02 wt %, 0.5 wt %, or 1.0 wt %.

80. The ophthalmic composition of claim 75, wherein the tropicamide or the pharmaceutically acceptable salt of tropicamide is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

81. The ophthalmic composition of any one of claims 64-80, wherein the ophthalmic composition has a pH of about 4.8 to about 6.4.

82. The ophthalmic composition of any one of claims 64-81, wherein the ophthalmic composition comprises one of: at least about 80%, at least about 85%, at least about 90%, at least about 93%, at least about 95%, at least about 97%, at least about 98%, or at least about 99% of the pilocarpine or the pharmaceutically acceptable salt of pilocarpine based on initial concentration after an extended period of time under a storage condition.

83. The ophthalmic composition of any one of claim 68-70 or 75-77, wherein the ophthalmic composition comprises one of: at least about 80%, at least about 85%, at least about 90%, at least about 93%, at least about 95%, at least about 97%, at least about 98%, or at least about 99% of the aceclidine or the pharmaceutically acceptable salt of aceclidine based on initial concentration after an extended period of time under a storage condition.

84. The ophthalmic composition of any one of claim 71-75 or 78-80, wherein the ophthalmic composition comprises one of: at least about 80%, at least about 85%, at least about 90%, at least about 93%, at least about 95%, at least about 97%, at least about 98%, or at least about 99% of the tropicamide or the pharmaceutically acceptable salt of tropicamide based on initial concentration after an extended period of time under a storage condition.

85. The ophthalmic composition of any one of claims 64-84, wherein the ophthalmic composition further has a potency of one of: at least 80%, at least 85%, at least 90%, at least 93%, at least 95%, at least 97%, at least 98%, or at least 99% after an extended period of time under a storage condition.

86. The ophthalmic composition of any one of claims 82-85, wherein the extended period of time is one of: about 1 week, about 2 weeks, about 3 weeks, about 1 month, about 2 months, about 3 months, about 4 months, about 5 months, about 6 months, about 8 months, about 10 months, about 12 months, about 18 months, about 24 months, about 36 months, about 4 years, or about 5 years.

87. The ophthalmic composition of any one of claims 82-86, wherein the storage condition has a storage temperature of about 0° C. to about 30° C., 2° C. to about 10° C., or about 16° C. to about 26° C.

88. The ophthalmic composition of any one of claims 64-87, wherein the ophthalmic composition further comprises an osmolarity adjusting agent.

89. The ophthalmic composition of claim 88, wherein the osmolarity adjusting agent is sodium chloride.

90. The ophthalmic composition of claim 89, wherein the sodium chloride is present in the ophthalmic composition at a concentration of one of: about 0.01 wt % to about 1.0 wt %, about 0.05 wt % to about 1.5 wt %, about 0.075 wt % to about 2.0 wt %, or about 0.1 wt % to about 3.0 wt %.

91. The ophthalmic composition of any one of claims 64-90, wherein the ophthalmic composition further comprises a buffering agent.

92. The ophthalmic composition of claim 91, wherein the buffering agent is selected from borates, borate-polyol complexes, phosphate buffering agents, citrate buffering agents, acetate buffering agents, carbonate buffering agents, organic buffering agents, amino acid buffering agents, or combinations thereof.

93. The ophthalmic composition of any one of claims 64-92, wherein the ophthalmic composition has a dose-to-dose pilocarpine concentration variation of one of: less than 50%, less than 40%, less than 30%, less than 20%, less than 10%, or less than 5%.

94. The ophthalmic composition of claim 93, wherein the dose-to-dose pilocarpine concentration variation is based on one of: 10 consecutive doses, 8 consecutive doses, 5 consecutive doses, 3 consecutive doses, or 2 consecutive doses.

95. The ophthalmic composition of any of claim 68-70 or 75-77, wherein the ophthalmic composition has a dose-to-dose aceclidine concentration variation of one of: less than 50%, less than 40%, less than 30%, less than 20%, less than 10%, or less than 5%.

96. The ophthalmic composition of claim 95, wherein the dose-to-dose aceclidine concentration variation is based on one of: 10 consecutive doses, 8 consecutive doses, 5 consecutive doses, 3 consecutive doses, or 2 consecutive doses.

97. The ophthalmic composition of any of claim 71-75 or 78-80, wherein the ophthalmic composition has a dose-to-dose tropicamide concentration variation of one of: less than 50%, less than 40%, less than 30%, less than 20%, less than 10%, or less than 5%.

98. The ophthalmic composition of claim 97, wherein the dose-to-dose tropicamide concentration variation is based on one of: 10 consecutive doses, 8 consecutive doses, 5 consecutive doses, 3 consecutive doses, or 2 consecutive doses.

99. The ophthalmic composition of any one of claims 64-98, wherein the ophthalmic composition further comprises a pH adjusting agent.

100. The ophthalmic composition of claim 99, wherein the pH adjusting agent comprises DCI, HCl, NaOH, NaOD, CD₃COOD, C₆D₈O₇, CH₃COOH, C₆H₈O₇, or combinations thereof.

101. The ophthalmic composition of any one of claims 64-100, wherein the ophthalmic composition is not formulated as an injectable formulation.

102. The ophthalmic composition of any one of claims 64-101, wherein the ophthalmic composition further comprises one or more sodium phosphate buffers.

103. The ophthalmic composition of claim 102, wherein a first sodium phosphate of the one or more sodium phosphate buffers is monosodium phosphate anhydrous.

104. The ophthalmic composition of claim 103, wherein the monosodium phosphate anhydrous is present in the ophthalmic composition at a concentration of about 0.004 wt % to about 0.20 wt %.

105. The ophthalmic composition of any of claims 102-104, wherein a second sodium phosphate buffer of the one or more sodium phosphate buffers is disodium phosphate anhydrous.

106. The ophthalmic composition of claim 105, wherein the disodium phosphate anhydrous is present in the ophthalmic composition at a concentration of about 0.050 wt % to about 2.0 wt %.

107. The ophthalmic composition of any one of claims 64-106, wherein the ophthalmic composition comprises a preservative.

108. The ophthalmic composition of claim 107, wherein the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof.

109. The ophthalmic composition of any one of claims 64-106, wherein the ophthalmic composition is free of a preservative selected from benzalkonium chloride, cetrimonium, sodium perborate, stabilized oxychloro complex, SofZia, polyquaternium-1, chlorobutanol, edetate disodium, polyhexamethylene biguanide, or combinations thereof.

110. The ophthalmic composition of any one of claims 64-106, wherein the ophthalmic composition is substantially free of a benzalkonium chloride preservative.

111. The ophthalmic composition of any one of claims 64-106, wherein the ophthalmic composition is substantially free of any preservative.

112. The ophthalmic composition of any one of claims 64-111, wherein the ophthalmic composition is essentially free of citrate and acetate buffering agents.

113. The ophthalmic composition of any one of claims 64-112, wherein the ophthalmic composition further comprises EDTA.

114. The ophthalmic composition of claim 113, wherein the EDTA is present in the ophthalmic composition at a concentration of 0.01 wt % to about 0.50 wt %.

115. The ophthalmic composition of any one of claims 64-114, wherein the ophthalmic composition is essentially free of procaine and benactyzine, or pharmaceutically acceptable salts thereof.

116. The ophthalmic composition of any one of claims 64-115, wherein the ophthalmic composition further comprises a tonicity adjusting agent.

117. The ophthalmic composition of claim 116, wherein the tonicity adjusting agent comprises a halide salt of a monovalent cation.

118. The ophthalmic composition of any one of claims 64-117, wherein the ophthalmic composition further comprises an ophthalmically acceptable viscosity agent.

119. The ophthalmic composition of claim 118, wherein the ophthalmically acceptable viscosity agent comprises hydroxyethyl cellulose, hydroxypropyl cellulose, or hydroxypropylmethyl-cellulose (HPMC).

120. The ophthalmic composition of any of claims 64-119, wherein the ophthalmic composition further comprises 0.004 wt % to about 0.20 wt % citrate.

121. The ophthalmic composition of any one of claims 64-120, wherein the ophthalmic composition is a storage-stabilized composition.

122. An ophthalmic composition comprising about 0.010 wt % to about 0.1 wt % tropicamide or a pharmaceutically acceptable salt of tropicamide and water, wherein the ophthalmic composition further comprises a buffering agent to provide a pH of about 4.2 to about 7.9.

123. The ophthalmic composition of claim 122, wherein the tropicamide or the pharmaceutically acceptable salt of tropicamide is present in the ophthalmic composition at a concentration about 0.025 wt % to about 0.1 wt %.

124. The ophthalmic composition of claim 122, wherein the tropicamide or the pharmaceutically acceptable salt of tropicamide is present in the ophthalmic composition at a concentration of at least about 0.02 wt %.

125. The ophthalmic composition of any one of claims 122-123, wherein the ophthalmic composition has a pH of about 4.8 to about 6.4.

126. The ophthalmic composition of any one of claims 122-125, wherein the ophthalmic composition comprises one of: at least about 80%, at least about 85%, at least about 90%, at least about 93%, at least about 95%, at least about 97%, at least about 98%, or at least about 99% of tropicamide or the pharmaceutically acceptable salt of tropicamide based on initial concentration after an extended period of time under a storage condition.

127. The ophthalmic composition of any one of claims 122-126, wherein the ophthalmic composition further has a potency of one of: at least 80%, at least 85%, at least 90%, at least 93%, at least 95%, at least 97%, at least 98%, or at least 99% after an extended period of time under a storage condition.

128. The ophthalmic composition of any one of claims 126-127, wherein the extended period of time is one of: about 1 week, about 2 weeks, about 3 weeks, about 1 month, about 2 months, about 3 months, about 4 months, about 5 months, about 6 months, about 8 months, about 10 months, about 12 months, about 18 months, about 24 months, about 36 months, about 4 years, or about 5 years.

129. The ophthalmic composition of any one of claims 126-128, wherein the storage condition has a storage temperature of about 0° C. to about 30° C., 2° C. to about 10° C., or about 16° C. to about 26° C.

130. The ophthalmic composition of any one of claims 122-129, wherein the ophthalmic composition further comprises an osmolarity adjusting agent.

131. The ophthalmic composition of claim 130, wherein the osmolarity adjusting agent is sodium chloride.

132. The ophthalmic composition of claim 131, wherein the sodium chloride is present in the ophthalmic composition at a concentration of one of: about 0.01 wt % to about 1.0 wt %, about 0.05 wt % to about 1.5 wt %, about 0.075 wt % to about 2.0 wt %, or about 0.1 wt % to about 3.0 wt %.

133. The ophthalmic composition of any one of claims 122-132, wherein the ophthalmic composition further comprises a buffering agent.

134. The ophthalmic composition of claim 133, wherein the buffering agent is selected from borates, borate-polyol complexes, phosphate buffering agents, citrate buffering agents, acetate buffering agents, carbonate buffering agents, organic buffering agents, amino acid buffering agents, or combinations thereof.

135. The ophthalmic composition of any one of claims 122-134, wherein the ophthalmic composition has a dose-to-dose tropicamide concentration variation of one of: less than 50%, less than 40%, less than 30%, less than 20%, less than 10%, or less than 5%.

136. The ophthalmic composition of claim 135, wherein the dose-to-dose tropicamide concentration variation is based on one of: 10 consecutive doses, 8 consecutive doses, 5 consecutive doses, 3 consecutive doses, or 2 consecutive doses.

137. The ophthalmic composition of any one of claims 122-136, wherein the ophthalmic composition further comprises a pH adjusting agent.

138. The ophthalmic composition of claim 137, wherein the pH adjusting agent comprises DCl, HCl, NaOH, NaOD, CD₃COOD, C₆D₈O₇, CH₃COOH, C₆H₈O₇, or combinations thereof.

139. The ophthalmic composition of any one of claims 122-138, wherein the ophthalmic composition is not formulated as an injectable formulation.

140. The ophthalmic composition of any one of claims 122-139, wherein the ophthalmic composition further comprises one or more sodium phosphate buffers.

141. The ophthalmic composition of claim 140, wherein a first sodium phosphate of the one or more sodium phosphate buffers is monosodium phosphate anhydrous.

142. The ophthalmic composition of claim 141, wherein the monosodium phosphate anhydrous is present in the ophthalmic composition at a concentration of about 0.004 wt % to about 0.20 wt %.

143. The ophthalmic composition of any of claims 140-142, wherein a second sodium phosphate buffer of the one or more sodium phosphate buffers is disodium phosphate anhydrous.

144. The ophthalmic composition of claim 143, wherein the disodium phosphate anhydrous is present in the ophthalmic composition at a concentration of about 0.050 wt % to about 2.0 wt %.

145. The ophthalmic composition of any one of claims 122-144, wherein the ophthalmic composition comprises a preservative.

146. The ophthalmic composition of claim 145, wherein the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof.

147. The ophthalmic composition of any one of claims 122-144, wherein the ophthalmic composition is free of a preservative selected from benzalkonium chloride, cetrimonium, sodium perborate, stabilized oxychloro complex, SofZia, polyquaternium-1, chlorobutanol, edetate disodium, polyhexamethylene biguanide, or combinations thereof.

148. The ophthalmic composition of any one of claims 122-144, wherein the ophthalmic composition is substantially free of a benzalkonium chloride preservative.

149. The ophthalmic composition of any one of claims 122-144, wherein the ophthalmic composition is substantially free of any preservative.

150. The ophthalmic composition of any one of claims 122-149, wherein the ophthalmic composition is essentially free of citrate and acetate buffering agents.

151. The ophthalmic composition of any one of claims 122-150, wherein the ophthalmic composition further comprises EDTA.

152. The ophthalmic composition of claim 151, wherein the EDTA is present in the ophthalmic composition at a concentration of 0.01 wt % to about 0.50 wt %.

153. The ophthalmic composition of any one of claims 122-152, wherein the ophthalmic composition is essentially free of procaine and benactyzine, or pharmaceutically acceptable salts thereof.

154. The ophthalmic composition of any one of claims 122-153, wherein the ophthalmic composition further comprises a tonicity adjusting agent.

155. The ophthalmic composition of claim 154, wherein the tonicity adjusting agent comprises a halide salt of a monovalent cation.

156. The ophthalmic composition of any one of claims 122-155, wherein the ophthalmic composition further comprises an ophthalmically acceptable viscosity agent.

157. The ophthalmic composition of claim 156, wherein the ophthalmically acceptable viscosity agent comprises hydroxyethyl cellulose, hydroxypropyl cellulose, or hydroxypropylmethyl-cellulose (HPMC).

158. The ophthalmic composition of any of claims 122-157, wherein the ophthalmic composition further comprises 0.004 wt % to about 0.20 wt % citrate.

159. The ophthalmic composition of any one of claims 122-158, wherein the ophthalmic composition is a storage-stabilized composition.

160. An ophthalmic composition comprising about 0.25 wt % to about 2.0% wt % aceclidine or a pharmaceutically acceptable salt of aceclidine and water, wherein the ophthalmic composition further comprises a buffering agent to provide a pH of about 4.2 to about 7.9.

161. The ophthalmic composition of claim 160, wherein the ophthalmic composition has a pH of about 4.8 to about 6.4.

162. The ophthalmic composition of any one of claims 160-161, wherein the ophthalmic composition comprises one of: at least about 80%, at least about 85%, at least about 90%, at least about 93%, at least about 95%, at least about 97%, at least about 98%, or at least about 99% of aceclidine or the pharmaceutically acceptable salt of aceclidine based on initial concentration after an extended period of time under a storage condition.

163. The ophthalmic composition of any one of claims 160-162, wherein the ophthalmic composition further has a potency of one of: at least 80%, at least 85%, at least 90%, at least 93%, at least 95%, at least 97%, at least 98%, or at least 99% after an extended period of time under a storage condition.

164. The ophthalmic composition of any one of claims 162-163, wherein the extended period of time is one of: about 1 week, about 2 weeks, about 3 weeks, about 1 month, about 2 months, about 3 months, about 4 months, about 5 months, about 6 months, about 8 months, about 10 months, about 12 months, about 18 months, about 24 months, about 36 months, about 4 years, or about 5 years.

165. The ophthalmic composition of any one of claims 162-164, wherein the storage condition has a storage temperature of about 0° C. to about 30° C., 2° C. to about 10° C., or about 16° C. to about 26° C.

166. The ophthalmic composition of any one of claims 160-165, wherein the ophthalmic composition further comprises an osmolarity adjusting agent.

167. The ophthalmic composition of claim 166, wherein the osmolarity adjusting agent is sodium chloride.

168. The ophthalmic composition of claim 167, wherein the sodium chloride is present in the ophthalmic composition at a concentration of one of: about 0.01 wt % to about 1.0 wt %, about 0.05 wt % to about 1.5 wt %, about 0.075 wt % to about 2.0 wt %, or about 0.1 wt % to about 3.0 wt %.

169. The ophthalmic composition of any one of claims 160-168, wherein the ophthalmic composition further comprises a buffering agent.

170. The ophthalmic composition of claim 169, wherein the buffering agent is selected from borates, borate-polyol complexes, phosphate buffering agents, citrate buffering agents, acetate buffering agents, carbonate buffering agents, organic buffering agents, amino acid buffering agents, or combinations thereof.

171. The ophthalmic composition of any one of claims 160-170, wherein the ophthalmic composition has a dose-to-dose aceclidine concentration variation of one of: less than 50%, less than 40%, less than 30%, less than 20%, less than 10%, or less than 5%.

172. The ophthalmic composition of claim 171, wherein the dose-to-dose aceclidine concentration variation is based on one of: 10 consecutive doses, 8 consecutive doses, 5 consecutive doses, 3 consecutive doses, or 2 consecutive doses.

173. The ophthalmic composition of any one of claims 160-172, wherein the ophthalmic composition further comprises a pH adjusting agent.

174. The ophthalmic composition of claim 173, wherein the pH adjusting agent comprises DCl, HCl, NaOH, NaOD, CD₃COOD, C₆D₈O₇, CH₃COOH, C₆H₈O₇, or combinations thereof.

175. The ophthalmic composition of any one of claims 160-174, wherein the ophthalmic composition is not formulated as an injectable formulation.

176. The ophthalmic composition of any one of claims 160-175, wherein the ophthalmic composition further comprises one or more sodium phosphate buffers.

177. The ophthalmic composition of claim 176, wherein a first sodium phosphate of the one or more sodium phosphate buffers is monosodium phosphate anhydrous.

178. The ophthalmic composition of claim 177, wherein the monosodium phosphate anhydrous is present in the ophthalmic composition at a concentration of about 0.004 wt % to about 0.20 wt %.

179. The ophthalmic composition of any of claims 176-178, wherein a second sodium phosphate buffer of the one or more sodium phosphate buffers is disodium phosphate anhydrous.

180. The ophthalmic composition of claim 179, wherein the disodium phosphate anhydrous is present in the ophthalmic composition at a concentration of about 0.050 wt % to about 2.0 wt %.

181. The ophthalmic composition of any one of claims 160-180, wherein the ophthalmic composition comprises a preservative.

182. The ophthalmic composition of claim 181, wherein the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof.

183. The ophthalmic composition of any one of claims 160-180, wherein the ophthalmic composition is free of a preservative selected from benzalkonium chloride, cetrimonium, sodium perborate, stabilized oxychloro complex, SofZia, polyquaternium-1, chlorobutanol, edetate disodium, polyhexamethylene biguanide, or combinations thereof.

184. The ophthalmic composition of any one of claims 160-180, wherein the ophthalmic composition is substantially free of a benzalkonium chloride preservative.

185. The ophthalmic composition of any one of claims 160-180, wherein the ophthalmic composition is substantially free of any preservative.

186. The ophthalmic composition of any one of claims 160-185, wherein the ophthalmic composition is essentially free of citrate and acetate buffering agents.

187. The ophthalmic composition of any one of claims 160-186, wherein the ophthalmic composition further comprises EDTA.

188. The ophthalmic composition of claim 187, wherein the EDTA is present in the ophthalmic composition at a concentration of 0.01 wt % to about 0.50 wt %.

189. The ophthalmic composition of any one of claims 160-188, wherein the ophthalmic composition is essentially free of procaine and benactyzine, or pharmaceutically acceptable salts thereof.

190. The ophthalmic composition of any one of claims 160-189, wherein the ophthalmic composition further comprises a tonicity adjusting agent.

191. The ophthalmic composition of claim 190, wherein the tonicity adjusting agent comprises a halide salt of a monovalent cation.

192. The ophthalmic composition of any one of claims 160-191, wherein the ophthalmic composition further comprises an ophthalmically acceptable viscosity agent.

193. The ophthalmic composition of claim 192, wherein the ophthalmically acceptable viscosity agent comprises hydroxyethyl cellulose, hydroxypropyl cellulose, or hydroxypropylmethyl-cellulose (HPMC).

194. The ophthalmic composition of any of claims 160-193, wherein the ophthalmic composition further comprises 0.004 wt % to about 0.20 wt % citrate.

195. The ophthalmic composition of any one of claims 160-194, wherein the ophthalmic composition is a storage-stabilized composition.

196. An ophthalmic composition for treating presbyopia comprising an ophthalmic agent and deuterated water.

197. The ophthalmic composition of claim 196, wherein the ophthalmic agent is pilocarpine or a pharmaceutically acceptable salt of pilocarpine, aceclidine or a pharmaceutically acceptable salt of aceclidine, tropicamide or a pharmaceutically acceptable salt of tropicamide, or combinations thereof.

198. The ophthalmic composition of claim 197, wherein the aceclidine or the pharmaceutically acceptable salt of aceclidine is present in the ophthalmic composition at a concentration of about 0.25 wt % to about 2.0% wt %.

199. The ophthalmic composition of claim 197, wherein the aceclidine or the pharmaceutically acceptable salt of aceclidine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

200. The ophthalmic composition of claim 197, wherein the pilocarpine or the pharmaceutically acceptable salt of pilocarpine is present in the ophthalmic composition at a concentration of about 0.5 wt % to about 2.5 wt %, about 0.01 wt % to 0.45 wt %, or about 0.001 wt % to about 2 wt %.

201. The ophthalmic composition of claim 197, wherein the pilocarpine or the pharmaceutically acceptable salt of pilocarpine is present in the ophthalmic composition at a concentration of at least about 0.25 wt %, 0.5 wt %, or 1.0 wt %.

202. The ophthalmic composition of claim 197, wherein the pilocarpine or the pharmaceutically acceptable salt of pilocarpine is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

203. The ophthalmic composition of claim 197, wherein the tropicamide or the pharmaceutically acceptable salt of tropicamide is present in the ophthalmic composition at a concentration about 0.010 wt % to about 0.1 wt % or about 0.025 wt % to about 0.1 wt %.

204. The ophthalmic composition of claim 197, wherein the tropicamide or the pharmaceutically acceptable salt of tropicamide is present in the ophthalmic composition at a concentration of at least about 0.02 wt %, 0.5 wt %, or 1.0 wt %.

205. The ophthalmic composition of claim 197, wherein the tropicamide or the pharmaceutically acceptable salt of tropicamide is present in the ophthalmic composition at a concentration of one of: about 0.001 wt % to about 0.5 wt %, about 0.001 wt % to about 0.40 wt %, about 0.001 wt % to about 0.30 wt %, about 0.001 wt % to about 0.20 wt %, about 0.001 wt % to about 0.10 wt %, about 0.001 wt % to about 0.09 wt %, about 0.001 wt % to about 0.08 wt %, about 0.001 wt % to about 0.07 wt %, about 0.001 wt % to about 0.06 wt %, about 0.001 wt % to about 0.05 wt %, about 0.001 wt % to about 0.04 wt %, about 0.001 wt % to about 0.03 wt %, about 0.001 wt % to about 0.025 wt %, about 0.001 wt % to about 0.02 wt %, about 0.001 wt % to about 0.01 wt %, about 0.001 wt % to about 0.008 wt %, or about 0.001 wt % to about 0.005 wt %.

206. The ophthalmic composition of claim 196, wherein the ophthalmic agent is atropine, atropine sulfate, noratropine, atropine-N-oxide, tropine, tropic acid, atropine methonitrate, diphenhydramine, dimenhydrinate, dicyclomine, flavoxate, oxybutynin, tiotropium, hyoscine, scopolamine (L-hyoscine), hydroxyzine, ipratropium, tropicamide, cyclopentolate, pirenzepine, homatropine, solifenacin, darifenacin, benzatropine, mebeverine, procyclidine, aclidinium bromide, trihexyphenidyl/benzhexol, tolterodine, anisodamine, or combinations thereof.

207. The ophthalmic composition of claim 196, wherein the ophthalmic agent is aflibercept, ranibizumab, pegaptanib, cyclopentolate, phenylephrine, homatropine, scopolamine, cyclopentolate/phenylephrine, phenylephrine/scopolamine, tropicamide, ketorolac/phenylephrine, hydroxyamphetamine/tropicamide, cysteamine, ocriplasmin, mitomycin, dapiprazole, lidocaine, proparacaine, tetracaine, benoxinate, azithromycin, bacitracin, besifloxacin, boric acid, chloramphenicol, ciprofloxacin, erythromycin, ganciclovir, gatifloxacin, gentamicin, idoxuridine, levofloxacin, moxifloxacin, natamycin, norfloxacin, ofloxacin, bacitracin/polymyxin b, tobramycin, polymyxin b/trimethoprim, povidone iodine, trifluridine, gramicidin/neomycin/polymyxin b, sulfacetamide sodium, sulfisoxazole, bacitracin/neomycin/polymyxin b, oxytetracycline/polymyxin b, phenylephrine/sulfacetamide sodium, vidarabine, bromfenac, nepafenac, ketorolac, cyclosporine, flurbiprofen, suprofen, diclofenac, alcaftadine, azelastine, bepotastine, cromolyn, emedastine, epinastine, ketotifen, levocabastine, lodoxamide, nedocromil, naphazoline, naphazoline/pheniramine, naphazoline/zinc sulfate, olopatadine, oxymetazoline, pemirolast, phenylephrine/zinc sulfate, tetrahydrozoline, tetrahydrozoline/zinc sulfate, fluorescein, fluorescein/proparacaine, benoxinate/fluorescein, indocyanine green, trypan blue, acetylcholine, apraclonidine, betaxolol, bimatoprost, brimonidine, brinzolamide, brimonidine/brinzolamide, carbachol, carteolol, demecarium bromide, dipivefrin, dorzolamide, dorzolamide/timolol, echothiophate iodide, epinephrine, epinephrine/pilocarpine, latanoprost, levobunolol, levobetaxolol, metipranolol, physostigmine, pilocarpine, tafluprost, timolol, travoprost, unoprostone, artificial tear, dexamethasone, difluprednate, fluocinolone, fluorometholone, loteprednol, medrysone, prednisolone, rimexolone, triamcinolone, fluorometholone/sulfacetamide sodium, dexamethasone/neomycin, dexamethasone/tobramycin, dexamethasone/neomycin/polymyxin b, loteprednol/tobramycin, prednisolone/sulfacetamide sodium, bacitracin/hydrocortisone/neomycin/polymyxin b, hydrocortisone/neomycin/polymyxin b, chloramphenicol/hydrocortisone/polymyxin b, neomycin/polymyxin b/prednisolone, gentamicin/prednisolone, ketorolac/phenylephrine, diphenhydramine, dimenhydrinate, dicyclomine, flavoxate, oxybutynin, tiotropium, hyoscine, scopolamine (L-hyoscine), hydroxyzine, ipratropium, pirenzepine, solifenacin, darifenacin, benzatropine, mebeverine, procyclidine, aclidinium bromide, trihexyphenidyl/benzhexol, tolterodine, aceclidine, anisodamine, or combinations thereof.

208. The ophthalmic composition of claim 196, wherein the ophthalmic agent is a miotic agent.

209. The ophthalmic composition of claim 208, wherein the miotic agent is dapiprazole, thymoxamine, brimonidine, nicotine, apraclonidin, phentolamine, pharmaceutically acceptable salts thereof, or combinations thereof.

210. The ophthalmic composition of any one of claims 196-209, wherein the ophthalmic composition has a pH of about 4.2 to about 7.9.

211. The ophthalmic composition of any one of claims 196-209, wherein the ophthalmic composition has a pH of about 4.5 to about 7.5.

212. The ophthalmic composition of any one of claims 196-209, wherein the ophthalmic composition has a pH of about 5.5 to about 6.5.

213. The ophthalmic composition of any one of claims 196-209, wherein the ophthalmic composition has a pH of one of: less than about 7.3, less than about 7.2, less than about 7.1, less than about 7, less than about 6.8, less than about 6.5, less than about 6.4, less than about 6.3, less than about 6.2, less than about 6.1, less than about 6, less than about 5.9, less than about 5.8, less than about 5.2, less than about 4.8, or less than about 4.5 after an extended period of time under a storage condition.

214. The ophthalmic composition of claim 197, wherein the ophthalmic composition comprises one of: at least about 80%, at least about 85%, at least about 90%, at least about 93%, at least about 95%, at least about 97%, at least about 98%, or at least about 99% of the pilocarpine or the pharmaceutically acceptable salt of pilocarpine based on initial concentration after an extended period of time under a storage condition.

215. The ophthalmic composition of claim 197, wherein the ophthalmic composition comprises one of: at least about 80%, at least about 85%, at least about 90%, at least about 93%, at least about 95%, at least about 97%, at least about 98%, or at least about 99% of the aceclidine or the pharmaceutically acceptable salt of aceclidine based on initial concentration after an extended period of time under a storage condition.

216. The ophthalmic composition of claim 197, wherein the ophthalmic composition comprises one of: at least about 80%, at least about 85%, wherein the ophthalmic composition comprises one of: at least about 80%, at least about 85%, at least about 90%, at least about 93%, at least about 95%, at least about 97%, at least about 98%, or at least about 99% of the tropicamide or the pharmaceutically acceptable salt of tropicamide based on initial concentration after an extended period of time under a storage condition.

217. The ophthalmic composition of any one of claims 196-216, wherein the ophthalmic composition further has a potency of one of: at least 80%, at least 85%, at least 90%, at least 93%, at least 95%, at least 97%, at least 98%, or at least 99% after an extended period of time under a storage condition.

218. The ophthalmic composition of any one of claims 213-217, wherein the extended period of time is one of: about 1 week, about 2 weeks, about 3 weeks, about 1 month, about 2 months, about 3 months, about 4 months, about 5 months, about 6 months, about 8 months, about 10 months, about 12 months, about 18 months, about 24 months, about 36 months, about 4 years, or about 5 years.

219. The ophthalmic composition of any one of claims 213-218, wherein the storage condition has a storage temperature of about 0° C. to about 30° C., 2° C. to about 10° C., or about 16° C. to about 26° C.

220. The ophthalmic composition of any of claims 196-219, wherein the ophthalmic composition further comprises an osmolarity adjusting agent.

221. The ophthalmic composition of claim 220, wherein the osmolarity adjusting agent is sodium chloride.

222. The ophthalmic composition of claim 221, wherein the sodium chloride is present in the ophthalmic composition at a concentration of one of: about 0.01 wt % to about 1.0 wt %, about 0.05 wt % to about 1.5 wt %, about 0.075 wt % to about 2.0 wt %, or about 0.1 wt % to about 3.0 wt %.

223. The ophthalmic composition of any of claims 196-222, wherein the ophthalmic composition further comprises a buffering agent.

224. The ophthalmic composition of claim 223, wherein the buffering agent is selected from borates, borate-polyol complexes, phosphate buffering agents, citrate buffering agents, acetate buffering agents, carbonate buffering agents, organic buffering agents, amino acid buffering agents, or combinations thereof.

225. The ophthalmic composition of any of claim 197, wherein the ophthalmic composition has a dose-to-dose aceclidine concentration variation of one of: less than 50%, less than 40%, less than 30%, less than 20%, less than 10%, or less than 5%.

226. The ophthalmic composition of claim 225, wherein the dose-to-dose aceclidine concentration variation is based on one of: 10 consecutive doses, 8 consecutive doses, 5 consecutive doses, 3 consecutive doses, or 2 consecutive doses.

227. The ophthalmic composition of claim 197, wherein the ophthalmic composition has a dose-to-dose pilocarpine concentration variation of one of: less than 50%, less than 40%, less than 30%, less than 20%, less than 10%, or less than 5%.

228. The ophthalmic composition of claim 227, wherein the dose-to-dose pilocarpine concentration variation is based on one of: 10 consecutive doses, 8 consecutive doses, 5 consecutive doses, 3 consecutive doses, or 2 consecutive doses.

229. The ophthalmic composition of claim 197, wherein the ophthalmic composition has a dose-to-dose tropicamide concentration variation of one of: less than 50%, less than 40%, less than 30%, less than 20%, less than 10%, or less than 5%.

230. The ophthalmic composition of claim 229, wherein the dose-to-dose tropicamide concentration variation is based on one of: 10 consecutive doses, 8 consecutive doses, 5 consecutive doses, 3 consecutive doses, or 2 consecutive doses.

231. The ophthalmic composition of any of claims 196-229, wherein the ophthalmic composition further comprises a pH adjusting agent.

232. The ophthalmic composition of claim 231, wherein the pH adjusting agent comprises DCl, HCl, NaOH, NaOD, CD₃COOD, C₆D₈O₇, CH₃COOH, C₆H₈O₇, or combinations thereof.

233. The ophthalmic composition of any of claims 196-232, wherein the ophthalmic composition comprises one of: less than 5% of water (H₂O), less than 4% of H₂O, less than 3% of H₂O, less than 2% of H₂O, less than 1% of H₂O, less than 0.5% of H₂O, less than 0.1% of H₂O, or 0% of H₂O.

234. The ophthalmic composition of any of claims 196-233, wherein the ophthalmic composition is not formulated as an injectable formulation.

235. The ophthalmic composition of any of claims 196-234, wherein the ophthalmic composition further comprises one or more sodium phosphate buffers.

236. The ophthalmic composition of claim 235, wherein a first sodium phosphate of the one or more sodium phosphate buffers is monosodium phosphate anhydrous.

237. The ophthalmic composition of claim 236, wherein the monosodium phosphate anhydrous is present in the ophthalmic composition at a concentration of about 0.004 wt % to about 0.20 wt %.

238. The ophthalmic composition of any of claims 235-237, wherein a second sodium phosphate buffer of the one or more sodium phosphate buffers is disodium phosphate anhydrous.

239. The ophthalmic composition of claim 238, wherein the disodium phosphate anhydrous is present in the ophthalmic composition at a concentration of about 0.050 wt % to about 2.0 wt %.

240. The ophthalmic composition of any one of claims 196-239, wherein the ophthalmic composition comprises a preservative.

241. The ophthalmic composition of claim 240, wherein the preservative is selected from benzalkonium chloride, cetrimonium, sodium perborate, stabilized oxychloro complex, SofZia, polyquaternium-1, chlorobutanol, edetate disodium, polyhexamethylene biguanide, or combinations thereof.

242. The ophthalmic composition of any one of claims 196-239, wherein the ophthalmic composition is free of a preservative selected from benzalkonium chloride, cetrimonium, sodium perborate, stabilized oxychloro complex, SofZia, polyquaternium-1, chlorobutanol, edetate disodium, polyhexamethylene biguanide, or combinations thereof.

243. The ophthalmic composition of any one of claims 196-239, wherein the ophthalmic composition is substantially free of a benzalkonium chloride preservative.

244. The ophthalmic composition of any one of claims 196-239, wherein the ophthalmic composition is substantially free of any preservative.

245. The ophthalmic composition of any of claims 196-244, wherein the ophthalmic composition is essentially free of citrate and acetate buffering agents.

246. The ophthalmic composition of any of claims 196-245, wherein the ophthalmic composition further comprises EDTA.

247. The ophthalmic composition of claim 246, wherein the EDTA is present in the ophthalmic composition at a concentration of 0.01 wt % to about 0.50 wt %.

248. The ophthalmic composition of any of claims 196-247, wherein the ophthalmic composition is essentially free of procaine and benactyzine, or pharmaceutically acceptable salts thereof.

249. The ophthalmic composition of any of claims 196-248, wherein the ophthalmic composition further comprises a tonicity adjusting agent.

250. The ophthalmic composition of claim 249, wherein the tonicity adjusting agent comprises a halide salt of a monovalent cation.

251. The ophthalmic composition of any of claims 196-250 wherein the ophthalmic composition further comprises an ophthalmically acceptable viscosity agent.

252. The ophthalmic composition of claim 251, wherein the ophthalmically acceptable viscosity agent comprises hydroxyethyl cellulose, hydroxypropyl cellulose, or hydroxypropylmethyl-cellulose (HPMC).

253. The ophthalmic composition of any of claims 196-252, wherein the ophthalmic composition further comprises 0.004 wt % to about 0.20 wt % citrate.

254. The ophthalmic composition of any of claims 196-253, wherein the ophthalmic composition is a storage-stabilized composition.

255. The ophthalmic composition of any of claims 196-253, wherein deuterated water is replaced with water.
